SEQUENCE LISTING (119 sequences)

SEQ ID NO: 1 - DNA, 1502 bp, Arabidopsis thaliana
  source 1..1502: mol_type = genomic DNA
agattcgttttatttttcaatcgtttcaatttctaaaattctcctctctcaacgcaatcaccaaatccaaatttagggtttcttttctctttctgcgatcgatctctgaaaactcataactgttgtttaatcgtgaagatggcatcaaacggatctttctctgctcagagaaacgcaaacgctagaacaacgatgaagagaagaagcgacaatcgcggttacggtggcggaatcggaggttatcaagaggagacgaaccgttacgcgccgccgcaaaaacgtttcagatcacaagcgcaacagcaattccgatctggtcataatcctctgtatcatcatcatggtagtaataataataacgtgtctagggtttcttctcaatcatataacaattgcggtgttgatgtgattgcttctaattcaagttttgcattacgtaacaatgattctaacaccaacaattatcagaaaccctttgttgctggttatggaaaccctaatccccaaattgttcctcttccattaccttatcgtaagcttgatgacaatctctctttagattcgttacctgattgggttccgaattcaagaaccctaactccgaattatcctgttaggtcatcgaattttgtaccaaacactcctgtttttacgaatgttcaaaatccgatgaatcattcgaacatggtttcagttgtgtctcaatctatgcatcaaccaattgttttgagcaaagaattgactgatttgcttagtcttcttaacaatgagaaagagaagaaaactttagaagcaagtaatagtgattctttgcctgttggattaagctttgataatccatcgtcacttaatgttcgacatgaatctgtgattaagtctctgtattctgatatgccgagacaatgttcatcgtgtgggttgagattcaagtgtcaagaagagcatagtaaacacatggattggcatgtaaggaagaacaggtcggtgaaaaccactacaagactgggtcagcaaccgaagaaatcgaggggatggttagcaagtgcatctttatggctttgtgcagcgacgggaggagagacggtagaagtagcatcttttggaggagaaatgcagaagaagaaaggaaaggacgaagaaccaaaacaactcatggtgcctgcggatgaagatcagaagaattgcgcattatgcgtagagccgtttgaagagttctttagccatgaagatgatgattggatgtataaagatgcagtctacttaaccaagaatggtcgtatagttcatgttaagtgtatgcctgaaccccgacctgctaaggatttgagagagccttcgagggtaatgtctgttacagttccatcggttgcgaaagctatagcctgctgattgagatttttaagatctctgttttagtcattcgtttttttaagtaatgtactttacttgaacaagttgtaacacaaatacagttctatcaattgaatctcctattttactct

SEQ ID NO: 2 - DNA, 1251 bp, Arabidopsis thaliana
  source 1..1251: mol_type = other DNA
atggcatcaaacggatctttctctgctcagagaaacgcaaacgctagaacaacgatgaagagaagaagcgacaatcgcggttacggtggcggaatcggaggttatcaagaggagacgaaccgttacgcgccgccgcaaaaacgtttcagatcacaagcgcaacagcaattccgatctggtcataatcctctgtatcatcatcatggtagtaataataataacgtgtctagggtttcttctcaatcatataacaattgcggtgttgatgtgattgcttctaattcaagttttgcattacgtaacaatgattctaacaccaacaattatcagaaaccctttgttgctggttatggaaaccctaatccccaaattgttcctcttccattaccttatcgtaagcttgatgacaatctctctttagattcgttacctgattgggttccgaattcaagaaccctaactccgaattatcctgttaggtcatcgaattttgtaccaaacactcctgtttttacgaatgttcaaaatccgatgaatcattcgaacatggtttcagttgtgtctcaatctatgcatcaaccaattgttttgagcaaagaattgactgatttgcttagtcttcttaacaatgagaaagagaagaaaactttagaagcaagtaatagtgattctttgcctgttggattaagctttgataatccatcgtcacttaatgttcgacatgaatctgtgattaagtctctgtattctgatatgccgagacaatgttcatcgtgtgggttgagattcaagtgtcaagaagagcatagtaaacacatggattggcatgtaaggaagaacaggtcggtgaaaaccactacaagactgggtcagcaaccgaagaaatcgaggggatggttagcaagtgcatctttatggctttgtgcagcgacgggaggagagacggtagaagtagcatcttttggaggagaaatgcagaagaagaaaggaaaggacgaagaaccaaaacaactcatggtgcctgcggatgaagatcagaagaattgcgcattatgcgtagagccgtttgaagagttctttagccatgaagatgatgattggatgtataaagatgcagtctacttaaccaagaatggtcgtatagttcatgttaagtgtatgcctgaaccccgacctgctaaggatttgagagagccttcgagggtaatgtctgttacagttccatcggttgcgaaagctatagcctgctga

SEQ ID NO: 3 - protein, 416 aa, Arabidopsis thaliana
MASNGSFSAQRNANARTTMKRRSDNRGYGGGIGGYQEETNRYAPPQKRFRSQAQQQFRSGHNPLYHHHGSNNNNVSRVSSQSYNNCGVDVIASNSSFALRNNDSNTNNYQKPFVAGYGNPNPQIVPLPLPYRKLDDNLSLDSLPDWVPNSRTLTPNYPVRSSNFVPNTPVFTNVQNPMNHSNMVSVVSQSMHQPIVLSKELTDLLSLLNNEKEKKTLEASNSDSLPVGLSFDNPSSLNVRHESVIKSLYSDMPRQCSSCGLRFKCQEEHSKHMDWHVRKNRSVKTTTRLGQQPKKSRGWLASASLWLCAATGGETVEVASFGGEMQKKKGKDEEPKQLMVPADEDQKNCALCVEPFEEFFSHEDDDWMYKDAVYLTKNGRIVHVKCMPEPRPAKDLREPSRVMSVTVPSVAKAIAC

SEQ ID NO: 4 - DNA, 4943 bp, Arabidopsis thaliana
  source 1..4943: mol_type = genomic DNA
taaaatccccaattcacaaccctaattgatcggagattgtccaaactctctgacatttttgccgaattcgaagcattccttctatcttctccgtcttcttcgcagaatcgcgttatgcatctcaatttttagattgtaacttagggttactgattgttgtaatgctctgaattcccctctaacgaggatttgttgtcactgcaatcgatttttgccgattaggtttaatttagggcattatttgcgtacctcttgtattgtatagttctttggaatggagaatcctcgtagaccgtttgatagatccagagatccaggtccgatgaagaaacccaggctcagcgaggaatcaatccggcctgttaattccaacgcgcggcagtttttgtcgcagagaacgcttggtacggctacggcggtgacagtgccacctgcttcgtcaaggtttcgagttagtggtagagaaactgagagtagtattgtcagtgatccgagccgagaggcttaccagccacagccggttcatccacattatgagcttgtgaatcagtacaagtctgcactggctgagctcactttcaactcgaagccgataataacgaaccttacaataattgctggagagaatgttcatgctgctaaagccgtcgtaaccgccatttgcaacaacatcctcgaggtgaacacccagttttcatgcccactgttttgttgttataatccgtgctttagttctcaaattcttgtttgagtaataataattgtacgatataaggggaataaggtaaaaggtggtggttgagatttgtgttatctgtatacgaggttcaatgagaaattgagttttttgaatcaaccctagtatccttttctgggatctatgtgtttttatatttcttcatcaagtcgcaaatcatttctttgaaacaaagttcatagtactgacacaatgttgttaacaatctaaaaaatagctgttgcccatgcaaccattttgttcatgttcatatatcaagaatgcatatatcagctcatcatgtgattgtactgagttttggttatatgagtgtgtagtctctttatcagcgttagagttttttgtatctcactttaccatttagcaagggctttcataagctctgtttttattgaggttggattttctgttggcggtttctgcttaccgcatgctctccacaaagccaagtcaacttttattcagtctttgggtagcaaggaagcactcaatagaaggagcctgatccatcaagtctgtgtggagagtgaagacaagaattgctattgcagtgttgtttacatcgtctaatggttatctgtgtctattgttggatttctagttgtcagaatatgtgttagtattagcttgaatgtcatcttggtctgtgcttttgattctactttaagttattgatttctttcattacctttagaggctcatgtggaaatctcgatgttactctctatgagatcaaattggccttttttcttctgggttatctgatttgaagaatatcatgcaggtaccaagtgatcagaagctgccaacactgtacctgttagacagcattgtgaagaacatcggaagggattacattaagtactttggtgccaggctgcctgaggtttggtgctttgttgatagtcatggttatagaatgtttttttcctagcaaattcatctctgatcaccctctttttacttctctgaaggtttttgttaaggcttacaggcaagttgaccctcctatgcattctaacatgcggcatctttttggaacctggaaaggagtctttcatccgcaaacacttcagcttattgagaaagaacttggctttaatgctaaaagtgatggttcagcagctgttgtatccacagccagagctgagccgcaatctcagcgcccaccacatagcatccatgtgaatcccaagtatctagaaaggcagcgccttcagcagtcgggacgggtaagcaatgaccaaagtaaacttgcagccactgaaagttattagtgatgatacaccctaaccttcagctctactagagatttcgctgttctaagtgttcataaatgtaaccttcaggagtactaaacatttctctgttctaagtgttcacaaatgtagggaacatttctttttcggtttagatagcaatcaatttctttcaaagggtcctgggtttctggatataggtagaggctacttatcgactttattaaaatttccctgagtatttgtcggttttctttgcagacaaaaggaatggttactgatgtcccagagaccgcacccaatttgactagggattcagatagactggagagagtttcaagcatagcatctggaggctcgtgggtaggtccagctaaagtaaatgtaagatgatttttccttggcatggttacagtttttttacctaagtgctttccactgattgtgtgtcgaattcagaacattcggcgacctcaaagagatttacttagtgagcccctctacgagaaagatatcgagtcaatagcaggggaatatgattatgcttctgatctaccacataactctagatcggtgattaagaatgttggttcaaggattactgatgacggatgtgagaaacaatggtacggagctacgaacagggatcctgatctgatttctgaccaaagggatgggttacattccaaaagcagaacttccaactatgctacggctagagtagaaaatttggaatcaagtggtcccagtagaaatataggtgtgccttatgatagttggaaaaactctgaggaagaagagtttatgtgggatatgcactcaaggttatccgaaactgatgtagccactatcaacccaaagaacgagcttcatgcaccagatgaatcagagagactggtaagctatgatagtaatttgcattcctttttattgtgatgtatctcattttaatttgttagcttttaaccatctcccatcttgtaggaatctgaaaaccacctcttaaaacgaccaaggttttcagctctcgacccaaggtttgacccagcgaattcaacaaattcatattccagcgagcaaaaagatccaagtagtattgggcactgggcgttttcttctaccaatgcaacctcgactgcaactagaaagggaattcagcctcaaccaagggttgcaagctctggaattttgccaagctcaggatctgggtcagatagacaatcgcctttacatgattcaacttctaaacagaatgtgaccaagcaagatgttagaagagctcactcactcccccaacgtgatcctagggcttcccggtttccagcaaaacaaaatgttcccagggatgactctgtacggctcccatcttcaagtagtcagtttaaaaacacaaatatgcgtgaactaccagtggaaatctttgactctaagtcagcagctgaaaatgccccaggccttactcttgcctcagaagctacaggacaacccaacatgagtgatttgctagaagctgtcatgaagagtggaatcctttctaataattcaacatgtggtgcgatcaaggaggaaagtcatgatgaggtcaacccgggggcactaaccctgccagcagcctctaagcctaagactttgccaatttcattggctactgacaatttattagctcgtttaaaagttgaacaatcctccgcaccacttgtctcatgtgcagcgtctcttactggcatcacctcagtacaaacgtcaaaagagaaaagtaaggcctcagatcctctttcatgcctcttaagctctttagtttcgaagggcttgatatctgcttcgaagacagagctgccatcggctccttccataacgcaggaacacagtcctgatcattccaccaatagctctatgtctgtatctgtggttccagcagatgctcagccatctgttttggtgaaggggccatctactgcccctaaggtaaaagggttagctgcaccatctgaaacatcaaaatctgaacccaaggacctcataggattgaagtttagggcggacaaaatccgtgaacttcacccttcggtcattagtagtctattcgatgaccttccccatctctgcacctcctgcagcgttcgccttaaacaaaaggaagaacttgataggcacatggagctgcatgataagaagaagctcgagttaagtggcacaaacagtaaatgtagggtttggtttccaaaggttgataactggatagcagcaaaagcgggagagttagaacctgaatatgaagaagttctgagtgaacctgagagtgctattgaggattgtcaagccgtagctgctgatgagacccaatgcgcatgtatattatgtggagaagtatttgaggattatttcagtcaagaaatggctcaatggatgttcaagggagcttcttacctgaccaatcctccagccaacagtgaggccagtggtcctatcgtccatacaggttgtctgaccacgagttcattgcagagcttggaagttggaattgcaatcaagcaggtaacttgtagctctagtcaatttgcgacttttggtcattctagggctctctgatctcaaactttgttctttaatcctgcatgaatccacctttacgtgtttgtttctcgttcggttcctattttgctaggaaattgttgagtagcctccatataaagacctatggtggaggaagaagtggcatcacagaggaaacaggttcgtgattcctctgagttcctcatgatgctagttttgtagaaaagtgtcgagcttttgagttaaggaaaaaagatgtgcagcatttttaattttattttttcttatttgtctctcctttttcatcatatacaaatacaaaaagagcaaagaggccttcccaatatcggacatttccatattctatgcctttc

SEQ ID NO: 5 - DNA, 2892 bp, Arabidopsis thaliana
  source 1..2892: mol_type = other DNA
atggagaatcctcgtagaccgtttgatagatccagagatccaggtccgatgaagaaacccaggctcagcgaggaatcaatccggcctgttaattccaacgcgcggcagtttttgtcgcagagaacgcttggtacggctacggcggtgacagtgccacctgcttcgtcaaggtttcgagttagtggtagagaaactgagagtagtattgtcagtgatccgagccgagaggcttaccagccacagccggttcatccacattatgagcttgtgaatcagtacaagtctgcactggctgagctcactttcaactcgaagccgataataacgaaccttacaataattgctggagagaatgttcatgctgctaaagccgtcgtaaccgccatttgcaacaacatcctcgaggtaccaagtgatcagaagctgccaacactgtacctgttagacagcattgtgaagaacatcggaagggattacattaagtactttggtgccaggctgcctgaggtttttgttaaggcttacaggcaagttgaccctcctatgcattctaacatgcggcatctttttggaacctggaaaggagtctttcatccgcaaacacttcagcttattgagaaagaacttggctttaatgctaaaagtgatggttcagcagctgttgtatccacagccagagctgagccgcaatctcagcgcccaccacatagcatccatgtgaatcccaagtatctagaaaggcagcgccttcagcagtcgggacggacaaaaggaatggttactgatgtcccagagaccgcacccaatttgactagggattcagatagactggagagagtttcaagcatagcatctggaggctcgtgggtaggtccagctaaagtaaataacattcggcgacctcaaagagatttacttagtgagcccctctacgagaaagatatcgagtcaatagcaggggaatatgattatgcttctgatctaccacataactctagatcggtgattaagaatgttggttcaaggattactgatgacggatgtgagaaacaatggtacggagctacgaacagggatcctgatctgatttctgaccaaagggatgggttacattccaaaagcagaacttccaactatgctacggctagagtagaaaatttggaatcaagtggtcccagtagaaatataggtgtgccttatgatagttggaaaaactctgaggaagaagagtttatgtgggatatgcactcaaggttatccgaaactgatgtagccactatcaacccaaagaacgagcttcatgcaccagatgaatcagagagactggaatctgaaaaccacctcttaaaacgaccaaggttttcagctctcgacccaaggtttgacccagcgaattcaacaaattcatattccagcgagcaaaaagatccaagtagtattgggcactgggcgttttcttctaccaatgcaacctcgactgcaactagaaagggaattcagcctcaaccaagggttgcaagctctggaattttgccaagctcaggatctgggtcagatagacaatcgcctttacatgattcaacttctaaacagaatgtgaccaagcaagatgttagaagagctcactcactcccccaacgtgatcctagggcttcccggtttccagcaaaacaaaatgttcccagggatgactctgtacggctcccatcttcaagtagtcagtttaaaaacacaaatatgcgtgaactaccagtggaaatctttgactctaagtcagcagctgaaaatgccccaggccttactcttgcctcagaagctacaggacaacccaacatgagtgatttgctagaagctgtcatgaagagtggaatcctttctaataattcaacatgtggtgcgatcaaggaggaaagtcatgatgaggtcaacccgggggcactaaccctgccagcagcctctaagcctaagactttgccaatttcattggctactgacaatttattagctcgtttaaaagttgaacaatcctccgcaccacttgtctcatgtgcagcgtctcttactggcatcacctcagtacaaacgtcaaaagagaaaagtaaggcctcagatcctctttcatgcctcttaagctctttagtttcgaagggcttgatatctgcttcgaagacagagctgccatcggctccttccataacgcaggaacacagtcctgatcattccaccaatagctctatgtctgtatctgtggttccagcagatgctcagccatctgttttggtgaaggggccatctactgcccctaaggtaaaagggttagctgcaccatctgaaacatcaaaatctgaacccaaggacctcataggattgaagtttagggcggacaaaatccgtgaacttcacccttcggtcattagtagtctattcgatgaccttccccatctctgcacctcctgcagcgttcgccttaaacaaaaggaagaacttgataggcacatggagctgcatgataagaagaagctcgagttaagtggcacaaacagtaaatgtagggtttggtttccaaaggttgataactggatagcagcaaaagcgggagagttagaacctgaatatgaagaagttctgagtgaacctgagagtgctattgaggattgtcaagccgtagctgctgatgagacccaatgcgcatgtatattatgtggagaagtatttgaggattatttcagtcaagaaatggctcaatggatgttcaagggagcttcttacctgaccaatcctccagccaacagtgaggccagtggtcctatcgtccatacaggttgtctgaccacgagttcattgcagagcttggaagttggaattgcaatcaagcaggaaattgttgagtag

SEQ ID NO: 6 - protein, 963 aa, Arabidopsis thaliana
MENPRRPFDRSRDPGPMKKPRLSEESIRPVNSNARQFLSQRTLGTATAVTVPPASSRFRVSGRETESSIVSDPSREAYQPQPVHPHYELVNQYKSALAELTFNSKPIITNLTIIAGENVHAAKAVVTAICNNILEVPSDQKLPTLYLLDSIVKNIGRDYIKYFGARLPEVFVKAYRQVDPPMHSNMRHLFGTWKGVFHPQTLQLIEKELGFNAKSDGSAAVVSTARAEPQSQRPPHSIHVNPKYLERQRLQQSGRTKGMVTDVPETAPNLTRDSDRLERVSSIASGGSWVGPAKVNNIRRPQRDLLSEPLYEKDIESIAGEYDYASDLPHNSRSVIKNVGSRITDDGCEKQWYGATNRDPDLISDQRDGLHSKSRTSNYATARVENLESSGPSRNIGVPYDSWKNSEEEEFMWDMHSRLSETDVATINPKNELHAPDESERLESENHLLKRPRFSALDPRFDPANSTNSYSSEQKDPSSIGHWAFSSTNATSTATRKGIQPQPRVASSGILPSSGSGSDRQSPLHDSTSKQNVTKQDVRRAHSLPQRDPRASRFPAKQNVPRDDSVRLPSSSSQFKNTNMRELPVEIFDSKSAAENAPGLTLASEATGQPNMSDLLEAVMKSGILSNNSTCGAIKEESHDEVNPGALTLPAASKPKTLPISLATDNLLARLKVEQSSAPLVSCAASLTGITSVQTSKEKSKASDPLSCLLSSLVSKGLISASKTELPSAPSITQEHSPDHSTNSSMSVSVVPADAQPSVLVKGPSTAPKVKGLAAPSETSKSEPKDLIGLKFRADKIRELHPSVISSLFDDLPHLCTSCSVRLKQKEELDRHMELHDKKKLELSGTNSKCRVWFPKVDNWIAAKAGELEPEYEEVLSEPESAIEDCQAVAADETQCACILCGEVFEDYFSQEMAQWMFKGASYLTNPPANSEASGPIVHTGCLTTSSLQSLEVGIAIKQEIVE

SEQ ID NO: 7 - DNA, 4010 bp, Arabidopsis thaliana
  source 1..4010: mol_type = other DNA
atctctctcttcccccacaagggtggcgattcgaggagtttccatggtggaacagaaaccctagagcctttatgctgccactaattatcactcttcactattgaattggggatggactcagagaaaatcctcaatccaagacttgtttcgattaatagcaccagccgcaaaggtatgtccgttgagttgccgcagaaaccgccgccaccgccgtcgttgttagaccggtttaaagctttgcttaatcagcgtgaagatgagttcggtggtggtgaagaggttttgcctccgagtatggatgagattgttcagctttatgaggttgttttgggtgagcttactttcaattctaagcctattatcactgatctcactattatcgctggtgaacaacgggaacatggtgaaggaatcgctaatgctatttgcactcgaatccttgaggtttgctttcttcttatttgtttccactttctagccttattgtatagtatgtaagtgtagaagtgggaaaaaaattaggtttttgattgtcagagattagtgtttggtttgtggtttagtgaaatggctgcttctgagttacctgtgggtgtatatatgcaggctcctgttgagcaaaagttgccatctctgtatcttttagacagcattgttaagaacattgggcgggattatggtcgctatttctcatcccgtctacccgaggtgagcagtctacaagaagatgtttacttaaatgatttgaatagggctgaagcatacccatgagctgttttgatcctattctgctttaggtcttctgccttgcatataggcaagcacatccgagtttacatccgtcaatgcgtcacctgtttgggacttggtcgagtgtttttccaccacctgtactacgcaagattgatatgcaattgcaactttcatctgcagcaaaccaatcttccgtaggggcttctgagcctagtcaaccaacgagaggtatacatgttaatccaaaatatcttcgcaggctggagccctcagccgccgaaaatgtaagcctttctctttgtcattatgaacagagatgacattctttttttaggtgcatctgcaaccttgaagctatggtgtgttaattttatataagtgtactaagtaaaacgggtattagattagtttggcctcatagtggtcttatccattgtatttctttaaagttttgttagttagacgagatagatgtttacattcttcaagtaatcaatcttgactggaagggagttgttgttatggcctctctcagttatacatgatgagtaggtcactgggactctggtacattagaaatgatcttgttagcacatgtagagggtttttttttggttatagtacagtagatcactgtttatgcatcatgtgtcgtttggaactgtttttatatgttttttttcaaagagtgactatacctgagatgcatatttccttaattgctcttgcttgacatgcagaatttgagaggaattaattccagtgcaagagtttatggccaaaattcactcggtgggtataatgatttcgaagatcagttagagagcccatcttcactgtctagtacaccagatggttttaccaggaggtctaatgatggagctaatccttctaatcaagcttttaactatggaatgggtcgagctactagcagagatgatgaacatatggaatggcgaagaaaggaaaacttaggccaaggaaatgatcacgaaagacctagagcgttgatagatgcatatggagttgacaccagcaaacacgtaacgatcaataaaccaattcgcgatatgaatggcatgcacagtaagatggttacaccatggcagaacactgaggaagaagagtttgattgggaagatatgagccctaccctagatcgtagcagagctggtgaatttttgcgatcatctgtaccagcattaggaagtgtaagggcaagacccagagtaggaaacacaagtgattttcatttagattctgacattaagaatggtgtaagtcatcagctacgagaaaattggagtttgtcacaaaattatccacatacatccaaccgtgttgatacaagagcaggaaaagacttgaaagtattggcttcatctgtagggttagtttcatcaaacagtgaattcggggctcctccatttgatagcattcaagatgttaactcacgttttggccgggctcttcctgatgggacatggccccatttgagtgctagaggacccaactctctccctgtaccttctgctcatttgcatcatttggcaaatcccggtaatgcgatgtcaaatcgcttgcaaggtaagcctttatataggcccgaaaaccaggtctctcagagtcatcttaatgatatgactcaacagaatcagatgctagttaattatttgccatcctcatctgctatggctccaaggccaatgcaatcgttgttaacccacgtaagtcatgggtaccctccacatgggtcaaccattagaccatctttatcaatccaaggtggggaagccatgcatcctctttcttcaggagttttatctcagataggagcctctaatcaaccaccaggtggcgcattctctggtctgattggctctctcatggctcaaggtttgatctcattgaacaatcagcctgctggtcaggtaattattcaaatcatgtccttgagagtcggtttgttcttagttattgctttatcttgaattgttggtttatatattcagggaccattggggttggagtttgatgcagacatgctcaagatacgtaatgagtctgcaatcagtgctttatatggggatctcccgaggcaatgcacaacctgtggtctacgcttcaagtgccaagaagagcacagtaagcacatggattggcatgtaaccaagaacaggatgtccaagaaccataagcagaacccttctcggaaatggtttgtaagtgctagcatgtggctcagtggtgcagaagcacttggagctgaagcagtaccagggttcttgcccacggagccaacaacagaaaagaaggatgacgaagatatggcagttcctgcagatgaagaccaaacttcatgtgcattatgtggtgagccatttgaagacttttatagtgatgaaaccgaggaatggatgtacaaaggtgcggtatacatgaatgctcctgaagaatcaacaacagatatggacaagtctcagttaggtcctatagtgcatgcaaagtgcaggccagagtccaatggggtaagccattccatgcattttcattagcttcatttgtgtctagttgaatccattttcattttcgctgctagacatttatccagtgacgacctcttgatttcccgatcatggctttttcagggtgatatggaagagggtagtcagaggaaaaagatgcggagttagatcccttagaccgaattttaggtctttttcttgactacatttaagttacattcttatttcttcatcatctatatgcacttagtcacagaataccaccaatgaagagaaatgatggagagagtcttctttctcctaaaactgtaccattgaaaaaaagaggtttacacccacatatattatcattctttatgcctctaaattcctgttgtgccttaggacatggatatcgtgtaactgttctgcattctctgctacacctaacctggggtgcttgtagtagtaaaatcagatgtcgcaacagtagtttccccatgtaaatggaatacaatgtctctgtttcggattgtctttttctttttcatctttagatattgcagggaaagcccgagaagactgatattcgaatgtgaacagatttcttatgatgaatggatctcatagatgctgttgctaagagaaccagtttcaggtttcttctttagagctttccctgtctgagttttacctttctatgttaggctgttagctaaaaatgttagttaacataactctctttaagcgtat

SEQ ID NO: 8 - DNA, 2427 bp, Arabidopsis thaliana
  source 1..2427: mol_type = other DNA
atggactcagagaaaatcctcaatccaagacttgtttcgattaatagcaccagccgcaaaggtatgtccgttgagttgccgcagaaaccgccgccaccgccgtcgttgttagaccggtttaaagctttgcttaatcagcgtgaagatgagttcggtggtggtgaagaggttttgcctccgagtatggatgagattgttcagctttatgaggttgttttgggtgagcttactttcaattctaagcctattatcactgatctcactattatcgctggtgaacaacgggaacatggtgaaggaatcgctaatgctatttgcactcgaatccttgaggctcctgttgagcaaaagttgccatctctgtatcttttagacagcattgttaagaacattgggcgggattatggtcgctatttctcatcccgtctacccgaggtcttctgccttgcatataggcaagcacatccgagtttacatccgtcaatgcgtcacctgtttgggacttggtcgagtgtttttccaccacctgtactacgcaagattgatatgcaattgcaactttcatctgcagcaaaccaatcttccgtaggggcttctgagcctagtcaaccaacgagaggtatacatgttaatccaaaatatcttcgcaggctggagccctcagccgccgaaaataatttgagaggaattaattccagtgcaagagtttatggccaaaattcactcggtgggtataatgatttcgaagatcagttagagagcccatcttcactgtctagtacaccagatggttttaccaggaggtctaatgatggagctaatccttctaatcaagcttttaactatggaatgggtcgagctactagcagagatgatgaacatatggaatggcgaagaaaggaaaacttaggccaaggaaatgatcacgaaagacctagagcgttgatagatgcatatggagttgacaccagcaaacacgtaacgatcaataaaccaattcgcgatatgaatggcatgcacagtaagatggttacaccatggcagaacactgaggaagaagagtttgattgggaagatatgagccctaccctagatcgtagcagagctggtgaatttttgcgatcatctgtaccagcattaggaagtgtaagggcaagacccagagtaggaaacacaagtgattttcatttagattctgacattaagaatggtgtaagtcatcagctacgagaaaattggagtttgtcacaaaattatccacatacatccaaccgtgttgatacaagagcaggaaaagacttgaaagtattggcttcatctgtagggttagtttcatcaaacagtgaattcggggctcctccatttgatagcattcaagatgttaactcacgttttggccgggctcttcctgatgggacatggccccatttgagtgctagaggacccaactctctccctgtaccttctgctcatttgcatcatttggcaaatcccggtaatgcgatgtcaaatcgcttgcaaggtaagcctttatataggcccgaaaaccaggtctctcagagtcatcttaatgatatgactcaacagaatcagatgctagttaattatttgccatcctcatctgctatggctccaaggccaatgcaatcgttgttaacccacgtaagtcatgggtaccctccacatgggtcaaccattagaccatctttatcaatccaaggtggggaagccatgcatcctctttcttcaggagttttatctcagataggagcctctaatcaaccaccaggtggcgcattctctggtctgattggctctctcatggctcaaggtttgatctcattgaacaatcagcctgctggtcagggaccattggggttggagtttgatgcagacatgctcaagatacgtaatgagtctgcaatcagtgctttatatggggatctcccgaggcaatgcacaacctgtggtctacgcttcaagtgccaagaagagcacagtaagcacatggattggcatgtaaccaagaacaggatgtccaagaaccataagcagaacccttctcggaaatggtttgtaagtgctagcatgtggctcagtggtgcagaagcacttggagctgaagcagtaccagggttcttgcccacggagccaacaacagaaaagaaggatgacgaagatatggcagttcctgcagatgaagaccaaacttcatgtgcattatgtggtgagccatttgaagacttttatagtgatgaaaccgaggaatggatgtacaaaggtgcggtatacatgaatgctcctgaagaatcaacaacagatatggacaagtctcagttaggtcctatagtgcatgcaaagtgcaggccagagtccaatgggggtgatatggaagagggtagtcagaggaaaaagatgcggagttag

SEQ ID NO: 9 - protein, 808 aa, Arabidopsis thaliana
MDSEKILNPRLVSINSTSRKGMSVELPQKPPPPPSLLDRFKALLNQREDEFGGGEEVLPPSMDEIVQLYEVVLGELTFNSKPIITDLTIIAGEQREHGEGIANAICTRILEAPVEQKLPSLYLLDSIVKNIGRDYGRYFSSRLPEVFCLAYRQAHPSLHPSMRHLFGTWSSVFPPPVLRKIDMQLQLSSAANQSSVGASEPSQPTRGIHVNPKYLRRLEPSAAENNLRGINSSARVYGQNSLGGYNDFEDQLESPSSLSSTPDGFTRRSNDGANPSNQAFNYGMGRATSRDDEHMEWRRKENLGQGNDHERPRALIDAYGVDTSKHVTINKPIRDMNGMHSKMVTPWQNTEEEEFDWEDMSPTLDRSRAGEFLRSSVPALGSVRARPRVGNTSDFHLDSDIKNGVSHQLRENWSLSQNYPHTSNRVDTRAGKDLKVLASSVGLVSSNSEFGAPPFDSIQDVNSRFGRALPDGTWPHLSARGPNSLPVPSAHLHHLANPGNAMSNRLQGKPLYRPENQVSQSHLNDMTQQNQMLVNYLPSSSAMAPRPMQSLLTHVSHGYPPHGSTIRPSLSIQGGEAMHPLSSGVLSQIGASNQPPGGAFSGLIGSLMAQGLISLNNQPAGQGPLGLEFDADMLKIRNESAISALYGDLPRQCTTCGLRFKCQEEHSKHMDWHVTKNRMSKNHKQNPSRKWFVSASMWLSGAEALGAEAVPGFLPTEPTTEKKDDEDMAVPADEDQTSCALCGEPFEDFYSDETEEWMYKGAVYMNAPEESTTDMDKSQLGPIVHAKCRPESNGGDMEEGSQRKKMRS

SEQ ID NO: 10 - DNA, 1482 bp, Arabidopsis thaliana
  source 1..1482: mol_type = genomic DNA
agattcgttttatttttcaatcgtttcaatttctaaaattctcaacgcgatcaccccaatccaaatttagggtttcttttgtctttctgcgatcgatctctggtaactcataacttgttgttgaatcgcgaagatggcatcaaacggatccttctctgctcagagaaacgcaaacgctggaacaacgatgaagagacgaaacgacaatcgcggttacggtggcggaatcggatgttatcaagaggagaggaaccgttacgcgccgccgcaaaaacgtttcagatcgcaactgcaacagcaattccgatctggtcataatcctctgtatcattatggtagtaatactaataataacgtgtctagggtttcttctcagtcatataacaattacggtgttgatgtgattgcttctaattcaagttttgctttacctaacaatgattctaacaccaacaattatcagaaaccctttgttgtttatggaaaccctaatccccaaattgttcctcttccattaccttatcgtaagcttgatcctttagattcgttgcctcaatgggttccaaattcaactccgaattatcctgttaggtcatcgaattttgtaccaaacacacctgattttacgaatgttcaaaatccgatgaatcattcgaacatggtttcagttgtgtctcaatcgatgcatcaaccaattgttttaagcaaagaattgactgatttgcttagtcttcttaacaatgagaaagagaagaaaacttcagaagcaagtaataatgattcattgcctgttggattaagctttgacaatccatcatcgcttaatgttcgacatgaatctgtgattaagtctctgtattctgatatgccgaggcaatgtacttcgtgtggggtgagattcaaatgtcaagaagagcatagtaaacatatggattggcacgtaaggaagaacaggtcggtgaaaaccactacaagacttggtcagcaaccgaagaaatcgaggggatggttagcgagtgcatctttatggctttgtgcaccaacgggaggagggaccgtagaagtagcatcttttggaggaggagaaatgcagaagaagaatgaaaaggatcaagtacaaaaacaacacatggtgcctgcggatgaagatcagaagaattgcgcgttatgcgtagagccgtttgaagagttctttagccatgaagctgatgactggatgtacaaagatgcagtctacttaaccaagaatggtcgtatagttcatgttaagtgtatgcctgaaccccgacctgctaaggatttgagagagccgtcgagggtaatgtctgttacagttccatcggttgcgaaagctatactctgctgattgagattttttatatctctgttttagtcattcgtttttttaaaagtaaatgtacattacattaatcaagatgtaacacaaatacagttctatcaattgaatctcctattttactc

SEQ ID NO: 11 - DNA, 1233 bp, Arabidopsis thaliana
  source 1..1233: mol_type = other DNA
atggcatcaaacggatccttctctgctcagagaaacgcaaacgctggaacaacgatgaagagacgaaacgacaatcgcggttacggtggcggaatcggatgttatcaagaggagaggaaccgttacgcgccgccgcaaaaacgtttcagatcgcaactgcaacagcaattccgatctggtcataatcctctgtatcattatggtagtaatactaataataacgtgtctagggtttcttctcagtcatataacaattacggtgttgatgtgattgcttctaattcaagttttgctttacctaacaatgattctaacaccaacaattatcagaaaccctttgttgtttatggaaaccctaatccccaaattgttcctcttccattaccttatcgtaagcttgatcctttagattcgttgcctcaatgggttccaaattcaactccgaattatcctgttaggtcatcgaattttgtaccaaacacacctgattttacgaatgttcaaaatccgatgaatcattcgaacatggtttcagttgtgtctcaatcgatgcatcaaccaattgttttaagcaaagaattgactgatttgcttagtcttcttaacaatgagaaagagaagaaaacttcagaagcaagtaataatgattcattgcctgttggattaagctttgacaatccatcatcgcttaatgttcgacatgaatctgtgattaagtctctgtattctgatatgccgaggcaatgtacttcgtgtggggtgagattcaaatgtcaagaagagcatagtaaacatatggattggcacgtaaggaagaacaggtcggtgaaaaccactacaagacttggtcagcaaccgaagaaatcgaggggatggttagcgagtgcatctttatggctttgtgcaccaacgggaggagggaccgtagaagtagcatcttttggaggaggagaaatgcagaagaagaatgaaaaggatcaagtacaaaaacaacacatggtgcctgcggatgaagatcagaagaattgcgcgttatgcgtagagccgtttgaagagttctttagccatgaagctgatgactggatgtacaaagatgcagtctacttaaccaagaatggtcgtatagttcatgttaagtgtatgcctgaaccccgacctgctaaggatttgagagagccgtcgagggtaatgtctgttacagttccatcggttgcgaaagctatactctgctga

SEQ ID NO: 12 - protein, 410 aa, Arabidopsis thaliana
MASNGSFSAQRNANAGTTMKRRNDNRGYGGGIGCYQEERNRYAPPQKRFRSQLQQQFRSGHNPLYHYGSNTNNNVSRVSSQSYNNYGVDVIASNSSFALPNNDSNTNNYQKPFVVYGNPNPQIVPLPLPYRKLDPLDSLPQWVPNSTPNYPVRSSNFVPNTPDFTNVQNPMNHSNMVSVVSQSMHQPIVLSKELTDLLSLLNNEKEKKTSEASNNDSLPVGLSFDNPSSLNVRHESVIKSLYSDMPRQCTSCGVRFKCQEEHSKHMDWHVRKNRSVKTTTRLGQQPKKSRGWLASASLWLCAPTGGGTVEVASFGGGEMQKKNEKDQVQKQHMVPADEDQKNCALCVEPFEEFFSHEADDWMYKDAVYLTKNGRIVHVKCMPEPRPAKDLREPSRVMSVTVPSVAKAILC

SEQ ID NO: 13 - DNA, 7233 bp, Brassica napus
  source 1..7233: mol_type = genomic DNA
atggagaattcgcgtagacctttcgatagatcgagagatccagggcctatgaagaagcccaggctcagcgatgtgaattcgaacgcgcggcagttaacgtcgcagagaacgatcggtgccgcgtcgtcttcgagatttcgagctgctgggggaagagaattggagagtgatccgaacggggaggcttatcagccgcaaccggttcatccacattacgagcttgtgaaccagtataagtcggctttatctgagctcacgatcaactcgaagccgatcattacgaaccttacgataattgctggagagaattttcatgctgctaaggccgttgttgccaccgtttgcaacaacatcctcgaggtgaatcatgatcactgttatagtttgttttactctttgatccattttttcttgaatctctttttttttttgaggaattgtataatctaatggtaaataaggtgaaggtggtggttgagattgtgattgtgatctgtatacgaggtttaatgagaaaaaaatccaatttttaaagcaacccttgtgtctctctgaattatatatgctttttttttaatacttgtgaatcaaagtttttagtttttgtaaagtatctgtattcacgaaactatgttgttaactatgtcaaatgagatgttgcccgtgcaacagtttgatccataatcatttctttttgagaatgcatatatgagctcctcatgtcattttactgagtttttcttatcttataggagtgtgtagtctctttgacaacgttggggttgctcaattgtatctcactttattatttgaggaagagctcttctctataagctctgttttttttgtgaggttgtattttctgttagtgccttctgattaccgaatactctccagaaagagaagacagtgtttattcgtatttggatagcagagaagcattggagggagcctgttcagtcaagtcgtgggaagacaaggattactctctccttttttttgtcactttagcttagaagctgagacttctaatagtgtaacgatgccatttttggccatgtagttttcgctgatgttctttaatgtctttctgtctccatctttagtttccttgtttgtcagaatatattttagtagtagtttgaatgtcatcatattttttgatccaaattttcgtagtatttcttatcctttagggtctccttgttggttactctctatgagatcaaattggccttttcttcctgggtatctgatttgaagagtattatgcaggtaccaagtgatcagaagctgccaacattgtacctattggacagcattgtgaagaacattggaagggattacattaagtactttgctgccaagcttcctgaggttggtgttttaaattgatagtcatggttatataatttcttttctttttttctggctgcgtcatctttgattttcctctttttaattctgtgcaggtatttgttaaggcttacagacaagttgaccctcctatgcgttctaatatgcggcatctctttggaacttggaaaggagtctttcatccgaacacacttcagcaaattgagaaagaacttggttttaatgctaaaagtgatggctcagctgctgttagtagcacaggccgagctgacctgcaatctcagcgcccaccaaatagcatccatgtgaatccaaagtatctagaaaggcaacgccttcaacagtcaggaagggtaagcaacgtaaactttctgcaactgaaaattatcagtgaggacaaacctgaagtaaacacgagtttatatactgatatgtaacttctgtaaaagtttcctgagtattcttcttttgatttttttgtgcaggcaaaaggaatggttactgatgtccccgagatcgcatccaatttggcaagggattcagatagactggaaagagtttcaagcatagcatctggaggctcgtgggcaggtccagctaaagtaaatgtaagaatgagttttccttggcatggttacagtttttaacctatgttctgtctactgattgtgtgttgaactcagaccattcgacgacctcaaagagactcactcagtgaacccctctacgagagagatatggagtcaatatcaggggaatatgattatacttctgaactaccgcataattctagaccagtgattaagaaagttaatgcaagggttactgacgacgggtgtgagaaacagtggtacgaaaccgtgagcaggggtcctgatctgatttctgaccaaagggatgtgttacatactaaaagcagactttccaactacgcgactgcgagactagaaaatttggaatcaagtggtcctagtagaaacacaggtgtgccgtatgatagttggaaaaactctgaggaagaagagtttatgtgggacatgcactcaaggttatctgaaacagatgtggccactgtcaacccaaagaatgagcttcacacaccagaggaatcagagagattggttagctatgatagtcatttgcattacttttaattatgaagttttctatttagtttcttgactttttaaatcttttcccatcttgtaggaatctgacaatcacctcttaaaacgaccaagattatcagctctcgacccaaggtttgatccgacttcgacaaactcatattccagcgagcaaaaagatggaagtgtttatggccgcttgagttcaacctctaatgcagctagaaagggtattcagcctcaatctaaggtgtcagatagacactcgcctttacgtgattcaacttttaaacagaatgtgaccaagcaagattctagaagaactcattccctcactcaacgtgatcctagggcttcacggtttccagcgaaatctcaaagtgtcctcagggatgactttgtgcggcctcagttaaaaaaatcaaattcgattgacctacctgattcatttcagggggaaaagttcgactctaagtcagcaggacttactattgcttcagaagctacaggacaacgcaacatgagtgatttgctggaagcagtcatgaagagtggaatcctttctaataatttaactaatggtgcaatgaaggaggagagttcccaggttaacccaagggcactaaccgtgtcagcagcctctaagcctacggcttcgccaaattcagtggctgctaaaaaaatatcagctcgtttaaaattggaacgatcctctgcaccgcttggttcggacccagtgtctcttaccggtgtcacatcagcacagacatcaaaagagagtagtaaagcctcagatcctatttcgtccctcttgagctccttggtttcaaagggcttgatatctgcttccaagacggagctgccatctgttgtacagtgtgcggctgctccttccatatcacaagaccatagtcctgatcattctaccaatagctccacttctatatctgtggttccgtctgatgcacagtcattggttgtggtggagaagggtccttttactgctccgaaggtagcaccagccgaaacggcagtttcagaacccgaaaacctcataggattgaagtttagggcagataagatccgtaaacttcacccctctgtgattagtagtctctttgatgaccttccacatctctgcacctcctgtggcgttcgccttaaagaaaaggaagaacttgacaggcacatggagatgcacgataagaagaagcttgagttacgtggcacaaacagtaaatgtagggtttggtttccaaaggctgatgattgggtagcagcgaaagcgggagagctagaacctgaatatgatgaaattctgagtgaacctgaaagtgaggatggtccagccgtagctgctgatgagaaccagtgcgcgtgtatattatgtggagagatgtttgaggactgtttcagtcaaggaacgggtcaatggctattcaagggagcttcttaccttaccattcctccatccaatagtgaagccaatggaccaatcgtccatgcaggttgtctgaccaagagttctttgccgagcttgggaattgggaatgcaataaaacaggtataacttagtactatagtgaatctctagggctctcttcgattgttcaatccagcatgaatccatctttacgtgttcctttcttgtttggttccattctgctaggaaatgattgagtagactccatacgaagggccaagaaacctatgctggaggaagcgggtggcatcacaggggaaacaggtttgttgttatccattcctcatgatgctctcgtgttgtagaaaagtgtcaaaattttgagtcgagtaaaaaaaaagatgtgcagtatttaaaagcaaaatcttatgtatgcttttgtctcccctttttttttcattttatacaaaatacaaaagagcaaaaaggcttttcccagtactgcacttcacatgttttgtgtcactaaagaatatgcaagcaacttgggttttcttatgaaaacagatttgtcgagttaagtatcatgaacatccatcgcaacaaatgaccatatctgtgtaccttagcaatgagtagcttctgtttaaagattgttttatcatttcatctcagatgtttgtagctaaaaaaacaaacaccacatccggagcacatatgtttccttaaaaaatatcatatgtatgtaagggtaaaacattcgatgtgaactaaaagaaatattaaaatattaaaaaaaaacatcagaaacgatatagtttaattcttttcggggatgcatagtaggcccattgggcatctggtcacgtcttcagaagaagaatgtcgtagaagttatccttgttcctaaaagcaccatcgcctcaaaaccgttaaactacccgcggtaattaaaaaaaaccggcatcgcgacaatccaacggctgaaaaagtttttcttgaagaagaggccaaaaaaagatcgtcacctcatttctcgaaccttcccaccacttcatcatctctataaacacacactttcctcttcccaatacctccctcactctgtatccaactttcttcaacagaccatggcttctttcaccagcaaatacgccggtaagcaacttcacctcttcctatctctccgtgatcccttttatcaactgtgtgcacgattcttttgcgttaaagtttcgatctcttcgtcttttgtgatgtgggttttaggacttagtgttgaggtttattacatcactcactcagtagctttgaacataagctacaactctgttatgttcaatcttttgtttttttggtgtactttgatacttgcgaagattttgataaacatctttttacgcacctcaagtcgatagatcagtactgcatcaggttatgagttgagttttaggacttagggtttaacgatccgaggtttactctgtaccttacatcactgtgaagctttggacataaactaaaaatctgttccgttcggttcattaacttatactgttttgtattatttttcactcgcagatgagctgatcgctaacgctgcctacatcggcacacccggaaagggcatcctcgcagcagacgaatcaaccggcacaatcggaaaacgtctctctagcatcaacgtagagaacgtcgagtccaacaggcgcgctctccgtgagcttctcttcaccactccaggcgctcttccttgcctcagtggcgtcatcctctttgaagaaactctataccaaaaaagctccaaaggtactttactcccagtcttgttatttatattttaattatcttgtttattatttagtagttaccttaattaggtggaattttaatagtttttttagattatttggttttcatataaataggcttcttctagagtttactcaattgattgattaataagatttttaaagagtttactcattttaacggtacttcatcccagtcttgttatttatattttaattatcttgcttaatatttagtagttatcttaattaggtggagttttaatactccctcattttcataatagtttgatggtttagaagaggtttgttgtttcaaaatagatgaagttttgatgtacttatattatttttaacttgactaaaactgtttaatcaattagattttgcagttattttttaattggttgaatgaattttaaattttatttgtaaagttaattttttgaaaaaaataaaaaatttaatctttgtgcaatgaggcatctcaaaaagagagtagttttttagattatttggttttcatataaataggcttctagagtttattcatttgattaattaataagatttttaaagagtttttgattgtctatatacaaaatgttttttttttttttttttgcgctgtctataccaaaagttgcttatgagttttgtttttgattccacagggacgccatttgtggatattctgaaggaaggaggagtcttacctggcatcaaagtcgacaagggcaccgtttctctacccggaaccaacggtgagaccaccacacaaggtctcgacggtctcggcgagcgttgcaagaagtactacgaggccggtgctcgtttcgccaaatggcgtgcggttctcaagatcggccccaccgagccatctgagctagccatccgcgagaacgcttacgggctcgctagatacgctgccatctgtcaggagaacggtcttgtccccatcgtggagcctgagattctcgttgacggttctcacgatattcagaagtgcgcggaggtgacggagcgtgtcttagcagcttgctacaaggccctgagcgatcaccatgtgatgctcgaaggaacgcttctgaaaccgaacatggtgactccagggtcagagagtggtaaagttgcgccggaggtggtggcggagtatactgtccgtgcgctgcagaggacggtgcctgcggctgttccggctgtggtgttcttgtcgggagggcagagcgaggaagaggcgacgaggaacctgaacgcgatgaaccagctgaagactaagaagccttggtcgctgtcgttttcgtttgggagggcgttgcagcagagcacgttgaagacttggggagggaaagaggagaatgtgaagaaggcgcaagaggcgttcttggtgaggtgcaaggctaactctgaggctacgcttggtgtttacaaaggtgatgctcagcttggtgaaggtgcagcggagagccttcacgtcaaggactacaagtactaa

SEQ ID NO: 14 - DNA, 3843 bp, Brassica napus
  source 1..3843: mol_type = other DNA
atggagaattcgcgtagacctttcgatagatcgagagatccagggcctatgaagaagcccaggctcagcgatgtgaattcgaacgcgcggcagttaacgtcgcagagaacgatcggtgccgcgtcgtcttcgagatttcgagctgctgggggaagagaattggagagtgatccgaacggggaggcttatcagccgcaaccggttcatccacattacgagcttgtgaaccagtataagtcggctttatctgagctcacgatcaactcgaagccgatcattacgaaccttacgataattgctggagagaattttcatgctgctaaggccgttgttgccaccgtttgcaacaacatcctcgaggtaccaagtgatcagaagctgccaacattgtacctattggacagcattgtgaagaacattggaagggattacattaagtactttgctgccaagcttcctgaggtatttgttaaggcttacagacaagttgaccctcctatgcgttctaatatgcggcatctctttggaacttggaaaggagtctttcatccgaacacacttcagcaaattgagaaagaacttggttttaatgctaaaagtgatggctcagctgctgttagtagcacaggccgagctgacctgcaatctcagcgcccaccaaatagcatccatgtgaatccaaagtatctagaaaggcaacgccttcaacagtcaggaagggcaaaaggaatggttactgatgtccccgagatcgcatccaatttggcaagggattcagatagactggaaagagtttcaagcatagcatctggaggctcgtgggcaggtccagctaaagtaaataccattcgacgacctcaaagagactcactcagtgaacccctctacgagagagatatggagtcaatatcaggggaatatgattatacttctgaactaccgcataattctagaccagtgattaagaaagttaatgcaagggttactgacgacgggtgtgagaaacagtggtacgaaaccgtgagcaggggtcctgatctgatttctgaccaaagggatgtgttacatactaaaagcagactttccaactacgcgactgcgagactagaaaatttggaatcaagtggtcctagtagaaacacaggtgtgccgtatgatagttggaaaaactctgaggaagaagagtttatgtgggacatgcactcaaggttatctgaaacagatgtggccactgtcaacccaaagaatgagcttcacacaccagaggaatcagagagattggaatctgacaatcacctcttaaaacgaccaagattatcagctctcgacccaaggtttgatccgacttcgacaaactcatattccagcgagcaaaaagatggaagtgtttatggccgcttgagttcaacctctaatgcagctagaaagggtattcagcctcaatctaaggtgtcagatagacactcgcctttacgtgattcaacttttaaacagaatgtgaccaagcaagattctagaagaactcattccctcactcaacgtgatcctagggcttcacggtttccagcgaaatctcaaagtgtcctcagggatgactttgtgcggcctcagttaaaaaaatcaaattcgattgacctacctgattcatttcagggggaaaagttcgactctaagtcagcaggacttactattgcttcagaagctacaggacaacgcaacatgagtgatttgctggaagcagtcatgaagagtggaatcctttctaataatttaactaatggtgcaatgaaggaggagagttcccaggttaacccaagggcactaaccgtgtcagcagcctctaagcctacggcttcgccaaattcagtggctgctaaaaaaatatcagctcgtttaaaattggaacgatcctctgcaccgcttggttcggacccagtgtctcttaccggtgtcacatcagcacagacatcaaaagagagtagtaaagcctcagatcctatttcgtccctcttgagctccttggtttcaaagggcttgatatctgcttccaagacggagctgccatctgttgtacagtgtgcggctgctccttccatatcacaagaccatagtcctgatcattctaccaatagctccacttctatatctgtggttccgtctgatgcacagtcattggttgtggtggagaagggtccttttactgctccgaaggtagcaccagccgaaacggcagtttcagaacccgaaaacctcataggattgaagtttagggcagataagatccgtaaacttcacccctctgtgattagtagtctctttgatgaccttccacatctctgcacctcctgtggcgttcgccttaaagaaaaggaagaacttgacaggcacatggagatgcacgataagaagaagcttgagttacgtggcacaaacagtaaatgtagggtttggtttccaaaggctgatgattgggtagcagcgaaagcgggagagctagaacctgaatatgatgaaattctgagtgaacctgaaagtgaggatggtccagccgtagctgctgatgagaaccagtgcgcgtgtatattatgtggagagatgtttgaggactgtttcagtcaaggaacgggtcaatggctattcaagggagcttcttaccttaccattcctccatccaatagtgaagccaatggaccaatcgtccatgcaggttgtctgaccaagagttctttgccgagcttgggaattgggaatgcaataaaacagaccatggcttctttcaccagcaaatacgccgatgagctgatcgctaacgctgcctacatcggcacacccggaaagggcatcctcgcagcagacgaatcaaccggcacaatcggaaaacgtctctctagcatcaacgtagagaacgtcgagtccaacaggcgcgctctccgtgagcttctcttcaccactccaggcgctcttccttgcctcagtggcgtcatcctctttgaagaaactctataccaaaaaagctccaaagggacgccatttgtggatattctgaaggaaggaggagtcttacctggcatcaaagtcgacaagggcaccgtttctctacccggaaccaacggtgagaccaccacacaaggtctcgacggtctcggcgagcgttgcaagaagtactacgaggccggtgctcgtttcgccaaatggcgtgcggttctcaagatcggccccaccgagccatctgagctagccatccgcgagaacgcttacgggctcgctagatacgctgccatctgtcaggagaacggtcttgtccccatcgtggagcctgagattctcgttgacggttctcacgatattcagaagtgcgcggaggtgacggagcgtgtcttagcagcttgctacaaggccctgagcgatcaccatgtgatgctcgaaggaacgcttctgaaaccgaacatggtgactccagggtcagagagtggtaaagttgcgccggaggtggtggcggagtatactgtccgtgcgctgcagaggacggtgcctgcggctgttccggctgtggtgttcttgtcgggagggcagagcgaggaagaggcgacgaggaacctgaacgcgatgaaccagctgaagactaagaagccttggtcgctgtcgttttcgtttgggagggcgttgcagcagagcacgttgaagacttggggagggaaagaggagaatgtgaagaaggcgcaagaggcgttcttggtgaggtgcaaggctaactctgaggctacgcttggtgtttacaaaggtgatgctcagcttggtgaaggtgcagcggagagccttcacgtcaaggactacaagtactaa

SEQ ID NO: 15 - protein, 1280 aa, Brassica napus
MENSRRPFDRSRDPGPMKKPRLSDVNSNARQLTSQRTIGAASSSRFRAAGGRELESDPNGEAYQPQPVHPHYELVNQYKSALSELTINSKPIITNLTIIAGENFHAAKAVVATVCNNILEVPSDQKLPTLYLLDSIVKNIGRDYIKYFAAKLPEVFVKAYRQVDPPMRSNMRHLFGTWKGVFHPNTLQQIEKELGFNAKSDGSAAVSSTGRADLQSQRPPNSIHVNPKYLERQRLQQSGRAKGMVTDVPEIASNLARDSDRLERVSSIASGGSWAGPAKVNTIRRPQRDSLSEPLYERDMESISGEYDYTSELPHNSRPVIKKVNARVTDDGCEKQWYETVSRGPDLISDQRDVLHTKSRLSNYATARLENLESSGPSRNTGVPYDSWKNSEEEEFMWDMHSRLSETDVATVNPKNELHTPEESERLESDNHLLKRPRLSALDPRFDPTSTNSYSSEQKDGSVYGRLSSTSNAARKGIQPQSKVSDRHSPLRDSTFKQNVTKQDSRRTHSLTQRDPRASRFPAKSQSVLRDDFVRPQLKKSNSIDLPDSFQGEKFDSKSAGLTIASEATGQRNMSDLLEAVMKSGILSNNLTNGAMKEESSQVNPRALTVSAASKPTASPNSVAAKKISARLKLERSSAPLGSDPVSLTGVTSAQTSKESSKASDPISSLLSSLVSKGLISASKTELPSVVQCAAAPSISQDHSPDHSTNSSTSISVVPSDAQSLVVVEKGPFTAPKVAPAETAVSEPENLIGLKFRADKIRKLHPSVISSLFDDLPHLCTSCGVRLKEKEELDRHMEMHDKKKLELRGTNSKCRVWFPKADDWVAAKAGELEPEYDEILSEPESEDGPAVAADENQCACILCGEMFEDCFSQGTGQWLFKGASYLTIPPSNSEANGPIVHAGCLTKSSLPSLGIGNAIKQTMASFTSKYADELIANAAYIGTPGKGILAADESTGTIGKRLSSINVENVESNRRALRELLFTTPGALPCLSGVILFEETLYQKSSKGTPFVDILKEGGVLPGIKVDKGTVSLPGTNGETTTQGLDGLGERCKKYYEAGARFAKWRAVLKIGPTEPSELAIRENAYGLARYAAICQENGLVPIVEPEILVDGSHDIQKCAEVTERVLAACYKALSDHHVMLEGTLLKPNMVTPGSESGKVAPEVVAEYTVRALQRTVPAAVPAVVFLSGGQSEEEATRNLNAMNQLKTKKPWSLSFSFGRALQQSTLKTWGGKEENVKKAQEAFLVRCKANSEATLGVYKGDAQLGEGAAESLHVKDYKY

SEQ ID NO: 16 - DNA, 4223 bp, Brassica napus
  source 1..4223: mol_type = genomic DNA
atggagaattcgcgtcgagcgtttgatagatcgagagatccggggccgattaagaaaccgaggctcagcgatgaaccaatccgtccggtgaatcccaacgcgcggcagtttccgtcgcagagaccagtcgctacggcggtgggagctctaccggcgtcgacaaggtttcgagctggcggtggtggtggtggtgggagagaagcggagagtagtagcgaggcgtatgagccgcaactggttcatccgcattacgagcttgtgaatcagtataagtcggcgttatccgagctcacgttcaactcgaagccgataattacgaacctcacgataatcgctggagagaatgttcatgctgcaaaggccgtcgtgtccaccatttgcaataacatcatagaggtgaacacctagttttcatgcccacttgttataataccatgtttgtgtcattgtatagttgctatataagggaaataaggtgaaggtggtggttgagattgtgatctgtatacgaggtttaatgagaaaaaaatctatttttgaaagcaaccctagtgtcttttctggaatctatgtgcttttttataagtatttatcaagtcgcagatcattcttgtgaatcaaagtttttactttatgccatctattggatcacattctgtaaaagatctgtttcccgacactatgttattaactatctcagattagctgttgcccatgcaaaagtttggtccatattcattttgtaagaatgcatatatcaactcctcatgtcattgtactgagttttgtttagaggagtgtgtagtctcttttacaacgttggagttgttcaattgttttttttttttcactttgccatttgacaagagctctgtaagctatgtgttaagtgaatttgtattttcttttagtggcttctgcttaccgaaagctttctccgggaagcggaaacattctttttagtcatctttgggtagccgagaatcattggagcctgttcagtaaagtttgtgggaagacaaggattgctatttcgatgtttgtcactttagcatagacattgacacttctaatagtgtttctctgccatttttgggcatgtagatattgctgacgttgtttaatggcttcatgtgtcccatttttagattccttgtttgtcaggatatatgttagtagtagcctgaatggcattatattttgtgatttgaattctgctaaagataagttagtgatatttggtttcttatcctttagtgtcccctctattgtggaaatccttgatgttactctctatgagatcaaatttgccattttctgggtatctgatatgaagaatattatgcaggtaccaagtgatcagaagctgccaacgttgtacctgttagacagcattgtgaagaacattggaagagattacattaagtactttgctgccaggctgcctgaggttggtgttttgttggtcatagttatatagtttcttttgcctagcaacttcatctgatctctctcttttcacttctgtgcaggtatttgttaaggcttacagacaagttgaccctcctatgcgttctaacatgcggcatctttttggaacctggaaaggagtctttcatccgcaaatacttcagcaaattgagaaagaacttggctttaatgctaaaagtgatggcccagcagctgctgttaccactggcagaactgatctgcaatctcagcgaccacaaaatagcattcatgtgaatcccaaatatctagaaaggcaacgccttcaacagtcagggcgggtaagaaaattaaacttgcggccactgaaatttattggtgaccacaaaaccaaagaaattccattaaagtttcctgagtatctgttttggtttatgtgcagacaaaagtaatgattacggatgtccctgagatcgcatccaatttgactcgggatccagatagactggagagagtttcaagcacagcatctggaggcttgtgggcaggtccagctaaagtgaatgtaagatgattttttccttggcttagttacagtcttaacatatgttctgtcttctgattgtgacgttgtatgttgaattcagaccattcgacgacctcaaaaagactcactcagtgaacccctctacgagaaggatatggagtcaatatcagaggaatatgaatatgcttctgatctaccgcataattctagatcagtggttaagaaagttggttctaggattactgatgaggggagtggaaaacaatggtacgaagctatgagcaggggtcctgatttgatttccgaccagagggaagggttacacaccaaaagcagagtttccaactacgctactgcgagacttgaaaattcggaatcaagtggtcccagtagaaatataggcgtgccttatgatagttggaaaaactctgaggaagaagagtttatgtgggacatgcattcaagggtatccgaaacagatgtggccactatcaacccgaagattgagtttcaggcttcagatgaatcagagagactggttagctatgatagtaatttgcattactttttataatggtgtaactcatttagtttgttgtcttttaaccttttcccatcttgtaggaatctaaaaatcacctcttaaaacggccaaggtattcagctgtcgatccaaggcttgatcctgtgaattcttattccagagagcaaaaagattcaagtattcttggcccctggacgtcttctcctagatctttgcatgattcagaagtttttccttctattagtgcagcctcaaatgcagctagaaagggaattcagcctcaatctaggattgctagctctggaattttgccaatctcaggacctgtgtcagataaacagtcgaaacagaatgtgagcaagcaagattctggaagagcgcactccctcactcaacgtgatcctagggcttcccggtttccagcaaaatctcaaagtgactccgtgcggcccttatcttcaagtagccagtttaagaacacaaatacgcttgaactacccgattcatctcaagttgaaaagtttgactctaagtcaacggctgaaaatgctcgaggacaagccaacatgaatgatttgctggcagctgtcatgaagagtggaatcctttctaataatgcaacttgtggtggaagcaaggaggagatgtctcaaaacggggctgacccaagggcaataaccctgtcagcagtatctaaggataagactttgccaacttcagttgctggcgcagtgtctcttactggtgtcacatcagcacaggcatcaaaagagaatagtaaggcctcagatcctatttcgtccctcttgagctccttagtttcaaagggcttgatatctgcttcaaagccggagccgccagcttcaaagacggagctgccatctcgcactcaatgtgcggctccttccgtttcacaggaccatagtcctgatcactctaccagtagttctatgtctgtggttccatcagatgctcagccagtggttttggtgaagaagggtccttccacttccccgaaggtagcaccagcagaaacagcaaaaacggaacccgaaaacctcataggattgaagttcagagcagataaaatccgtgaacttcacccctcggtgatcagtagtctttttgatgaccttccacacctctgcatctcctgtggcgttcgccttaagcaaaaagaagaacttgacaggcacatggaggagctgcacgataagaagaagctcgagttaaatggcacgaacagtaaatgtagggtttggtttccaaaggctgatgactggatagcagcgaaatctggagagcttgaacctgaatgtgaggaagatctgagtgaacccgagagtgcagctgaggatggtccagccatagctgctgatgagaaccagagcgcatgtatattatgtggagagatgtttgaggaatatttcagtcaagaaatggatcaatggatgttcaaaggagcttcttaccttgccattcctccggacgagagtgaggccaatggaccgatcgtccatgcaggttgtttgaccaagagttctctaccgggcttgggagttgggaatgcaatcaaacaggtaacttatctttaaaggctcgctgttctaaaactccattctttaatccagcatgaatccacatttacgtgttgttccattctgctaggaaatgatcgagtag

SEQ ID NO: 17 - DNA, 2832 bp, Brassica napus
  source 1..2832: mol_type = other DNA
atggagaattcgcgtcgagcgtttgatagatcgagagatccggggccgattaagaaaccgaggctcagcgatgaaccaatccgtccggtgaatcccaacgcgcggcagtttccgtcgcagagaccagtcgctacggcggtgggagctctaccggcgtcgacaaggtttcgagctggcggtggtggtggtggtgggagagaagcggagagtagtagcgaggcgtatgagccgcaactggttcatccgcattacgagcttgtgaatcagtataagtcggcgttatccgagctcacgttcaactcgaagccgataattacgaacctcacgataatcgctggagagaatgttcatgctgcaaaggccgtcgtgtccaccatttgcaataacatcatagaggtaccaagtgatcagaagctgccaacgttgtacctgttagacagcattgtgaagaacattggaagagattacattaagtactttgctgccaggctgcctgaggtatttgttaaggcttacagacaagttgaccctcctatgcgttctaacatgcggcatctttttggaacctggaaaggagtctttcatccgcaaatacttcagcaaattgagaaagaacttggctttaatgctaaaagtgatggcccagcagctgctgttaccactggcagaactgatctgcaatctcagcgaccacaaaatagcattcatgtgaatcccaaatatctagaaaggcaacgccttcaacagtcagggcggacaaaagtaatgattacggatgtccctgagatcgcatccaatttgactcgggatccagatagactggagagagtttcaagcacagcatctggaggcttgtgggcaggtccagctaaagtgaataccattcgacgacctcaaaaagactcactcagtgaacccctctacgagaaggatatggagtcaatatcagaggaatatgaatatgcttctgatctaccgcataattctagatcagtggttaagaaagttggttctaggattactgatgaggggagtggaaaacaatggtacgaagctatgagcaggggtcctgatttgatttccgaccagagggaagggttacacaccaaaagcagagtttccaactacgctactgcgagacttgaaaattcggaatcaagtggtcccagtagaaatataggcgtgccttatgatagttggaaaaactctgaggaagaagagtttatgtgggacatgcattcaagggtatccgaaacagatgtggccactatcaacccgaagattgagtttcaggcttcagatgaatcagagagactggaatctaaaaatcacctcttaaaacggccaaggtattcagctgtcgatccaaggcttgatcctgtgaattcttattccagagagcaaaaagattcaagtattcttggcccctggacgtcttctcctagatctttgcatgattcagaagtttttccttctattagtgcagcctcaaatgcagctagaaagggaattcagcctcaatctaggattgctagctctggaattttgccaatctcaggacctgtgtcagataaacagtcgaaacagaatgtgagcaagcaagattctggaagagcgcactccctcactcaacgtgatcctagggcttcccggtttccagcaaaatctcaaagtgactccgtgcggcccttatcttcaagtagccagtttaagaacacaaatacgcttgaactacccgattcatctcaagttgaaaagtttgactctaagtcaacggctgaaaatgctcgaggacaagccaacatgaatgatttgctggcagctgtcatgaagagtggaatcctttctaataatgcaacttgtggtggaagcaaggaggagatgtctcaaaacggggctgacccaagggcaataaccctgtcagcagtatctaaggataagactttgccaacttcagttgctggcgcagtgtctcttactggtgtcacatcagcacaggcatcaaaagagaatagtaaggcctcagatcctatttcgtccctcttgagctccttagtttcaaagggcttgatatctgcttcaaagccggagccgccagcttcaaagacggagctgccatctcgcactcaatgtgcggctccttccgtttcacaggaccatagtcctgatcactctaccagtagttctatgtctgtggttccatcagatgctcagccagtggttttggtgaagaagggtccttccacttccccgaaggtagcaccagcagaaacagcaaaaacggaacccgaaaacctcataggattgaagttcagagcagataaaatccgtgaacttcacccctcggtgatcagtagtctttttgatgaccttccacacctctgcatctcctgtggcgttcgccttaagcaaaaagaagaacttgacaggcacatggaggagctgcacgataagaagaagctcgagttaaatggcacgaacagtaaatgtagggtttggtttccaaaggctgatgactggatagcagcgaaatctggagagcttgaacctgaatgtgaggaagatctgagtgaacccgagagtgcagctgaggatggtccagccatagctgctgatgagaaccagagcgcatgtatattatgtggagagatgtttgaggaatatttcagtcaagaaatggatcaatggatgttcaaaggagcttcttaccttgccattcctccggacgagagtgaggccaatggaccgatcgtccatgcaggttgtttgaccaagagttctctaccgggcttgggagttgggaatgcaatcaaacaggaaatgatcgagtag

SEQ ID NO: 18 - protein, 943 aa, Brassica napus
MENSRRAFDRSRDPGPIKKPRLSDEPIRPVNPNARQFPSQRPVATAVGALPASTRFRAGGGGGGGREAESSSEAYEPQLVHPHYELVNQYKSALSELTFNSKPIITNLTIIAGENVHAAKAVVSTICNNIIEVPSDQKLPTLYLLDSIVKNIGRDYIKYFAARLPEVFVKAYRQVDPPMRSNMRHLFGTWKGVFHPQILQQIEKELGFNAKSDGPAAAVTTGRTDLQSQRPQNSIHVNPKYLERQRLQQSGRTKVMITDVPEIASNLTRDPDRLERVSSTASGGLWAGPAKVNTIRRPQKDSLSEPLYEKDMESISEEYEYASDLPHNSRSVVKKVGSRITDEGSGKQWYEAMSRGPDLISDQREGLHTKSRVSNYATARLENSESSGPSRNIGVPYDSWKNSEEEEFMWDMHSRVSETDVATINPKIEFQASDESERLESKNHLLKRPRYSAVDPRLDPVNSYSREQKDSSILGPWTSSPRSLHDSEVFPSISAASNAARKGIQPQSRIASSGILPISGPVSDKQSKQNVSKQDSGRAHSLTQRDPRASRFPAKSQSDSVRPLSSSSQFKNTNTLELPDSSQVEKFDSKSTAENARGQANMNDLLAAVMKSGILSNNATCGGSKEEMSQNGADPRAITLSAVSKDKTLPTSVAGAVSLTGVTSAQASKENSKASDPISSLLSSLVSKGLISASKPEPPASKTELPSRTQCAAPSVSQDHSPDHSTSSSMSVVPSDAQPVVLVKKGPSTSPKVAPAETAKTEPENLIGLKFRADKIRELHPSVISSLFDDLPHLCISCGVRLKQKEELDRHMEELHDKKKLELNGTNSKCRVWFPKADDWIAAKSGELEPECEEDLSEPESAAEDGPAIAADENQSACILCGEMFEEYFSQEMDQWMFKGASYLAIPPDESEANGPIVHAGCLTKSSLPGLGVGNAIKQEMIE

SEQ ID NO: 19 - DNA, 1233 bp, Brassica napus
  source 1..1233: mol_type = genomic DNA
atgtcttcatccaacggagtcttcgctcatcagagaaacgcgcatcgtaacggtggaagaaccatgctgaagagaccaatcgacaatcgcggatgcagaatcggaggttttcaggaggaggaggagcgaagccgttacgccccgcttcagaaacgccccagatatcagcaggcgcaaccgcaattccaatcggctaaacctaacggcggcgggactcctcctatgtatcgccatcatcatgatagtaacttgtctagggtttctaattacggtgatgtgatcaattctaattcaagtaatgtctttccttttgctcttcgtaacaatcgtcattcgcaatctaacaaaccgtatggttatgggaaccctaatccccaatcggttccgcttcctttaccgtatcgtaaactagatggtttcgattcgttaccggaatgggttcctaatttcgtacctaacacaaaccgtgagataaccggagtccggtttaacggtaaccaactttctcaagcaccgcctgtgtttacgaatttgcctaattcaatgcctttgaatcattcaaacatggtttcagttgtgtcgcaatcattaccccaacaacctgtcgtgagcaaagaattggctgatttacttagcgttcttaacaacaatgagaaagaatgtgatctcgggcttgattttgataaaagtaatcttaacgtgcgacatgaatctgttgttaagtctctttattccgacatgccgaggcaatgttcctcgtgtggtgttagattcaagggtcaagaagagcatagcaagcacatggactggcatgtgaggaagaatagaatggctaaagacgcgaccaaagccgccacaaccacgagggcaagccagaaaccgaagaagtcgcgtgattggtttgctagcttgtctctatggctcagtgcggcaacaggagctgccattgaaggggctaaacctctttttggagaaacacagaagacgaaagaagaggagaaacaacagcaacgctatgtccctgctgatgagaaccagaagatgtgtgcgttatgtttggagtcgttggaagagtttttcagccatgaagaggatgattggatgtacagagatgctgcctacttgaacatgaacgggtctggtcctatagtgcatgttaactgtatgccggaaccacgaaaagggcctgcaaagggattgacaaagcctgttgcgagtgcagtggcctgctga

SEQ ID NO: 20 - DNA, 1233 bp, Brassica napus; identical to SEQ ID NO: 19
  source 1..1233: mol_type = other DNA

SEQ ID NO: 21 - protein, 410 aa, Brassica napus
MSSSNGVFAHQRNAHRNGGRTMLKRPIDNRGCRIGGFQEEEERSRYAPLQKRPRYQQAQPQFQSAKPNGGGTPPMYRHHHDSNLSRVSNYGDVINSNSSNVFPFALRNNRHSQSNKPYGYGNPNPQSVPLPLPYRKLDGFDSLPEWVPNFVPNTNREITGVRFNGNQLSQAPPVFTNLPNSMPLNHSNMVSVVSQSLPQQPVVSKELADLLSVLNNNEKECDLGLDFDKSNLNVRHESVVKSLYSDMPRQCSSCGVRFKGQEEHSKHMDWHVRKNRMAKDATKAATTTRASQKPKKSRDWFASLSLWLSAATGAAIEGAKPLFGETQKTKEEEKQQQRYVPADENQKMCALCLESLEEFFSHEEDDWMYRDAAYLNMNGSGPIVHVNCMPEPRKGPAKGLTKPVASAVAC

SEQ ID NO: 22 - DNA, 3171 bp, Brassica napus
  source 1..3171: mol_type = genomic DNA
atgcaatcggagaaaatcctcaacccaagccttcttctcagcaccaccaccggccgcaaatcgatgtccgtcgttaaaccgccgccgccgccgtcgatactcgaccggttcaaggctctgctcaatcagcgcgaggacgagttcggcggcgaggatccgagttcggaagaggttgttcagctctacgaggttgttctgggcgagctcactttcaattccaagcctatcatcactgatctcactatcatcgccgatgagcagcgcgagcacggcgaagggatcgctaatgctatctgcactcgaatcctcgaggtctgggactttgtttccaccgctaaagccttcttcttgtgactgtggtttcgtgtgtatgcaggcaccggttgagcaaaagctgccgtctttgtatcttttagacagtattgtcaagaacattgggagggactatgctcgttacttctcgtctcgtttaccagaggtgagtgagcacagtctatataatgagatccttgtttaattggtttgtgtttgattcttgtctgctgttttaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccggctcaaccaacaaggggcatacatgttaatccgaaatatcttcgtaggctggagccctctgccgctgagagtgtaagcttttctctttgatcactatgaacagagatgacgttcttctatggattcaggaagcctggtataagattactctagcctcatagtggaacccttgttctgtatagataagattacctccagtaatgaagttttgcgtaattgtcgtatttttaagagttagatttactttcttctagtactcaactggagtaaaaaatgagtgttgtggtctctcaattattcatgatgagtaggtcactgggactcaggtgcatttcttttagttagacgcaatagataatgcattgtttttcgttgtgaacttgttttcttataggttttttctaagatgatttacaactaacatgcatttttcttgaactgtgccggcttggtatgcagaacttgagaggaataagttccagtgcaaaagtgtatagtcaaaactcagtcggcgggtatgatgattttgaagatcagttagatagcccatcttctctggccggaccaaggtctagtacagctggaactttgaccctcgcatcttctgcaattggaggtaagaatcttcattctttcagtctcaagacgtgtaaaacttgttattgactttcagtagcccttcatctttcgacttttatgactctgcagcagatgggtttcctaggaggtttagtgatggagttaattcttctaatcaagcctataactatggaatgggtcgggctactggcagagatgatgaacacatggactggcgaagaaaggacaacttcggccaaggaaatgaccatgaaagacccagagctttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgttcttcctgatgggacatggccccatttgggtgttagaggatccaactctctccctgtaccttctgctcagcatcatttggcaaatcctggtaatgcgatgttaaatcacttgaatggtaagcctttatataggccagaaaaccaggtctcccacatgacccaacagaatcaagtgctaggaaattacttaccgtcttcatctgcaatggcaccaagggcaatgcaatcgctgttaccccacggtcatggttaccctccactagggtcaaccattagaccatcgttatcagtccatggtggggaagccatgcatcctcattcttcaggggtttccatctctcagaatccttcgttgggagcctctaaccaaccaccgggcggtgcattctctggtctgattggctctctcatggctcaaggtttgatctcgttgaacaatcagcctactggccaggtaaattatttaaagcatgtcacaccttctaatacatgagagtcggactctttcttctagctttatcttaaataattgattttatatatacagggggcttcggtgatggagtttgatgcggacatgctcaagatacgtaacgagtctgcaataactgctctatatggggatcttcccaggcaatgcacaacatgtggtctacgctttaagtgccaagaagagcacagtaagcacatggattggcatgtaaccaagaacaggatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgagccgtttgaagacttctatagtgatgagacggaggaatggatgtacaaaggtgctgtatacatgaatgctcctgacggatcaacaacagatgtggacaagtctttgttaggtcctattgtacatgctaagtgtaggccagagactaatggggtaagcatttccttgcattctcattagcttcctttggaatacatgtctagttataagtttgattatattatcattttcctgctataagctttatccagtggtagcctcgtgatttccctaagaatggatatttgtttcagggtgatatggaagagggtagtcaaagaaaaaggatgcggagttag

SEQ ID NO: 23 - DNA, 2322 bp, Brassica napus
  source 1..2322: mol_type = other DNA
atgcaatcggagaaaatcctcaacccaagccttcttctcagcaccaccaccggccgcaaatcgatgtccgtcgttaaaccgccgccgccgccgtcgatactcgaccggttcaaggctctgctcaatcagcgcgaggacgagttcggcggcgaggatccgagttcggaagaggttgttcagctctacgaggttgttctgggcgagctcactttcaattccaagcctatcatcactgatctcactatcatcgccgatgagcagcgcgagcacggcgaagggatcgctaatgctatctgcactcgaatcctcgaggcaccggttgagcaaaagctgccgtctttgtatcttttagacagtattgtcaagaacattgggagggactatgctcgttacttctcgtctcgtttaccagaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccggctcaaccaacaaggggcatacatgttaatccgaaatatcttcgtaggctggagccctctgccgctgagagtaacttgagaggaataagttccagtgcaaaagtgtatagtcaaaactcagtcggcgggtatgatgattttgaagatcagttagatagcccatcttctctggccggaccaaggtctagtacagctggaactttgaccctcgcatcttctgcaattggagcagatgggtttcctaggaggtttagtgatggagttaattcttctaatcaagcctataactatggaatgggtcgggctactggcagagatgatgaacacatggactggcgaagaaaggacaacttcggccaaggaaatgaccatgaaagacccagagctttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgttcttcctgatgggacatggccccatttgggtgttagaggatccaactctctccctgtaccttctgctcagcatcatttggcaaatcctggtaatgcgatgttaaatcacttgaatggtaagcctttatataggccagaaaaccaggtctcccacatgacccaacagaatcaagtgctaggaaattacttaccgtcttcatctgcaatggcaccaagggcaatgcaatcgctgttaccccacggtcatggttaccctccactagggtcaaccattagaccatcgttatcagtccatggtggggaagccatgcatcctcattcttcaggggtttccatctctcagaatccttcgttgggagcctctaaccaaccaccgggcggtgcattctctggtctgattggctctctcatggctcaaggtttgatctcgttgaacaatcagcctactggccagggggcttcggtgatggagtttgatgcggacatgctcaagatacgtaacgagtctgcaataactgctctatatggggatcttcccaggcaatgcacaacatgtggtctacgctttaagtgccaagaagagcacagtaagcacatggattggcatgtaaccaagaacaggatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgagccgtttgaagacttctatagtgatgagacggaggaatggatgtacaaaggtgctgtatacatgaatgctcctgacggatcaacaacagatgtggacaagtctttgttaggtcctattgtacatgctaagtgtaggccagagactaatgggggtgatatggaagagggtagtcaaagaaaaaggatgcggagttag

SEQ ID NO: 24 - protein, 773 aa, Brassica napus
MQSEKILNPSLLLSTTTGRKSMSVVKPPPPPSILDRFKALLNQREDEFGGEDPSSEEVVQLYEVVLGELTFNSKPIITDLTIIADEQREHGEGIANAICTRILEAPVEQKLPSLYLLDSIVKNIGRDYARYFSSRLPEVFCLAYRQAHPSLHPSMRHLFGTWSGVFPSPVLRKIEMQLKLTSATNSQSSLGASEPAQPTRGIHVNPKYLRRLEPSAAESNLRGISSSAKVYSQNSVGGYDDFEDQLDSPSSLAGPRSSTAGTLTLASSAIGADGFPRRFSDGVNSSNQAYNYGMGRATGRDDEHMDWRRKDNFGQGNDHERPRALIDAYGVDTSKHTSISRPIRNVNGIHSKMVTPWQNTEEEEFDWEDMSPTLERSRAGEFLRSSVPALGSVRPRPRLGNIHDFQLDSDIKNGMNSRFGRGPAMISRVGPSGADVLPDGTWPHLGVRGSNSLPVPSAQHHLANPGNAMLNHLNGKPLYRPENQVSHMTQQNQVLGNYLPSSSAMAPRAMQSLLPHGHGYPPLGSTIRPSLSVHGGEAMHPHSSGVSISQNPSLGASNQPPGGAFSGLIGSLMAQGLISLNNQPTGQGASVMEFDADMLKIRNESAITALYGDLPRQCTTCGLRFKCQEEHSKHMDWHVTKNRMSKNHKQKPSRKWFVSGSMWLSGAEALGAEAVPGFLPVEPTTEKKDDEEMAVPADEDQTSCALCGEPFEDFYSDETEEWMYKGAVYMNAPDGSTTDVDKSLLGPIVHAKCRPETNGGDMEEGSQRKRMRS

SEQ ID NO: 25 - DNA, 4126 bp, Brassica napus
  source 1..4126: mol_type = genomic DNA
atggagaattcgcgtagaccgtttgatagatccagagatccggggccgattaagaaacccaggctcagcgacgaaccaatccggcagcttcagtcgcagagaccgctcggtggtgccgcggcgtcgtcgtcaaggtttcgagctggtggtggtgggagagaagcggagagtagtagcgaggcgtatgagccgcaacttgttcatccgcattacgagcttgtgaatcagtataagtcggctttatccgagctcacgttcaactcgaagccgataattacgaacctgacgataatcgctggggagaatgttcatgctgcaaaggccgtcgtgtccaccatttgcaataacatcatagaggtgaacacctagttttcatccccacttgttataatccatgtttgtgttgttactatataaaaaggaaaaggtgaaggtggtggttgagattgtgatctgtatacgaggtttaatgagaaaaagaactatttttgaaagcaaccctagtgtcttttctggaatctatgtgcttttttataagtatttatcaaagtcgcagatcattcttgtgaatcaaagtttttactttatgccatcttttggatcacattctgtaaaagatctgtttcccgacactatgttttctaaccatctcaaattagctgttgcccatgcaacagtttggtccatattcattttttttaagaatgcatcatgtcattgtactgagttttgtagtgtgtagtcttttttacaacgttggagttgttcaattctttttttactttgccatttgacaagagctctgtaagctatgtgttaagtgagtttgtattttcttttagtggcttctgcttaacgaaagctctctccaggaagtgaaatcaatttgtagccatctttgggtagacgagaatcattggagcctgttcagtaaagtttgtgggaaaacaaggattgctatttcactgtctcttcagcttagacattgacacttctaatagtgtttctctgccatttttgggcatgtagatattgctgacgttgtttaatggcttcatgtgtcccatttagattccttgtttgtcaggataatgttagtagtagcttgaatggcattatattattggtgatttgaattctggctgaagttaagttagtaatatttggttcttatcctttagtgtcccctctattgtggaaatccttgatgttactctctatgagatcaaatttgccattttctgggtatctgatgtgaagaatattatgcaggtaccaagtgatcagaagctgccaacgttgtacctgttggacagcattgtgaagaacattggaagagattacattaagtactttgctgccaggctgcctgaggttggtgttttgttggtagtcatagttatatagtttcttttgcctagaaacttcatctgatatctctcttttcacttctgtgcaggtatttgttaaggcctatagacaagttgaccctcctatgcgttctaacatgcggcatctttttggaacctggaaaggagtctttcatccgcaaatacttctgcaaattgagaaagaacttggctttaatgctagaagtgatggctcagcagctgctgttaccactggcagaactgatctgcaatctcagcgaccaccaaatagcatccatgtgaatcccaaatatctagaaaggcaacgcctccaacagtcagggcgggtaagtgaattaagcttgcggccactgaattttattggtgatcacaaaacaaagtaacttcattgaactttcctgggtatcttttcggtttatgtgcagacaaaagtaatggttaccgatgtccctgatatcgcatccaatttgactagggatccagatagactggagagagtttcaagcacagcttctggaggcttgtgggcaggtccagctaaagtgaatgtaagatggtttttccttgatttggttacagttttaacatattttctgtcttctgattgtgacattctgtgttgaattcagaccattcgacgacctcagaaagactcactcagtgaacccctctacgagaaggatatggagtcaatatcagaggaatatgaatatgcttctgatctaccgcacaattctagatcagtggttaagaaagttggtcctaggattactgatgatgggagtggaaaacaatggtacgaagctatgagcaggggtcctgatctgatttccgaccagagggaagggttacacaccaaaagcagagtttctaactacgctactgcgagacttgaaaattcggaatcaagtggtcccagtagaaatataggcgtgccttatgatagttggaaaaactctgaggaagaagagtttatgtgggacatgcattcaagggtatccgaaacagatgtggccactatcaacccgaagattgagtttcaggcttcagatgaatcagagagactggttagctttgatagtaatttgcattactttttataatggtgtaactcatttagtttcttgtcttttaaccttttcccatcttgtaggaatctaaaaatcacctcttaaaacggccaaggtattcagctgtcgatccaaggcttgatccggtgaattcttattccagagagcaaaaagattcaagtattcttggcccctggacgtcttctcctagatctttgcatgattcagaagtttttccttctattagtgcagcctcaaatgcagctagaaagggaattcagcctcaatctaggattgctagctctggaattttgccaatctcaggacctgtgtcagataaacagtcgaaacagaatgtgagcaagcaagattctggaagagctcactccctcactcaacgtgatcctagggcttcccggtttccagcaaaatctcaaagtgaatccgtgcggcccttatcttcaagtagccagtttaagaacacgaatacgcttgaactgcccgattcatctctagttgaaaagtttgactctaagtcaacagctgaaaatactcgaggacaagccaacatgaatgatttgttggcagctgtcatgaagagtggaatcctttctaataatgcaacttgtggtggaagcaaggaggagatgtctcaaaacggggctgacccaagggcactaaccctgtcagcagtgtctaaggataagactttgccaacttcagccgctggcgcagtgtcacttactgatgtcacatcagcacagacctcaaaagagaagagtaaggcctcagatcctatttcgtccctcttgagctccttagtttcaaagggcttgatatctgcttcaaagacggagctgccatctctcgctcaatgtgcggctccttccgtttcacaggaccatagtcctgatcattctaccagtagttctatgtctgtggttccatcagatgctcagccagtggttttggtgaagaaggatccttccactgccccgaaggtagcaccagtggaaacagcaaaaccggaacctgaaaacctcataggattgaagttcagagcagataaaatccgtgaacttcacccctcggtgattagtagtctctttgatgaccttccacatctctgcacctcctgtggcgttcgccttaagcaaaaggaagaacttgacaggcacgtggagaagctgcacgataagaagaagctcgagttaagtggcgcgaacagtaaatgtagggtttggtttccaaaggctgatgactggatagcagagaaatctagagagctagaacctgaatatgaggaagttctgagtgaatccgagagtgctggtgaggatgctcccgccgtagctgctgatgagaaccagagcgcatgtatattatgtggagagatgtttgaggactatttcagtcaagaaatggatcaatggatgttcaaaggagcttcttaccttgccattcctccggacgagagtgaggccaatggaccgatcgtccatgcagcttgtttgaccaagagttctttaccgggcttgggtgttgggaatgcaatcaaacaggtaacttatctttaaaggctcgctcatctgaaagtccattctttaatccagcatgaatccacatttgcgtgttgttccattctgctaggaaatgattgagtag

SEQ ID NO: 26 - DNA, 2772 bp, Brassica napus
  source 1..2772: mol_type = other DNA
atggagaattcgcgtagaccgtttgatagatccagagatccggggccgattaagaaacccaggctcagcgacgaaccaatccggcagcttcagtcgcagagaccgctcggtggtgccgcggcgtcgtcgtcaaggtttcgagctggtggtggtgggagagaagcggagagtagtagcgaggcgtatgagccgcaacttgttcatccgcattacgagcttgtgaatcagtataagtcggctttatccgagctcacgttcaactcgaagccgataattacgaacctgacgataatcgctggggagaatgttcatgctgcaaaggccgtcgtgtccaccatttgcaataacatcatagaggtaccaagtgatcagaagctgccaacgttgtacctgttggacagcattgtgaagaacattggaagagattacattaagtactttgctgccaggctgcctgaggtatttgttaaggcctatagacaagttgaccctcctatgcgttctaacatgcggcatctttttggaacctggaaaggagtctttcatccgcaaatacttctgcaaattgagaaagaacttggctttaatgctagaagtgatggctcagcagctgctgttaccactggcagaactgatctgcaatctcagcgaccaccaaatagcatccatgtgaatcccaaatatctagaaaggcaacgcctccaacagtcagggcggacaaaagtaatggttaccgatgtccctgatatcgcatccaatttgactagggatccagatagactggagagagtttcaagcacagcttctggaggcttgtgggcaggtccagctaaagtgaataccattcgacgacctcagaaagactcactcagtgaacccctctacgagaaggatatggagtcaatatcagaggaatatgaatatgcttctgatctaccgcacaattctagatcagtggttaagaaagttggtcctaggattactgatgatgggagtggaaaacaatggtacgaagctatgagcaggggtcctgatctgatttccgaccagagggaagggttacacaccaaaagcagagtttctaactacgctactgcgagacttgaaaattcggaatcaagtggtcccagtagaaatataggcgtgccttatgatagttggaaaaactctgaggaagaagagtttatgtgggacatgcattcaagggtatccgaaacagatgtggccactatcaacccgaagattgagtttcaggcttcagatgaatcagagagactggaatctaaaaatcacctcttaaaacggccaaggtattcagctgtcgatccaaggcttgatccggtgaattcttattccagagagcaaaaagattcaagtattcttggcccctggacgtcttctcctagatctttgcatgattcagaagtttttccttctattagtgcagcctcaaatgcagctagaaagggaattcagcctcaatctaggattgctagctctggaattttgccaatctcaggacctgtgtcagataaacagtcgaaacagaatgtgagcaagcaagattctggaagagctcactccctcactcaacgtgatcctagggcttcccggtttccagcaaaatctcaaagtgaatccgtgcggcccttatcttcaagtagccagtttaagaacacgaatacgcttgaactgcccgattcatctctagttgaaaagtttgactctaagtcaacagctgaaaatactcgaggacaagccaacatgaatgatttgttggcagctgtcatgaagagtggaatcctttctaataatgcaacttgtggtggaagcaaggaggagatgtctcaaaacggggctgacccaagggcactaaccctgtcagcagtgtctaaggataagactttgccaacttcagccgctggcgcagtgtcacttactgatgtcacatcagcacagacctcaaaagagaagagtaaggcctcagatcctatttcgtccctcttgagctccttagtttcaaagggcttgatatctgcttcaaagacggagctgccatctctcgctcaatgtgcggctccttccgtttcacaggaccatagtcctgatcattctaccagtagttctatgtctgtggttccatcagatgctcagccagtggttttggtgaagaaggatccttccactgccccgaaggtagcaccagtggaaacagcaaaaccggaacctgaaaacctcataggattgaagttcagagcagataaaatccgtgaacttcacccctcggtgattagtagtctctttgatgaccttccacatctctgcacctcctgtggcgttcgccttaagcaaaaggaagaacttgacaggcacgtggagaagctgcacgataagaagaagctcgagttaagtggcgcgaacagtaaatgtagggtttggtttccaaaggctgatgactggatagcagagaaatctagagagctagaacctgaatatgaggaagttctgagtgaatccgagagtgctggtgaggatgctcccgccgtagctgctgatgagaaccagagcgcatgtatattatgtggagagatgtttgaggactatttcagtcaagaaatggatcaatggatgttcaaaggagcttcttaccttgccattcctccggacgagagtgaggccaatggaccgatcgtccatgcagcttgtttgaccaagagttctttaccgggcttgggtgttgggaatgcaatcaaacaggaaatgattgagtag

SEQ ID NO: 27 - protein, 923 aa, Brassica napus
MENSRRPFDRSRDPGPIKKPRLSDEPIRQLQSQRPLGGAAASSSRFRAGGGGREAESSSEAYEPQLVHPHYELVNQYKSALSELTFNSKPIITNLTIIAGENVHAAKAVVSTICNNIIEVPSDQKLPTLYLLDSIVKNIGRDYIKYFAARLPEVFVKAYRQVDPPMRSNMRHLFGTWKGVFHPQILLQIEKELGFNARSDGSAAAVTTGRTDLQSQRPPNSIHVNPKYLERQRLQQSGRTKVMVTDVPDIASNLTRDPDRLERVSSTASGGLWAGPAKVNTIRRPQKDSLSEPLYEKDMESISEEYEYASDLPHNSRSVVKKVGPRITDDGSGKQWYEAMSRGPDLISDQREGLHTKSRVSNYATARLENSESSGPSRNIGVPYDSWKNSEEEEFMWDMHSRVSETDVATINPKIEFQASDESERLESKNHLLKRPRYSAVDPRLDPVNSYSREQKDSSILGPWTSSPRSLHDSEVFPSISAASNAARKGIQPQSRIASSGILPISGPVSDKQSKQNVSKQDSGRAHSLTQRDPRASRFPAKSQSESVRPLSSSSQFKNTNTLELPDSSLVEKFDSKSTAENTRGQANMNDLLAAVMKSGILSNNATCGGSKEEMSQNGADPRALTLSAVSKDKTLPTSAAGAVSLTDVTSAQTSKEKSKASDPISSLLSSLVSKGLISASKTELPSLAQCAAPSVSQDHSPDHSTSSSMSVVPSDAQPVVLVKKDPSTAPKVAPVETAKPEPENLIGLKFRADKIRELHPSVISSLFDDLPHLCTSCGVRLKQKEELDRHVEKLHDKKKLELSGANSKCRVWFPKADDWIAEKSRELEPEYEEVLSESESAGEDAPAVAADENQSACILCGEMFEDYFSQEMDQWMFKGASYLAIPPDESEANGPIVHAACLTKSSLPGLGVGNAIKQEMIE

SEQ ID NO: 28 - DNA, 7431 bp, Brassica napus
  source 1..7431: mol_type = genomic DNA
atggagaattcgcgtagacctttcgatagatcgagagatccagggcctatgaagaagcccaggctcagcgatgtgaattcgaacacgcggcagcttacgtcgtcttcaaggtttcgagctgctgggggaagagaattggagagtggtgttgttagtgatccgagcggggaggcttatcagccgcaacctgttcatacacattacgagcttgtgaaccagtataaggcagcactatccgagctcacgatcaactcgaagccgatcattacgaacctcacgataattgctggagagaatgttcacgctgctaaggccgttgttgccaccgtttgcaacaacatcctcgaggtgaatcatgatcactgttatagtttgttatactctttgatccattttttcttgaatctctttttttttttgaggaattgtattatatataaggtaaataaggtggaaggtggtggttgagattgtgatctgtatacgaggtttaatgagaaaaattcaattttgaaatcaacccttgtgtcttctctgaattatatatgctttcttttagttttatacttgtgaatcaaagtttctagtttatgtcatctactggatcccattctggaaaatatctatgtcaaatgagctgttgcccatgcaatattttgcttcatattcttttttcgagaatgcatatatcagcttctcaggtccttgtactgagtttttcttataggagtgtgtagtctctttgacaacgttggggttgttccattgtatctcactttattatttgtctatttgaggaagagctctctataagctctgttttatgtgaggttgtattttctgttagtgccttctgattaccgaatactctcaagaaagagaagacagcgtttattcgtatttggatagcagagaagcattggagcctgttcagtcaagtcgtgggaagacaaggattactctctcttttttttttgtcacttcagcttagaagctgagacttctaatagtgtaacgctgccaatttttcgctgatgttctttaatggccttctgtgtgcatctttagtttccttgtttgtcagaatatactttagtagtagtttgaatgtcatcatattttttgctccaaattttagtagtagttcttattcgttagggtctccttgttggttctctatgagatcaaattggctttttcttctgggtagctgatttgaagaacattatgcaggtaccaagtgatcagaaactgccaacattgtacctgttggacagcattgtgaagaacattggtagggattacattaagtactttgctgccaagctgcctgaggttggtgttattattgatagtcatggttacatagcttcttttctatttttctggctgcgccatctctgattttcctctttttaattctgtgcaggtatttgttaaggcttacagacaagttgaccctcctatgcgttctaatatgcggcatctctttggaacttggaaaggagtctttcatccgaacacacttcagcaaattgagaaagaacttggttttaatgctaaaagtgatggctcagctgctgttagtagcacgggccgagctgacctgcaatctcagcgcccaccaaatagcatccatgtgaatcccaagtatctagaaaggcaacgccttcaacagtcaggaagggtaagcaacgtaaactttctgcaactgaatcagtgaggacaaacctgaagtaaacacgagtttatatactgatatgcaacgtctgtaaaagtttcctgagtattcttcttttgatttttttttgtgcaggcaaaaggaatggttactgatgtccccgagatcgcatccaatttgacaagggattcagatagactggaaagagtttcaagcatagcatctggaggctcgtgggcaggtccagctaaagtaaatgtaagaatgagttttccttgtcatggttacagtttttaacttatgttctgtctactgattgtgtgttgaactcagaccattcgacgacctcaaagagactcactcagtgaacccctctacgagagagagattgagtcaatatcaggggaatatgattatgcttctgaactaccgcataattctagaccagtgattaagaaagttggtccaagggttactgatgacgggtgtgagaaacagtggtacgaaaccgtgagcaggggtccggatctgatttctgaccagagggatgtgttacataccaaaagcagaatttccaactacgcgactgcgagactagaaaatttggaatcaagtggtcctagtagaaacacaggtgtgccgtatgatagttggaaaaactctgaggaagaagagtttatgtgggacatgcactcaaggttatctgaaacagatgtggccactgtcaacccaaagaatgagcttcacatactagatgaatcagagagattggttagctatgatagtcatttgcattacttttaattatgaagttttctatttagtttcttgactttttaaatcttttcccatcttgtaggaatctgacaatcacctcttaaaacgaccaagattatcagctctcgacccaaggtttgatccgagttcgacaaactcatattccagcgagcaaaaagatccaagtgtttttggccgcttgagtttaacctctaatgcatctagaaagggaattcagcctcaatctaatgtgtcagatagacagtcgcctttacatgattcaacttttaaaccgaatgtgaccaagcaagattctagaagacctcattccctccctcaacgtgatcctagggcttcccggtttccagcgaaatctcaaagtgtcctcagggatgactctgtgcggcctcagtttaagaaaacaaatgcgattgacctacctgatacatttcagggggaaaagttcgactccaagtcagcaggtgaaaatgccccaggacttactattgcttcagaagctacaggacaacgcaacatgagtgatttgctggaagcagtcatgaagagtggaatcctttctaataatttaactaatggtgcaatcaaggaggagagttccaaggttaacccaagggcactaaccgtgtcaacagcctctaagtctaaggcttcgccaaattcagtggctgctaaaaaattatcagctcgtttgaaagtggaacgatcctctgcaccgcttggttcggacccagtgtctcttaccggtgtcacatcagcacagacatcaaaagagagtagtaaagcctcagatcctatttcgtccctcttgagctccttggtttcaaaggggttgatatctgcttccaagacggagctgccatctcttgtacaatgtgcggctgctcctttcatatcacaagaccatagtcctgatcattctaccaatagctctacgtcggtatctgttgttccgtctgatgcacagccattggttgtggtgaagaagggtccatccactgtcccgaaggtagcaccagccgaaacggcagtttcagaacacgaaaacctcataggattgaagtttagggcagataagatccgtgaacttcacccctcggtgattagtagtctctttgatgaccttccacatctatgcacctcctgtggcgttcgccttaaagaaaaggaagaacttgacaggcacatggagatgcacgataagaagaagcttgagttacgtggcacaaacagtaaatgtagggtttggtttccaaaggctgatgattgggtagcagcgaaagcgggagagctagaacctgaatatgatgaaattctgagtgaacctgaaagtgaggatggtccagccgtagctgctgatgaggaccagtgcgcgtgtatattatgtggagagatgtttgaggactgtttcagtcaaggaacggctcaatggctattcaagggagcttcttaccttaccattcctccatccaatagtgaagccaatggaccaatcgtccatgcaggttgtctgaccaagagttctttgccgagcttgggaattgggaatgcaataaaacaggtataacttagtactatagtgaatctctagggctctctgatctcaaactcgattgttcaatccagcatgaatccatctttacgtgtagcatgaatccatctttacgtgttcctttcttgtttggttccattctggtaggaaatgattgagtagactccatatgaagggccaagaaacctatgctggaggaagcggtggcatcacaagggaaacaggtttgttgtgatccattcctcatgatgctctcgtgttgtagaaaagtgtcaaaattttgagtcgagtaaaaaaaggtgtgcagtatttaaaagcaaaatcttatgtatgctttgtctccccttttttttcattttatacaaaatacaaaagagcaaaaagactttcccagtactgcacttcacatgttttgtgtcactaaagaatatgcaggtacttgggatttcttatgaaaacagatttgtcgagttaagtattatcatgaacatccatagcaacaaatgaccatatctgtgtagcttagcaatgagtcttctgttgaaagattgttttatcatttcatcatgtttgtagctaaaaaacaaacaccgcatccggagcacacatgtttcctagaggcgtaaatccggaaaacccgaaaaacccgatccgaatgccaacgggtaccgaacgcctaggtgtaatattttgtttgaccaaagataaatatgtaaggataacattcgatgtgaactaaaagaaattttgaaatattaaaaaaaaaaacatcagaaatgatatagtttaattcttttcggggatgcatagtaggcccattgggcatctggtcacgtctccagaagaagaatgtcgtagaagttatccttgtaaaagcacatcgcctcaaaaccgtcaaaaactacccgcggtataattaaaaaaaacggcatcgcgacaatccaacggctgaaaaagttttgcttgaagaagagaccaaaaaatcgtcacttccatttctcgaaccttcccaccacttcatcatctctataaacacacactttcctcttcccaatacctctctcactctgtatccaactttcttcaacagaccatggcttctttcaccagcaaatacgccggttagcaactttacctctttctatctctccgtgatccgtttatcaactgtgtgcacgatgccacgattgttttgcgttaaagtttcgatctcttcttcttttgtgatgtgggttttaggacttagtgtttaacgatttgaggtttattacatcactcactcagtagctttgaacataagctacaactctgttctgttggtttcatgaagtttcaatcttttgttttgttgtatactttgatacttgcgaggattttgataaactatctttttacgcacctcaagtcgatagatcagtactacttcaggttatgagttgagttttaggacagggtttaacgatctgaggtttactctgtaccttacatcactatgaagctttggacataaactaaaactctgttgggttcggttcggttcattaacttatactgttttgtcttatttttcactcgcagatgagctgatcgctaacgctgcctacatcggcacacccggaaagggcatcctcgctgcagacgaatcaaccggcacaatcggaaagcgtctttcaagcatcaacgtagagaacgtcgagtctaacagacgcgctctacgtgagcttctcttcactactccaggcgcacttccttgtctaagtggcgtcatcctctttgaagaaactctataccaaaaaagctccaaaggtactttaatcccagtcttgttatttatattttaattatcttgtttatttagtagttatcttaattaggtggaatcttaatagtttttagattatttggttttcatataaataggcttcttctagagtttactcatttgattgattaataaaatttactcattttaacgtacttcatcccagtcttgttatttatattttaattatcttgcttaatatttagtagttatcttaattaggtggagttttaatactccctccttttcataatagtttgatggtctagaagattagaagatgtttgttgtttcaaaatagatgaagttttgatatatttatattatttttaaatttactaaaactgtctaatcaattagattttgcaattatttttttaattggttgaatgagttttaaattatatttgtaaagttattttttggagaaaaataaatttcttaacctttgtgcaatgagccagagggagtagtttttcttaatctaatataattgataattagattttttggcttattagatcatataataatctaaaaaaactactcatataaataaaataaggttctagagttatttattagatcatataattgttatttatttggttttcatataaataaggttctagagtttactcatttgattgattaataagatttttaaagagcttttaattgtctataccaaaagtttttttttttttttttggctgtctataacaaaagttgcttattaggtttgtttttgatcacacagggaccccttttgtggatattctgaaggaaggtggagtcttacctggcatcaaagtcgacaagggcaccatttctctaccaggaaccaacggcgagaccaccacacagggtctcgacggtctcggcgagcgttgcaagaagtactacgaggccggtgctcgtttcgccaagtggcgcgcggtgctcaagatcggccccaacgagccatcagagctagccatccgcgagaacgcttacgggctcgctagatacgctgccatctgccaggagaacggtcttgtccccatcgtggagcctgagattctcgttgacggttctcacgatattcagaagtgcgcggaggcgacagagcgtgtcttagcagcttgctacaaggccctgagcgatcaccatgtcatgctcgaaggaacgcttctgaaaccgaacatggtgactccgggatcagagagtggtaaagttgcgccggaggttgttgcggagtacactgtccgcgctcttcagaggacggtgcctgcggctgttccggctgtggtgttcttgtcgggaggacagagcgaggaagaggcgacgaggaacctgaacgcgatgaatcagctgaagacgaagaagccttggtcgctttcgttttcgtttgggagggcgttgcagcagagcacgttgaagacttggggagggaaagaggagaatgtgaagaaggcgcaagaggcgttcttggttaggtgcaaggctaactctgaggctacgcttggtgtttacaaaggtgatgctcagcttggtgaaggtgcggcagagagccttcacgttaaggactacaagtactga

SEQ ID NO: 29 - DNA, 3846 bp, Brassica napus
  source 1..3846: mol_type = other DNA
atggagaattcgcgtagacctttcgatagatcgagagatccagggcctatgaagaagcccaggctcagcgatgtgaattcgaacacgcggcagcttacgtcgtcttcaaggtttcgagctgctgggggaagagaattggagagtggtgttgttagtgatccgagcggggaggcttatcagccgcaacctgttcatacacattacgagcttgtgaaccagtataaggcagcactatccgagctcacgatcaactcgaagccgatcattacgaacctcacgataattgctggagagaatgttcacgctgctaaggccgttgttgccaccgtttgcaacaacatcctcgaggtaccaagtgatcagaaactgccaacattgtacctgttggacagcattgtgaagaacattggtagggattacattaagtactttgctgccaagctgcctgaggtatttgttaaggcttacagacaagttgaccctcctatgcgttctaatatgcggcatctctttggaacttggaaaggagtctttcatccgaacacacttcagcaaattgagaaagaacttggttttaatgctaaaagtgatggctcagctgctgttagtagcacgggccgagctgacctgcaatctcagcgcccaccaaatagcatccatgtgaatcccaagtatctagaaaggcaacgccttcaacagtcaggaagggcaaaaggaatggttactgatgtccccgagatcgcatccaatttgacaagggattcagatagactggaaagagtttcaagcatagcatctggaggctcgtgggcaggtccagctaaagtaaataccattcgacgacctcaaagagactcactcagtgaacccctctacgagagagagattgagtcaatatcaggggaatatgattatgcttctgaactaccgcataattctagaccagtgattaagaaagttggtccaagggttactgatgacgggtgtgagaaacagtggtacgaaaccgtgagcaggggtccggatctgatttctgaccagagggatgtgttacataccaaaagcagaatttccaactacgcgactgcgagactagaaaatttggaatcaagtggtcctagtagaaacacaggtgtgccgtatgatagttggaaaaactctgaggaagaagagtttatgtgggacatgcactcaaggttatctgaaacagatgtggccactgtcaacccaaagaatgagcttcacatactagatgaatcagagagattggaatctgacaatcacctcttaaaacgaccaagattatcagctctcgacccaaggtttgatccgagttcgacaaactcatattccagcgagcaaaaagatccaagtgtttttggccgcttgagtttaacctctaatgcatctagaaagggaattcagcctcaatctaatgtgtcagatagacagtcgcctttacatgattcaacttttaaaccgaatgtgaccaagcaagattctagaagacctcattccctccctcaacgtgatcctagggcttcccggtttccagcgaaatctcaaagtgtcctcagggatgactctgtgcggcctcagtttaagaaaacaaatgcgattgacctacctgatacatttcagggggaaaagttcgactccaagtcagcaggtgaaaatgccccaggacttactattgcttcagaagctacaggacaacgcaacatgagtgatttgctggaagcagtcatgaagagtggaatcctttctaataatttaactaatggtgcaatcaaggaggagagttccaaggttaacccaagggcactaaccgtgtcaacagcctctaagtctaaggcttcgccaaattcagtggctgctaaaaaattatcagctcgtttgaaagtggaacgatcctctgcaccgcttggttcggacccagtgtctcttaccggtgtcacatcagcacagacatcaaaagagagtagtaaagcctcagatcctatttcgtccctcttgagctccttggtttcaaaggggttgatatctgcttccaagacggagctgccatctcttgtacaatgtgcggctgctcctttcatatcacaagaccatagtcctgatcattctaccaatagctctacgtcggtatctgttgttccgtctgatgcacagccattggttgtggtgaagaagggtccatccactgtcccgaaggtagcaccagccgaaacggcagtttcagaacacgaaaacctcataggattgaagtttagggcagataagatccgtgaacttcacccctcggtgattagtagtctctttgatgaccttccacatctatgcacctcctgtggcgttcgccttaaagaaaaggaagaacttgacaggcacatggagatgcacgataagaagaagcttgagttacgtggcacaaacagtaaatgtagggtttggtttccaaaggctgatgattgggtagcagcgaaagcgggagagctagaacctgaatatgatgaaattctgagtgaacctgaaagtgaggatggtccagccgtagctgctgatgaggaccagtgcgcgtgtatattatgtggagagatgtttgaggactgtttcagtcaaggaacggctcaatggctattcaagggagcttcttaccttaccattcctccatccaatagtgaagccaatggaccaatcgtccatgcaggttgtctgaccaagagttctttgccgagcttgggaattgggaatgcaataaaacagaccatggcttctttcaccagcaaatacgccgatgagctgatcgctaacgctgcctacatcggcacacccggaaagggcatcctcgctgcagacgaatcaaccggcacaatcggaaagcgtctttcaagcatcaacgtagagaacgtcgagtctaacagacgcgctctacgtgagcttctcttcactactccaggcgcacttccttgtctaagtggcgtcatcctctttgaagaaactctataccaaaaaagctccaaagggaccccttttgtggatattctgaaggaaggtggagtcttacctggcatcaaagtcgacaagggcaccatttctctaccaggaaccaacggcgagaccaccacacagggtctcgacggtctcggcgagcgttgcaagaagtactacgaggccggtgctcgtttcgccaagtggcgcgcggtgctcaagatcggccccaacgagccatcagagctagccatccgcgagaacgcttacgggctcgctagatacgctgccatctgccaggagaacggtcttgtccccatcgtggagcctgagattctcgttgacggttctcacgatattcagaagtgcgcggaggcgacagagcgtgtcttagcagcttgctacaaggccctgagcgatcaccatgtcatgctcgaaggaacgcttctgaaaccgaacatggtgactccgggatcagagagtggtaaagttgcgccggaggttgttgcggagtacactgtccgcgctcttcagaggacggtgcctgcggctgttccggctgtggtgttcttgtcgggaggacagagcgaggaagaggcgacgaggaacctgaacgcgatgaatcagctgaagacgaagaagccttggtcgctttcgttttcgtttgggagggcgttgcagcagagcacgttgaagacttggggagggaaagaggagaatgtgaagaaggcgcaagaggcgttcttggttaggtgcaaggctaactctgaggctacgcttggtgtttacaaaggtgatgctcagcttggtgaaggtgcggcagagagccttcacgttaaggactacaagtactga

SEQ ID NO: 30 - protein, 1281 aa, Brassica napus
MENSRRPFDRSRDPGPMKKPRLSDVNSNTRQLTSSSRFRAAGGRELESGVVSDPSGEAYQPQPVHTHYELVNQYKAALSELTINSKPIITNLTIIAGENVHAAKAVVATVCNNILEVPSDQKLPTLYLLDSIVKNIGRDYIKYFAAKLPEVFVKAYRQVDPPMRSNMRHLFGTWKGVFHPNTLQQIEKELGFNAKSDGSAAVSSTGRADLQSQRPPNSIHVNPKYLERQRLQQSGRAKGMVTDVPEIASNLTRDSDRLERVSSIASGGSWAGPAKVNTIRRPQRDSLSEPLYEREIESISGEYDYASELPHNSRPVIKKVGPRVTDDGCEKQWYETVSRGPDLISDQRDVLHTKSRISNYATARLENLESSGPSRNTGVPYDSWKNSEEEEFMWDMHSRLSETDVATVNPKNELHILDESERLESDNHLLKRPRLSALDPRFDPSSTNSYSSEQKDPSVFGRLSLTSNASRKGIQPQSNVSDRQSPLHDSTFKPNVTKQDSRRPHSLPQRDPRASRFPAKSQSVLRDDSVRPQFKKTNAIDLPDTFQGEKFDSKSAGENAPGLTIASEATGQRNMSDLLEAVMKSGILSNNLTNGAIKEESSKVNPRALTVSTASKSKASPNSVAAKKLSARLKVERSSAPLGSDPVSLTGVTSAQTSKESSKASDPISSLLSSLVSKGLISASKTELPSLVQCAAAPFISQDHSPDHSTNSSTSVSVVPSDAQPLVVVKKGPSTVPKVAPAETAVSEHENLIGLKFRADKIRELHPSVISSLFDDLPHLCTSCGVRLKEKEELDRHMEMHDKKKLELRGTNSKCRVWFPKADDWVAAKAGELEPEYDEILSEPESEDGPAVAADEDQCACILCGEMFEDCFSQGTAQWLFKGASYLTIPPSNSEANGPIVHAGCLTKSSLPSLGIGNAIKQTMASFTSKYADELIANAAYIGTPGKGILAADESTGTIGKRLSSINVENVESNRRALRELLFTTPGALPCLSGVILFEETLYQKSSKGTPFVDILKEGGVLPGIKVDKGTISLPGTNGETTTQGLDGLGERCKKYYEAGARFAKWRAVLKIGPNEPSELAIRENAYGLARYAAICQENGLVPIVEPEILVDGSHDIQKCAEATERVLAACYKALSDHHVMLEGTLLKPNMVTPGSESGKVAPEVVAEYTVRALQRTVPAAVPAVVFLSGGQSEEEATRNLNAMNQLKTKKPWSLSFSFGRALQQSTLKTWGGKEENVKKAQEAFLVRCKANSEATLGVYKGDAQLGEGAAESLHVKDYKY

SEQ ID NO: 31 - DNA, 1257 bp, Brassica napus
  source 1..1257: mol_type = genomic DNA
atgccttcatccaacggagtcttcgctcatcagagaaacacacatcggaacgctggaaagatgttgaagagaccgttcgacaatcccggcggctacggcggcagaatcggaggtttccaggaggaggagcgaagccgttacgcgccgccgcctcagaaacgccccagatatcagacgcaaccgcaattccaatcggctaaacctaacggcggcgggactcctcctatgtatcgtcatcatcatcatgatagtaacttgtctagggtttctaattacggtgatgtgatcaattctaattcaagtaatatctttccttttgctcttcgtaacaatggtcattcgcaatctaacaaaccgtatggttatgggaaccctaatccccaatcggttccgcttcctttaccgtatcgtaaactcgatggtttcgattcgttgccggaatgggttcctaatttcgtacctaacacaaaccgtgagataaccggagtccggtttaacgctaaccaaccttctcaagcaccgcctgtgtttacgaatttgcccaattcaattcctatgaatcattcaaacatggtttcagttgtgtcacaattattaccacaaccgcctatcgtgagcaaagaattggcagatttacttagcgttcttaacaacaatgagaaagagaaagagagagaatgtgatgttgggcttgattttgataaaattgaccttaacgtgcgacgtgaatctgttgttaagtctctttattccgacatgccgaggcaatgttcctcgtgtggggttagattcaagggtcaagaagagcatagcaagcacatggactggcatgtgaggaagaacaggatggctaaagacgccaccaaagccaccacaactacgagggtaagccagaaatcgaagcagtcgcgcgactggtttgcgagctcgtctctttggctcagtgcagcaacaggagctgccattgaaggggctaaacctgttttcggagagacccagaagacaaaagaagaggagaaacaacagcaacacattgttcctgctgatgaaagccagaagatgtgtgcgttatgcttggagtcgttggaagagtttttcagccatgaagaggatgattggatgtacagagatgctgcctacttgaacatgaacgggtctggtcctatagttcatgttaactgtatgcctgaaccacgaaaagggcctgcaaaggaatcggcaaagctcgttacggttgcgagtgcagtggtctgctga

SEQ ID NO: 32 - DNA, 1257 bp, Brassica napus; identical to SEQ ID NO: 31
  source 1..1257: mol_type = other DNA

SEQ ID NO: 33 - protein, 418 aa, Brassica napus
MPSSNGVFAHQRNTHRNAGKMLKRPFDNPGGYGGRIGGFQEEERSRYAPPPQKRPRYQTQPQFQSAKPNGGGTPPMYRHHHHDSNLSRVSNYGDVINSNSSNIFPFALRNNGHSQSNKPYGYGNPNPQSVPLPLPYRKLDGFDSLPEWVPNFVPNTNREITGVRFNANQPSQAPPVFTNLPNSIPMNHSNMVSVVSQLLPQPPIVSKELADLLSVLNNNEKEKERECDVGLDFDKIDLNVRRESVVKSLYSDMPRQCSSCGVRFKGQEEHSKHMDWHVRKNRMAKDATKATTTTRVSQKSKQSRDWFASSSLWLSAATGAAIEGAKPVFGETQKTKEEEKQQQHIVPADESQKMCALCLESLEEFFSHEEDDWMYRDAAYLNMNGSGPIVHVNCMPEPRKGPAKESAKLVTVASAVVC

SEQ ID NO: 34 - DNA, 3202 bp, Brassica napus
  source 1..3202: mol_type = genomic DNA
atgcaatcggagaaaatcatcaacccaagccttcttctcggcaccaccaccggccgcaaatcgatgtccgtcgttaaaccgccgccgccgccttcgatactcgaccggttcaaggctctgcttaatcagcgcgaggatgagttcggtggcgaggatccgagttcggaagaggttgttcagctctacgaggttgttctgggcgagctcactttcaattccaagcctatcatcaccgatctcactatcatcgccgatgagcagcgggagcacggcgaaggaatcgctaatgctatctgcactcgaatcctcgaggtctgggactttgtttccaccactaaagcccttcttctttgtgcaaacatcttgtgactgtggtttcgtgtgtatgcaggcaccagttgagcaaaagctgccatctttgtatcttttagacagtattgtcaagaacattgggagggactatgctcgttacttctcgtctcgtttaccagaggtgagtgagcacagtctatattataatgagatccttgcttaaatggtttgtatttgattcatgtctgctattttaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccgactcaaccaacaaggggcatacatgttaacccgaaatatcttcgtaggctggaaccctctgccgctgagagtgtaagctcttttctctttgatcactatgaacagagatgacgttcttctatggatttcttataatattcaggaagcctggtataagattattctagcctcatagtggacccatgttctgtatagataagattgcctccagtaataaaattttgcgtaattgccgtattttaagagttagatttactttcttctagtactcaactggagtaaaaaatgagtgttgtggtctctctcttattcatgatgagtaggtcactgggactccggtgcatttcttttagttagacgcaatagataatgcattgtttttcgttgtgaacttgttttcttataggttttttctaagatgatttacaactaacattcatttttcttgaactgtgccggcttggtatgcagaacttgagaggaataagttccagtgcaaaagtgtatagtcaaaactcagtcggcgggtatgatgattttgaagatcagttagatagcccatcttctctggccggacccaggtctattacagctggaactttgaccctcgcatcttctgcaattggaggtaacaatcttcattctttcagtctcaagacgtgtaaaacttgttattgactttcagtagccctgcatctttcgacttttatgactctgcagcagatgggtttcctaggaggtttagtgatggagttaattcttctaatcaagcctataactatggaatgggtcgggctactggcagagatgatgaacacatggactggcgaagaaaggaaaactttggccaaggaaatgaccatgaaagacccagagcgttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgctcttcctgatgggacatggccccatttgggtgttagaggatccaactctctccctgtgccttctgctcacgtgcatcatttggcaaatcctggtaatgcgatgttaaatcacttgaatggtaagcctttatataggccagaaaaccaggtctcccacatgacccaacagagtcaagtgctaggaaattacttaccgtcttcatctgcaatggcaccaagggcaatgcaatcgctgttaccccacggtcatggttaccctccactagggtcaaccattagaccatctttatcagtccatggtggggaagccatgcatcctcattcttcaggggtttccatctctcagaatccttcgttgggagcctctaatcaaccaccgggcggtgcattctctggtctgattggctctctcatggctcaaggtttgatctcgttgaacaatcagcctactggccaggtaaattatttaaagcatgtcacaccctctaatacatgagagtcggactctttcttctagctttatcttaaataattgattttatatatacagggggcttcggtgatggagtttgatgcggacatgctcaagatacgtaacgagtctgcaataactgctctatatggggatcttcccaggcaatgcacaacatgtggtctacgcttcaagagccaagaagagcacagtaagcacatggattggcatgtaaccaagaacagaatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgaggcgtttgaagacttctatagtgatgagacggaggaatggatgtacaaaggtgctgtgtacatgaatgctcctgacggatcaacaacagatgtggacaagtctttgttaggtcctattgtacatgctaagtgcaggccagagtctaatggggtaagcatttccttgcattctctcattagcttcctttggaataaatgtctagttataagttaaatggcagttgatcatattatagctttatccagtggtagcctcgtgatttccctaagaatggatattttttttcagggtgatatggaagagggtagtcaaagaaaaaggatgcggagttag

SEQ ID NO: 35 - DNA, 2325 bp, Brassica napus
  source 1..2325: mol_type = other DNA
atgcaatcggagaaaatcatcaacccaagccttcttctcggcaccaccaccggccgcaaatcgatgtccgtcgttaaaccgccgccgccgccttcgatactcgaccggttcaaggctctgcttaatcagcgcgaggatgagttcggtggcgaggatccgagttcggaagaggttgttcagctctacgaggttgttctgggcgagctcactttcaattccaagcctatcatcaccgatctcactatcatcgccgatgagcagcgggagcacggcgaaggaatcgctaatgctatctgcactcgaatcctcgaggcaccagttgagcaaaagctgccatctttgtatcttttagacagtattgtcaagaacattgggagggactatgctcgttacttctcgtctcgtttaccagaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccgactcaaccaacaaggggcatacatgttaacccgaaatatcttcgtaggctggaaccctctgccgctgagagtaacttgagaggaataagttccagtgcaaaagtgtatagtcaaaactcagtcggcgggtatgatgattttgaagatcagttagatagcccatcttctctggccggacccaggtctattacagctggaactttgaccctcgcatcttctgcaattggagcagatgggtttcctaggaggtttagtgatggagttaattcttctaatcaagcctataactatggaatgggtcgggctactggcagagatgatgaacacatggactggcgaagaaaggaaaactttggccaaggaaatgaccatgaaagacccagagcgttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgctcttcctgatgggacatggccccatttgggtgttagaggatccaactctctccctgtgccttctgctcacgtgcatcatttggcaaatcctggtaatgcgatgttaaatcacttgaatggtaagcctttatataggccagaaaaccaggtctcccacatgacccaacagagtcaagtgctaggaaattacttaccgtcttcatctgcaatggcaccaagggcaatgcaatcgctgttaccccacggtcatggttaccctccactagggtcaaccattagaccatctttatcagtccatggtggggaagccatgcatcctcattcttcaggggtttccatctctcagaatccttcgttgggagcctctaatcaaccaccgggcggtgcattctctggtctgattggctctctcatggctcaaggtttgatctcgttgaacaatcagcctactggccagggggcttcggtgatggagtttgatgcggacatgctcaagatacgtaacgagtctgcaataactgctctatatggggatcttcccaggcaatgcacaacatgtggtctacgcttcaagagccaagaagagcacagtaagcacatggattggcatgtaaccaagaacagaatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgaggcgtttgaagacttctatagtgatgagacggaggaatggatgtacaaaggtgctgtgtacatgaatgctcctgacggatcaacaacagatgtggacaagtctttgttaggtcctattgtacatgctaagtgcaggccagagtctaatgggggtgatatggaagagggtagtcaaagaaaaaggatgcggagttag

SEQ ID NO: 36 - protein, 774 aa, Brassica napus
MQSEKIINPSLLLGTTTGRKSMSVVKPPPPPSILDRFKALLNQREDEFGGEDPSSEEVVQLYEVVLGELTFNSKPIITDLTIIADEQREHGEGIANAICTRILEAPVEQKLPSLYLLDSIVKNIGRDYARYFSSRLPEVFCLAYRQAHPSLHPSMRHLFGTWSGVFPSPVLRKIEMQLKLTSATNSQSSLGASEPTQPTRGIHVNPKYLRRLEPSAAESNLRGISSSAKVYSQNSVGGYDDFEDQLDSPSSLAGPRSITAGTLTLASSAIGADGFPRRFSDGVNSSNQAYNYGMGRATGRDDEHMDWRRKENFGQGNDHERPRALIDAYGVDTSKHTSISRPIRNVNGIHSKMVTPWQNTEEEEFDWEDMSPTLERSRAGEFLRSSVPALGSVRPRPRLGNIHDFQLDSDIKNGMNSRFGRGPAMISRVGPSGADALPDGTWPHLGVRGSNSLPVPSAHVHHLANPGNAMLNHLNGKPLYRPENQVSHMTQQSQVLGNYLPSSSAMAPRAMQSLLPHGHGYPPLGSTIRPSLSVHGGEAMHPHSSGVSISQNPSLGASNQPPGGAFSGLIGSLMAQGLISLNNQPTGQGASVMEFDADMLKIRNESAITALYGDLPRQCTTCGLRFKSQEEHSKHMDWHVTKNRMSKNHKQKPSRKWFVSGSMWLSGAEALGAEAVPGFLPVEPTTEKKDDEEMAVPADEDQTSCALCGEAFEDFYSDETEEWMYKGAVYMNAPDGSTTDVDKSLLGPIVHAKCRPESNGGDMEEGSQRKRMRS

SEQ ID NO: 37 - DNA, 6216 bp, Glycine max
  source 1..6216: mol_type = genomic DNA
aagatcacagttgaatgtgggaagggagcttcactcctcactggtggccttcattcacacatgatataataatccctatcgcagcctacttataaacactccacctccttccatctctctctttatatttttttattaattcatgtaaattaggattagggtttatctcaaatttgcaccgtacaattttaaatcagaacttccgttctcattcatcccccccttcccaataaaaccgacgcgacaatcaataaccaggatgctattttcttgctgcaaatccagttcctcaattgagagcgcattagggttttagattcgaatgagcatggagagcacgcgcagatcgttggacagatccagagaaccaggtcccaagaagcctcgattgatcgacgaactcagcgcccgacagttacctcaacgcactgcggcagttactacgctggcgtctacgaggtttcgagcaaatgacagagactccgaaatcagcgatttgggtcgtggcggtggcggcggcgggtatcagccgcagcctccgccgcaccaagagcttgtgactcaatacaagaccgcacttgctgagctcactttcaattcgaagccgataattaccaacttgactattattgcaggagagaatctctctgctgccaaggcaattgctgctgctgtttgtgctaacattatagaggtgaacaaaatgtgcggttgtttatgttaatgtttattgaattgaattgtaatggatttggaatgaagaaagagcaattcgagttaatctctctgattttttataagtggtgttgctattgtttgtcatgctatgcggtgaattaagtttgaattagtgttgtaccttggtggaatgctagtcagaaagagggcattcttgattcgttgaaattgaatttttttcatctcatttcattggccctgtagatatagattgggtcttaagtggtacactctgccctgcaccttaaaagaacaaaaaacatatttacttttccaatcaatgtcctttggccaaatgacggtggtttggtattttgattgggtgtgtaactggtaatgagcaccattggagagtggtttttgtatgcacaggggattgagaatcctggaaaacatggaatttttcatttacaagagagaagtgaaccccaacagtttccaatctcaatcagtgctaaagttcacttttggtgaaatttgcagctgttttccttgttttcatatgtcagggcaatcttatttctctcattgtgtctttgtattatggaaaaaggtcttgtttaattgaaatgagttcatgactttccaggttccaagtgatcaaaaactgccatctctttacctcttagacagtattgttaagaacattggacgggattatataaagtattttgcttccagactacctgaggtcagatatttattatctttaaccatttaccttcatgcctttattttgatatgcttattattgtatatgatgaatcctttagattgtgaagttatgaaattttatactgaactactttgatttgtgatataagttatttacacgaatcaagagattgtaagaattattgtatttcccttacaaataaataatctatttataatgaaatggaaccatcttttccctttgcatttttcctgtgatatatgttttctaattctatacacctatttttaactaggtactactactttacattttaggtattctgcaaggcttacagacaggttgatccttgtgtgcattcaagtatgaaacatctttttggaacttggaaaggagtctttcctcctcagtcccttcagatgattgagaaggaacttggctttgcacctgcagtcaatggttcggcttcagtgtctgctacagtcaggagtgatttgcagtcacaacgcccacctcatagtatccatgtgaatcccaagtatttagaaaggcagcgtcttcagcagtcaagtagggtaggtgaaccattgcatcaaaacttgtccctttaaggtttgaaatgatggattttggtaggaatcagtctagattcgacactgttacagaattttttgtgatgaactgaaatttgcagtcatgtgttcattggatgatgaaacaggactggaagacatgtcatttatgagacactatacagtattcttgcaataagtgtatttggattgcaattttccattgaagtaatttgaatttctttattcttcttaatcatgaattatcctgattttgtggttctgtttaaggtttgacatatgacatgtgctattaacctttttatacattattttgaacatcttaatattatttttatatacatattttagatttctgttttcactttgttactttatcttgaagtctgattcacaattcaagtcttttgatccaccaactcttgtacttatttcttttgccatgtcctttttttatgtcatgaagagcaaaggagtagttagtgatatgactggagctgttttaaactcaaatgaggattcggagaggccagatagagctgctgcacgaccctggctggatccgaggattaacatgcttgtatgtgaagtagcttttcttttcctctttatactattcactctaatgaggattaacatgcttgtatgtgaaggagctgttttaaactcaaatgagggttgttgtcatcttttaatctagtggtgtatattgcagaataatcagcacacacacagagatgcatttaatgattctgttactgaaaagagcattgatggatcatacggaagcagtgaatatagttctgtcatttcaagtaatttggtctcaggggctggaagaactggaagtaagctcattgatttaggacatgacaaaacatggttcaaaacagatgggggtgatgcagatactacatctgggcaaagaaatggttttggtctcaagcgtagtttttcaaatcaagaagcaccaaagtccatgaatttggatgcacatcgtcagccaagacaaagcataactaacttacggaacaatgtgatgtcaggtaactggaaaacttctgaagaagagttcacgcgggatgagatgaacaatggtttgattgatcatggacccaatgtattaagcaatttgagcacagacacatggatggctgatgatgaaaatttggtaagcatgccgaaagggtcagttgcactttgccatttattcatccaattgcagtttttctcctactttataacatgaagtgcattcctctttgatttaacatgattttttttgaaatggtgtcccatgtcctccaaattagtaaaactcgtgctgatttgaaaagcacttgtcccacgagtaattgtttcacgtaacaattaagaccagttaatggtgtctaattttaatcagaagactccacttacggtataagactaagcttatcactttttgcactattagaaacatcatttagagtcgtcgtatcccattaaaaagtcacttgtgtaagcttctaatcaatccctatcttgaaagatgggggacattacagaaaatcataaaggagggggcaaattgcttggtagttagtgcaatagaactagtagtgtggtaacatgaaattaaccctattatttaacttttgtatcaaagaagttcttttataatcgttctagggctgaatgcctgaattcccttaagataatatgcctaattttgttgattaaaacaaacactaatttatcaaagtcgagcattccaaagttctaaatatcctaatacaaatgaagaaaaaaaccctaataattaaaccacttaatccctaattaaaaagccttaaatttcagatgcatcacaaattaatacaatgatcttaagttatatatatatatatatatatatatatattattggttatacttgactgatttattttttccgtgtgcaggaaggtgaagatcacctccacctccaaatcacacgcccaattgggacaaaggtcgatagagaaatatccactgttaagaaacaactacctggtttggggggccatccaccatcatcatggcaattgcagaagcaccactcaattgataagttgaatctaaagccagattactctgaaggatttgtgtccactcttagtggcttaccaaccaatgcaagttctttggctgtcaagaaggggaaccaatcctttatgtccaatgcagtagtaggaatgaccaaatttgtgggacaacaatttgattcgggtgaaactgaatccccttctgggcagtcacctttgcggcaacagtctccatcactgccaggaacagtacaccaccctcattcaatgcaaaacttggctaaccaagaattgccaccgagtctcaaaacctctcaacttttgggaggtcagataatcagtcagcatattagagatcactcacctacccttcgtcccattgtaaaggttggtaacttgcgaagatcacaagaaaaggatatgcagggcccattatcttccatgacctctcttcgaccaaagcttcagcaaaagcagctgaacccatctcagactgaagttactgctacaactaagctgccccagtctaaagtttctttaaccagagaaacttcagaacagttgaccacaaataatttgtcagctgcacctgtcaaaactggaatcatccccaagaaatcaattaccagtaatctggatccaaggaagcgtccatctcagactggggttcagcctactcagtctggtaggcctaccacattaatttcttcagggtctgctgtggcatcaccatcttcattagatcctctgcataatgattcatctacactgccaaaaaaacctaaaggaaaggctggacaaccaccacaaaggctttctactcaaccacctgcttcctctaatgttagcagttcctcagctccgatcttaaatgcggccaaaaataataaaccaaatccaattgcaaaccttttaagcacattggttgcaaagggtttgatatctgccgaaacagaatcaccaactacggtaccaagcgtggcaccaaagggatccaaagatcaaactgagatcattaccaccagttgttctttaccagttacatcaatttctggttctgcagctgtcccagtatcgtcttccgatgatgaggtagatgctgctacaaaaacctgtcttgcctcacctcaatcaactagcacggaaatcagaaatctcattggctttgattttagacctaatgtaattcgagaattccacccatctgtaattagggaattatgggatgatatccctcatcattgcaaagtttgcggtattaagcttaaacaagaagaactctttaatagacacttggagtggcatgccacaagagaacatggtccaattaaggcatcaagaagttggtatgcgaagtcaagtgattggattgctggcaaggcagaatattcatctgagtccgagtttaatgattctgttgatgtacacgatgaaaaaacaggcagcagtcaattggatacgatggttctagcagatgaaaaccagtgtctgtgtgtattgtgcggcgagctatttgaagatgtgtactgtcatgaaaggaatgagtggatgttcaaagggaccatttacatgaactattcagatgtcaatagtgagatggaaagtggaaatgtgggtcccatcattcatgccaaatgcttatcagagaactcaatcatcaccaatttggtaaggcctattatctgatattagtggacgtcttccagtctgatgtttaaattccaaagcttcaatttgtctacttgaaggagtgtgtttaaatttttggtagtaatttgattggacctgctctattttttgcaggataatgattaatggtatgcttctatagcatttgagctttggcatagtcagaaaagcttaccaagggtagcccatccagtttcacattgcatactaaattttgatggcttcttatacttaaaatttattgcctctaactggttcttagagaggtcaaacaaattttgttctttttctttatttttcatgtacccgggaaaaaaatgcgtagtggccaaaatatataaatgcgagataaatttttgcagctaaacttactcgttgatgtggttgtgctaactttttttcacaatggagaggcggattttctgcatttaacatatctggcgataatacttgg

SEQ ID NO: 38 - DNA, 3135 bp, Glycine max
  source 1..3135: mol_type = other DNA
atgagcatggagagcacgcgcagatcgttggacagatccagagaaccaggtcccaagaagcctcgattgatcgacgaactcagcgcccgacagttacctcaacgcactgcggcagttactacgctggcgtctacgaggtttcgagcaaatgacagagactccgaaatcagcgatttgggtcgtggcggtggcggcggcgggtatcagccgcagcctccgccgcaccaagagcttgtgactcaatacaagaccgcacttgctgagctcactttcaattcgaagccgataattaccaacttgactattattgcaggagagaatctctctgctgccaaggcaattgctgctgctgtttgtgctaacattatagaggttccaagtgatcaaaaactgccatctctttacctcttagacagtattgttaagaacattggacgggattatataaagtattttgcttccagactacctgaggtattctgcaaggcttacagacaggttgatccttgtgtgcattcaagtatgaaacatctttttggaacttggaaaggagtctttcctcctcagtcccttcagatgattgagaaggaacttggctttgcacctgcagtcaatggttcggcttcagtgtctgctacagtcaggagtgatttgcagtcacaacgcccacctcatagtatccatgtgaatcccaagtatttagaaaggcagcgtcttcagcagtcaagtaggagcaaaggagtagttagtgatatgactggagctgttttaaactcaaatgaggattcggagaggccagatagagctgctgcacgaccctggctggatccgaggattaacatgcttaataatcagcacacacacagagatgcatttaatgattctgttactgaaaagagcattgatggatcatacggaagcagtgaatatagttctgtcatttcaagtaatttggtctcaggggctggaagaactggaagtaagctcattgatttaggacatgacaaaacatggttcaaaacagatgggggtgatgcagatactacatctgggcaaagaaatggttttggtctcaagcgtagtttttcaaatcaagaagcaccaaagtccatgaatttggatgcacatcgtcagccaagacaaagcataactaacttacggaacaatgtgatgtcaggtaactggaaaacttctgaagaagagttcacgcgggatgagatgaacaatggtttgattgatcatggacccaatgtattaagcaatttgagcacagacacatggatggctgatgatgaaaatttggaaggtgaagatcacctccacctccaaatcacacgcccaattgggacaaaggtcgatagagaaatatccactgttaagaaacaactacctggtttggggggccatccaccatcatcatggcaattgcagaagcaccactcaattgataagttgaatctaaagccagattactctgaaggatttgtgtccactcttagtggcttaccaaccaatgcaagttctttggctgtcaagaaggggaaccaatcctttatgtccaatgcagtagtaggaatgaccaaatttgtgggacaacaatttgattcgggtgaaactgaatccccttctgggcagtcacctttgcggcaacagtctccatcactgccaggaacagtacaccaccctcattcaatgcaaaacttggctaaccaagaattgccaccgagtctcaaaacctctcaacttttgggaggtcagataatcagtcagcatattagagatcactcacctacccttcgtcccattgtaaaggttggtaacttgcgaagatcacaagaaaaggatatgcagggcccattatcttccatgacctctcttcgaccaaagcttcagcaaaagcagctgaacccatctcagactgaagttactgctacaactaagctgccccagtctaaagtttctttaaccagagaaacttcagaacagttgaccacaaataatttgtcagctgcacctgtcaaaactggaatcatccccaagaaatcaattaccagtaatctggatccaaggaagcgtccatctcagactggggttcagcctactcagtctggtaggcctaccacattaatttcttcagggtctgctgtggcatcaccatcttcattagatcctctgcataatgattcatctacactgccaaaaaaacctaaaggaaaggctggacaaccaccacaaaggctttctactcaaccacctgcttcctctaatgttagcagttcctcagctccgatcttaaatgcggccaaaaataataaaccaaatccaattgcaaaccttttaagcacattggttgcaaagggtttgatatctgccgaaacagaatcaccaactacggtaccaagcgtggcaccaaagggatccaaagatcaaactgagatcattaccaccagttgttctttaccagttacatcaatttctggttctgcagctgtcccagtatcgtcttccgatgatgaggtagatgctgctacaaaaacctgtcttgcctcacctcaatcaactagcacggaaatcagaaatctcattggctttgattttagacctaatgtaattcgagaattccacccatctgtaattagggaattatgggatgatatccctcatcattgcaaagtttgcggtattaagcttaaacaagaagaactctttaatagacacttggagtggcatgccacaagagaacatggtccaattaaggcatcaagaagttggtatgcgaagtcaagtgattggattgctggcaaggcagaatattcatctgagtccgagtttaatgattctgttgatgtacacgatgaaaaaacaggcagcagtcaattggatacgatggttctagcagatgaaaaccagtgtctgtgtgtattgtgcggcgagctatttgaagatgtgtactgtcatgaaaggaatgagtggatgttcaaagggaccatttacatgaactattcagatgtcaatagtgagatggaaagtggaaatgtgggtcccatcattcatgccaaatgcttatcagagaactcaatcatcaccaatttgcatttgagctttggcatagtcagaaaagcttaccaagggtag

SEQ ID NO: 39 - protein, 1044 aa, Glycine max
MSMESTRRSLDRSREPGPKKPRLIDELSARQLPQRTAAVTTLASTRFRANDRDSEISDLGRGGGGGGYQPQPPPHQELVTQYKTALAELTFNSKPIITNLTIIAGENLSAAKAIAAAVCANIIEVPSDQKLPSLYLLDSIVKNIGRDYIKYFASRLPEVFCKAYRQVDPCVHSSMKHLFGTWKGVFPPQSLQMIEKELGFAPAVNGSASVSATVRSDLQSQRPPHSIHVNPKYLERQRLQQSSRSKGVVSDMTGAVLNSNEDSERPDRAAARPWLDPRINMLNNQHTHRDAFNDSVTEKSIDGSYGSSEYSSVISSNLVSGAGRTGSKLIDLGHDKTWFKTDGGDADTTSGQRNGFGLKRSFSNQEAPKSMNLDAHRQPRQSITNLRNNVMSGNWKTSEEEFTRDEMNNGLIDHGPNVLSNLSTDTWMADDENLEGEDHLHLQITRPIGTKVDREISTVKKQLPGLGGHPPSSWQLQKHHSIDKLNLKPDYSEGFVSTLSGLPTNASSLAVKKGNQSFMSNAVVGMTKFVGQQFDSGETESPSGQSPLRQQSPSLPGTVHHPHSMQNLANQELPPSLKTSQLLGGQIISQHIRDHSPTLRPIVKVGNLRRSQEKDMQGPLSSMTSLRPKLQQKQLNPSQTEVTATTKLPQSKVSLTRETSEQLTTNNLSAAPVKTGIIPKKSITSNLDPRKRPSQTGVQPTQSGRPTTLISSGSAVASPSSLDPLHNDSSTLPKKPKGKAGQPPQRLSTQPPASSNVSSSSAPILNAAKNNKPNPIANLLSTLVAKGLISAETESPTTVPSVAPKGSKDQTEIITTSCSLPVTSISGSAAVPVSSSDDEVDAATKTCLASPQSTSTEIRNLIGFDFRPNVIREFHPSVIRELWDDIPHHCKVCGIKLKQEELFNRHLEWHATREHGPIKASRSWYAKSSDWIAGKAEYSSESEFNDSVDVHDEKTGSSQLDTMVLADENQCLCVLCGELFEDVYCHERNEWMFKGTIYMNYSDVNSEMESGNVGPIIHAKCLSENSIITNLHLSFGIVRKAYQG

SEQ ID NO: 40 - DNA, 7456 bp, Glycine max
  source 1..7456: mol_type = genomic DNA
aaagcaaaaataaaaaacaaaaaacagcagaatctatggtcaaactatagagtatagacagaagaatttaataatgaaacccatccatatataatagagcctccttcagaaatatgaaaaaaaaaatcacaagtctttgtctgaacaaccagctttactattcaactgttcttcttctttcatctctcgcctatcgaattcgaacccgacacaagatcaaatttttagggctcctcaatcctctctctctctctctctaatctatccctcttcttcttcgcttgtatgaaccggaattagggttttgcctctattatcattgtataatttagaattaggtccttttttgtttcaattttgtaaatttataatttttttgttcgcttaattttgatttttcagttttgatgtaaagaatacaataacagcgatgcaattgctatcctgtaatctagtttctacatcttcgtcgcgcaattgtatgtaaattagggttaggattagggtttttggattgtttttggtgtttgattttgtgggcatggagaattcgcgtagaccgttcgatagatcgcgagaaccgggtccgaagaagccccggttgatggaagagctggatcgggcttcgaattcgggtgcccgacagtttcatcagcgacaagtggcttctgggatcaccacgttgtcgtctgcaaggtttagaacaaacgacagagatcttgaaagcagtgattttggtcgccggggcggtgccggtggcggtggcggcggttatcagccgcagccactgccatttcaggagcttgtggctcagtataaggcagctcttgcagaacttactttcaactcgaaaccgataattaccaacttgactatcattgcaggggagaatcaagctgcggagaaggccattgcagccactgtttgtgctaacattctagaggtgaacttagtttacagtactgtgcagctgtttctctgttcaattgttgttttgttagggttatgtagccaattgctgattgaattgaattgttgtaacatacttcgaatagaaaggaaaacgtgactttttttttttaatgttggtgttcctgatgttgtgatgaatggtgttgtttattgtcatggtatgtgatgtgtgagtattttgcggagaaagcgacaaggttaccgtgtattgtaatttttgcaactaatggaatttggtggtttagtgttgtaaccgttgcggaagttgcatggtgatgtgtgggaagagttgcaattgccatatgaagagatcaaatatttgcaaatgggctttattatgattttcccatttcatttcattggtccactcaaggcttttatctacgagttggctttgggtttctatctctactgtgtaggaagggtaaaacatatatagctttacagtcaaaatgaaaatggagtgaaaaaaattatcattttgtaatttgcaagattatggcagtctggtaatccattggcaactgacaagcagcatcattagagtggtataccgcagttgtcctggtattttcttgactttgtatgcagaggttgatatagatctgtaaaaaaatttaattgaatacaatgtatgaggaaaaaaaattaccaatgcattgttgccaatctcaataaatgttctctcactctctccctctttgtgtcttatatcgtaatatacattgtaattgtacagtattttgtcacttctcgtgaaacggtgcttctgttttgtatatcaccagccaatgcatcctttcaagtcttacctctctggtttatttttctttttaggttataggaaaatacagtttacctgcagtttttattttttgagtggcggtggtggttatattttagagagggactttgtttagcctagttgtacaaagacattatatttttatcatttcatttttgcactagtggatttgctttgattgagggtgttgtatgtagcatgcaacttatgataacagagatgtatccctcaatttgaatcagatagcagaaggagcaagttgctatttattatgatctggaagattggaccacattcgtttgtaggaattgagattgtgaccaagacgtttcatatttgttgagtttccaaagactactgtaatagatcttttacattattaaggcttttcatatatgttgtactagttccatgaagtgcaacatatgatctaaggaatggaataactaacaattttgaacaggcatgacattttatggaaacttttatgtaactttagatccaattgccaatgataatattcctggattttgattttatttgctgctgttttccttgtgcgagttgtatattattggtttctgttacttttcataacatatgtcaggctgtctttttcttgttctttggtcattagttttctttttgttatactaaaaaaaggtcctttttcatgaattgtgttgatacttttccaggttcctagtgatcaaaagctgccatcactttatctcttagacagtattgttaagaatattgggcgggattatataaaatactttgctgccaggctacctgaggtcagatatttattcttcttttagcctgttattgatttccatttatcttctctcttatagtgtgcttcttatacatttttttatgacagtctatgatgaatctgtcagattattatgttatgcaaaatgtactcatgtcctgcacgtctttctcaagatatcttgtctgatttttagatgcctatttttaagttggtagtttctaattactcctttacattttaggtattcatcaaagcatacagacaggttgatccttctgtccatcaaagtatgaggcatctttttggaacttggaaaggagtctttcctcctcagacccttcaggttattgagaaggaattgggcttcactcctgcagtcaatggctcatcttctgcttctgctacactcaggagtgattcacagtcacaacgtccacctcatagtatccatgtgaatcccaaatacttagaaagacagcgtctacagcagtccagcagggtaggtgaaatgattgcacctcaatttattcttcaaagttttaaaacattgatttgactgggataggtcaagccatgatgcaaatacagaatttatgttgatgaattggcacttgaattcattccttgcatgatgaaactggattggatggatgacttgatatcatatttttaattatgtttgttgcttgttgggacaccacaatattaatgcaaacattgtatactgtattgaacctttccaacagactaatttacacacctttatcctccttatcattcatgaaacacttttttggctgacaatttgtcttgtgggatatctaagacaattcctctctatacaactagtcaaccaactctagcctggaagcataaactcttcccccccagttgggatgggtttgttatagagccaacttcttatacctgtcactgttcttagtgttctattatccatacaatgctggccttgcaaagatgataatttaactgttataccaatagacattctgtcatgttacttaggttgccatattctttcttatttcacatctccattgcttaatttttttggaatctactttctctgtcctgaagactaaaggagttgttgatgatatcactggagcgatttcaaatacaaatgatgacccagagatgcctggtagaactttgggcgttggacgaccatgggtggatcctagtgttactgttaacatccattcacgtgaagtaaaatttctttgcccttattttgttcactctgtcagttttcatgatttaatgtgataatgtacattgcagaatgagcaatgcgctcacagaggtgcattcaatgattctgttctggaaaagagcattggtgcatcctatggaagcaatgaatatggttccaatatttcaaggaatttgggcttgggtatccgccgacctggtggtagggtgactgagtcaggacatgacaaaagttggtacagcaaatctggtgtcgttgcagggaccatgtctgggcaaggaaatggtttaggtcttaagtatagttttttgaatactgaagcaccaaagtccatgattttggatgtgcatcatcagcctacacagaacataagtagcacacgtaccagtgtgatctctgcgagctggaaaaattctgaggaagaggagtacacatgggatgagatgaactctggcctgactgttcatggtgcatccactgttagcaacttgagcaaaaatagttggactgctgatgatgagaatttggtaagcttggtagaatttggtgatttgggtatcagactgatgcagaacagaactgggacgatattaaaaaccaagatttagttgaaaaattctatatgaaattgagtttcaagataaaataatatgccaactgtaggtggccaggacaccctaatttatacaacccaagcttaaaagactcttaaaatcctaataaaaaagaaaaagaaatgaagtcctacctagatttgggattgcatatcccgggagtctgggacccaaacaacatgacccttaataactgagttaaaccctaaatccctacaaacaaagatccctaaattgttgtctcatcagttaatacaataatcctggttttatctgctcatttagcattgttaataatagtatgtagcacattattgatatattatgtatatatttttctcacgtgcaggaagctgaagattgcctcgaaatcagaaatccttttcgggctaatgttgatagagaaatgtccattgaatctcaagccactgaaaagaaacaattaccttcatctcatcatcatccatcactatcatggcaattgcaggagcagcaatctattgatgaattgaatcggaaggcaggtcactcggacagatttgtgtccacacatggtgccataccagccaatgcaaattcttctgctgccaggatggtcaatcagcctttcctgtcaaatgcaacaataggattgccaggaattgcaggccaatttcattctttgggagctgaaaacccttcagggcagtcacctttacaacaaaggtctccgtcaccgccagtaagtgtgcacccttctcatccactgcaaaatttggctgagcaagaccttccccagaatcataaagcatctcaatttctgggaggtctgcagagccaatatattaaagattcttcacccgcccctcctcctagtattcaggttggtaacttacaaagttcacaactgaaaaacttgcagggtccattctcttcaacgacttttcaggcaaggcatcagcaccaactgggctcttctcacaatgaagttactgttaagactgagaagccacccatgtccgaagtgcctttagccagagaaactaaaagtaatctggatacagggaatcttccatctcggttaggggttcggccttcacggtcaggtggcccttcccctgccacattaatttcttcagtgtccaaaattgcatcaccatcttcgttaggtccttccagcgataatgtatctgttcttccaaaaatacctctaagaaaggctggaccacctagggcatctactctaccaccagcttcctctaatgtcagcagtgcctcagcccagacatcaagtgataccaataataccttaaatccaattgcaaaccttttaagctctttagttgcaaaaggtttgatatctgcagaaacagagtcaccagctaaggtgccttcagagttgcttactcgattggaagaacaaagcgatagcattaccaccactagctctttgcctgtggcttcagtgtctggttctgccactgtccctgttccttctaccaaggatgaggtagacgacactgcaagaacgcctatatcattgtctgaatcaaccagcccaggaatcagaaatctcattggcttagagtttaagcctgatgtaattcgagaattccattcatctgtagttagtggattatttgataattttccacatcagtgcagcatatgtggccataagcttagattccaagaacagttcaatagacacttgaagtggcatgccacaagagaaagcgaagaaaatggtttaattagtgcatctagatggtatctgaagtcaaatgattggattcttggcaaggctgagtatccatctgagaatgagtttactgattctgtagatacatatggcaaagaagcagacaaaagtcaagaggatgctatggttctagcagatgaaaaacaatgcttgtgtgtgttgtgtggtgagctatttgaagatttttactgtcaggaaactggtgagtggatgttcaaaggggctgtttacctggccaactctgatagcaaaagtgagatgggaattagagatgtgagtactggaaggggtcctatcattcatgcaagttgcttatcagataactcagtttccagtgtcctcgagacggtaaggcatattacttgatttatctggacagaaatggggttatctagtttaagttcaaagcttcctttccccctctatttgcttactttgaattgaaggacttccattgctaataatatttgggcccattatgttttgcagggacaggattaaagcaagtgtagagcattggagctctgctgtagcaagaacttggaagccgattgaacccaggtttactttgcattttccaaatttgatggctcttgatgctaaatttatttctctaattggtttctagagctgtcaaacaaattttgtgcactttttttgttttccttagtttcttttgtactcaggaagaagaggaaattccttagtgttcaaagtatataaatgcgatagtttttgcagctcaacttttgctcattgatgtggttgcctaacttcattttaaatgaagtggatttttgttttctgaacagcatctggttgtaatatggagcaaagcactatggccatagttatcgcaaatgttggatggcattctctgtatcattggaatcgcaacaattctgattccctagtatgtgctttccttcccttcccccccccccccctcctttgctaggggttttggaatttggggctttcctaatatccaaatttcagttttatgaacaacaatggatcttggttttagtttggccgagtactgagattttccttattggtggtcaatgaagaaaaagatgtacattgccgagccattattgtttaatgttgattctgccaagtacaccatttagttggagaattaggtaccctgatgaatgatactcgcacaacgtttgtttgtacaagaaatttctgcaacgcgtagtttgcgttttgatttttttgccatttacttttgctactgtcacatttctccctgttccttgttaaagagttgatatgaaaaggttttttttgc

SEQ ID NO: 41 - DNA, 3108 bp, Glycine max
  source 1..3108: mol_type = other DNA
atggagaattcgcgtagaccgttcgatagatcgcgagaaccgggtccgaagaagccccggttgatggaagagctggatcgggcttcgaattcgggtgcccgacagtttcatcagcgacaagtggcttctgggatcaccacgttgtcgtctgcaaggtttagaacaaacgacagagatcttgaaagcagtgattttggtcgccggggcggtgccggtggcggtggcggcggttatcagccgcagccactgccatttcaggagcttgtggctcagtataaggcagctcttgcagaacttactttcaactcgaaaccgataattaccaacttgactatcattgcaggggagaatcaagctgcggagaaggccattgcagccactgtttgtgctaacattctagaggttcctagtgatcaaaagctgccatcactttatctcttagacagtattgttaagaatattgggcgggattatataaaatactttgctgccaggctacctgaggtattcatcaaagcatacagacaggttgatccttctgtccatcaaagtatgaggcatctttttggaacttggaaaggagtctttcctcctcagacccttcaggttattgagaaggaattgggcttcactcctgcagtcaatggctcatcttctgcttctgctacactcaggagtgattcacagtcacaacgtccacctcatagtatccatgtgaatcccaaatacttagaaagacagcgtctacagcagtccagcaggactaaaggagttgttgatgatatcactggagcgatttcaaatacaaatgatgacccagagatgcctggtagaactttgggcgttggacgaccatgggtggatcctagtgttactgttaacatccattcacgtgaaaatgagcaatgcgctcacagaggtgcattcaatgattctgttctggaaaagagcattggtgcatcctatggaagcaatgaatatggttccaatatttcaaggaatttgggcttgggtatccgccgacctggtggtagggtgactgagtcaggacatgacaaaagttggtacagcaaatctggtgtcgttgcagggaccatgtctgggcaaggaaatggtttaggtcttaagtatagttttttgaatactgaagcaccaaagtccatgattttggatgtgcatcatcagcctacacagaacataagtagcacacgtaccagtgtgatctctgcgagctggaaaaattctgaggaagaggagtacacatgggatgagatgaactctggcctgactgttcatggtgcatccactgttagcaacttgagcaaaaatagttggactgctgatgatgagaatttggaagctgaagattgcctcgaaatcagaaatccttttcgggctaatgttgatagagaaatgtccattgaatctcaagccactgaaaagaaacaattaccttcatctcatcatcatccatcactatcatggcaattgcaggagcagcaatctattgatgaattgaatcggaaggcaggtcactcggacagatttgtgtccacacatggtgccataccagccaatgcaaattcttctgctgccaggatggtcaatcagcctttcctgtcaaatgcaacaataggattgccaggaattgcaggccaatttcattctttgggagctgaaaacccttcagggcagtcacctttacaacaaaggtctccgtcaccgccagtaagtgtgcacccttctcatccactgcaaaatttggctgagcaagaccttccccagaatcataaagcatctcaatttctgggaggtctgcagagccaatatattaaagattcttcacccgcccctcctcctagtattcaggttggtaacttacaaagttcacaactgaaaaacttgcagggtccattctcttcaacgacttttcaggcaaggcatcagcaccaactgggctcttctcacaatgaagttactgttaagactgagaagccacccatgtccgaagtgcctttagccagagaaactaaaagtaatctggatacagggaatcttccatctcggttaggggttcggccttcacggtcaggtggcccttcccctgccacattaatttcttcagtgtccaaaattgcatcaccatcttcgttaggtccttccagcgataatgtatctgttcttccaaaaatacctctaagaaaggctggaccacctagggcatctactctaccaccagcttcctctaatgtcagcagtgcctcagcccagacatcaagtgataccaataataccttaaatccaattgcaaaccttttaagctctttagttgcaaaaggtttgatatctgcagaaacagagtcaccagctaaggtgccttcagagttgcttactcgattggaagaacaaagcgatagcattaccaccactagctctttgcctgtggcttcagtgtctggttctgccactgtccctgttccttctaccaaggatgaggtagacgacactgcaagaacgcctatatcattgtctgaatcaaccagcccaggaatcagaaatctcattggcttagagtttaagcctgatgtaattcgagaattccattcatctgtagttagtggattatttgataattttccacatcagtgcagcatatgtggccataagcttagattccaagaacagttcaatagacacttgaagtggcatgccacaagagaaagcgaagaaaatggtttaattagtgcatctagatggtatctgaagtcaaatgattggattcttggcaaggctgagtatccatctgagaatgagtttactgattctgtagatacatatggcaaagaagcagacaaaagtcaagaggatgctatggttctagcagatgaaaaacaatgcttgtgtgtgttgtgtggtgagctatttgaagatttttactgtcaggaaactggtgagtggatgttcaaaggggctgtttacctggccaactctgatagcaaaagtgagatgggaattagagatgtgagtactggaaggggtcctatcattcatgcaagttgcttatcagataactcagtttccagtgtcctcgagacgggacaggattaa

SEQ ID NO: 42 - protein, 1035 aa, Glycine max
MENSRRPFDRSREPGPKKPRLMEELDRASNSGARQFHQRQVASGITTLSSARFRTNDRDLESSDFGRRGGAGGGGGGYQPQPLPFQELVAQYKAALAELTFNSKPIITNLTIIAGENQAAEKAIAATVCANILEVPSDQKLPSLYLLDSIVKNIGRDYIKYFAARLPEVFIKAYRQVDPSVHQSMRHLFGTWKGVFPPQTLQVIEKELGFTPAVNGSSSASATLRSDSQSQRPPHSIHVNPKYLERQRLQQSSRTKGVVDDITGAISNTNDDPEMPGRTLGVGRPWVDPSVTVNIHSRENEQCAHRGAFNDSVLEKSIGASYGSNEYGSNISRNLGLGIRRPGGRVTESGHDKSWYSKSGVVAGTMSGQGNGLGLKYSFLNTEAPKSMILDVHHQPTQNISSTRTSVISASWKNSEEEEYTWDEMNSGLTVHGASTVSNLSKNSWTADDENLEAEDCLEIRNPFRANVDREMSIESQATEKKQLPSSHHHPSLSWQLQEQQSIDELNRKAGHSDRFVSTHGAIPANANSSAARMVNQPFLSNATIGLPGIAGQFHSLGAENPSGQSPLQQRSPSPPVSVHPSHPLQNLAEQDLPQNHKASQFLGGLQSQYIKDSSPAPPPSIQVGNLQSSQLKNLQGPFSSTTFQARHQHQLGSSHNEVTVKTEKPPMSEVPLARETKSNLDTGNLPSRLGVRPSRSGGPSPATLISSVSKIASPSSLGPSSDNVSVLPKIPLRKAGPPRASTLPPASSNVSSASAQTSSDTNNTLNPIANLLSSLVAKGLISAETESPAKVPSELLTRLEEQSDSITTTSSLPVASVSGSATVPVPSTKDEVDDTARTPISLSESTSPGIRNLIGLEFKPDVIREFHSSVVSGLFDNFPHQCSICGHKLRFQEQFNRHLKWHATRESEENGLISASRWYLKSNDWILGKAEYPSENEFTDSVDTYGKEADKSQEDAMVLADEKQCLCVLCGELFEDFYCQETGEWMFKGAVYLANSDSKSEMGIRDVSTGRGPIIHASCLSDNSVSSVLETGQD

SEQ ID NO: 43 - DNA, 6922 bp, Glycine max
  source 1..6922: mol_type = genomic DNA
gcgtggcacgcttatgacttaactgatgagaggagtgcaaatttggggctccagagttcagtcccatacttccatcccatcccaccccacgctccctcaaaacaaaaagaagggtttcaaatttcaattcataactcaccaatcccatagcgtcgccgtttctacaccgtccgatttccattccccatttccaatttcaattcccctctctatgccgccgcttcctacgccgtaatttcgtaacggcgacgcatcctcccaaaccctaaccctaaccctaacctccccatctctccgaaaccgatatttatttatgttttcacaaaacatgattctccctcccgaaaaccctagacccgcggggttcgcttccaaacccatgggcaatgagatcgcgaagcctccgccgtcgattctcgttggccgcttcaaggctctcctcaagcagcgcgacgatgaactccgagccacatccgtccccgtcccgccgccgtccaccgacgaaatcgtccagatttacgaactcctgctgtcggaactcacgtgcaatttgaaacccatcatcaccgatctcacaattatcgccgaacagcagagggagcacgccaaaggtatcgctgacgcaatctgcgctcggattctcgaggtccgtctccgttgccgccgaaatttcttcttctacctcttgagatttgtacaatgtcatggaatcgttttattagttcgggtcaaaattatccctctcttgttgcttcattttttttaatgtcacaattcgtgctccaatgcttcgtttttttttttagttctgcgtttttttttatgcttttggcttttttctgaaaaatgaaggtcgcccataggccgagggaacgattgggttatacgctcaaactgaccgttgcttcgaacgttatattaaagcgtaaagcaaaaataatttgttgctactggtaatttaaagaataggaaatagtacaatctaaatgagtagatgtggggagtttctaacgcattgtgaagattgtgtacaatgtgtttgagggcttttttcaatttcaggaaagtcaagggcatacgaattgaaattgaattttggaatactacttctggaaaggctcaggaatgccattaaaggaaagttgttctttcatttttgctgaaatgaagtaatttctgcaaaataagaaagggttgttgaatgaacgagtatttaattcagtatgaaaacgttttataattctttaataagtatcgggtcatgcataatgagatcatagtatacaaataatacaaaatgtaaagaaattaacgactttttaaatcacccaagtccattgtgagtttttgttgatgcgtagtagaacattagtatttctactttcagaattggaatctctagtttatttatatatctctggctatgccactgtcacatgggaattcaattttttggagatatgcttttgcaggttccagtagatcaaaagcttccttcactctatctgttggacagtattgtgaagaattttgggcaggaatacattagatacttctctttacgtcttcctgaagtaggtcaactagagttgctgtattgtttttaattttattatttattccacaactgaaacgtctgcttcatttttctcctcaggttttctgtgaggcatacaggcaggttcagcctagtttgcattctgcaatgcgccatctttttggtacttggtcaaaagtctttccaccttcagtcctgcacaagattgaagctgagttgcaattttctcaagcagttaatactcaatcatccaccccgaatcctgtcagggcttctgaatcatctcgaccaagtcatggcatacatgttaatccaaaatacttgcggcagttggagcgctctactgtggatagtgtaagcttttttatgacatctctgtcttccacacaaactatacaaggactaggctaagtcttgattagcttgctggtaccatggggttgtatatttcactaatggaattgtttattgttttcatgtaaccataacaaaaatgtaatttttgtggtaaatatctcattttttctcatggtaggatctgtctgtatccatcattcctttcctgcatgtctgtccatctctctgtcttcccaaaacttttccccattttcttttgggataaactatgttctccaaaccttgtatttgctattttgatgttaagtttagcattggggtgggggtggggggggtggaggaatcaatgtagacacaacccatatcttgtgatggattgtaaagctctttggaaaatttattttatttttgcttacctctcgccccctctttccttaagagcttttatgaactatgactaagggcctgtatgtttactatctaaattcaattttacagtgtgacacacgttcaaatggaaaatctggttaaaatgaattggtactagcatcccaataacatttagcagttcaaatacaaaacttccataatcaattttaccaaacattaaaacaaatgatcactaagagcatgtttgcttcctggtttaggttgaatggatccatgtccatcagaagctactacatgtagcttctgcatatttaaggtgttagacatggagagagggtagttgatccatgttaaataacctgttccgaacatgcactaagtcctacacatgcaatcttttgtttatttattgtgatttactgaatttcctaactctaatatgggatattttttccctgattcctctctaaattgattattccggtgtccttagattacagtgacagattaaatacttctgccttgtctccctttgagagcctccaatttgcatattgttaatggcaacaaaatctgtttattgaatagtatgtctggcaacatatcagacagacataatttatctgttcttctctgtattatttttaattattttgacatctgggagatttgtcatcctgtcaagtgatatttttccctcccctccccttctctagtcagccgtatagttttctcttctcatattttatgtctgggggcgaatgaagtaagtactgtatccattgattctaggaagaaactgctgctgaatatttagcattcaatagagaaaattttcaacccttgtttgcaagaaaaaatcttgcaaacagatataatgagtggcattgtgacacagtatttgttttatcctcacaggctagtaagacacatcaatttttatcaagcagtagcaggcttggaatatcctcgtcgtcccctttaagaattggagttgataggcctttgtctgcatctatagatgaatatgcagtggacaatcctggtgttgattatggagtagccaaagcattaggtagagatgtggatttgactgaatggcagcgaaagctgtattctggtgatggtcgtaatcgatttccaacttcttttacgtacagccttagtaatggacatcagcgtcaaagttcaagagctttaattgatgcatatggcagtgacaaaagtcaagaaacttcaagcagtaagtctttgttagttgaacgtctggatagaaatggtatagacaaagtgttatcaacatcatggcagaatactgaagaggaggagtttgactgggaaaatatgagtccgaccttaatagaccatagtaggaataacagtttattgccttcaacttttggtttctctagggaaaggcctggcgttgcagctaatgcaactttgtcagagcaagatactaggaaggggtggtctagtggatctcagcttcctccagtagatgattcttctgccatagcagaagatgcatttgcctcttcaactgtatgtttcctcacttctttactttattctttctgatagatattgttttagtatggtttatgatattgaatttgaatttcccaattaaagtatatacctctaataagatgccattgatgctttgggttctggtgataattttctgatgatgaggatctggatagctttgtgtggataggcttaatcaaatgcatgaaattgtttcatacttgtctggcaattgatctctaatttagttcacggatattcactaatgccacatttttttactgtaagtggatagctttgtgtgggtagggcttaatcaaatgcatgaaattctttcatacttctctggcaattggtctcttctctaattttagttcacacattcactaatactacattttttacagtagaatactggcttttagaattgtttttttggtgcatattctgaatcactatttggcattgactccccttattttattttaataaaaactagaaattagttcaatgtaggttttctgtgtattgtccttgcctcaaaagagcttttgcgtgaggatgtgttggagagagatgtaatataaaaccatttacgtgtcttcacgtaacagtttaaactttagtaacgataatttgttgtgaaacatcattttagtttatttaatttgaaaggaaataggagttgccaataatttgctaatggaattggaaatttgtttattcttgctgcagttttgtcgtgcacccccaggtcaggtacctggatcccaaaatcaaatcaatcatagcctgggctcctctcaacctcatgatgcttggaagatttctcatcatccgtctaacatttttagtaatagggggcgggcaaggaatcttatgatccctccaattgacaacattcgcaatacagataataacccatattgggttcgacctgcagtgtcgaggatggaagctcacccatcagttttgccagcaccttttgaaatgaggccttctgtaaatgtgaatgtcactcggccacccatcataaatccgttgcaaaaacatgttaggagtcagtttgatgccatgaatactagtaatcctattgcgaaccatgttgtaaataaatcttcatttatgccagaacagtcatttgacagtgttgagaacaaggatgcaagtattttaaagatacatcagttgcctaatcagctttctggagtaatttcgtccaaccagcaaaatcatgggcaggcaccacaactacagttttttccttctcaggatccatcaacctcacaatttagtcatgggagttcatcgcagggacatggtgtttctataagtacagccatgtcaaatccattaccagttctaccattccctttaccatttcaaagtatttcaaataaccctttacatttacagggtggagcccatccacctttacctcctggtcgccctcctgctccatctcaaatgataccccatccaaatgctggtgcatttatgccaagccaacagccaactgttggatatactaatttaattagttctctaatgtctcaaggcgtgatctcattggctaaccagctgcctgcacaggtatctaatatttatatctatcagaagctcatttctttcattttttttcctttttgatttgattgcgccacccaaggttaattaaatattttttattaggattctgttggaactgagtttaatccagatattctgaagattcgtcatgagtctgcagtcaatgccttatatggtgatctccctagacaatgcacaacttgtgcgctccgattcaaatgccaagaggaacacagtagtcatatggattggcatgtgactaagaatcggatgtctaaaagccgtaagcagaaaccctctcgtaagtggtttgttagtgaccggatgtggctcagtggtgcagaggcattgggaacagaatcagctccaggttttttgcctaccgaaaccatagaagaaatgaaagatcatgaggagttggcagtgccggcagaagaagaccagaatacatgtgcattatgcggagagccttttgatgagttttacagtgatgaaatggaggaatggatgtatagaggagctgtatacctcaatgcacctttaggaataacagcaggcatggataggtcacagttaggccccataattcatgctaaatgcagatcagaatctaatatggccacctctgaagatcttgggctggatgaaaaggtatctttttctctatctatattattatgcactgcactagcctcagttttttgtcaagcatttgctccattttaatattttttggtcgcaatattttcatctgtatggtgattatgcatgtctgcatatgcttcaatgaatcttcagtaaatattcttctttttttttctttatatgcagggtgccgatgaagagggttcccaaaggaaacgaatgcggagttgaccaatgtaaccagttttgtgcattgtaacttatgattttccattagtgtatcttagagtagagttgaatcttgcaataccatgaccattctgccaaagtcatgtacatgtatagttggactggtaacgcgcatattttcttacagtcaaaggttacattttaacttgaaattattttgtctcttgcaattttgtccccaaatcaaaaactattagtacattgttatttctcggatggggcgcaggattttaaactatgcagcgatatactatcaggttgatcagaaattattatacttgctactgattagctggtaattggcatagaattaattgtttggacctactgtttgtatttttatcgaactcatgcataagtgagagcaaagtcatagatactcttaccaatttcttcaaataagtttgttgtgctagccatgtctctcccttcgcccttattcgcttcattatggttgctagctgtcagggaatacaaatacaggtcgaaggcttcacatggactaattttcttttcatctaatctaggatgaaacatgacattctattcacttcaagatatgcaagcgtattaattttaaaatactaatttatattaatt

SEQ ID NO: 44 - DNA, 2769 bp, Glycine max
  source 1..2769: mol_type = other DNA
atgttttcacaaaacatgattctccctcccgaaaaccctagacccgcggggttcgcttccaaacccatgggcaatgagatcgcgaagcctccgccgtcgattctcgttggccgcttcaaggctctcctcaagcagcgcgacgatgaactccgagccacatccgtccccgtcccgccgccgtccaccgacgaaatcgtccagatttacgaactcctgctgtcggaactcacgtgcaatttgaaacccatcatcaccgatctcacaattatcgccgaacagcagagggagcacgccaaaggtatcgctgacgcaatctgcgctcggattctcgaggttccagtagatcaaaagcttccttcactctatctgttggacagtattgtgaagaattttgggcaggaatacattagatacttctctttacgtcttcctgaagttttctgtgaggcatacaggcaggttcagcctagtttgcattctgcaatgcgccatctttttggtacttggtcaaaagtctttccaccttcagtcctgcacaagattgaagctgagttgcaattttctcaagcagttaatactcaatcatccaccccgaatcctgtcagggcttctgaatcatctcgaccaagtcatggcatacatgttaatccaaaatacttgcggcagttggagcgctctactgtggatagtgctagtaagacacatcaatttttatcaagcagtagcaggcttggaatatcctcgtcgtcccctttaagaattggagttgataggcctttgtctgcatctatagatgaatatgcagtggacaatcctggtgttgattatggagtagccaaagcattaggtagagatgtggatttgactgaatggcagcgaaagctgtattctggtgatggtcgtaatcgatttccaacttcttttacgtacagccttagtaatggacatcagcgtcaaagttcaagagctttaattgatgcatatggcagtgacaaaagtcaagaaacttcaagcagtaagtctttgttagttgaacgtctggatagaaatggtatagacaaagtgttatcaacatcatggcagaatactgaagaggaggagtttgactgggaaaatatgagtccgaccttaatagaccatagtaggaataacagtttattgccttcaacttttggtttctctagggaaaggcctggcgttgcagctaatgcaactttgtcagagcaagatactaggaaggggtggtctagtggatctcagcttcctccagtagatgattcttctgccatagcagaagatgcatttgcctcttcaactttttgtcgtgcacccccaggtcaggtacctggatcccaaaatcaaatcaatcatagcctgggctcctctcaacctcatgatgcttggaagatttctcatcatccgtctaacatttttagtaatagggggcgggcaaggaatcttatgatccctccaattgacaacattcgcaatacagataataacccatattgggttcgacctgcagtgtcgaggatggaagctcacccatcagttttgccagcaccttttgaaatgaggccttctgtaaatgtgaatgtcactcggccacccatcataaatccgttgcaaaaacatgttaggagtcagtttgatgccatgaatactagtaatcctattgcgaaccatgttgtaaataaatcttcatttatgccagaacagtcatttgacagtgttgagaacaaggatgcaagtattttaaagatacatcagttgcctaatcagctttctggagtaatttcgtccaaccagcaaaatcatgggcaggcaccacaactacagttttttccttctcaggatccatcaacctcacaatttagtcatgggagttcatcgcagggacatggtgtttctataagtacagccatgtcaaatccattaccagttctaccattccctttaccatttcaaagtatttcaaataaccctttacatttacagggtggagcccatccacctttacctcctggtcgccctcctgctccatctcaaatgataccccatccaaatgctggtgcatttatgccaagccaacagccaactgttggatatactaatttaattagttctctaatgtctcaaggcgtgatctcattggctaaccagctgcctgcacaggattctgttggaactgagtttaatccagatattctgaagattcgtcatgagtctgcagtcaatgccttatatggtgatctccctagacaatgcacaacttgtgcgctccgattcaaatgccaagaggaacacagtagtcatatggattggcatgtgactaagaatcggatgtctaaaagccgtaagcagaaaccctctcgtaagtggtttgttagtgaccggatgtggctcagtggtgcagaggcattgggaacagaatcagctccaggttttttgcctaccgaaaccatagaagaaatgaaagatcatgaggagttggcagtgccggcagaagaagaccagaatacatgtgcattatgcggagagccttttgatgagttttacagtgatgaaatggaggaatggatgtatagaggagctgtatacctcaatgcacctttaggaataacagcaggcatggataggtcacagttaggccccataattcatgctaaatgcagatcagaatctaatatggccacctctgaagatcttgggctggatgaaaagggtgccgatgaagagggttcccaaaggaaacgaatgcggagttga

SEQ ID NO: 45 - protein, 922 aa, Glycine max
MFSQNMILPPENPRPAGFASKPMGNEIAKPPPSILVGRFKALLKQRDDELRATSVPVPPPSTDEIVQIYELLLSELTCNLKPIITDLTIIAEQQREHAKGIADAICARILEVPVDQKLPSLYLLDSIVKNFGQEYIRYFSLRLPEVFCEAYRQVQPSLHSAMRHLFGTWSKVFPPSVLHKIEAELQFSQAVNTQSSTPNPVRASESSRPSHGIHVNPKYLRQLERSTVDSASKTHQFLSSSSRLGISSSSPLRIGVDRPLSASIDEYAVDNPGVDYGVAKALGRDVDLTEWQRKLYSGDGRNRFPTSFTYSLSNGHQRQSSRALIDAYGSDKSQETSSSKSLLVERLDRNGIDKVLSTSWQNTEEEEFDWENMSPTLIDHSRNNSLLPSTFGFSRERPGVAANATLSEQDTRKGWSSGSQLPPVDDSSAIAEDAFASSTFCRAPPGQVPGSQNQINHSLGSSQPHDAWKISHHPSNIFSNRGRARNLMIPPIDNIRNTDNNPYWVRPAVSRMEAHPSVLPAPFEMRPSVNVNVTRPPIINPLQKHVRSQFDAMNTSNPIANHVVNKSSFMPEQSFDSVENKDASILKIHQLPNQLSGVISSNQQNHGQAPQLQFFPSQDPSTSQFSHGSSSQGHGVSISTAMSNPLPVLPFPLPFQSISNNPLHLQGGAHPPLPPGRPPAPSQMIPHPNAGAFMPSQQPTVGYTNLISSLMSQGVISLANQLPAQDSVGTEFNPDILKIRHESAVNALYGDLPRQCTTCALRFKCQEEHSSHMDWHVTKNRMSKSRKQKPSRKWFVSDRMWLSGAEALGTESAPGFLPTETIEEMKDHEELAVPAEEDQNTCALCGEPFDEFYSDEMEEWMYRGAVYLNAPLGITAGMDRSQLGPIIHAKCRSESNMATSEDLGLDEKGADEEGSQRKRMRS

SEQ ID NO: 46 - DNA, 3508 bp, Glycine max
  source 1..3508: mol_type = genomic DNA
gattataaaagattaaaattcaaaagattagatacgcgagaaaaaagacaagattcactttgaattgaaaaatttgctcctatttatgaaacccctcggccaaggcacggcatacatgttaatccgaaatacttgcggcagtcaactgtggatagtgtaagctgttttgtgacatctctgtcttcaatacaagggacgaggctgacgcttcattagctgataccctaactgtaagtttgtacatttcattaatggaattgtttattgtttacacgtaaccatcgttcctttcctgcatcttggtttgtctctctgtaatgtttccccattttcttttggtataaattatgttctccaaactttgtatttgccgaagactggaattttgatgctatgtttagcaatggtggatggagacatgtcacgtatattttgttgggttgtcaagttgtgaaaaagttttattagattgtctttagaccacatggttcccgttatggaatcaagtgttattaggttttgcaaatcaattttacccagtatatcacactttctaatggaaaatctgtggttaaaactaatttgtgcatgtatcccaataacatttaacagttcaaaatcaaatttgacaaattaaatactttttccgtgtctcctcctttgagagtctccaatttgcatgttgtgcgcggcaacaaaattctgtttatggattagtatggttggaaacatattagacatacataatttatcttttttttctctctgtaatttcttaattgttttgacatctgggaaattttttatcctggttagtgatattttttttcctaacccctttctagtcatgtagtttttttctcgtcatgttttatgtgaagtaagtaatgaatccattgattctaggaagtgattgctgctcaatatttagcattcattcaacagagaaaatttccaacccttatttgggggaaaattcatgcaaacagatataatgagtggcattgtgacacagtatttgttttatccttacaagttggtagtgaatgctaataattcaaactttggctttgtaggtagtaagacacatcaatttttatcaagcagtagcaggctttgagtattgctgtccccttcaggaattggacttgataggcctttgtctgcatccatgagtgaatatgcagttgctagctggttatttgagagagactatcctcatccggctgttgatttaggagtagccaaagcattaggtagacatgtggaggttagtgaacggcaacgaaaacgaaaacaatttccagcttctatcacacacagctttatcaatggacagcagcatcaaagtccaagagctttaattgaggcatatggtagtgacaaaagccaacaaacttcaagcagcaagtctttgttagttgaaccactggatataaatggtatagacaacatgtcaacttcattgcagaatactgaagaggaggagtttgattgggaagatatgagtccaaccttattgtcttcaactattggtttctccagggaaaggcctgttgttccagctaatgcaactttgtcagagcaagatgccagcaagggatggggtattagagatgtacgtaatataaaaccatatatgtgtttttagttttcaccaacagtttaaactttagtagttcatgaaattttttatgtaatttgaaaggaaataagacttgccaataccttgcctactagaattgtaaatttatttactctctttgcagtttcgtcatgcaccattgggtgaggtacctagattccaaaatcaaatcaattatagtctaggctcttctcaacctcatgatccttcgaagatttctcttcatctgtctaactcatcacaacataactttaataatagggggcgagcaaggagtctcacgatccctcctgttgataacattcgaaatactgatataaacccatatctggttcgacctacatcttttgaaataaggccttctacaaatttgaatgtcactcgacctcctaccttaaatccaataactccattgcaaaaacgtgttaggaatcagtttgaagctacacatactagtgaccctattgtgaaccatgtaaataaatcttcgtttatgccagaacagtcatttgaccgtattgagaacaaggatgcaagtatttctaagatacatcagttgcctaatcagcatcccggactaatttcatccaaccagcaaaatcacgggcaagcaccacagctgcagttttttccatctcaggatccaacgccctctcaatttagtcctgggagttcattgcagctacatgatgcttctattagtacagccatgtcaaatccgttacatggtatacaattcccttcaccagtccaaagctttgcaaataaccctttcattagatatactagtttaattagttctctaatgtctcaaggcgtgatctcattggctaaccagctgcctgcacaggtatctaatattaatatctatcataagttcatttcttttttatcctttgttgatttgattgttccatatcaagagttaattaagtatttgttttttaggattctattggaactgagtttaatccagatattctgaaggttcgtcacgagtctccagcgaacgccttgtatcgtgatctccctagacaatgcaaaacttgtgggctccgattcagatgccaagagaaacacagtagtcatatggattggcatgtgactaagaatcggatgtctaaaagctgtaagcagaagccctctcgcaagtggtttgcgagtcttaagctgtggcttagtggtgcagaggcattgggaacagaatcagttccacattttttggcaacagaaagcatagattatgaagaaaggaaagatcatgaagagtttgcagtgccaacagaagaggaccagagtacatgtgcgttatgtggagagtcttttgatgagttttacagtgatgaaatggaggaatggatgtatagaggagctgcatacctcaatgcacccgctagaaaaacatcagcaggcatggataggtcacagttaggccccataattcatgctaaatgcagatcagactctaatctgcagtgtgccaatgaagaggatactcgaaggaaacgaatgcggagttgacaaaagttgaccagttttgtgcattgtaacttgtgatttttcattgtaatcttgcaataccatggccgttctgccaaagtcatgtacatgcatagttggtacatgttggtcccttgcatttttggccccaagtcaaaactattgttagtatattgctatttttgtgatggggtatagataggagtttaaacaattcagcgagaagctatctgtttgatcagaaatcattcttgctactagtttacttggtaattgacatagcacttactgtttgtatttttttttttttatatcaatatgcataaatgggagcaaacttcaaagatattcaaataaatttgc

SEQ ID NO: 47 - DNA, 1746 bp, Glycine max
  source 1..1746: mol_type = other DNA
atgagtgaatatgcagttgctagctggttatttgagagagactatcctcatccggctgttgatttaggagtagccaaagcattaggtagacatgtggaggttagtgaacggcaacgaaaacgaaaacaatttccagcttctatcacacacagctttatcaatggacagcagcatcaaagtccaagagctttaattgaggcatatggtagtgacaaaagccaacaaacttcaagcagcaagtctttgttagttgaaccactggatataaatggtatagacaacatgtcaacttcattgcagaatactgaagaggaggagtttgattgggaagatatgagtccaaccttattgtcttcaactattggtttctccagggaaaggcctgttgttccagctaatgcaactttgtcagagcaagatgccagcaagggatggggtattagagattttcgtcatgcaccattgggtgaggtacctagattccaaaatcaaatcaattatagtctaggctcttctcaacctcatgatccttcgaagatttctcttcatctgtctaactcatcacaacataactttaataatagggggcgagcaaggagtctcacgatccctcctgttgataacattcgaaatactgatataaacccatatctggttcgacctacatcttttgaaataaggccttctacaaatttgaatgtcactcgacctcctaccttaaatccaataactccattgcaaaaacgtgttaggaatcagtttgaagctacacatactagtgaccctattgtgaaccatgtaaataaatcttcgtttatgccagaacagtcatttgaccgtattgagaacaaggatgcaagtatttctaagatacatcagttgcctaatcagcatcccggactaatttcatccaaccagcaaaatcacgggcaagcaccacagctgcagttttttccatctcaggatccaacgccctctcaatttagtcctgggagttcattgcagctacatgatgcttctattagtacagccatgtcaaatccgttacatggtatacaattcccttcaccagtccaaagctttgcaaataaccctttcattagatatactagtttaattagttctctaatgtctcaaggcgtgatctcattggctaaccagctgcctgcacaggattctattggaactgagtttaatccagatattctgaaggttcgtcacgagtctccagcgaacgccttgtatcgtgatctccctagacaatgcaaaacttgtgggctccgattcagatgccaagagaaacacagtagtcatatggattggcatgtgactaagaatcggatgtctaaaagctgtaagcagaagccctctcgcaagtggtttgcgagtcttaagctgtggcttagtggtgcagaggcattgggaacagaatcagttccacattttttggcaacagaaagcatagattatgaagaaaggaaagatcatgaagagtttgcagtgccaacagaagaggaccagagtacatgtgcgttatgtggagagtcttttgatgagttttacagtgatgaaatggaggaatggatgtatagaggagctgcatacctcaatgcacccgctagaaaaacatcagcaggcatggataggtcacagttaggccccataattcatgctaaatgcagatcagactctaatctgcagtgtgccaatgaagaggatactcgaaggaaacgaatgcggagttga

SEQ ID NO: 48 - protein, 581 aa, Glycine max
MSEYAVASWLFERDYPHPAVDLGVAKALGRHVEVSERQRKRKQFPASITHSFINGQQHQSPRALIEAYGSDKSQQTSSSKSLLVEPLDINGIDNMSTSLQNTEEEEFDWEDMSPTLLSSTIGFSRERPVVPANATLSEQDASKGWGIRDFRHAPLGEVPRFQNQINYSLGSSQPHDPSKISLHLSNSSQHNFNNRGRARSLTIPPVDNIRNTDINPYLVRPTSFEIRPSTNLNVTRPPTLNPITPLQKRVRNQFEATHTSDPIVNHVNKSSFMPEQSFDRIENKDASISKIHQLPNQHPGLISSNQQNHGQAPQLQFFPSQDPTPSQFSPGSSLQLHDASISTAMSNPLHGIQFPSPVQSFANNPFIRYTSLISSLMSQGVISLANQLPAQDSIGTEFNPDILKVRHESPANALYRDLPRQCKTCGLRFRCQEKHSSHMDWHVTKNRMSKSCKQKPSRKWFASLKLWLSGAEALGTESVPHFLATESIDYEERKDHEEFAVPTEEDQSTCALCGESFDEFYSDEMEEWMYRGAAYLNAPARKTSAGMDRSQLGPIIHAKCRSDSNLQCANEEDTRRKRMRS

SEQ ID NO: 49 - DNA, 7878 bp, Glycine max
  source 1..7878: mol_type = genomic DNA
gaactctttcttcttcgttttttttttccaaccaagatcacagcctacttataaacactccacctccttccaccttcttccatctctctcttttattttttattttttattaattcatgtaaattaggattagggtttatctcaattcgccctctacaattttaaatccgagcttctcttcgattcattccccctccccaataaaaccggcccgagaaccaataaaggttaggattagggttttaaattcgtttgcatttgattcgaatgaacatggagagatcgttggacagatcgagagaacccggtcccaagaagcctcgattaatcgaggaactcagcgcccgacaattacctctacgccctgcggcagttactacgttgccgtctacgaggttccgagcatatgacagagactccggcggcggcggagggtatcagccgcagcctccgccgcaccaagagcttgtgattcagtacaagaccgcgcttgctgagctcactttcaattcgaagccaataattaccaacttgactattattgctggagagaacttgtctgctgccaaggcaattgctgccaccgtttgtgctaatattatagaggtgaacaaaatatgcagttgtttatgtcaagtttattgaattgaatagtaatggatttggaatgaagaaagagcaattcaattagttttattgtttgtcatgctatgcgttgatgaattgggttagtgttgtacttagatggaaatgaatgctagtcagaaaaagggcattcatagtttgttcaaattgaatttcatctcatttcattggccctgtagatgtagattggttcttaagtggtacactctgccctgcaccttaaaagaacgaaaaacatatttagttttccaatcagagttacaaagaaaaacaaatactgtcctttggccaaattacggtagcttggtattttgactacgtgtgtaactggtaattagcaccattggggagtggttttggtatgcacaggggattgagaatcctggtaaacatggatttttccatttacaagagagaagtgaaccctaaaagtttccaatctcaatcagtgctaaagtccacttttggtgaaatttgatactaacttttaattggattagatatcgttgattgcagctgtttcccttgttttcatatgtcagggaaatcttatttctctcatggcgtctttgtattatggaaaaaaggtcttgtttaattgaaatgagttcatgactttccaggttccgagtgatcaaaaactgccatctctttatctcttagacagtattgtgaagaacattggacgggattatataaaatattttgcttccagactacctgaggtcagatatttattatctttaaccactttaccttcatacctttattttgatatgcttatatgatgaatcctttagattgtgaagttatcaaaatgtatactgaactattctgataaaagtcagttacatgaatcaagagattgtaagaattcttgtatttcccttaaaaataaataatctatttataatgaaatggaaccaaattttccctttgcatttttcctgcaatatatgttttctaattctatacacctatttttaactaggtactactactttacattttaggtattctgcaaggcttacagacaggttgatccttgtgttcattcaagtatgaaacatctttttggaacttggaaaggagtctttcctcctcagtcccttcagatgattgagaaggaacttggctttgcacctgcagtcaatagttcagcttcagtatctgctacagtcaggagtgatttgcagtcacaacgcccacctcatagtatccatgtgaatcccaagtatttagaaaggcagcgtcttcagcagtcaagtagggtaggtgcaattatgtcaaaacttgtcctttaaaggtttgaaatgatgcattttgataggaatcagtcaagattggacagtgatacagaagcttttgtgatggaactgaaatttagagtcatgtgtttcttggatgatgaaacaggactggaagacatgtcatttttaatcttgaatatgagacaatacagtatttttgcaataagtgtaattgaattggaattttcattgaactaatttgaatttctttttttttcttcttaatcatgaattatcctgattttgcggttctgtttatggtttgacatgtgacatatgtgctattaacctttttagacattagttaaaagtaatattttgaacatcatcttcatattatttttatgtacatattttagatttttgttttcactttgtaactgtatcttaaagtctgattcacaattcaagtcttttggtccaccatctcttgtacttatttcttttgccatgtcctttttttatgtcatgaagagtaaaggagtagttaatgatatgactgaagctgttttaaactcaaatgaggatttggagaggccagatagagctttgagtgctgctcgaccatggctggatcctaggattaacatgcttgtatgtgaagtagcttttcttttcctctttatactattcactctatcagttgtcatatttttaatgtagtgctgtatattgcagaataatcagcacacacatagagatgcatttaatgattctgttcctgaaaagagcatggatgggtcatcatacggaggcagtgaatatagttctgtcatttcaagtaatttgggttcaggggctggaagaactggaagtaagctcattgatttaggacatgacagaacatggttcaaaacagatggtggtgatgcagatactacatctgggcaaagaaatggtttcaatctgaagcatagtttttctaatcatgaagcaccaaagtccatgaatttggatgcacattgtcagccgagacaaagcataactaacaaacagaacgatgtgatgtcatgtaactggaaaacttctgaagaagaggagttcatgtgggatgagattgacaatggtttgattgatcatggacccaatgtatcaaaaactttgagcacggacacatggatggcagatgttgaaaatttggtaagcttgaggaaagggtcagttgcactttgccatttattcatccaattgcagtttttctcctttctcctaatttataaaatgaagtgcattcctctttgatttaacatgatttttttggggggggaatggtgtcccatgtcctccaaattaataaaattctatctgatttgaaaagcacttgtcccaccagtaattttttcacgtaacaattaagtccagttaatggtgtctaattttaatcagacggtataaacagtggcagatccactcattggggtgtgggggcaattgctcccacaaagtaaatcttttacttgaatatattgaataaatattttttttgtcccgataaaagaataggtcttgtccccacaagataattttttttaataagatgaatataaatagttgtaaaagataaaagagaaaataaattgatactaattttactcactttatcctttttaattttactgttttaaattttttattactatttttaataaagcttatacaattttttgtttgagaaaatattagactatttttttcccccacttaaaaaaatttctggatccgccactgttataagcttatcactttttgcacagatagaacatcatttagagtcttttatcccattaaaaagtcacttgtgtaagcttcttctaattatgagaatcaatccctatcttgaaagatggggacatttcagaaaatcatattttaaaggaagggccaaattgcttggtagttagtggcttagtgcaatagaactattagtgtggtaacatgcaattacccctaatatttaacttttgtatcaaagaagttcttttataatcgttgtagggccgattgcctgaattccctaagataatatgcctaattttgttgattaaatcaaacactaatttatcaaatccgagcaatccaaagttctaaatatcctaatacaaacgaagaaaaaaaccctgatttaatttgttataagataacccggttccaaaacattcaaaccctaataattaaaccacttaatccctaattacaaagcctcaaattatggatgcatcacaaattaatacaatcatctgaaactatatatatatatatatatatatatatatatatatatatatatatatatatatatatatatatatattattggttatacttggctgatttatcttttccatgtgcaggaaggtgaagatcacctccaaattacacgcccttttggggcaaaggtcaatagagaaatatccactgttaagaatcaattacctggtttggggggccatccaccatcatcgtggcaattgcagaatctaaagccaggctactcagaaggatttgtgtcctctcatagtgccttaccagccaatgcaagttctttgactgtcaagaaggggaaccaatcctttatgtcgaattcggtagtaggaagggtcaaatttgtgggacaacaatttgattctggggaaactgaatccccttcttggcagtcacctttgcggcaacagtctccatcactgccaggaacagtacaccaccctcattcaatgcaaaattttgctgaccaagaattaccccagaatctcaaaacctctcaatttttgggaggtccgataactcagcatattagagatcactcccttacgctacgcccgattgttcaggttggtaacttgcgaagatcacaggaaaaggacatgcagggcccgttatcttccatgacttcttttcgaccaaagcttcagcaaaagcagctggacccgtctcagtctaaagtttctttaaccagagaaacttcagcacagttgaccacaaataatttgtcagctgcacctgtcaagagtggagtcatccccaagaaatcaattacctgtgatccggatccaaggaagcatccatctcagactggggttcagcctactcagtctggtagacctaccacattagatcctctgcataatgactcatctacactgccaaaaaacactcaaggaaaggctggacaaccaccacaaaggctttctactcaaccacccgcttcctctaacattagcagttcctcagctccgaccttaaatactgccaaaaataataaactaaatccaatttcaaaccttttaagctcattggttgcaaagggtttgatatctgcagaaacagaatcaccaactatggtaccaagtgaggttccgaagggatccaaagatcaaactgaaataattaccaccagttgttctttaccagttacatcaatttctggttctgcagctgtcccagtatcgtcttccggagatgaggtagattctgctacaaaaacctctctcgcctcacctcaatcaactagcacggaaatcagaaatcttgttggctttgattttagacctaatgtaattcgagaattccacccatcagtaattagggaattatgggatgatttccctcataattgcaaagtttgtggtattaagcttaaacaagaagaactgtttaatagacatttggagtggcatgccacaagagaacatggtccaattaaggcatcaagaagttggtatgctgagtctagtgactggattgctggcaaggcagaatattcatctgagtctgggtttaatgattctgttgatgtacacgaacaaaaaacagacagcagtcaattggatacgatggttctagcagatgaaaaccagtgtctgtgtgtattgtgtggcgagctatttgaagatgcgtactgtcatgaaaggaacgagtggatgttcaaaggggccgtttacatgaactattcagatgtcaactgtgagatggaaagtagaaatgtgggtcccatcattcatgccaaatgcttatcagagaactcaatcgtcaccaatttggtaaggcctattatctgatattggttgacgtcttccagtttaatgtttaaacttcaaagcttcaatttgtctacttgaaggagtgtgtttaaattttggcttattgcacttttttccctcaactttttaatttttgatgcatttttatcccttacaaaataatttagtgtactttattctcaacttttaagaaacttgtgaattttaccttctatcatttatccattaataattgctaaaattggagtagggttgaaaaaaggtcaaatggcccgtcagggcttgcagcttggcctgacctatttaggctctaggctaatgcttttcataaagtctttttaactaaaaaggtcaagctgtaggctttaaaaaaagtctattaaacgggatagactgacttgtttatacaatagtatatataatattatatatatatatatatatatatatatatatatttatatttaatatataattattaataattttataataaaaataatatatgtaaggatcaaggtttgttggcccagttagacctattggcttgtttataggtccttctataaaataggttgcttaaacggacttttaaacaagactaaggcttaaagaaaggttaggtaggccatgggctttcaatcaagccaggctcaggcttaataaaagcctattaagcctgatagctatttataatatatatatatatatatatatatatatatatatatatataatattttttattggattttgctaactacacatccttgttttataggcctttctacaaaaacagattgcataaacattcaggtcagatcagacttttatataaacaagattaaggctttaaaaaaggcctgtaaaaggtaaaggtcagcctgcctgtttccaattgataggtttcttgtcaaccctcctctctcctgttcaattgttttaaacagtatttttcctacctttaggataagatcaaattaaaggtatgtaaaaaaaaaaaggaaattaaggggaagcctccgtttcagtattaggggtctaaaatataataattaattgaacgagcttcgcaagacgtgaataagtgaatcactagtgtatttgttttggctcctatttctcctatcccttttctttatccaaagttaataagatttcttgtgtaaaaataaatttaattaataaatagagaaaaaattaaatgccaagtcaaacgtcgggattcatatctcaagaatgtgagttcacattctactttcgatatatgaatcttattggtgagcgataagtaagtgtctctattagtacttaatgagccttgcgggttgagacatgtcatgacttggtaagccttgagactcaattctcctaaattttttataataaaaaaatcaaatatcagatgtgaatgcaatgctaatttaacacgatctagaaagaaaattataggatctggaagcagcagcccaacaatgattcccatggatgtttcttgtctcaatagtgcatgtttaaattaaagttggaagaattcaaaagtatatttatcttagaagctacaatttttttcttcttcgactggtgtattaatccaaacataagattaacatgattattagccataaaaatcgtggatctattttttaggcaagccgcaagtccttttacatatatgacttattgtattatataatacttctctttttatataatgtttaaaggttttgacacatcaaataatgtaatttgttgaatttcaaagaaaacactaggtaacttttaaatatatcctttatctctctaaaatgtatttaatgcaatattgattttgtaaaattacctaatttttgtaacaattttttctctataaaatgttatataat

SEQ ID NO: 50 - DNA, 3000 bp, Glycine max
  source 1..3000: mol_type = other DNA
atgaacatggagagatcgttggacagatcgagagaacccggtcccaagaagcctcgattaatcgaggaactcagcgcccgacaattacctctacgccctgcggcagttactacgttgccgtctacgaggttccgagcatatgacagagactccggcggcggcggagggtatcagccgcagcctccgccgcaccaagagcttgtgattcagtacaagaccgcgcttgctgagctcactttcaattcgaagccaataattaccaacttgactattattgctggagagaacttgtctgctgccaaggcaattgctgccaccgtttgtgctaatattatagaggttccgagtgatcaaaaactgccatctctttatctcttagacagtattgtgaagaacattggacgggattatataaaatattttgcttccagactacctgaggtattctgcaaggcttacagacaggttgatccttgtgttcattcaagtatgaaacatctttttggaacttggaaaggagtctttcctcctcagtcccttcagatgattgagaaggaacttggctttgcacctgcagtcaatagttcagcttcagtatctgctacagtcaggagtgatttgcagtcacaacgcccacctcatagtatccatgtgaatcccaagtatttagaaaggcagcgtcttcagcagtcaagtaggagtaaaggagtagttaatgatatgactgaagctgttttaaactcaaatgaggatttggagaggccagatagagctttgagtgctgctcgaccatggctggatcctaggattaacatgctttgctgtatattgcagaataatcagcacacacatagagatgcatttaatgattctgttcctgaaaagagcatggatgggtcatcatacggaggcagtgaatatagttctgtcatttcaagtaatttgggttcaggggctggaagaactggaagtaagctcattgatttaggacatgacagaacatggttcaaaacagatggtggtgatgcagatactacatctgggcaaagaaatggtttcaatctgaagcatagtttttctaatcatgaagcaccaaagtccatgaatttggatgcacattgtcagccgagacaaagcataactaacaaacagaacgatgtgatgtcatgtaactggaaaacttctgaagaagaggagttcatgtgggatgagattgacaatggtttgattgatcatggacccaatgtatcaaaaactttgagcacggacacatggatggcagatgttgaaaatttggaaggtgaagatcacctccaaattacacgcccttttggggcaaaggtcaatagagaaatatccactgttaagaatcaattacctggtttggggggccatccaccatcatcgtggcaattgcagaatctaaagccaggctactcagaaggatttgtgtcctctcatagtgccttaccagccaatgcaagttctttgactgtcaagaaggggaaccaatcctttatgtcgaattcggtagtaggaagggtcaaatttgtgggacaacaatttgattctggggaaactgaatccccttcttggcagtcacctttgcggcaacagtctccatcactgccaggaacagtacaccaccctcattcaatgcaaaattttgctgaccaagaattaccccagaatctcaaaacctctcaatttttgggaggtccgataactcagcatattagagatcactcccttacgctacgcccgattgttcaggttggtaacttgcgaagatcacaggaaaaggacatgcagggcccgttatcttccatgacttcttttcgaccaaagcttcagcaaaagcagctggacccgtctcagtctaaagtttctttaaccagagaaacttcagcacagttgaccacaaataatttgtcagctgcacctgtcaagagtggagtcatccccaagaaatcaattacctgtgatccggatccaaggaagcatccatctcagactggggttcagcctactcagtctggtagacctaccacattagatcctctgcataatgactcatctacactgccaaaaaacactcaaggaaaggctggacaaccaccacaaaggctttctactcaaccacccgcttcctctaacattagcagttcctcagctccgaccttaaatactgccaaaaataataaactaaatccaatttcaaaccttttaagctcattggttgcaaagggtttgatatctgcagaaacagaatcaccaactatggtaccaagtgaggttccgaagggatccaaagatcaaactgaaataattaccaccagttgttctttaccagttacatcaatttctggttctgcagctgtcccagtatcgtcttccggagatgaggtagattctgctacaaaaacctctctcgcctcacctcaatcaactagcacggaaatcagaaatcttgttggctttgattttagacctaatgtaattcgagaattccacccatcagtaattagggaattatgggatgatttccctcataattgcaaagtttgtggtattaagcttaaacaagaagaactgtttaatagacatttggagtggcatgccacaagagaacatggtccaattaaggcatcaagaagttggtatgctgagtctagtgactggattgctggcaaggcagaatattcatctgagtctgggtttaatgattctgttgatgtacacgaacaaaaaacagacagcagtcaattggatacgatggttctagcagatgaaaaccagtgtctgtgtgtattgtgtggcgagctatttgaagatgcgtactgtcatgaaaggaacgagtggatgttcaaaggggccgtttacatgaactattcagatgtcaactgtgagatggaaagtagaaatgtgggtcccatcattcatgccaaatgcttatcagagaactcaatcgtcaccaatttggtaaggcctattatctga

SEQ ID NO: 51 - protein, 999 aa, Glycine max
MNMERSLDRSREPGPKKPRLIEELSARQLPLRPAAVTTLPSTRFRAYDRDSGGGGGYQPQPPPHQELVIQYKTALAELTFNSKPIITNLTIIAGENLSAAKAIAATVCANIIEVPSDQKLPSLYLLDSIVKNIGRDYIKYFASRLPEVFCKAYRQVDPCVHSSMKHLFGTWKGVFPPQSLQMIEKELGFAPAVNSSASVSATVRSDLQSQRPPHSIHVNPKYLERQRLQQSSRSKGVVNDMTEAVLNSNEDLERPDRALSAARPWLDPRINMLCCILQNNQHTHRDAFNDSVPEKSMDGSSYGGSEYSSVISSNLGSGAGRTGSKLIDLGHDRTWFKTDGGDADTTSGQRNGFNLKHSFSNHEAPKSMNLDAHCQPRQSITNKQNDVMSCNWKTSEEEEFMWDEIDNGLIDHGPNVSKTLSTDTWMADVENLEGEDHLQITRPFGAKVNREISTVKNQLPGLGGHPPSSWQLQNLKPGYSEGFVSSHSALPANASSLTVKKGNQSFMSNSVVGRVKFVGQQFDSGETESPSWQSPLRQQSPSLPGTVHHPHSMQNFADQELPQNLKTSQFLGGPITQHIRDHSLTLRPIVQVGNLRRSQEKDMQGPLSSMTSFRPKLQQKQLDPSQSKVSLTRETSAQLTTNNLSAAPVKSGVIPKKSITCDPDPRKHPSQTGVQPTQSGRPTTLDPLHNDSSTLPKNTQGKAGQPPQRLSTQPPASSNISSSSAPTLNTAKNNKLNPISNLLSSLVAKGLISAETESPTMVPSEVPKGSKDQTEIITTSCSLPVTSISGSAAVPVSSSGDEVDSATKTSLASPQSTSTEIRNLVGFDFRPNVIREFHPSVIRELWDDFPHNCKVCGIKLKQEELFNRHLEWHATREHGPIKASRSWYAESSDWIAGKAEYSSESGFNDSVDVHEQKTDSSQLDTMVLADENQCLCVLCGELFEDAYCHERNEWMFKGAVYMNYSDVNCEMESRNVGPIIHAKCLSENSIVTNLVRPII

SEQ ID NO: 52 - DNA, 6710 bp, Glycine max
  source 1..6710: mol_type = genomic DNA
gaactctttcttcttcgtttttttttccaaccaagatcacagcctactcataaacactccacctccttccaccttcttccatctctctcttttatttttttatttttattaattcatgtaaattaggattagggtttatctcaaatcgccctctacaattttaaatccgaacttctcttcgattcattccccctccccaataaaaccggcgcgagaaccaataaaggttaggattagggttttaaattcgtttgcatttgattcgaatgaacatggagagatcgttggacagatcgagagaacctggtcccaagaagcctcgattgatcgaggaactcaacgcccgacagttacctcaacgccctacggcagttactacgttgccgtctacgaggtttcgagcatatggcagagactccgaaatcagcgatttgggtcgtggcggtggcggcgggtaccagccgcagcctccgccgcaccaagagcttgtgactcagtacaagactgcgcttgctgagctcactttcaattcgaagccgataattaccaacttgactattattgccggagagaacctttctgctgccaaggcaattgctgctgctgtttatgataatattctagaggtgaacaaaatgtgtagttgtttacgttaagtttgttgagttgaattgtaatggatttggaatgaagaaagagcaatccgagttaatctctctgactagtgaggagtggtgttattgtttgccatgctatacgttgatgaattgggttagtgttgtacctagatggaaatgaatgctagtcagaaaaagggcattcataatttgttgaaatttaatttcatctcatttcattggccctgtagatgtagattggttcttaaatggtacactctgccctgcaccttaaaagaacgaaaaacataattagttttccaatcagagttacaaagaaaaacaaatactgtcctttggccaaatgacggtagcttggtattttgactacgtgggtaactggtaattagcaccattggggagtggttttggtatgcacaggggattgagaatcctggaaaacatggattttttcatttgcaagagaaaagtgaaccctaacagtttccaatctcaatgctaaagtccacttttggtgaaatttgatactaacttttaattggattagatatcgttgattgcagttgtttcccttgttttcatatgtcagggcaatcttatttctctcattgtgtctttgtattatggaaataaggtcttgcttaattgaaatgtgttcttgactttccaggttccaagtgatcaaaaactgccatctctttatctcttagacagtattgttaagaatatcggacgggattatataaaatattttgcttacagactacctgaggtcagatatttattatctttgaccattttaccttcacacctttattttgatatgcttatatgatcaatgctttagattgtgaagttatgaaattttatagtgaactgttctgataaaagtcagttacatgaatcaagagattgtaagaattcttgtatttgcctaaaaaataaataatctaattataatgaaatagaaccaatatttccctttgcatttttcgtgtgatatatgttttctaattctatacacctatttttaactaggtactactactttacattttaggtattctgcaaggcttacaaacaggttgatccttgtgttcattcaagtatgcaacatctttttggaacttggaaaggagtctttcctcctcagtcccttcagatgattgagaaggaacttggctttgcacctgcagtcaatagttcagcttcagtatctgctacagtcagaagtgatttgcagtcacaacgcccacctcatagtatccatgtgaatcccaagtatttagaaaggcagcgtcttcagcagtcaagtagggtaagtgaaaccattgcatcaaaacttgtcctttaaaggtttaaaatgatggattttgatgggaatcagtcaagattggatactgttacagaattttttgtgatgtactgaaatttgcagtcatttgttccttggatgatgaaacaggactggaagacatgctgtttttcatcttgaatatgagacactatacagtattcttgcgataagtgtatttgtattggaattatccgttgaactaatttgaatttctttattcttcttaatcatgaattatcttgattatgtggttctgtttatggtttgacatgtgacatgtgctactgctattaaacattttagacattagtaatgttttgaacaccatcttcatattatttttatgtacatattttagatttcttttttgattttgtatctgtatcttaaagtttgattcacaattcaagtcttttggtcgaccatctcttgtacttatttctttttccatgcccttttttatgtcatgaagagtaaaggggtagttaatgatatgactggagctgttttaaactcaaatgaggattcggagaggccagatagagctttgagtgctgcacgaccatggctggatcctaggattaacatgcttgtatgtgaagtagcttttcttttcctctttatactattcactctatcagttgtcatattttaatgtagtggtgtatattgcagaataatcagcacacacatagagatgtatttaatgattctgttcctgaaaagagcatggatgggtcatcatacggaggcagtgaatatagttctgtcatttcaagtaatttggtctcaggggctggaagaactggaagtaagctcattgatttaggacatgacaaaacatggttcaaaacagatggtggtgatccagatactacatctgggcaaagaaatggtttcaatctcaagcgtagttattcaaatcgtgaagcaccaaagctcacgaatttggatgcacatcgtcagccaagacaaagcacaactgacatacggaacaatttgatgtcaggtaactggaaaacttctgaagaagaggagttcatgtggggtgagatgaacattggtttgactgatcatggagccaatgtatcaagcaatttgagcacagacacatggatggcagatgatgagaatttggtaagcttgccaaaagggtcagttgcactttgccatttattcatccaattgcagtttttctcctttctcctaatttataaaacgaagtgcattcctctttgttttaacatgattttttgtaattattttgcaattgtgtcccatgtcctccaaattaataaaactcttgctgatttgaaaagcacttgccccaccagtaattttttcacgtaacaattaagaccagttaatggtgtctaattttaatcagatccacttaaggtataagcttatcactttttgcacaattagaacatcattcatagtcgttttatcccattgaaagtcacttgtgtaagcttattctaattatgaggatcaatccctatcttgaaagatggggaacatttcagaaagtcatattaaaggaaggggtaaattgcttggtagttagtgcaataggattaatagtgtggtaacatgcaattttccctaatatttaactcttgtatcaaataaattcttttgtaatcattctagggctgaatgcctgaattctctaagataatatccctaattttgttgattaaaacaaacactaatttatcaaaaccgagcaatccgaagttctaaatatcctaatacaaacaaataaaaaatcctaatttaatttgcataagatatcccggttccaaaacattcaaaccctaataattaaaccacttaatccctaattacaaagcctttaattacggatgtgtcacaaattaatacattgatctgaaattatatatatattttgttggttatacttgactgatttattttttccatgtgcaggaaggtgaagatcacctccaaatcacacgcccttttggagcaaaggtcgatagagaaatatccactgctaagaaacaaccacctggttttgggggtcatccaccgtcatcctggcaattgcagaagcaccactcaattgataagttgaatctaaagccaggttactcagaaggatttgtgtcaactcttagtggattaccagccaacccaagttctttggctgtcaagaaggggaaccaatcctttacatcgaatgcagtagtaggaatggccaaatttgtgggacaacaatttgattctggggaaactgaatccccttctgggcagtcacctttgcggcaacagtctccatcactgccaggagcagtacaccatactcattcaatgcaaaattttgctgaccaagaattgccccagaatctcaaaacctctcgatttttgggaggtccgataagtcagcatattagagatcgctcacctaccggtcatccaattgttcaggttggtaacttgcgaagatcacaggaaagggacatgcacggcccattatcttccatgacttcttttcgaccaaagcttcagcaaaagcagctgaacccatctcagactgaagttactgctaaaactaagctgccccagtctaaagtttctttaaccagagaaacttcagaacagttgagcacaaataatttgtcagctgtacctgtcaagagtggaatcatccccaagaaatcaatttccagtaatctggattcaagggaggatccatctcagactggggttcaacctactcagtctggcaggcctaccacattaatttcttcagggtctgctgtggcatcaccatcttcattagatcctctgcataatgattcatctactctgccaaaaaaacctcaaggaaaggctggacaaccaccacaaaggctttctactcaaccacctgcttcctctagtgttagcagttcctcagctccaaccttaaatgctgccaaaaataataaattaaatccaattgcaaaccttttaagctcattggttgcaaagggcttgatatctgcagaaacagaatcaccaactacggtaccaagtgaagcgccgaagggatccaaagatcaaactgaaataattaccaccagttgttctttaccagttacatcaatttctggttctgcagctatcccagtatcatcttccggagataaggtagatgctgctacaaaaatctctcatgcctcacctcaatcaactagcacggaaatcagaaatctcatcggctttgattttagacctaatgtaattcgagaattccacccatcagtaattagggaattatgggatgatttccctcataattgcaaagtttgtggtattaagcttaaacaagaactgtttaatagacacttggagtggcatgccgcaagagaacatggtccaattaaggcatcaagaagttggtatgctaagtcaattgattggattgctggcaggacagaatattcatctgagtccgagtttactgactctgttgatttacaagacaaaaaaatagacagcagtcaattggatacgatggttctagcagatgaaaaccagtgtctgtgtgtattgtgtggcgagctatttgaagatgtgtgctgtcatgacaggaacgagtggatgttcaaaggggccgtttacatgaacttttcagatgtcaattgtgagatggaaagtagaaatgtgggtcccatcattcatgccaaatgcttatcagagaactcagtgatcaccaattcggtaaggcctattatctgatattagtggacgtcttccagttgaaaaatgcatgttaggatagtaaaattcgtgcatctctcacgaataacgtggaaaacagatttttttttctccttccgcttatttctctcaattttcctcttttttttttttcgggttgtttcatcctcccaccatgcacctaacatatgcagcaattatgagagaatggagtgctgaaaggcatgcttaagaatgcgaaattagaagccatgcttaacctatgccactggcatgtcgtgcaccatggaaggcaggtgtgagaattgagaagcacaagcacatggttttcaattggttttgttgtgtgtattgcagcgtgagcaacagataatataataccatattttcattttatttagtaaatctttttgctgtaagaaaaaaaaattatttaataagctgtgcgtataattagaacaaataacctgggcacgtgtgggggaaagatttgcacgctgttggtgcaattcgtgggattcgtaacatatagcacctctccttccagtttgatgtttaaacatcaaagcttcaatttgtctacttgaaggggtgtgtttaaattttttatagtaatttgattagacctgttctatttttgcaggataatgattaatggtttgcttctatagcatttgagctatggcgtagtcagaagcttaccaagggtaacccacccagtttcacattgcataataaattttgatggtcttttatacttaaaatttattgcctctaactggttcttagagcggtcaaacaaattttgtgcttttatttttcttgtacccaaaaaaagaattgcgtagtggtcaaaatatgtaaatgcgagataagtttttgcagctaaacttggtcgttgatgtggttgtgttaactttatttcaactatggagaggcggattttctgcatttgttaatctaaaataattgcgtagttg

SEQ ID NO: 53 - DNA, 3099 bp, Glycine max
  source 1..3099: mol_type = other DNA
atgaacatggagagatcgttggacagatcgagagaacctggtcccaagaagcctcgattgatcgaggaactcaacgcccgacagttacctcaacgccctacggcagttactacgttgccgtctacgaggtttcgagcatatggcagagactccgaaatcagcgatttgggtcgtggcggtggcggcgggtaccagccgcagcctccgccgcaccaagagcttgtgactcagtacaagactgcgcttgctgagctcactttcaattcgaagccgataattaccaacttgactattattgccggagagaacctttctgctgccaaggcaattgctgctgctgtttatgataatattctagaggttccaagtgatcaaaaactgccatctctttatctcttagacagtattgttaagaatatcggacgggattatataaaatattttgcttacagactacctgaggtattctgcaaggcttacaaacaggttgatccttgtgttcattcaagtatgcaacatctttttggaacttggaaaggagtctttcctcctcagtcccttcagatgattgagaaggaacttggctttgcacctgcagtcaatagttcagcttcagtatctgctacagtcagaagtgatttgcagtcacaacgcccacctcatagtatccatgtgaatcccaagtatttagaaaggcagcgtcttcagcagtcaagtaggagtaaaggggtagttaatgatatgactggagctgttttaaactcaaatgaggattcggagaggccagatagagctttgagtgctgcacgaccatggctggatcctaggattaacatgcttaataatcagcacacacatagagatgtatttaatgattctgttcctgaaaagagcatggatgggtcatcatacggaggcagtgaatatagttctgtcatttcaagtaatttggtctcaggggctggaagaactggaagtaagctcattgatttaggacatgacaaaacatggttcaaaacagatggtggtgatccagatactacatctgggcaaagaaatggtttcaatctcaagcgtagttattcaaatcgtgaagcaccaaagctcacgaatttggatgcacatcgtcagccaagacaaagcacaactgacatacggaacaatttgatgtcaggtaactggaaaacttctgaagaagaggagttcatgtggggtgagatgaacattggtttgactgatcatggagccaatgtatcaagcaatttgagcacagacacatggatggcagatgatgagaatttggaaggtgaagatcacctccaaatcacacgcccttttggagcaaaggtcgatagagaaatatccactgctaagaaacaaccacctggttttgggggtcatccaccgtcatcctggcaattgcagaagcaccactcaattgataagttgaatctaaagccaggttactcagaaggatttgtgtcaactcttagtggattaccagccaacccaagttctttggctgtcaagaaggggaaccaatcctttacatcgaatgcagtagtaggaatggccaaatttgtgggacaacaatttgattctggggaaactgaatccccttctgggcagtcacctttgcggcaacagtctccatcactgccaggagcagtacaccatactcattcaatgcaaaattttgctgaccaagaattgccccagaatctcaaaacctctcgatttttgggaggtccgataagtcagcatattagagatcgctcacctaccggtcatccaattgttcaggttggtaacttgcgaagatcacaggaaagggacatgcacggcccattatcttccatgacttcttttcgaccaaagcttcagcaaaagcagctgaacccatctcagactgaagttactgctaaaactaagctgccccagtctaaagtttctttaaccagagaaacttcagaacagttgagcacaaataatttgtcagctgtacctgtcaagagtggaatcatccccaagaaatcaatttccagtaatctggattcaagggaggatccatctcagactggggttcaacctactcagtctggcaggcctaccacattaatttcttcagggtctgctgtggcatcaccatcttcattagatcctctgcataatgattcatctactctgccaaaaaaacctcaaggaaaggctggacaaccaccacaaaggctttctactcaaccacctgcttcctctagtgttagcagttcctcagctccaaccttaaatgctgccaaaaataataaattaaatccaattgcaaaccttttaagctcattggttgcaaagggcttgatatctgcagaaacagaatcaccaactacggtaccaagtgaagcgccgaagggatccaaagatcaaactgaaataattaccaccagttgttctttaccagttacatcaatttctggttctgcagctatcccagtatcatcttccggagataaggtagatgctgctacaaaaatctctcatgcctcacctcaatcaactagcacggaaatcagaaatctcatcggctttgattttagacctaatgtaattcgagaattccacccatcagtaattagggaattatgggatgatttccctcataattgcaaagtttgtggtattaagcttaaacaagaactgtttaatagacacttggagtggcatgccgcaagagaacatggtccaattaaggcatcaagaagttggtatgctaagtcaattgattggattgctggcaggacagaatattcatctgagtccgagtttactgactctgttgatttacaagacaaaaaaatagacagcagtcaattggatacgatggttctagcagatgaaaaccagtgtctgtgtgtattgtgtggcgagctatttgaagatgtgtgctgtcatgacaggaacgagtggatgttcaaaggggccgtttacatgaacttttcagatgtcaattgtgagatggaaagtagaaatgtgggtcccatcattcatgccaaatgcttatcagagaactcagtgatcaccaattcggtaaggcctattatctga

SEQ ID NO: 54 - protein, 1032 aa, Glycine max
MNMERSLDRSREPGPKKPRLIEELNARQLPQRPTAVTTLPSTRFRAYGRDSEISDLGRGGGGGYQPQPPPHQELVTQYKTALAELTFNSKPIITNLTIIAGENLSAAKAIAAAVYDNILEVPSDQKLPSLYLLDSIVKNIGRDYIKYFAYRLPEVFCKAYKQVDPCVHSSMQHLFGTWKGVFPPQSLQMIEKELGFAPAVNSSASVSATVRSDLQSQRPPHSIHVNPKYLERQRLQQSSRSKGVVNDMTGAVLNSNEDSERPDRALSAARPWLDPRINMLNNQHTHRDVFNDSVPEKSMDGSSYGGSEYSSVISSNLVSGAGRTGSKLIDLGHDKTWFKTDGGDPDTTSGQRNGFNLKRSYSNREAPKLTNLDAHRQPRQSTTDIRNNLMSGNWKTSEEEEFMWGEMNIGLTDHGANVSSNLSTDTWMADDENLEGEDHLQITRPFGAKVDREISTAKKQPPGFGGHPPSSWQLQKHHSIDKLNLKPGYSEGFVSTLSGLPANPSSLAVKKGNQSFTSNAVVGMAKFVGQQFDSGETESPSGQSPLRQQSPSLPGAVHHTHSMQNFADQELPQNLKTSRFLGGPISQHIRDRSPTGHPIVQVGNLRRSQERDMHGPLSSMTSFRPKLQQKQLNPSQTEVTAKTKLPQSKVSLTRETSEQLSTNNLSAVPVKSGIIPKKSISSNLDSREDPSQTGVQPTQSGRPTTLISSGSAVASPSSLDPLHNDSSTLPKKPQGKAGQPPQRLSTQPPASSSVSSSSAPTLNAAKNNKLNPIANLLSSLVAKGLISAETESPTTVPSEAPKGSKDQTEIITTSCSLPVTSISGSAAIPVSSSGDKVDAATKISHASPQSTSTEIRNLIGFDFRPNVIREFHPSVIRELWDDFPHNCKVCGIKLKQELFNRHLEWHAAREHGPIKASRSWYAKSIDWIAGRTEYSSESEFTDSVDLQDKKIDSSQLDTMVLADENQCLCVLCGELFEDVCCHDRNEWMFKGAVYMNFSDVNCEMESRNVGPIIHAKCLSENSVITNSVRPII

SEQ ID NO: 55 - DNA, 6761 bp, Glycine max
  source 1..6761: mol_type = genomic DNA
ttttatttttcttttgtaagtcaaaactcaaaactgttctatttcacctctcccccaccccctcaagattgttttattcctcaaaatggaaaaaaaataaaatgccattgcataccaaaaaagaaaaaaaaaattaaacgaaagcgaagagggacatcccgatccgtggcacgcttatgagatgaggaaagtgcaatttgggggcttcagagatcagtcccatactccaatcccatcccacgccctctcagaaacaaaacaaggctttcaactttcaattcaaaactccccaatcgcatagtgtcgccgtttctacaccgtccgattcaaaccccattcccttccccatctccaatttcaattcccctctctatgccgccgcatccccgaaacggcgacgcatcctcctaaaccctaaccctaaccctaacctcctccttcctcttcctctctccgaaaccaatatttatgttctctcaaaacgtgattctccctcccgaaaaccctagacccacggcgttcgcttccaaacccatgagcaatgagatcgcgaagcctctgccgtcgattctcgttggccgcttcaaggctctactcaagcagcgcgacgacgaactccgagtcgccgccggcgaccccgtaccgccggcgtccaccgacgaaatcgtccagatttacgaactcctcttgtcggaactcacgtgcaatctgaaacctatcatcaccgatctcacaattattgccgaacagcagagggagcacgcaaaaggcatcgctgacgcaatttgcgctcggattctcgaggtccgtctccgttgccgccgaaacttcttcttcttcttcttctacctctcgagatttgtacaatgtcatggaatcgttttatttgttggggtcaaaattatccctctcctgttgcttcattttctttttaatgtcacaattcgtgttccaatgcttcgtttttttttttagttctgcgttttttttatgcttttggattcttttttttttcctggaaaatgaaggtcacccaatgaccgagggaacgattggattatgctcaaattgaccgttgttttggacgttatattaaagcgtaatgaaaaaaataatgtgttggcactggtaattgaaagaataggaaatagtactatctaaatgagtagatatggggagtttctcacgcattgtgaatatttggtgcaatgcgtttgagggcttttttcaatttcaggaaagtcaaggggatacgaattgaaaattgaaatttggaatactacttgttctggaaaggctcaggactgcgattaaaggaaaagttgttcttttagttttgctgaaataaagtaatttctgcaaaaaagaaaggttattgaatgaacgagtgtttaaatccgtaggaaaacgttttgtaattttttaacagtatcgcagcatgcataaaatgagataatagtattcaaataatacagaatgtaaagaaattaacgactttttaaattatcaagtccattgcgagtttttgttgattcataatagaacattagtatttctactttctgaatttgaatctctactttatttatatatctggctatgccactgttacatgggaattcaattttttggagatattcttttgcaggtgccagtagatcaaaagcttccttcactctatctgttggacagtattgtgaagaattttgggcaggaatacattagatacttctctttacgtcttcctgaagtaggtcaactagagttgctggattgtttttaactttatcattttattccgcaactgaaacgttggcttcatttttctcctcaggttttctgtgaggcgtacaggcagattcagcctactttgcattctgctatgcgccatctttttggtacttggtcaaaagtctttccaccttcagtcctgcgcaagattgaaactgagttgcaattttctcaagcagttaatactcaatcatcaaccctgaatcctgtcagggcttctgaatcatctcgaccaagccacgccatacatgttaatccaaaatacttgcggcagttggagcgctcaactgtggatagtgtaagctttttttatgacatctctgtcttccacacaaactatacaaggactaggctgaggctttattagctagctggtaccatagagttgtatatttcagtaacggaattgtttattgctttcatgtaaccataacaaaaatgtaatttttgtggtaaatatctcattttatctcatggtgggatctctctgtatccatcattcctttcttgcatgtctgtccatctctctgtcttcccaataacttttccccattttcttttgggaaagattatgttctcttttatgctaagtttagtatttggggggggggggggggggcggggaatctatttagacacaacccgtatcttgtgatggattgtaaactgctttggaaaaaaaaatttattaccttttttgtttgcttacctctttcctccagagcctttatgaattatgactaagggcctgtatgtttaccatctaaaatcaattttacagtgtgacacacattcaaatggaaaatctggttaagatgaatttgtacaagcatcccaataacatttaacagttcaaaaccaaaacttccataatcaattaccaaacttcaaaacaaatgattgctaagagcatgtttgcttcctggtttaggttgaatggatccatgtccatcagaagctactagatgtagcttatgcatatttaaggtgctagacatggagagagggtagttgatccacgttaaataacctgctccaaacatagactaaattttacacatgcaatattttatttatttatcgtgatttaatgaattttctaactctaatataggtcattttttccctgattcctctttaaattgatgatcctggtgtccttagaacacaatgacagattaaatacttctgccttgtctccctttgagagcctccactttgcatattgctaatggcaaaaaagtctgtttattgaatagtttggctggcaacatatcagacatacataatttatctgtttgtctctattatttttaattattttgacatctgagagatttgtcttcctggctcatattttatgtttggaagtgaaggaagtaagtactgtatccattcattctaggaagaaattgctgctgaatatttagcattcaatagagaaaattttcaacccttatttgggaaaaaaaatcttgcaaacagatataacaagtggcattgtgacagtgtttgttttatccacacaggctagtaagacacatcaatttttatcaagcagtagcagccttggaatatcctcgtcgtccccttcaagaattggagttgataggcctttgtctgcatctatggatgaatatgcagtggacaattctgctgtcaggttaattgagagaaactcgcctcatcctgctgttgattatggagtagccaaagcattaggtagagatgtggatttgactgaatggcagcaaaagcagtatcctggtgatggtcgtaatcgatttccaacttctgttacatacagccttagtaatggacatcagcggcaaagtccaagagctttaattgatgcatacggcagtgacaaaagccaagaaacttcaagcagtaagcctttgttagttgaacgtctggatagaaatggtatagacaaagtgttgtcaacatcttggcagaatactgaagaggaggagtttgactgggaaaatatgagtccaaccttaacagaccatagtaggaataacagtttattgccttcaacttttggtttctctagggaaaggcctggtgttgcagctaatgcaactttgtcggagcaagatactaggaaggggtggtctagtggatctcagcttcctccagtagatgattcttctgccatagcagaagatgcatttgcctcttcgactgtatgtttcctcacttctttactttattccttctgatttatattgttttaatatggtttatgatattgaatttgaatttcccaattaaagtatatacctctaatcagatgccattgatgctttgggttctgatgataattttctgatgatgagggtctggatagctttgtgtgggtaggcttaatcagatgcatgaaattcgttcacacttgtctggcaattggtctcttctctaatttagttcacagatgttcactaatgccacattttttacaataaaatactgttttttagaagaaaaaaaattctgttcatattctgcatcactatttggcattgactacccttattttttataattaaaagtagaaattagttcaaggtaggttttccatatattgtcattgcttcaatccaaaagagcttttgcatgaagatgtgttagagatgtaatataaaaccatttatgtgtcttcacctaacagtttaaactttagtaggtataatttgttgttgaaagtttattttagtttatttaatttgaaaggaaataggagttgcaagtagcttgctaatagaattggaaatttgtttattcttgctacagtttcgtcgcacacccccgggtcaggtacctggatcccaaaatcaaatcaatcatagcctgggctcctctcaacctcatgatgcttggaagatttcacatcatccgtctaacatttttagtaatagggggcgagcaaggaatcttatgatccctcctatggacaacattcgcaatacagataataacccgtattgggttcgaccatcaatgtcgaggatggaagctcggccatcagttttgccagcaccttttgaaatgaggccttctgtaaatgtgaatgtaactagaccgcccatcataaatccaataaatccattgcaaaaacatgttaggagtcagttcaatgcaataaatactagtaatcctattgcaaaccatgtaaataaatcttcatttatgccaaaacagtcatttgacagtgttgagaacaaggatgcaagtatttcaaagatacatcagttgcctaatcagcttcctggagtaatttcttcaaaccagcaaaatcatgggcaggcaccacaactacagttttttccatctcaggatccatcaacctcacaattttgtcatgggagttcattgcagggacatggtgcttctataagtacagccatgtcaaatcccttaccagttataccattccctttaccatttcaaagtattgcaaataaccctttacatttacagggtggagcccatccatctttacctccaggtcgccctcctgctccatctcaaatgatacctcatccaaatgttggtgcatatatgtcaagccaacagccaactgttggatatactaatttaattagttctctaatgtcgcaaggtgtgatctcattggctaaccagctgcctgcacaggtatctaatattaacatctatcagaagttgatttctttcagttttttcccattttgatttgattgtgacacccaagagttaattaaatatttgtttttaggattctgttggaactgagtttaatccagatattctgaaggttcgtcatgagtctgcagtcaatgccttatatggtgatctccctagacaatgcacaacttgtgggctgagattcaaatgccaagaggaacacagtagtcatatggattggcatgtgactaagaatcggatgtctaaaacccgtaagcagaaaccctctcggaagtggtttgttagtgacaggatgtggctcagtggtgcagaggcattgggaacagaatcagctccaggttttttgccaacagaaaccatagaagaaaggaaagatgatgaagagttggcagtgcctgcagaagaagaccagaatacatgtgcattatgcggagagccttttgatgagttttacagtgatgaaatggaggaatggatgtatcgaggagcagtatacctcaatgcacctacgggaacaacagcaggcatggataggactcagttaggccccataattcatgctaaatgcagatcagaatctaatatggccacctctgaagatcttggaccagatgaaaaggtatctttttctctatctattattatgcactgcattagcctcagtttttttttgtcataagattatcgtcaaccatttgttccattttaatattccttggtcgcaatttttttacctgtatggtgattatgcatgcctgcatatgcttcaaggaatcttcagtaaatactctcttatttatttaatttatttatatgcagggtgccgatgaagagggttcccaaaggaaacgaatgcgaagttgatcaatgttgaccagttttgtgcattgtaacttatgattttccgttggtgtatctcatagagttgaatcttgcaataccatggccattctgccaaagtcatgtacatgtatagttggactagtaacgttcatattttcttgcagccaacggttacattttaacttaaaattatttcgtcccttgcatttttgtccacaaatcaagtattgttagtacattgttatttctcggatggggcacagggttttaaactatgcagcgaggtactatcaggttttggcagaaaatattatacttgctactaattagctggtaattgatatagaattagttgtttggacctactgtttgtattttatcaaactcatgcataaatgagagcaaactacaaagatactctatttcaatttttt

SEQ ID NO: 56 - DNA, 2814 bp, Glycine max
  source 1..2814: mol_type = other DNA
atgttctctcaaaacgtgattctccctcccgaaaaccctagacccacggcgttcgcttccaaacccatgagcaatgagatcgcgaagcctctgccgtcgattctcgttggccgcttcaaggctctactcaagcagcgcgacgacgaactccgagtcgccgccggcgaccccgtaccgccggcgtccaccgacgaaatcgtccagatttacgaactcctcttgtcggaactcacgtgcaatctgaaacctatcatcaccgatctcacaattattgccgaacagcagagggagcacgcaaaaggcatcgctgacgcaatttgcgctcggattctcgaggtgccagtagatcaaaagcttccttcactctatctgttggacagtattgtgaagaattttgggcaggaatacattagatacttctctttacgtcttcctgaagttttctgtgaggcgtacaggcagattcagcctactttgcattctgctatgcgccatctttttggtacttggtcaaaagtctttccaccttcagtcctgcgcaagattgaaactgagttgcaattttctcaagcagttaatactcaatcatcaaccctgaatcctgtcagggcttctgaatcatctcgaccaagccacgccatacatgttaatccaaaatacttgcggcagttggagcgctcaactgtggatagtgctagtaagacacatcaatttttatcaagcagtagcagccttggaatatcctcgtcgtccccttcaagaattggagttgataggcctttgtctgcatctatggatgaatatgcagtggacaattctgctgtcaggttaattgagagaaactcgcctcatcctgctgttgattatggagtagccaaagcattaggtagagatgtggatttgactgaatggcagcaaaagcagtatcctggtgatggtcgtaatcgatttccaacttctgttacatacagccttagtaatggacatcagcggcaaagtccaagagctttaattgatgcatacggcagtgacaaaagccaagaaacttcaagcagtaagcctttgttagttgaacgtctggatagaaatggtatagacaaagtgttgtcaacatcttggcagaatactgaagaggaggagtttgactgggaaaatatgagtccaaccttaacagaccatagtaggaataacagtttattgccttcaacttttggtttctctagggaaaggcctggtgttgcagctaatgcaactttgtcggagcaagatactaggaaggggtggtctagtggatctcagcttcctccagtagatgattcttctgccatagcagaagatgcatttgcctcttcgacttttcgtcgcacacccccgggtcaggtacctggatcccaaaatcaaatcaatcatagcctgggctcctctcaacctcatgatgcttggaagatttcacatcatccgtctaacatttttagtaatagggggcgagcaaggaatcttatgatccctcctatggacaacattcgcaatacagataataacccgtattgggttcgaccatcaatgtcgaggatggaagctcggccatcagttttgccagcaccttttgaaatgaggccttctgtaaatgtgaatgtaactagaccgcccatcataaatccaataaatccattgcaaaaacatgttaggagtcagttcaatgcaataaatactagtaatcctattgcaaaccatgtaaataaatcttcatttatgccaaaacagtcatttgacagtgttgagaacaaggatgcaagtatttcaaagatacatcagttgcctaatcagcttcctggagtaatttcttcaaaccagcaaaatcatgggcaggcaccacaactacagttttttccatctcaggatccatcaacctcacaattttgtcatgggagttcattgcagggacatggtgcttctataagtacagccatgtcaaatcccttaccagttataccattccctttaccatttcaaagtattgcaaataaccctttacatttacagggtggagcccatccatctttacctccaggtcgccctcctgctccatctcaaatgatacctcatccaaatgttggtgcatatatgtcaagccaacagccaactgttggatatactaatttaattagttctctaatgtcgcaaggtgtgatctcattggctaaccagctgcctgcacaggattctgttggaactgagtttaatccagatattctgaaggttcgtcatgagtctgcagtcaatgccttatatggtgatctccctagacaatgcacaacttgtgggctgagattcaaatgccaagaggaacacagtagtcatatggattggcatgtgactaagaatcggatgtctaaaacccgtaagcagaaaccctctcggaagtggtttgttagtgacaggatgtggctcagtggtgcagaggcattgggaacagaatcagctccaggttttttgccaacagaaaccatagaagaaaggaaagatgatgaagagttggcagtgcctgcagaagaagaccagaatacatgtgcattatgcggagagccttttgatgagttttacagtgatgaaatggaggaatggatgtatcgaggagcagtatacctcaatgcacctacgggaacaacagcaggcatggataggactcagttaggccccataattcatgctaaatgcagatcagaatctaatatggccacctctgaagatcttggaccagatgaaaagggtgccgatgaagagggttcccaaaggaaacgaatgcgaagttga

SEQ ID NO: 57 - protein, 937 aa, Glycine max
MFSQNVILPPENPRPTAFASKPMSNEIAKPLPSILVGRFKALLKQRDDELRVAAGDPVPPASTDEIVQIYELLLSELTCNLKPIITDLTIIAEQQREHAKGIADAICARILEVPVDQKLPSLYLLDSIVKNFGQEYIRYFSLRLPEVFCEAYRQIQPTLHSAMRHLFGTWSKVFPPSVLRKIETELQFSQAVNTQSSTLNPVRASESSRPSHAIHVNPKYLRQLERSTVDSASKTHQFLSSSSSLGISSSSPSRIGVDRPLSASMDEYAVDNSAVRLIERNSPHPAVDYGVAKALGRDVDLTEWQQKQYPGDGRNRFPTSVTYSLSNGHQRQSPRALIDAYGSDKSQETSSSKPLLVERLDRNGIDKVLSTSWQNTEEEEFDWENMSPTLTDHSRNNSLLPSTFGFSRERPGVAANATLSEQDTRKGWSSGSQLPPVDDSSAIAEDAFASSTFRRTPPGQVPGSQNQINHSLGSSQPHDAWKISHHPSNIFSNRGRARNLMIPPMDNIRNTDNNPYWVRPSMSRMEARPSVLPAPFEMRPSVNVNVTRPPIINPINPLQKHVRSQFNAINTSNPIANHVNKSSFMPKQSFDSVENKDASISKIHQLPNQLPGVISSNQQNHGQAPQLQFFPSQDPSTSQFCHGSSLQGHGASISTAMSNPLPVIPFPLPFQSIANNPLHLQGGAHPSLPPGRPPAPSQMIPHPNVGAYMSSQQPTVGYTNLISSLMSQGVISLANQLPAQDSVGTEFNPDILKVRHESAVNALYGDLPRQCTTCGLRFKCQEEHSSHMDWHVTKNRMSKTRKQKPSRKWFVSDRMWLSGAEALGTESAPGFLPTETIEERKDDEELAVPAEEDQNTCALCGEPFDEFYSDEMEEWMYRGAVYLNAPTGTTAGMDRTQLGPIIHAKCRSESNMATSEDLGPDEKGADEEGSQRKRMRS

SEQ ID NO: 58 - DNA, 9400 bp, Oryza sativa
  source 1..9400: mol_type = genomic DNA
atcgaaacctctcttccccctttcgcctccgcgacgcatatgcctccgcctccgcctcgtcgcggcgcccccaaaccctagcccggcgcggcgagtcccccatcggcggcgggagcggcgggagtaggcgggaaggcgggatctcctccgtcccacatggcggcggcggcggcggttgcgggggcgggcggggtggggcacgtggtggagaggttcagggcgaggctgcgggaggagcaggaggaggaggaggggggaggcggggaggtggcggcggcggcggtggtgcgggtgtacgtggaggtgctctcggagctcacgttcaactgcaagcccatcatcaccgagctcaccatcatcgccgggcagcacgcggccctcgccgcgagggggatcgccgacgccatctgcgcccgcatcgcggaggttgctgttttctctctctcccccctcatccggggggctcttcgcttgtatgtacatagcctaggttttggggatgtgtgggcttgcgtagactactcgtccagtggatatgcttgcttgctgattgttgtgaatgctagggtttggtgatgtgcttctgctgaaaattttagatatatacatgtttttgaattttggagccagtgtaaaagttcgggtttggtgcgctttgtagatgacattttgttcctagggttttgtaatcgttgcatttgatgtgtgatttttgttgacttgatgcacatatagtgaacaatcggatcaatccctgtttgtagtggctccgattgttgcaaatttggttgctagggttcagtaatcagagtgagattcaacaagttttaaagtgaagtgatggaaacggagtgtatgcgattggcacctagcaccagtctgtgatatctaagtgatgtatctataactagaattgagtttcagataatcaagcgtataggtggtaatggcattcacagcctccatgcataggctatcttgttccaaatgaagttgctaggatctttggttctggaagaaattttttgtctctaaaactgaaccaaaaaaagggaaaataatggtgattggttgctcggaatttacatggctgggaatttttctaaaattttcttttagctgtttttttggcacaacataattgctgcttgaaatggaatatcagtaaaacgatttagagttattgttggactcttgggcagtattgttttgctttgtattgtctgttaatagtctattagttatttagttttgatttcttttctgaagagaacattaaaactgcaattttaaatggcaacaatatggtaaccaatcctaagagaaatgagttaataaactaaactaacagaacaccatgtagctttctttacagattctgttcatcatttcataagagactgcttgatttaattttaatcatatgcatgctttctattaattatctatgaacgaactgttcctttacatgatttcttattcaattttgttacaggtgtctgccgaccagaaattaccatctttgtatctgctagacagtattgtgaagaacataggacgagaatatgtagggcattttgccgctaggttgcagaaggtattttgtgatgcctataggaaagttcaccgcaatcagcatgctgcaatgcgacacctctttggtacctggtcacaagtgttcccttcctctgtgctacgcggaattgaggatgagcttcagttttcccccttagagaataagcgctctgccacagcgactgatatccgacagtcagagtcgatatctcctagactgtctcatgccattcatgtaaatccaaaatatttagaagcacaacaacaatttaagcagtcaacttcggtatgtgttttttacttcttgttaataccaacttgattatttgctacagctagttgttttgactatttgttgtgtgatttaagcatggtgtctatttatttcattgcctagcttgagatatatgtgatgcctttctgtttcagatcaattttatacattggagtttgtgcatatattttttaggtgcatcaaccaataactcgtggaaatcggcaaatgaatgatgtggaagaggatcaaataaatggtttaacatctaaaagttcgcgaggatggcctgcaactaattcaaagctccaggtactgggtctttcatcttccttttttttgcttcttgtcattctttcactctgaattaccagccaggtttgagacctactccctccgtcctagaaaaaaccaacctaggtggggatttgacaccacctcctcatgtccagattcattgtcctagatggtgtcaaatccccacctaagttagctttttttggggcggagggagtataagccaatactatacagatagatgctactgttgagtcctatctttttataagtacacagtaatgagttctgttttttgcgcaactcattctggttctcctgtgatcaccagaaaattaaactgcataaatcctaacactgaagacagtatctactagttgcaaatcagatccttaatgcatttgtgcttttttacaacatagaagcagttctgttgttttctttctcatatatgtactgattactgactcatcaataggggctttgaaggtgctctctttgtagcttgacaaaggagaacctcccttttgttctgttttgggagtggttatttgagatagcaacaaccatgtgatttaacacatttgtaaaccctggctctgtttgtagtcttcagtgagttcctataactcttgtgatgacatttgcattgtttgggacaatgtaaagcaccttcgttgtctccaggtgtaacaaaatccatccatctcaatattaaataagagaaaatgctttttacaaccccaaacttcactttcagaactctgcccttctcaagtcttaacattacccaaatttagcctagttttgggtgacatggcgatgtggcataagactttaaatattttctaattttaaaaatttgtaaatgacttcaaaattcttcaaaattataacttctatggagtatttataacatagatatatttctatatgagcaaaatatagtacaatttgcagttttccatgcagaaaatagtgcttaaatatgaacaaaaccatattttaggagataattaaactatattccactctgaacaaataggttctagatggtctaatgtacaaatgtttttatacttcaattactattttgtaaaattcaaatacgtaacccaaggatgaaattgcatttgaatttttataatttttaaaccacttcacaaaaaataatctgtaaagattctcagttgcatattggtgtattgaaacataaataaattatgactttatgagcatagcaaaatcaagtttatttgatgcacaaaaatggttttatctagatttaaaactattttctgtggggattatagtttattcaactcctatgaactttttgttgttgcaaaataccgtatagagcagtggcaatttttaagtatttctaagatttttattttaaaatttttgaaattcgattttttttcagcctttccactttgccatgtaatcttactccatgtctatcaaaagtcaaattgtcaaaaccacttcaaatttgtgcgaagaatgaaaaatgtgtggttttgaatgttcagggttgaattgtgtgcagttatgaagtttatggttgatttctggacactagcatattgcctgtgcgttgcaacagaattttaattaaagaaaattaccatgaacatttcattaacgtctcttaatttttgtatatatattttcaatcacatgtagaccttttttagtcccacattaatctgttttccttcccacaaaaaatatgagagttggtgggagtagttgacttgtgggccctctagtgccacatgtcatcttttctgtaaaaaaaattgggagttgtattgggggtggtggcagaaccaccacctgtctactcccttttataaggagtgtagattattaaatgggattttttcctatgaagttttccctgctcagtaatttttatttcaattcaagttttgatatgctgttgctactttgttgttttgtttatctaaaatgtaacatgtaagttttgtttataatctgtcttgaaatcatatggacagaagtctactatgctgtatgctgacgatcttgatcaacaagaggcattttgttcacatactgggttgataaggccaagttcgccacatcttttaagcaaacatccttccatactcaatacagaaggaccattagccaattcaagaagaaccatgtcaagatcaccacctcttgatgtgctccctagaaatgcatcccccaagagagctctggagaggccaccactatcgcattctgttctaggacctgaccccagaagattgcctgatcgaaatggttggtttgaacggaagtgggcttttgaggatggtgcacaacggccttcgatgagcatacttgatgaagaatatagaaagcaaagtgcaagggaacttattgatgcttatggaaattctcaaggaaaagatgtcgatgaaaggcttcccaagatgcaacgtctagattcaaatggcatggcaggcagacctagtgcacaaaaatggctaacttcggaggaagaagagtatacttgggaagatatgagtcctactttaactgatcgaaacagaaccagcgtgccctctttacctccccttggaaccctgagagctggatttcttgggccaaacagtggactactggaatctgatattgtgaggcacagttggccaggccaggctcctcgaccagcaattgatgggccaccactaaatctcgaagatagaattcctactaacggggtatgcttgtctgaactctattagttatgattattgttactcatgttgaaatgaactttgtgtatcttagcactaataatatttagtgaaataatttcaatcaatatgccatctcttgctttcaggaggtgtttttttattcttcaagtctattaactgttttttataagaaaaggcttccaatagattttgtgcatatacttagttaattaaagcttaaaagattacaatacggcatttcatgttgggtcttccttggcttcactgtttatggtgattatcttccttagcattacatgcttgcccaaagctactctgtttacttacatatactactaattggaggcatttaacacatttttgtttctgtaatgtatatctttgcatttcagcctttcagaactttggtgcaaagtaatttatgccatttttcaccaagagattttgataatgtttgctactgtatattttgtggtcatatgccatgctggctggcacatgatatctccttcattcttcagaagttttaattttgaatatacactagatattcctttaccattttattaccatgatacttaactgcctctacaagttaacattttgtccttgcaatgaacaaaattcatgcacagttcaacttataagagcggaatgctgtactgatctgaatttactttaattaccttttaattagcgttgccttttttgcagcccgttgacagaactaataacaggagatatccaggcaattttggtgttcagaatggtgcctttttagattaccagagttctgaacatactcttgatcctgggagaactaccatgccagttccaccttggcagcagactattggtcagccgttgcgggtacaagcgccccagcctgcatcaattctgaatagaatgccactgcctactgatagtgaagtgccagtcaagaggttggccactggtggtacatatgatgcgctgaatgtagatattccattgttggagaagcaaaggtcctcacctgctcatgctccaatggaatggcctcttaatacacagtcactaactatacaaccaattccacctgataccaaacatcctagaggcgcatcagatggtctagattctagaccatttattagccaagggtccagttcgtctgtttttgttccacagcatcatgccttggatcggagaacaatgaatgctgatgatttagctcaaccatcatatcagcatccagatttgttgtcattgagtcaacaaaatcagggcacagttttgggaaaccaaggtcaacctcatcacccgccgcaatttcatccccatcctcattctcaccttcaagagacaattagaagctttgcccccagtatgtctgttgcacctccacaaaatatattccatggacaaggaggtagcgcagctgcactcctgccaagttctttccctgtaccacctgcagtaccaccctatggcttgcagtctatgcctggatttcctttgccatcgttaccttcaggaccacctcctccatcgcagataggtccatcatcatcgcaagttgggggccctccacttgtctctggattgttaagtaaccttatgcagcatggtataatctcattgcaaccacccagccaacctcaggtactcatatgtgcacctcattgccagtctctgatattgttctatggatacgcatgcagttgattctatgacttacattatacctgtaatctgttcttttaggattctgtgggtgttgacttcaatgttgacctcaaggtacggaacgagtctgtcattaatgctctgtatcaggatcttccgaggcaatgcaaaacttgtggccttcgttttaaatgccaagaagaacatcgtgctcacatggattggcatgttacgaaaaacagaaattcaaagaatcgtaagcaaacttcacgtaaatattttgtcactgtgggtgaatggttaagagcagcagaaacagtaggaaacgatggtgttccttcttttgagcctgccgaaccagttgcagacgcaaaggaagagaaagaattggctgttcctgctgatgaggaccaaacaacatgtgctttatgtcaagagccatttgaagatttctacagtgatgaaactgaagagtggatgtacaaaggtgcagtttatatgaatgcaccagatggaaatattggtggtctagaaaggtcgcagttgggacctattgtccatgctaaatgtctatctgggccaaacaatacttaggccggcctagctgctggtgagtatccatgcattacttgcaacctcactatgaaagtatagctagtatatgttaatggccatatgctcacatgtgttcaactgctgctgtgatcatattatctgactagattataattttggatgtgatagacttctttaacagtgatctcacagggcatctttcttcattggttggggacagcattttacctcgttaatatgattattcattctacttttgtatattctcttttttttttgtcagttctgtggtacttaaattgtttagcactattctgtagtatctacatgccatctattcacctcagggatcctttggatcatttagtacggaaatgcgtttttgtttgatagtttgatcatttctctgtagatctgctaattaccacaagtaacaagatttctgatccttcagtgtgtagggtggacagaacctttggcatgttttggccaccaagtaaaatccattaactaattcagtctatgtgtgtggttatgattactatgattgtcattatgactgcacttactgtggaactgtcatgtttgctgatgatgtgaagataacatttattgttctgcaatggactgctaaaaataaaacagttgtggtttttaaaaatgaaaattaaatgaaatgagcttttttcatacaggtcgaatgagtggtcaattgttctacaatttagtgttatcaagtgtttcctgcatgcaaaagtaaaattgaaaaatctgagttattctagaacacctgaatactcttttttagtatgattcatgattttcaaaggaagattgggtttgagcatactcttctagtagtattatgaaacaatattcttcttgtatgttactagcacactaaaataacattttgtcaaacaaatgatttgtctttaacgcttggacacccaagacggtcagtctcgtagaattagcgggagttgtccatcaattcccgaaactagttgaaccagctcgtacctttttcctactggaaatgtatttttctgttgtccagaaccggaattatgcgctccattgtgcttttcttgttaactgatttacagccaatgttttatgtgtgccaggtcctttttgtttaagatttgtcatatatgctaatattgtgtttgtaattgttcatgcaatatgtaaaccccagacaattacatactgggaattgggatactgagcagtgtaattttgcatttaatataaatagaatgtgatggcctcaactttgatcaccatttggagctaactgttagcagtgtggatgttatatgggaagttaattccttcgtcctatatagaatgatcggtccttttggttcttgggctactctgggttgaggacttacataaactatgagtacaattatgcttattcacactattaagtatatgttgttacacataatttgaagtttgccagcatctgctgcttaaacccctaggagtgcatcattcaaacagacaaattttcaatatgttgcatgagttaactacttcacaattttattgcaggtactcttgcctcatttgtttcgtggctggggctgtagacaagacatgtaaactactggagagagcagattttggtatgcttagtatttaatcctcaattgacaaccatgggtttgcatagaatgttcatgtgatgatagtgtacaactgtaaaatgccttttgtaatatagtgattgagcatgagcatttttgctcgacagtcactatggtgcatgttcaatcgtttggtcccaccatctcacatgcagtgtgctgcttttccattattatagtatattttgaaatccccatgtctactaggtgataggcagccctagttcagaacggtgcaatcactccactgaacttgcgaaacaatatctttctttcttgtttcccttctttccttttgtgcaggacaatatcagtcggctacctggctggtaacttccaaaatcgaaaggacatgtaaggcgatcaaggatggaagcaaaatcatcagcttaatttccagaaaatgaaggagatgctctcttcttgaatttttcccccttttttcatactcttttgtatggcatggcgttctggtattaacacaacagttcattttggtcgcatgtatgactcatcttgctgatatgtttgcacatgtcaagttcagactagattgtttttaagtatgctgtttaaattgatctctggaactcttgtac

SEQ ID NO: 59 - DNA, 2916 bp, Oryza sativa
  source 1..2916: mol_type = other DNA
atggcggcggcggcggcggttgcgggggcgggcggggtggggcacgtggtggagaggttcagggcgaggctgcgggaggagcaggaggaggaggaggggggaggcggggaggtggcggcggcggcggtggtgcgggtgtacgtggaggtgctctcggagctcacgttcaactgcaagcccatcatcaccgagctcaccatcatcgccgggcagcacgcggccctcgccgcgagggggatcgccgacgccatctgcgcccgcatcgcggaggtgtctgccgaccagaaattaccatctttgtatctgctagacagtattgtgaagaacataggacgagaatatgtagggcattttgccgctaggttgcagaaggtattttgtgatgcctataggaaagttcaccgcaatcagcatgctgcaatgcgacacctctttggtacctggtcacaagtgttcccttcctctgtgctacgcggaattgaggatgagcttcagttttcccccttagagaataagcgctctgccacagcgactgatatccgacagtcagagtcgatatctcctagactgtctcatgccattcatgtaaatccaaaatatttagaagcacaacaacaatttaagcagtcaacttcggtgcatcaaccaataactcgtggaaatcggcaaatgaatgatgtggaagaggatcaaataaatggtttaacatctaaaagttcgcgaggatggcctgcaactaattcaaagctccagaagtctactatgctgtatgctgacgatcttgatcaacaagaggcattttgttcacatactgggttgataaggccaagttcgccacatcttttaagcaaacatccttccatactcaatacagaaggaccattagccaattcaagaagaaccatgtcaagatcaccacctcttgatgtgctccctagaaatgcatcccccaagagagctctggagaggccaccactatcgcattctgttctaggacctgaccccagaagattgcctgatcgaaatggttggtttgaacggaagtgggcttttgaggatggtgcacaacggccttcgatgagcatacttgatgaagaatatagaaagcaaagtgcaagggaacttattgatgcttatggaaattctcaaggaaaagatgtcgatgaaaggcttcccaagatgcaacgtctagattcaaatggcatggcaggcagacctagtgcacaaaaatggctaacttcggaggaagaagagtatacttgggaagatatgagtcctactttaactgatcgaaacagaaccagcgtgccctctttacctccccttggaaccctgagagctggatttcttgggccaaacagtggactactggaatctgatattgtgaggcacagttggccaggccaggctcctcgaccagcaattgatgggccaccactaaatctcgaagatagaattcctactaacgggcccgttgacagaactaataacaggagatatccaggcaattttggtgttcagaatggtgcctttttagattaccagagttctgaacatactcttgatcctgggagaactaccatgccagttccaccttggcagcagactattggtcagccgttgcgggtacaagcgccccagcctgcatcaattctgaatagaatgccactgcctactgatagtgaagtgccagtcaagaggttggccactggtggtacatatgatgcgctgaatgtagatattccattgttggagaagcaaaggtcctcacctgctcatgctccaatggaatggcctcttaatacacagtcactaactatacaaccaattccacctgataccaaacatcctagaggcgcatcagatggtctagattctagaccatttattagccaagggtccagttcgtctgtttttgttccacagcatcatgccttggatcggagaacaatgaatgctgatgatttagctcaaccatcatatcagcatccagatttgttgtcattgagtcaacaaaatcagggcacagttttgggaaaccaaggtcaacctcatcacccgccgcaatttcatccccatcctcattctcaccttcaagagacaattagaagctttgcccccagtatgtctgttgcacctccacaaaatatattccatggacaaggaggtagcgcagctgcactcctgccaagttctttccctgtaccacctgcagtaccaccctatggcttgcagtctatgcctggatttcctttgccatcgttaccttcaggaccacctcctccatcgcagataggtccatcatcatcgcaagttgggggccctccacttgtctctggattgttaagtaaccttatgcagcatggtataatctcattgcaaccacccagccaacctcaggattctgtgggtgttgacttcaatgttgacctcaaggtacggaacgagtctgtcattaatgctctgtatcaggatcttccgaggcaatgcaaaacttgtggccttcgttttaaatgccaagaagaacatcgtgctcacatggattggcatgttacgaaaaacagaaattcaaagaatcgtaagcaaacttcacgtaaatattttgtcactgtgggtgaatggttaagagcagcagaaacagtaggaaacgatggtgttccttcttttgagcctgccgaaccagttgcagacgcaaaggaagagaaagaattggctgttcctgctgatgaggaccaaacaacatgtgctttatgtcaagagccatttgaagatttctacagtgatgaaactgaagagtggatgtacaaaggtgcagtttatatgaatgcaccagatggaaatattggtggtctagaaaggtcgcagttgggacctattgtccatgctaaatgtctatctgggccaaacaatacttag

SEQ ID NO: 60 - protein, 971 aa, Oryza sativa
MAAAAAVAGAGGVGHVVERFRARLREEQEEEEGGGGEVAAAAVVRVYVEVLSELTFNCKPIITELTIIAGQHAALAARGIADAICARIAEVSADQKLPSLYLLDSIVKNIGREYVGHFAARLQKVFCDAYRKVHRNQHAAMRHLFGTWSQVFPSSVLRGIEDELQFSPLENKRSATATDIRQSESISPRLSHAIHVNPKYLEAQQQFKQSTSVHQPITRGNRQMNDVEEDQINGLTSKSSRGWPATNSKLQKSTMLYADDLDQQEAFCSHTGLIRPSSPHLLSKHPSILNTEGPLANSRRTMSRSPPLDVLPRNASPKRALERPPLSHSVLGPDPRRLPDRNGWFERKWAFEDGAQRPSMSILDEEYRKQSARELIDAYGNSQGKDVDERLPKMQRLDSNGMAGRPSAQKWLTSEEEEYTWEDMSPTLTDRNRTSVPSLPPLGTLRAGFLGPNSGLLESDIVRHSWPGQAPRPAIDGPPLNLEDRIPTNGPVDRTNNRRYPGNFGVQNGAFLDYQSSEHTLDPGRTTMPVPPWQQTIGQPLRVQAPQPASILNRMPLPTDSEVPVKRLATGGTYDALNVDIPLLEKQRSSPAHAPMEWPLNTQSLTIQPIPPDTKHPRGASDGLDSRPFISQGSSSSVFVPQHHALDRRTMNADDLAQPSYQHPDLLSLSQQNQGTVLGNQGQPHHPPQFHPHPHSHLQETIRSFAPSMSVAPPQNIFHGQGGSAAALLPSSFPVPPAVPPYGLQSMPGFPLPSLPSGPPPPSQIGPSSSQVGGPPLVSGLLSNLMQHGIISLQPPSQPQDSVGVDFNVDLKVRNESVINALYQDLPRQCKTCGLRFKCQEEHRAHMDWHVTKNRNSKNRKQTSRKYFVTVGEWLRAAETVGNDGVPSFEPAEPVADAKEEKELAVPADEDQTTCALCQEPFEDFYSDETEEWMYKGAVYMNAPDGNIGGLERSQLGPIVHAKCLSGPNNT

SEQ ID NO: 61 - DNA, 6256 bp, Oryza sativa
  source 1..6256: mol_type = genomic DNA
tgtgttcctctcctcttcatgtggagtcttgagtcttgacaagcaaagaaggaaggaaacctcctcctcctcttcttcggctccaaatcaaccaaacaaacacagcttccgactctctccctccctccctccgcctccgcccatccaatttcttaccccgttcgactccactcgactcgacccgacccgcaggagagcgggcgtcgcaaaccctagctccacccacacccacacgctcatgccccccaaaccctagaccatgcgcgcctccgccgcccccatggagacctccgcctccgcccgcagatccgccgcccccgaccccaagaagcctcgcctcgcccagcaccccgcccccagggatcccagatcttaccccgctgccgccaatggcaacgcccccgccgtcgaccaggcgcaggtggacgagctcgtcgcgcagtaccggaccgccctcggggagctcaccttcaactccaagcccatcatcaccaacctcaccatcatcgccggcgagaacctccacgccgccaagcccatcgcctctctcatctgcgccaacatcctcgaggtgacccttctcctcttccgctattccattccctttctctccatgtaaaactattgaggcgcctcctgtcctacttactactttatcatgtacgtcttgtgctagatacagcaaacgtgctcccatgagcagccatcttttttgtattctactctacatacataataacatagctaatcgagatgagatccctgtgctgtgcttgttgcccaaacatcaccttctttactaatgaactgcgcactgacctaatttctaacaaaaggtttaaaccacccgcatttcgcctatgttgatgtatgcaaatatcagggtaatgagtgtttcgctatggactgaatttctctctctcttacatcctatccctttctatgtgtgcgtgctcatgcactactcttgtatgacgttaaggcacatggttatatacgctttaccgttgcggaatccatttcaagttaccgtgtgacgattttatgcccacgatatcattcactgtatgaatatcagacttactcgtcatggcagaaggatatcaatggtagggttctatcaccctatgcaagtgttctatgatgctagaaaatatcataagtatcgacttatggttgcaaactgtaccaggcaggcttcagtttcattcctactcttccactatctgatccccagatttgtgctgcactgcatgttgaactgttctcatgctgataatgttctcaaattgtgtacctcatctccaccgttgcacacaagatggatccagatttcagaaatccatgtttatgattgaactctttagcgtgttcctgtaaaggtgaaaaaactgtataaattgtgatgagaggatgtggaaatactgcacctcataactggcctacgctggttggtgttgtcatgtagctgctggctacttaacttagatggttttcgcactgttcacttcttttgtagcctctgctcttcctaggtgatgatgcctactgataactcttgcatttcagctaggctcattatcttcacacgtttcgtgtgtttgaacaaaatgttgttggacttctcaacaagtagatgtaactcaactaaacaaaagatggaaaaacattgggccttctctatgtgagggtcataaagaaaatttgtattggtctgttgtgtagcagatttttctagtcttggagagtggagacacagctattatagcagtgacaccaggttttccattgttgtacttgtatcacattaacatttctgtgattctcttttttcattgtaaacctgtagtacctgcttatgtaattgtattcttataggtagaagggtatgctatttcatattattggctgcttgtgcttgtaggttccaagtgaacagaagctaccatctctgtatctacttgacagtattgtcaaaaatattggaaaagattatgttaaacatttttctgcaagattacccgaggttagtaaataatcgttcttgctcttctaacttttttttttgttgacctgatagttgagatgaccagcttcatggaaaaataatgaatcctggtttctttctgtatgagatcaaccttagttttcttgggcggttccttgttattgtaggtgttctgcaaagcatataaacaggttgattcttccatacacaatagcatgagacacttatttggaacatggaaaggagtgttttccccaacttcactccaagtgattgagaaggaacttggctttcaatcatctactaatggatcatcaggcgctgcaccatcaaagcctgattctcaatccaatcgtccatcccacagcattcatgtgaacccaaaatatttggaagcaaggcagcagctccagcaaccaaataaggcaagccaacttctttttttttttttttgtagatgtagctattcaatgctcttgtcatcctgagtatcgtatcaagactgatagatattgctcccattgaaaaaatatagatatgttcatagcattgtaactgctgctttatactatcacatcactcgtgagattttttcacctcattctattctcccctcgttctgtacagggacaggggatactgggagctggggccaagactactacaatttccgattctggtgatgatattgagaggactagcaggacagctgttgatagaggtgcagggcggcggctggatgctttgaaccctaggactgtaagttcatcatttgatcattgagtaacataagtgcttagatgtatttactttgttatatatataatgcatatgttattatttgactttgatatgcagaatgtacagcgggcacaaagggacccttttagcaatcctattcatgaaaagcaagatagagatatgagggtccttggattctccaacatttcgcagcaggctgttgtgggaactggtcttgttcgtgcaaaacctaaaggccaagatggaagtggcggaccatattatactgctggagttggttcttctgaagaacagtttgacaggcgcagcaatttttatgcaaacaaggatgttagaccatcaggatctgtacgtttagatggtgcacttttgccaactcctgttagtaattctgataggattggtaggccgtcatccaacaaaagctggaagaattctgaggaagaagagtacatgtgggatgatgtacgttctcaaggagcagattatggaggtaccagttcaacaagaaaaagagaatggatacctgatgatggaaatgttggaagctttcaaagggttaaatgggcagaggcgggaggacctttggaccctgaccaacacaaattagatagcttccaaagatttggaaatgcgacgggtcaagatagaagaattacaccatatatggtatgtgaaaagaagacagtactataaaatcatattcaatacttgatagatacagcaagttaatttcttgtacctaatgttgttagatagtttgaactgctggttcgctctcaaatttgcaatttataatattgcctcaaaatcttaggttttacctgtataccatgctaaattttgatgacatatatacacatattccatctttgcactacttttggacaagattataatctgattgttttttcaattgttcaggatcatgaagagtaccttcatggtaagcacgaagtggaaccaagaattgatagggaaatgctcccggaaggacagccgttctcgtcatctcgtggttcctctctctggttatcccatgaaaaacctctcccagacattgtgtcagatcctaggatatcagcgttttccaatcagccagcggacaggcctaccatttatgctggtaccttgtcaacaagtatcacttcatctgtacctgttgggttgtcagcagcctatgctggacgatcaagcctagaaagtgcagcaacaagatcaactgagactataggacagcaaaagaatagatattggtcaacttcctcaccacctgtacagtcaccttctgcatcatttgcacgccagagttcccctagtcctgtagagcttgattactcttccaaaccattctcacagttgggtcaaaattctctggaagactataaccaaagaactcatgccttggctcaaaaccttgcactttctcagggcagacctaatctgctgggtgctccttcacatgcctcccagcagattgaaaagcacccttccttgctgcagtcaaagccacatctcaggacacttgatcaaccgcaggcaaacttttcacctgaaaattcatcttcagtatttaaatcttcaatccagctgcctatttctgttggcgtggggcatcggcagcctgaagaagtatctctgtctagtgattccactcttatgagctcagatcatttaagtgctagcagtttgttggctggccttataaagagtggattcaaaccgaatgaccccaacgatcttgccagtttgagggcacaacctcctcttccaagtgggccacctcctcatgtttcgacatctttttcagctgcttcctcatccctgcaccttccagctagtgatactctgaagtctcaggcgccaaactctctgcgcccacctttgccccctgggttgccactttcgtcgccgtttgtgtgcccgacaacacagacctcggagaaagctgcaccactctctagtcttctcagttccctggttgcaaagggattgatttcttcgccttctgcagattcaactgtagcgatcccacaacagccctgtaaatcagaactgaacacaccagatgacactgcttcagctccatctctgccttttgtccagccttcagttaaaaaggaaacatccaatcaaaactcatctgccccatctaaggtgctggtgcatcaacctgttgaaatcaagaaggaacctgcagaaatcaagatggtagaccttattggctttgacttcaagcctgagatgctccgcaagtaccatgcacatgtgatcagcaccctctttgatgaccaaagtcatcaatgtaatacatgtggtctaagattcagtcttgaagaagagctctctgtgcacactgcgtgccatggatcgaagcaaagtgagactagaaagactggtatcgcacctgaaaaatggtaccctagcaaaaataattgggtcgatagatcacatgaagtgcagaacagcgccctggagtctgcttcttctgttgcagatttgagctctgaagaggaagtttgtgaattcatggttcctgcagatgagagtcagattatctgtgcactctgtggagagtcatttgatgacatttattccattgagaaggataactggatgtacaaggatgcggtctattttgattcttcgaagacagaaggcagcagtggcgacagtgctgagagtaaagagcgtgtacccatagtgcatgcaagatgtatgtcgataagctcaaatgatggcatggaggttgattagcatttgtgccaaacacaggggcagaagcagtagcaacagaaacagctcgaatgatgagattgaggtggattagctggaaatctggtgcttaacagaagctcaaatgaaatgatgccacaaaagtgggtcagcgtgaggggtgatccgcagcagcaactgcaatgatgctgtcgcggccatgacatgtaggtggattagtgtgaagttgacaacctgacgcgctgcacaagtaaattttggtcaaatgtttgtgttcatccctcagtcaccattaatcgtggataaggtgcttacacagaataaattccaagtccagctctggccttgtcaaccgatcatcctcgcaatcgagatcttcgggggtctgatatttgtatttttggttcaatcgaagggaaatgatctttctgttcatagaaggccagcttgtatgttaaaaaggagtgtattgtttcattacgagggaaagcaataggtgacgctgccattgagttactgttgttttttggcaaacttgtttcatcttccgaggactagagattttgtacctaaaaagaactgataatagaacatcatggaggcagaattaaactagttacttatggcttgcttgcttgatgtgggagttcttcattgtagttcgatgttccttcctccttgccattgtgatgcgtaaaattgttttgtcgtcggaagttgttaattcggcagatgatagatctgagtatt

SEQ ID NO: 62 - DNA, 3210 bp, Oryza sativa
  source 1..3210: mol_type = other DNA
atgcgcgcctccgccgcccccatggagacctccgcctccgcccgcagatccgccgcccccgaccccaagaagcctcgcctcgcccagcaccccgcccccagggatcccagatcttaccccgctgccgccaatggcaacgcccccgccgtcgaccaggcgcaggtggacgagctcgtcgcgcagtaccggaccgccctcggggagctcaccttcaactccaagcccatcatcaccaacctcaccatcatcgccggcgagaacctccacgccgccaagcccatcgcctctctcatctgcgccaacatcctcgaggttccaagtgaacagaagctaccatctctgtatctacttgacagtattgtcaaaaatattggaaaagattatgttaaacatttttctgcaagattacccgaggtgttctgcaaagcatataaacaggttgattcttccatacacaatagcatgagacacttatttggaacatggaaaggagtgttttccccaacttcactccaagtgattgagaaggaacttggctttcaatcatctactaatggatcatcaggcgctgcaccatcaaagcctgattctcaatccaatcgtccatcccacagcattcatgtgaacccaaaatatttggaagcaaggcagcagctccagcaaccaaataagggacaggggatactgggagctggggccaagactactacaatttccgattctggtgatgatattgagaggactagcaggacagctgttgatagaggtgcagggcggcggctggatgctttgaaccctaggactaatgtacagcgggcacaaagggacccttttagcaatcctattcatgaaaagcaagatagagatatgagggtccttggattctccaacatttcgcagcaggctgttgtgggaactggtcttgttcgtgcaaaacctaaaggccaagatggaagtggcggaccatattatactgctggagttggttcttctgaagaacagtttgacaggcgcagcaatttttatgcaaacaaggatgttagaccatcaggatctgtacgtttagatggtgcacttttgccaactcctgttagtaattctgataggattggtaggccgtcatccaacaaaagctggaagaattctgaggaagaagagtacatgtgggatgatgtacgttctcaaggagcagattatggaggtaccagttcaacaagaaaaagagaatggatacctgatgatggaaatgttggaagctttcaaagggttaaatgggcagaggcgggaggacctttggaccctgaccaacacaaattagatagcttccaaagatttggaaatgcgacgggtcaagatagaagaattacaccatatatggatcatgaagagtaccttcatggtaagcacgaagtggaaccaagaattgatagggaaatgctcccggaaggacagccgttctcgtcatctcgtggttcctctctctggttatcccatgaaaaacctctcccagacattgtgtcagatcctaggatatcagcgttttccaatcagccagcggacaggcctaccatttatgctggtaccttgtcaacaagtatcacttcatctgtacctgttgggttgtcagcagcctatgctggacgatcaagcctagaaagtgcagcaacaagatcaactgagactataggacagcaaaagaatagatattggtcaacttcctcaccacctgtacagtcaccttctgcatcatttgcacgccagagttcccctagtcctgtagagcttgattactcttccaaaccattctcacagttgggtcaaaattctctggaagactataaccaaagaactcatgccttggctcaaaaccttgcactttctcagggcagacctaatctgctgggtgctccttcacatgcctcccagcagattgaaaagcacccttccttgctgcagtcaaagccacatctcaggacacttgatcaaccgcaggcaaacttttcacctgaaaattcatcttcagtatttaaatcttcaatccagctgcctatttctgttggcgtggggcatcggcagcctgaagaagtatctctgtctagtgattccactcttatgagctcagatcatttaagtgctagcagtttgttggctggccttataaagagtggattcaaaccgaatgaccccaacgatcttgccagtttgagggcacaacctcctcttccaagtgggccacctcctcatgtttcgacatctttttcagctgcttcctcatccctgcaccttccagctagtgatactctgaagtctcaggcgccaaactctctgcgcccacctttgccccctgggttgccactttcgtcgccgtttgtgtgcccgacaacacagacctcggagaaagctgcaccactctctagtcttctcagttccctggttgcaaagggattgatttcttcgccttctgcagattcaactgtagcgatcccacaacagccctgtaaatcagaactgaacacaccagatgacactgcttcagctccatctctgccttttgtccagccttcagttaaaaaggaaacatccaatcaaaactcatctgccccatctaaggtgctggtgcatcaacctgttgaaatcaagaaggaacctgcagaaatcaagatggtagaccttattggctttgacttcaagcctgagatgctccgcaagtaccatgcacatgtgatcagcaccctctttgatgaccaaagtcatcaatgtaatacatgtggtctaagattcagtcttgaagaagagctctctgtgcacactgcgtgccatggatcgaagcaaagtgagactagaaagactggtatcgcacctgaaaaatggtaccctagcaaaaataattgggtcgatagatcacatgaagtgcagaacagcgccctggagtctgcttcttctgttgcagatttgagctctgaagaggaagtttgtgaattcatggttcctgcagatgagagtcagattatctgtgcactctgtggagagtcatttgatgacatttattccattgagaaggataactggatgtacaaggatgcggtctattttgattcttcgaagacagaaggcagcagtggcgacagtgctgagagtaaagagcgtgtacccatagtgcatgcaagatgtatgtcgataagctcaaatgatggcatggaggttgattag

SEQ ID NO: 63 - protein, 1069 aa, Oryza sativa
MRASAAPMETSASARRSAAPDPKKPRLAQHPAPRDPRSYPAAANGNAPAVDQAQVDELVAQYRTALGELTFNSKPIITNLTIIAGENLHAAKPIASLICANILEVPSEQKLPSLYLLDSIVKNIGKDYVKHFSARLPEVFCKAYKQVDSSIHNSMRHLFGTWKGVFSPTSLQVIEKELGFQSSTNGSSGAAPSKPDSQSNRPSHSIHVNPKYLEARQQLQQPNKGQGILGAGAKTTTISDSGDDIERTSRTAVDRGAGRRLDALNPRTNVQRAQRDPFSNPIHEKQDRDMRVLGFSNISQQAVVGTGLVRAKPKGQDGSGGPYYTAGVGSSEEQFDRRSNFYANKDVRPSGSVRLDGALLPTPVSNSDRIGRPSSNKSWKNSEEEEYMWDDVRSQGADYGGTSSTRKREWIPDDGNVGSFQRVKWAEAGGPLDPDQHKLDSFQRFGNATGQDRRITPYMDHEEYLHGKHEVEPRIDREMLPEGQPFSSSRGSSLWLSHEKPLPDIVSDPRISAFSNQPADRPTIYAGTLSTSITSSVPVGLSAAYAGRSSLESAATRSTETIGQQKNRYWSTSSPPVQSPSASFARQSSPSPVELDYSSKPFSQLGQNSLEDYNQRTHALAQNLALSQGRPNLLGAPSHASQQIEKHPSLLQSKPHLRTLDQPQANFSPENSSSVFKSSIQLPISVGVGHRQPEEVSLSSDSTLMSSDHLSASSLLAGLIKSGFKPNDPNDLASLRAQPPLPSGPPPHVSTSFSAASSSLHLPASDTLKSQAPNSLRPPLPPGLPLSSPFVCPTTQTSEKAAPLSSLLSSLVAKGLISSPSADSTVAIPQQPCKSELNTPDDTASAPSLPFVQPSVKKETSNQNSSAPSKVLVHQPVEIKKEPAEIKMVDLIGFDFKPEMLRKYHAHVISTLFDDQSHQCNTCGLRFSLEEELSVHTACHGSKQSETRKTGIAPEKWYPSKNNWVDRSHEVQNSALESASSVADLSSEEEVCEFMVPADESQIICALCGESFDDIYSIEKDNWMYKDAVYFDSSKTEGSSGDSAESKERVPIVHARCMSISSNDGMEVD

SEQ ID NO: 64 - DNA, 14778 bp, Zea mays
  source 1..14778: mol_type = genomic DNA
gaaacgtgaccctcgccgtttacgaacaaatcgccgacccgtctcctgctctctcccaatttcagtcagttcgctccgctcccacatgcctcctccgcctccctctcgccgtcccggcatacccaatccctaggcaccccgccgaaaccctagattctcctctcctgctccgatggctggggcagcggcgtcccccgtggcggcccacgtggtggaacggttccgctcgcggctgcgcgaggaagcgggcagcggggagccgggcgcctccgcggtcgtgcgcttgtacgcggaggcgctccgcgagcttaccttcaactgcaagccggtcatcaccgagctcaccatcgtcgcaggccagcacgccgcgctcgccgccgggggaatcgccggcgccgtctgcgcccgcgtcgccgaggtcattctgctgctctttgctaccccccatgactgttactcttgttttccagttaaggcggcccgcgttatgatttttggggcataaggaagcttggtcttggagcgtcgagtagtctggcgctcacatggttttggaggttgttgtgcttgttgtgaaatgtggattgcttggctttgttgttgaatgtgagaattcccataaaatggacgggagactttttatttgtaggttttacaatagcgtcagtttgtacaagatcattcctttttgtgagaagcgatgtacgttcagtatgtttgctctgattgctgtttacagttctggtagcatatttagatcctggggtttagaatctttgtggaactttgtagatcaattggtgcttgccattttaatttttcttaaaattcctgctcaaaactagtaggatgctgacatacatgcatgaaatatgtagcaacagagtaaacagttgagcttaagagtgtatgagcactgcattggagaaaatagtcgctatgactgataaacttcctttttgatcggaagttgagagttatcctatcgaattgagataaaccttaaacaatgttatagataaatgcgtcatgtgttacactggttcttattttgacttacatacgaaaagagttcagaattttgggtttattattgcttattaggatagttgtgaactaagtcagttacctgtttacttgttctgtatggtgtggtagatttggttttgtttagaaatatcttgtaattttctggctgcttttcttgaagataagacatacaagtcttcaattttaaatggaaacaatatgggaactaatcataagagcgaggattaagctactagtgggtttgcagtttttctttcagaactgcaggctgtttgagtttgttcatcaaccagttttttttcatatgcatgatttttaattaattacgactttcaaagttcttttgtgtgatgattgttcttctgcgatattcttgcaggtgccacctgatcagatattaccatctttgtatctgctagatagtattgtgaagaacataggtggggaatatgtagagcattttgcaattaggttgcaaagtgtatttgttgatgcatactacagagttcatccaaaccagtatgcttcaatgaggcgcctctttcgtacctggtggccagtgtttccttcttctgtgcttcacagcattgaggatgaccttcaattttcgccctcagagaataaccgtcccaccacatcgacaaatgtgcatcaaacagagtcgttatctcctagaccatctcatggcatacatgtaaatcctaaatacctagaagcacaacataagttcaagcgggtcaatgtggtaagactttgttaacatcatgtatatatatgagaatcactgttgtatttattttggttcaagcatgataattgtagtctaatgactgttaataatttgttgtattgttgcacctgttgcattctgtaacagtctatgcgatttaagttaactttaaatttattgacgatgtttgtccactttatttccttaaaattcaggcgcaccagtcagcagctcaggacactaggcaaatgtctgatgtgcaggaggatctgataaatggactaacatcgaatagttcacaaggacggccttcagtgtttcaggtattcagattgtctcttcccttatcgttcttgtcatttttgcaaccactatccaatttcaagcctggcttgcatgtaaaaaactgtcttctgattcttttgctccccttattggccgagaaaggaaaaacgtttttttgtcttaatctttttggggaaatactcaatttatttaagatactggttctggttactgattgcttcttccaaaagttcctacaacttggttccttccaagagttcctatgactcggttccttccaagagttcctatgactcagttccttccaagagttcctatgactcggttccttccaagagttcctacaactctacctggtttagtattcttgataacaaacaatcccacaccaaagcacatggctaaggcccgaccccggtcacaatcattctcacatgggctatggtgccgctgtgtatgggtggggcaggggttcgggagtttttttgatctgcatgagaagaggtctttttaatgtaatactgtgagggcgatcttacccctttcaggttgagttttttaaacaatcgatcttgtctgacatttttgatgctttattttacctcaagcgaaatgaagcaaaagtactcaaatctactccttccatttcaaaatgttggtcattttagttttgttctaaatcaaatgtctctagctttgaccatgtttagaaaaatgcaccaacatgtagagcatcaaattagtttcgttaaatttctaagagaacgaaaacctaatccattgatagtgggttgcaatatacatattccctaaccctaatgggatacatgggttgggctcgaggcccataacatatagacacacacacacatacaatctaacccctagtggaaactctagccatgacagattgagactggaccaaaactctgagaacaccgaggaggatggaagacccttagtgaagatgtctgcaaactgcgagctcacgaacgaattgaagatcgatgtcaacatgcttcgtgtgctgttgctggaccaagttgatggagaggtagacgacgttgacattgttgtagtagatatgtgtactcttggtgagaggaatatggagctcctaaaggagctagcggagccagttcacttcagccacgccgttggcaacgacactgtacttagccttagcgtgggaataggaggccacatgttggcacttgaaggaccaggagatgaggttgttcccaaggaaaacgatgtagccaaaggtggaccgttgggtctttgggcagcctgcccagtcagcatcggaggagcagacgatcaacacaagggtggggtgtcgaaggagaagttcaaaggtatcgaaagatctgcttgacaaccatgaggtgcagctcttagggtcatgcatgtgaagacaaacttgttagaccacataggcgatgtcaggctggggaaggtgaaatactgtagggcaccaacaaggctccgatagccgtgaggtgcggctcccggggatcattcatgtgaagttggacttgttggaccacataggcgatgtcaggccgggtgaaggtgagatactgtagggcaccaacaaggctccggtagtcagtaggatcaccaactagaggactgtcaccagagaccttggcctatgtgtccaccggagtcgcacacggtttaaactccgcatgccagcatgctttaagatatccaaagtgtactaccactgttgaagaaaaaggccagtcgagtgatgctcaacaacaacacccagtaaatgatggaggcctagatctttcatggtgaactgatgctgaagagttgtgatatttcaatgcagaagagttgaagaagagggagtgaggacaatgtcgtcaacttataggagtagacaatgtcactgtcacactagtagatgaacatgatagtatcagatttagcctcagtgaatcctaggaagagcaagtaggaagcaaatcggctgtacgaagtgttggttgttgctttagttcgtaaaaaggcttgttgagccttaggtccttgcttaagtccatacagagacttgttgagcttgcagacccagtcggggtgagcggagtcaacaaatccagcaggctggctgtaataagttgtctcagtaagggtgcgagaaaaaagcattttgacatctagctgatagactggctaagaatgggacaagcagactgcaccaatcaccattagactgaaggtctcatcataatcgacgctcgggcgctgggtgaatcctcggagaacccaatgagccctgtacctgtccagatagaaatccacttatgggtctaaacccacttgctagccagcacattgccgcatggagggtgcgacaccaagtcccaggtgtggttggcaagaagggtgtcacactcttcctccatggtgtaacactagtgaggatcagtgagggcgcgatggatagaagatgtcacaggggacactaacgaagaggaggatgcgaaaaggacaagacagtcgccaggatgaagaactctagcgtcctgataggtaaccattggttggacgtgacggtgtttgcggtgaagtagagggtgatacaccgccagctcaacctgagagctaggtggggcaccctcttgtgcacgtcgttgataaacatgcataagattgatgaagtggacaaagcacgtgagggtatcgacaacgccacaggtgagctcggggccacgacatgggacgttgggggcgcgtgggtgggggacgacagtgccacatagcgcaatcaggctcagtgcctgggcggcatcgggaccgcagggggtagcggtggagtcggggaacctgctgagaaaaatagaggtggctggctagagggagccatagggtcagttctagaaagacgtctagctcgatagtagagctagagggggatgtggggaagaagtccgtctcgtagaaatataacatggttggagatcaacaagcggttggtgaggttaagacatcgatatcccatatgatcagaagagtacccgagaaacacacaacgagaggatcaacaggaaagcttatgagaagctgtggcatagagactggggtagcacacacacacacccaaagacacgaaggtggtcatagaaaggagtggttccgaagacggcaaaatgaggcgtgtgggagcagagagagctttggtagatagtcaattgagcaggtaggtggcggtgtgaagactctcgacccaatagcgagcaagaacagaggcctgaaacaaggaacacatgatgtcgatagtagcacgaatcatatgctcaaccttgtcggtttgggaagaggatacgagcatgacattctagctgcataccatgtgacagtgacattgttcatccgccgcgctgcctcctcacatcgatgccgcccctgtcttccctcattcacccctaccgattgagagagatccctggtaggatcaggaaccctaccagtccgcctcattgcttaaggcgttgccccaatgcttcgagcggtggcaaggcattaatttgtggatgctttggcctttgtagggtttggggaacgatatagaggtcggaaaagagaggcttacagtgcgagacactccaagcggcgatgagtggaacctccaggcgatggccatcttccttctctccaggcggcgacggtggccctacgcccctgcctcttctctagcggctgatggttcccctccgtgagctcccctacacccctacctcttctccagcggcgggtggcccccctccacgagctccccttgtactccgcccttgcctcctgtgagctcgcgacgagcgagccgggggtgggcgccggtcctgcttctatctcatcctcacaactggaactgggtcacaaggcggtgagatcatgggagggacaatcggtttgtgtggttgcagttgggaactgggtcacggtcttgtatgttcgttgggccatgctgaaattgtttagaaactcagaggcgggtagtaacaagccggcgccgtcctactccggccctcgtgggcaaaatgccctgcatgttgctgtcctaaacaacaaaagaggtgacataaacaaacaatataatatatagtacacatcacggtcaatatataatattgtatggaataattaacactcttctacttgtcctcctagcctaatggcttggttttccaatatatttttatcatcatctttttatattctattggctacacgccttcacatattacttatgttggactttaaatcaaatatgttgatttatgttaaacttgtactcctatttgctacttagtactattatccatatctatttgagactacattcctatattttctatttttatttcttatttctatgtattactaaggcgtcgcctcaccttacatcttaacacttaaaaggtaaaaagggggttggctgccttccctcgtcttaccgccttaaaagggaaaaattgctttttaggtcccttgtcgttgttgcttgccaaaatatcctggcattcgttgcactcgcttaattgccccacttaaacaacgcgtagttgatgaaataccctagtgtagctatttttatattttcatggacccaataattcgaaatgtcacttttaccctctgtatacacaaaacagtgttaagctttgttacttcgatgttacacaatatatggagtcgttaacagcaaacaaaacaaaacattagcaatgcagttaggtagaagcaaagaataaatgagcacattcatttcagggtcaagcagattgggagatgcgttatctgattttttcctggtaactgaaggggaactttgtgaaaaagtgtgcaatctattctgttgatgatctcagtcatgcttcaaggttggagcattctctgtccagcatgaaggaagctagaaattcaattccaaactggaaagtctcacaatgctggctatcaggcctctaaggctgactggctagaccaggtggatggagagtggaacaagtatctatacaagacggcgctatctggactgggagaggaaggatgtgccaaaaacattcacagtagtaagtaatagcgaaccccatattaggtaaggagtaacctgctcacatcatcagttgatagtctgaaacaatggaataaatggaataagacaagagaaataggaccagttgacaccaaagaatcctaacaaaagcactgttaattctgaagcgttttttctaagcattgtgcagacaagttaccatcatgtagtggtactctatgttgaacacaatttcttcaaatcgagccaggtactagaaacaataacagtatagccaggtatagacggtcatgtctaagaatagtgacacataagaactacagatccatgatgatgctcaattcatcctcattatgtgttgctcatattagataagtacctagatagcttacaagagatggtatattcagattttagatatttctcctgaagggctttgcatagtagaggaacatccgagttttcaaaacttggcactccctgtttggctgctgaaagctaatcagagtgtgcaactgatcagaggacgctgtagcacgcctctctgtcatgtttgcagagggtaaaagtggcatttcgaattattgggtcatgaaaaatatatgaatagccacaatggggtattttatcaactacgcgctgcttaagtgggacaattaagtgagtacaacgaatgccggggtattttggcgagcggcaacgacaagcgacctaaaaaaagtaattttccaccttaaaaactatggtggaaacccaagcaaagaagtgcgaaaagagaaatggtggaaaaagtattagacttcaagcgcaaagggaaagtccatgaataatgagataaatcaagtatcaccaagtattatttatacccagaaacattgacaacaaagggatgcccacaaatcagtgtatgataaaaaacatgtatagcctgagaggagctagagaaggggagtcgaacatgctgacctagctaacaggcatgttatagatgctcacgagtaccccaaatacatccattgatagtgggctacatgggctgggctcgaggcccataacacacttaaactctaagtctcacaaattcaccatgaaatgtgtcttgaaagtgcttagcattcaagatgttaatttatttttctataaattcagtcgaagttagagaagtttgattttgtttaggttagaaatattacaacattacatttttgggatgtagtacttcaacatccataactgttcatctgcatttctttttcttccaatccaagttgtgaaatgctatttctgcctctggtttattttgtgatggctcataatctgtgttgtaaccatatgggcagaaatcttctttacagtatgctgatgatcctggtcaacaagagatgtttcgacctcttgctggagcaataagggcaacatcgccacatctcttgagaacacatgcatctgatgtgaatctagatgggccattagccaattcaaggagaaacttgtccagatcaccacctcttgatgtgttccctaggaatgtttccccaaagagaccactagagaggctaccaccatctcattctgtattgggacctgatcccagaaaattgcctgatcgaaatggcaggttaaggtgggctttggacgatggtgcacagtggcctacaataagtatgcttgatgaagagtacaggaagcaaagtgcaagggaacttattgatgcatatggaaattgtcaaggacgagatgctgatgaacgacttcctaaagtacagcgcctggattcaaatggtatgactagcaaatctggtgcacgaaattggttaacttcagaggaagagtattcatgggaggatatgagtccaactttgactgatcgagtcagaagcagcatgccatcttttcctcctggaaccatgagaattggatttcctggggcaaatgcagggctactggaatctgatgttgggaggcacaatttcctaagccaagctactagatcatcagttgatgtttcaccatttaatattgaagataggatttcagctgcaagtgtatggttgtctgatctctctttaaataatagtgaatcataacattagcataacgctttgtgtgtctatcgtcaccacctgcagtcctatgaaataatttttctcccttttatttaaaacaattatggttccacacaattggattgctgctttctattctaatatatctgtaaaaatcgttgttctaaaaggctggatgccaactagccatgtgctgtctagttcaattgggcgggctaggtggccccaggtacgcatgatgagctagttgggccagactgccattggagtggcggaagcagtagcttgcaaagggccaacaagaccatgaccttttgttccatctgcttccttcttctcccgctcatattcttagctaaggtcatataccacatggcccatgtatgcaagttagcctgtgcagacaatgcagtactttgctgccagcctagagcctagtgcccttcagatcaatgatgaagatgtgctatggaaaaactccaagtaacatcagatgtactaatatagacttcagcttgctgatctagagatattattgccaaatttcgactgacatatcctttgacaatccttgtttgactgaaaggacaaaagagtagttttgtaaaaattgtgcaaataaattagtcagagaaacaacattttgtgttaccgcatatgtatgctgttagggaaggaactttataagcgtagtttttcagcagtttattcctttgtattttacaatttgatgtaaaacaatcaatgccttcgcccttcggccactcatagattttaagtaggactagcacattatttcccagtagccttcagcaggttttaggcacgtgtgcattatttaactgttttcttgtcttatacttaactgaaaccctttgaacccttttgtccatgcaccaaatagaattcatatgcaaacttgtcaacatggtagtgttgtctctatgaatctatgaacacctactgacagggacaccttcctcttgcagcatgttgacatttccacaaggagatatccaagcaattttggtgttcagaatggagctcttttggagtaccagaattctgaacatactctaaatcatgggagaattgctaccatgccagctccaccttggcaacagcctattggtcttccattgcgaatacaaggacctgaacatccttcagtacttggtagaataccacagcctgtcgacggtgaaatgccagtcaagaggttggacattagtggcacgtacaatggtttgaatgtagataaacccttggtgaagcataggtcatcacctgctcctgccccgacagaatggctcccccttcaccatgcccaatcacaaactttaccactaattccacctgataccaaacatgttagaagtgcatcagatagtttagaaattagttcttttgttagccatggaggtagttcatcagtatttgttccacggcatcagtatgatgccgtggatcggaaaacaggtaccagtaacttagctcaaccaccatcaccatatcagaaccaagatttgttgccatcaagtcaacaaaatcaaggtacaattttgggaaaccaagctcatcctcatcgtcccctaaagttacatccccatgctcatcctcaccatcaggagacctttagaagctttgcacctggcatgtctgtatctccattccaaggacaaggaggtaatgcaaccatgacaccagtttctgttctgccaacgtctttttctgtacctcctgctgtgccaccctatggcgtgctgtctgcacctgggtttcctatgccaccattaccgcctggaccaccagcacctgtctcactgcagatgggttcatcatcatcacaagttggtggcccccaaccatttgtctctggactgttgagtaaccttatgcggcacggtgtaatttcactggaaccacctagtcaatcccaggtatttctcatgcttcatgcgttgtattatatgacattattctgttcatatttatactgttaattgaggggaaaattgtgaatatttttttgtaggattctgttggagttgacttcaatgtagacctcaaggtacggaatgaatctgtcaccaatgctttgtatcaggatcttccaaggcagtgcaaaacttgtggtcttcgatttaaatgccaagaggagcatcgtgctcacatggactggcatgttacaaagaacaggaattcaaagaatcgtaagaaatcttcacgtaaatattttgtcaccgctgttgaatggttaagagcagcagaaacagtaggaaacgatggagttcctgcttttgtgctctcagacccagtcccagacagacaggaagagaaagaaattgctgttcctgcggatgaggagcaaacatcctgtgcattatgtcaagagccatttgaagatttctacagtgatgagactgacgagtggatgtacagaggtgcagtttatatgaatgcaccagatggtaatatcgatggtttggaatggtcacagttgggacctattgtccatgctaaatgtcgatctgggcccggcaatacttcttagggtgggcatagctggtgagtatctattcattttttgtgctataataaatatatatgaacttttagatgtacatagtttttttcttgaggttaacctcactgtaaacaatggatattatctccttgcgagtatttagctgtattccaacacatttttagctatgattatgtgttatctgactgaacaaaaatgctggatgtgatatgcaatgttcatgaattttccattatttattgtcagtgttctctgttagatgaatatactctcacattcgggacccattattttttgtttggttttgcctggtgcagtggtgagagctgtcttagtgagcactaggttacgggttcgaagcagtctctccacatttgcggaaggaacgcttgcctcggtttatcccttccctagaccccgctaatgtgggagtctctggcactaggtctgccctttttgtcgtgctgaaccattttgcagatccatcatatggattctcaggatgtccaattctactggcatatataatgagaggaccttcagttgctctcttgaatagttagttactgtttgaaggattatgtaggtttaaaaatggaaatgtgaatgctcatgtttgttcaagacttcaagtaatatacttttttaattggtgtaactttggtaatgacgaaggtaatcatgctaattatgtgattttatatgctaatgactatgcttttttataattttctagcatgactatgcttttcatctctcaccttttagtatctaaggaaggataaaagcaggctttggactaatttgtcaaaaatgacgatgtttaggagttcaggactattgtagtcccttttcgttcgtacttgactatttttgagcaggctttgcactaattttgtaaacaatgaccattgtagtcccttttcattcgtacttgactatttttcccatcatggcattactaatctgcacaattattttggcatcttggatatattttctcaagcaggcgtgatctaatttataaaaaagtggcaataatcaggaggtcaggactagcacagttactttctatttgctcttcactaggtttgcccatcatggcatcactaatctgcgcagctattatgtttgccaatcaaagattcatacatttagttgaacgacgctaatactgtgtatctcattttgtcattccctgacaacgatatcctggtgcacagggcctttccttctttttttgtgaggatttttattttgtttttgtgctagctcttagcctgtttggactttggaagcatcgtatgtttttaattatgtagtcagtgttcgtgcaattagtaagctgcataggcacatacaagcacgtaaattctggtgacctctttctgatgacatcacgattcctgtgaggatattgactcatttttctgtcaaatatcgaatggatctgacatgaaattcattgaaagtaattgatgagcaatcatatttgttttacgttttctttgtcctacaattttttccctttggattatgaaatcgtcaatgaccggttataagttggaaggtggtaaaattgagatttactcacctatttgccattgatctgtaccatgtaatttagttccatgctgttgtatcagctactacctcttccttaaagaggtgcatagctagaatgagtcttttagggacttgcaacaaaacctttttcttctttggctccaaaaggatcctttctagtacatgccaaaataggaacctaagggctagtttggaaacctcattttcccacgggtttttcattttcccaagggaagttagaacattttcccttgggaaaataaaaatcctttgggaaaatcgagttcccaaactagccctaagtgtgcttaaagccttaaaaagcctgctgagttttcctgaatggtgtatgatccaatgagatgggttcattggtttactcctttggccatggtgatgatttgaatacataatatttgttttcatattagttgcatgtgatggcttcaattttacactttctgtccctaatttaatgttaataaaggcaccatgaagataacttatcatattgtttattagtcttgcgtgaacaaatcactcgaacttaaacttgtgattatgtacaacaacagcaacaaagccttttatctcaagctaaacttgtgattatctgatggttaataaattcctttagaagtgcctcattgaaagatgttgctatgttgattgataactatttttattttcgttgcagggatgatttgatagtttttttttcccgttgcggctgtaggcatgacctgtaaactaatggagactcagattttggtatggttagcttcttttccatctggctcccttaatagccagtgcatagaatgttttttggaacaaaaaggcaggagccagtgcatagaatgtcaatgtgacaatagtgtataactgtaaaatttttgtgtaattcagttgcacagtgctgctttttcattacaactatagtgtgatctatactgaagcttcatgtttagggtttctgcagcacagttgttgacaacggtatatgtatatgtaaccatggtttcactggacatgggaaacaatatatttcaatagtcctgttttcttccgttaatatcttgtgcagaatacatagcagttctctacgttggttggtagctttggaagggatcgcccaagatgaccatgagtgctggtgactatttatgcctctcctgaagtgaaagggactagatgctgtcattaccttattgccaaaactttttagtattttcctttgtaagttgagagcgttgtttgtgacatattctcggtcgcttatatagttccgtattgctgatatgttcatgctccataatgtggatgagaatgtcttattagactttatactttattctagttggtatatgctctggcgaactattcaatgaaacaatttcgcaatgttttttttgtc

SEQ ID NO: 65 - DNA, 2895 bp, Zea mays
  source 1..2895: mol_type = other DNA
atggctggggcagcggcgtcccccgtggcggcccacgtggtggaacggttccgctcgcggctgcgcgaggaagcgggcagcggggagccgggcgcctccgcggtcgtgcgcttgtacgcggaggcgctccgcgagcttaccttcaactgcaagccggtcatcaccgagctcaccatcgtcgcaggccagcacgccgcgctcgccgccgggggaatcgccggcgccgtctgcgcccgcgtcgccgaggtgccacctgatcagatattaccatctttgtatctgctagatagtattgtgaagaacataggtggggaatatgtagagcattttgcaattaggttgcaaagtgtatttgttgatgcatactacagagttcatccaaaccagtatgcttcaatgaggcgcctctttcgtacctggtggccagtgtttccttcttctgtgcttcacagcattgaggatgaccttcaattttcgccctcagagaataaccgtcccaccacatcgacaaatgtgcatcaaacagagtcgttatctcctagaccatctcatggcatacatgtaaatcctaaatacctagaagcacaacataagttcaagcgggtcaatgtggcgcaccagtcagcagctcaggacactaggcaaatgtctgatgtgcaggaggatctgataaatggactaacatcgaatagttcacaaggacggccttcagtgtttcagaaatcttctttacagtatgctgatgatcctggtcaacaagagatgtttcgacctcttgctggagcaataagggcaacatcgccacatctcttgagaacacatgcatctgatgtgaatctagatgggccattagccaattcaaggagaaacttgtccagatcaccacctcttgatgtgttccctaggaatgtttccccaaagagaccactagagaggctaccaccatctcattctgtattgggacctgatcccagaaaattgcctgatcgaaatggcaggttaaggtgggctttggacgatggtgcacagtggcctacaataagtatgcttgatgaagagtacaggaagcaaagtgcaagggaacttattgatgcatatggaaattgtcaaggacgagatgctgatgaacgacttcctaaagtacagcgcctggattcaaatggtatgactagcaaatctggtgcacgaaattggttaacttcagaggaagagtattcatgggaggatatgagtccaactttgactgatcgagtcagaagcagcatgccatcttttcctcctggaaccatgagaattggatttcctggggcaaatgcagggctactggaatctgatgttgggaggcacaatttcctaagccaagctactagatcatcagttgatgtttcaccatttaatattgaagataggatttcagctgcaagtcatgttgacatttccacaaggagatatccaagcaattttggtgttcagaatggagctcttttggagtaccagaattctgaacatactctaaatcatgggagaattgctaccatgccagctccaccttggcaacagcctattggtcttccattgcgaatacaaggacctgaacatccttcagtacttggtagaataccacagcctgtcgacggtgaaatgccagtcaagaggttggacattagtggcacgtacaatggtttgaatgtagataaacccttggtgaagcataggtcatcacctgctcctgccccgacagaatggctcccccttcaccatgcccaatcacaaactttaccactaattccacctgataccaaacatgttagaagtgcatcagatagtttagaaattagttcttttgttagccatggaggtagttcatcagtatttgttccacggcatcagtatgatgccgtggatcggaaaacaggtaccagtaacttagctcaaccaccatcaccatatcagaaccaagatttgttgccatcaagtcaacaaaatcaaggtacaattttgggaaaccaagctcatcctcatcgtcccctaaagttacatccccatgctcatcctcaccatcaggagacctttagaagctttgcacctggcatgtctgtatctccattccaaggacaaggaggtaatgcaaccatgacaccagtttctgttctgccaacgtctttttctgtacctcctgctgtgccaccctatggcgtgctgtctgcacctgggtttcctatgccaccattaccgcctggaccaccagcacctgtctcactgcagatgggttcatcatcatcacaagttggtggcccccaaccatttgtctctggactgttgagtaaccttatgcggcacggtgtaatttcactggaaccacctagtcaatcccaggattctgttggagttgacttcaatgtagacctcaaggtacggaatgaatctgtcaccaatgctttgtatcaggatcttccaaggcagtgcaaaacttgtggtcttcgatttaaatgccaagaggagcatcgtgctcacatggactggcatgttacaaagaacaggaattcaaagaatcgtaagaaatcttcacgtaaatattttgtcaccgctgttgaatggttaagagcagcagaaacagtaggaaacgatggagttcctgcttttgtgctctcagacccagtcccagacagacaggaagagaaagaaattgctgttcctgcggatgaggagcaaacatcctgtgcattatgtcaagagccatttgaagatttctacagtgatgagactgacgagtggatgtacagaggtgcagtttatatgaatgcaccagatggtaatatcgatggtttggaatggtcacagttgggacctattgtccatgctaaatgtcgatctgggcccggcaatacttcttag

SEQ ID NO: 66 - protein, 964 aa, Zea mays
MAGAAASPVAAHVVERFRSRLREEAGSGEPGASAVVRLYAEALRELTFNCKPVITELTIVAGQHAALAAGGIAGAVCARVAEVPPDQILPSLYLLDSIVKNIGGEYVEHFAIRLQSVFVDAYYRVHPNQYASMRRLFRTWWPVFPSSVLHSIEDDLQFSPSENNRPTTSTNVHQTESLSPRPSHGIHVNPKYLEAQHKFKRVNVAHQSAAQDTRQMSDVQEDLINGLTSNSSQGRPSVFQKSSLQYADDPGQQEMFRPLAGAIRATSPHLLRTHASDVNLDGPLANSRRNLSRSPPLDVFPRNVSPKRPLERLPPSHSVLGPDPRKLPDRNGRLRWALDDGAQWPTISMLDEEYRKQSARELIDAYGNCQGRDADERLPKVQRLDSNGMTSKSGARNWLTSEEEYSWEDMSPTLTDRVRSSMPSFPPGTMRIGFPGANAGLLESDVGRHNFLSQATRSSVDVSPFNIEDRISAASHVDISTRRYPSNFGVQNGALLEYQNSEHTLNHGRIATMPAPPWQQPIGLPLRIQGPEHPSVLGRIPQPVDGEMPVKRLDISGTYNGLNVDKPLVKHRSSPAPAPTEWLPLHHAQSQTLPLIPPDTKHVRSASDSLEISSFVSHGGSSSVFVPRHQYDAVDRKTGTSNLAQPPSPYQNQDLLPSSQQNQGTILGNQAHPHRPLKLHPHAHPHHQETFRSFAPGMSVSPFQGQGGNATMTPVSVLPTSFSVPPAVPPYGVLSAPGFPMPPLPPGPPAPVSLQMGSSSSQVGGPQPFVSGLLSNLMRHGVISLEPPSQSQDSVGVDFNVDLKVRNESVTNALYQDLPRQCKTCGLRFKCQEEHRAHMDWHVTKNRNSKNRKKSSRKYFVTAVEWLRAAETVGNDGVPAFVLSDPVPDRQEEKEIAVPADEEQTSCALCQEPFEDFYSDETDEWMYRGAVYMNAPDGNIDGLEWSQLGPIVHAKCRSGPGNTS

SEQ ID NO: 67 - DNA, 6028 bp, Zea mays
  source 1..6028: mol_type = genomic DNA
acggctcgcgttaccgtcccagccaagccgccgcttctgcctcctcgccgtcgctgacgcccaatccattccccccggatcctcagaggccagccgcttcgattacgcgcctcgccttgcaaaccctagctcccccgccctctccctctcccatgggcccgaaaccctagtacctagtatccccccttcatgcgcgcctccgcggtccccatggaggcctccgcttccgcccgcagatccgccgcgggccccgacccccgcgccaagaagccgcgcctcgcgccgccgcctcgggaccccagatcttacgccgccaccgcctcctccaatggcaacacaagcgccgccgagcaagcgctggtcgacgagctgctcggccagtaccggaccgcgctcggggagctcaccttcaactccaagcccatcatcaccaacctcaccatcatcgccggcgagaaccttcaggccgccaagcccatagccgctctcatctgcgccaacatcctcgaggtgacctttgtctttgactcctcttacgctcgtcaaactgccgtcttcccctcctctttctcaattgaccatgtaaaagtaattctaaggcgccatcaagtagtgacaagcacccactggctgtctgttatgctagatatatcacatgtgctcggtggtgcggtaggattaccaggccgctgtacattgtatacactagtgtccatgctagctatcaagatcaatagctgtgatgttgcgaagccagttgttctgcgtacaatagatgcaaaagcaaatgctgatttacctatcgttgctgagaaatctccccccatctgttctctccctcttgctctcttgtgtctacggaacacactttgtgatgctatataaagggcagaccctgtgctggaggctcccactttgtgatgctgtatatatggtttatatgatttcaaacgtgaagtttgtcgctcaacttcgattctcaagtgttcaattgttcatgggtttataaattccatactcctgtgacactgcaaaacaaatatgaacattgtggtttgaaccacactctactaattagagggctacagcagttaccactggtacggaaacatcataattatcagcttatggttgactatcgaaccaggcttcaaaaagcatttcattcctacgctccccacaaacttgtgcacagattacatcctcacgctgcagtaactactatactgttctcatgctgaccagtgacaaagttatcaacttgtgtaccccaatcagttgaatggcaatgtgttccaggtttcagaaagttctactacaaactgaagtttatgatacaccgtcatgtacatatgaaaaatatattgatgcactgataaaagggcaggatattgtgaagtataatatatgccaccacaagcgcggcctgcactgggcttctccttacactcctggatagccggctaactgctgagcgttggttgacttggtttgtgtattactttgtgtaaccttgctgcaatacacggtttgagacggcatgttatctatgctcattttttttccttggttgtattttaaaaggaaaaattactggatgctttagaagtaaggttgagtcttagtcactggataaaaggaatgacaaataaacaccattgaaccttctcatctatgtagctatgtgaattgtaagacacaccgttgtatatggtctgttgtcaaatcatatctgccagtgttcttgtcttgaatccacattgtcgaggcatatcagcatttggtttttcttgttgctctgcatttactataatccattatagggttatgcaattgttatccactatgttggagggggtgtatctattttatttgatggttacttgtccttgtaggttccaagtgaacagaaactaccatcactatatctacttgacagtattgtaaaaaatattggaaaagattatgttaaacatttctcagcaagattaccagaggttagtatttcatcctgtaacatcttcttgatatgggaatagagacggttccaatctctggaaacaatatttatttattgagaggatgtagcttggacattacaataactaagttctctttggttgtatttttcatttttgctaggtgttctgcaaggcatataaacaagttgatccttctatacatcatagcatgaggcacctttttggaacatggaaaggagtgtttcctctacctccgctccaaatgattgagaaggaacttggttttcagtcatctgccaatggatcatctagtgctgcaccatcaaggactgattctcaatccccgcgcccatccaatagcatacatgtaaatccaaaatatttggaagcgagacaacaactcaatcaaccaactaaggcaagaaaatttttctcaagttatcttgatacacctgttcactcttttgactcctgggacaaatatgtaccatgtgtagcattgtagctgccactttgttcttttgcaccccatttgattgtttctgatgctgttttcccttacttcggacaggggatacttggaagtggtgctaagacaactgtaattgctgatacaggcggtgatattgagaggcctaataggctaggtactgatagaaatgcggggagacgattagatgcttctaatgctagacctgtaagttctctgctatatcttggctcttttgacatctcttttaaaatgacagaaaagtgtcactgctccttgtgtcattcacatttttttattgtacttttaactgtaacatgcagaacatacaacgcactcagagggacccttctagcaattctcttcatgaaaaacaggcagttagagatgtgggaggccttggattctccaacatttcgcagcaggctgttgtgggaactggtcaagttcgctcaaaacctaaagctcaagatggaattggggggccttattatgcctctggtgttgggtcttctgaagaacaatttgacagacgcagcaacttttatgcaagcaaggatgctcgaccatcaggatctgtacgtttggaaagtgcacttcttccaactccatcaatcaatgccgatagagttggtaagccttcatccaacaaaagctggaaacactctgaggaagaagaatatgtctgggatgatgtacattctaaagcagcagaatatggtggtagccataatgtaataaaaggagaatggatgtctgatgatggcaatgctaagtttgcaaacctccaaagggctaaatgggcagatgcaggacctgtggaacgtatagatcccaacacacataaacttgataacgtgtcaagatttggacttggagcaggtcaagaaagaagaatttcagaatacatggtatgtgactcaatgattgaaatgcaattatattaagtatcgagtaaatattgatatgctaattattgctacataataatgccagagatgttttaaatatgcatggttcgaaattttctttcttgttggaaagtattataatactacttgtattttcaattgttcaggatcaggaagagtaccttcttggcaagcgtgaggtggaagcaagaattgataaggaaattaggtctgatggacaacagttcccagcagctcgaggttctcctctctgggtgtcccaggaaaaaacactcctagacattgggttggagcctaggatatcaaggttttcaactcagccagcagagaggtccaccatttatactggcaccatgtcagcaggtatcacttcatctgtacctgttgggttatcaggaaactatgctggaaggtcaagcctagatactgcaaatggtatgccaataagatcaactgaggcttttgggcagcagaagcatagatattggtcatcttcccctccacaagctgacacatcttcatctactgcaccctttgcacgtcagggttctccaaatcctgccgagtctgatttttatccttccagatcactttctcgccaaaatcctcaagaatatagcaaaagagcattgccggtacttgctaaagattctcatgttgtggttcacaatgatggatttactcagggccaacctagtctacaggcaacccggcagacacaaaagtaccctatcttacagtcgaagtcgcatattaagccatctgatcaattgcaggcaagtttttcgcgtgaaaattctccatcattgttcagaccttccatcgaacttggagaagtctctttacccagtgactccacccccataagctcagatctgacaagtgctagtaatttgttggctagtctcgtaaagagtgggttcaaaccaaataaccacagtgatgcacaggctcttggtccaagtggaccacttccagtggcttctttatcacttcaaaatgctgctggtgaaaataccactttgcaggcacatacaccaaacacttctcgtccaccattgccaccacctccatcaacacagagtgcggagaaagctgcgccgctctcaagtcttctcagttccctggttgcaaagggattaatctcttctccagcctatgattcatcgagtgctgtcatttcacagccaaataaagcatcaagcatgaatgttaaagatgccacagcttcagctgtgcctttgctagctctgagaccttcagttggtaaggcgtcatctaattctgattcatctgctccaacaaacgcttcattaaccaaagctatcgaaattaagatgagtgacctcattggcctggagttcaagccagagaaacttcgcaagtaccatgagcacgtgattagcagcctctttgacgacgaccaaagccatcagtgtaaaacatgtggcaataggttcaggcttgaagaactgagcttgcacacatcaagctgtgggcaaaggaagtctgagactatatatactggtatcgcacctaaaagatggtaccctagcaagaacatttacattgatggatcacatgaaattgaggacagcactgaggcttctgatggagatttaggttctgcagaagaagtgtgtgagtttatggtcccttcagacgagaggcaaattatctgtgcactatgtggggaaccatttgatgacatatattcttttgagaagggggactggatgtacaaggatgcagtgtttttggattaccccaagggagaaagcagctgcagaaacagtgttgagggtgaagagcatgtgcccattgtccatgtaagatgtatgccaagaggctcaaacgatggcatggaggttgactagaatgagtattgttcacagcagttatgctcttgaggggccttgaggtggatgagcctaaagctggggtttgcacgaggaactgtgcatcatcaggttgtctgttgcaattggactgatgacatacatgtaggatgattggtggaaggtggcaggcgttatcagaaacaaaactttggcgaagatctggaactgcccgtggatttctcagtgcccattacaaagtgcttacacaaagaataaagctttagaggaacaatttggccatgcaaatcagaattatcaaaatcttcatattttgggtctccatctgagaaggatctttatgttgatggaaagaaagctgtgcaggtgcatgtcaaaagaatgtatgatttctttacgagggaaaaggggtatggtaccccacatgtaactcggatactgctgttttttgacgatacatttctcgtcttttggagtaggaatttttgtgtagttaagaaactgataatagaacattatagaggtaggactaaacaagttagggtgtgtttggcgtttggctttaacttttgcccactaaaagccaaaagaaaaaacaaaggtatgaatttagaaagtagctttttctaaaagatgactttgtgcaaaactgaaaatatctttgaacttgcttttagtggatttctgatacaactgtaaaaatatatatggaagaatttttagtggcttttagttgtttctaccaaataatttttagctttttag

SEQ ID NO: 68 - DNA, 3486 bp, Zea mays
  source 1..3486: mol_type = other DNA
atgcgcgcctccgcggtccccatggaggcctccgcttccgcccgcagatccgccgcgggccccgacccccgcgccaagaagccgcgcctcgcgccgccgcctcgggaccccagatcttacgccgccaccgcctcctccaatggcaacacaagcgccgccgagcaagcgctggtcgacgagctgctcggccagtaccggaccgcgctcggggagctcaccttcaactccaagcccatcatcaccaacctcaccatcatcgccggcgagaaccttcaggccgccaagcccatagccgctctcatctgcgccaacatcctcgaggtgacctttgtctttgactcctcttacgctcgtcaaactgccgtcttcccctcctctttctcaattgaccatgtaaaagtaattctaaggcgccatcaagtagtgacaagcacccactggctgtctgttatgctagatatatcacatgtgctcggtggtgcggtaggattaccaggccgctgtacattgtatacactagtgtccatgctagctatcaagatcaatagctgtgatgttgcgaagccagttgttctgcgtacaatagatgcaaaagcaaatgctgatttacctatcgttccaagtgaacagaaactaccatcactatatctacttgacagtattgtaaaaaatattggaaaagattatgttaaacatttctcagcaagattaccagaggtgttctgcaaggcatataaacaagttgatccttctatacatcatagcatgaggcacctttttggaacatggaaaggagtgtttcctctacctccgctccaaatgattgagaaggaacttggttttcagtcatctgccaatggatcatctagtgctgcaccatcaaggactgattctcaatccccgcgcccatccaatagcatacatgtaaatccaaaatatttggaagcgagacaacaactcaatcaaccaactaaggcaagaaaatttttctcaagttatcttgatacacctgttcactcttttgactcctgggacaaatatgggatacttggaagtggtgctaagacaactgtaattgctgatacaggcggtgatattgagaggcctaataggctaggtactgatagaaatgcggggagacgattagatgcttctaatgctagacctaacatacaacgcactcagagggacccttctagcaattctcttcatgaaaaacaggcagttagagatgtgggaggccttggattctccaacatttcgcagcaggctgttgtgggaactggtcaagttcgctcaaaacctaaagctcaagatggaattggggggccttattatgcctctggtgttgggtcttctgaagaacaatttgacagacgcagcaacttttatgcaagcaaggatgctcgaccatcaggatctgtacgtttggaaagtgcacttcttccaactccatcaatcaatgccgatagagttggtaagccttcatccaacaaaagctggaaacactctgaggaagaagaatatgtctgggatgatgtacattctaaagcagcagaatatggtggtagccataatgtaataaaaggagaatggatgtctgatgatggcaatgctaagtttgcaaacctccaaagggctaaatgggcagatgcaggacctgtggaacgtatagatcccaacacacataaacttgataacgtgtcaagatttggacttggagcaggtcaagaaagaagaatttcagaatacatggatcaggaagagtaccttcttggcaagcgtgaggtggaagcaagaattgataaggaaattaggtctgatggacaacagttcccagcagctcgaggttctcctctctgggtgtcccaggaaaaaacactcctagacattgggttggagcctaggatatcaaggttttcaactcagccagcagagaggtccaccatttatactggcaccatgtcagcaggtatcacttcatctgtacctgttgggttatcaggaaactatgctggaaggtcaagcctagatactgcaaatggtatgccaataagatcaactgaggcttttgggcagcagaagcatagatattggtcatcttcccctccacaagctgacacatcttcatctactgcaccctttgcacgtcagggttctccaaatcctgccgagtctgatttttatccttccagatcactttctcgccaaaatcctcaagaatatagcaaaagagcattgccggtacttgctaaagattctcatgttgtggttcacaatgatggatttactcagggccaacctagtctacaggcaacccggcagacacaaaagtaccctatcttacagtcgaagtcgcatattaagccatctgatcaattgcaggcaagtttttcgcgtgaaaattctccatcattgttcagaccttccatcgaacttggagaagtctctttacccagtgactccacccccataagctcagatctgacaagtgctagtaatttgttggctagtctcgtaaagagtgggttcaaaccaaataaccacagtgatgcacaggctcttggtccaagtggaccacttccagtggcttctttatcacttcaaaatgctgctggtgaaaataccactttgcaggcacatacaccaaacacttctcgtccaccattgccaccacctccatcaacacagagtgcggagaaagctgcgccgctctcaagtcttctcagttccctggttgcaaagggattaatctcttctccagcctatgattcatcgagtgctgtcatttcacagccaaataaagcatcaagcatgaatgttaaagatgccacagcttcagctgtgcctttgctagctctgagaccttcagttggtaaggcgtcatctaattctgattcatctgctccaacaaacgcttcattaaccaaagctatcgaaattaagatgagtgacctcattggcctggagttcaagccagagaaacttcgcaagtaccatgagcacgtgattagcagcctctttgacgacgaccaaagccatcagtgtaaaacatgtggcaataggttcaggcttgaagaactgagcttgcacacatcaagctgtgggcaaaggaagtctgagactatatatactggtatcgcacctaaaagatggtaccctagcaagaacatttacattgatggatcacatgaaattgaggacagcactgaggcttctgatggagatttaggttctgcagaagaagtgtgtgagtttatggtcccttcagacgagaggcaaattatctgtgcactatgtggggaaccatttgatgacatatattcttttgagaagggggactggatgtacaaggatgcagtgtttttggattaccccaagggagaaagcagctgcagaaacagtgttgagggtgaagagcatgtgcccattgtccatgtaagatgtatgccaagaggctcaaacgatggcatggaggttgactag

SEQ ID NO: 69 - protein, 1161 aa, Zea mays
MRASAVPMEASASARRSAAGPDPRAKKPRLAPPPRDPRSYAATASSNGNTSAAEQALVDELLGQYRTALGELTFNSKPIITNLTIIAGENLQAAKPIAALICANILEVTFVFDSSYARQTAVFPSSFSIDHVKVILRRHQVVTSTHWLSVMLDISHVLGGAVGLPGRCTLYTLVSMLAIKINSCDVAKPVVLRTIDAKANADLPIVPSEQKLPSLYLLDSIVKNIGKDYVKHFSARLPEVFCKAYKQVDPSIHHSMRHLFGTWKGVFPLPPLQMIEKELGFQSSANGSSSAAPSRTDSQSPRPSNSIHVNPKYLEARQQLNQPTKARKFFSSYLDTPVHSFDSWDKYGILGSGAKTTVIADTGGDIERPNRLGTDRNAGRRLDASNARPNIQRTQRDPSSNSLHEKQAVRDVGGLGFSNISQQAVVGTGQVRSKPKAQDGIGGPYYASGVGSSEEQFDRRSNFYASKDARPSGSVRLESALLPTPSINADRVGKPSSNKSWKHSEEEEYVWDDVHSKAAEYGGSHNVIKGEWMSDDGNAKFANLQRAKWADAGPVERIDPNTHKLDNVSRFGLGAGQERRISEYMDQEEYLLGKREVEARIDKEIRSDGQQFPAARGSPLWVSQEKTLLDIGLEPRISRFSTQPAERSTIYTGTMSAGITSSVPVGLSGNYAGRSSLDTANGMPIRSTEAFGQQKHRYWSSSPPQADTSSSTAPFARQGSPNPAESDFYPSRSLSRQNPQEYSKRALPVLAKDSHVVVHNDGFTQGQPSLQATRQTQKYPILQSKSHIKPSDQLQASFSRENSPSLFRPSIELGEVSLPSDSTPISSDLTSASNLLASLVKSGFKPNNHSDAQALGPSGPLPVASLSLQNAAGENTTLQAHTPNTSRPPLPPPPSTQSAEKAAPLSSLLSSLVAKGLISSPAYDSSSAVISQPNKASSMNVKDATASAVPLLALRPSVGKASSNSDSSAPTNASLTKAIEIKMSDLIGLEFKPEKLRKYHEHVISSLFDDDQSHQCKTCGNRFRLEELSLHTSSCGQRKSETIYTGIAPKRWYPSKNIYIDGSHEIEDSTEASDGDLGSAEEVCEFMVPSDERQIICALCGEPFDDIYSFEKGDWMYKDAVFLDYPKGESSCRNSVEGEEHVPIVHVRCMPRGSNDGMEVD

SEQ ID NO: 70 - DNA, 7254 bp, Zea mays
  source 1..7254: mol_type = genomic DNA
gctgacgccaaatcaaatcaaaccttgctgccttctgcatccggtagcagcaagtcgccgcttgtgcccttcctcctcactttccctcgctgacgcccgatccccatgattcgattacgcgacccgccttgcaaaccctagcttctcccgctctctccctttccaatgcgccccaaaccctagcttcccgcccgcccacccgccggtcatgcgcgcctccgcctccgctgtccccatggaggcctccgcctccgcccgcagatccgccgctagccccgaccccggcgcgaagaagccgcgcctcgcgcagccgccggcacgggaccccagatcttacgccgctgccgccgccgcctccaatggcggcgcaagcgccgccgagcaagcgctggttgacgagctgctcggccagtaccggaccgcgcttggggagctcaccttcaattccaagcccatcatcaccaacctcaccatcatcgctggcgagaacctccaggccgctaagcccatcgccgccgtaatctgcgccaacatcctcgaggtgacctttgtctttgactcctcttacgctcgtcaaactgccgtctccccttcttcctcaattcaccatgtaaaagtaactctaaggcgccaagcatccactggcggtcccttatgctagatacagcacgtgtgcttggtggtgtggtatgatttccaagcctctgtgcattgtgcacactagtgtccacgctagctatcaagatcagtcgctgtgatgtcgcaaacgagttgtgtgtacagtagatgcaaaaggaaatgctgatttacctatctttgctgagaaatctctttcccatctcttttctctccctcttgctctcttgtgtccgctgagcttgttttgtgatactaatatacgtgatttatatgatttcaaacgcaaagttcctcgttcaactttgtttgaactttgtttctgaagtgtttattgatgttgggtaccagtcgaaattctatgaagaactacaacatggcaacataattaaattatggggcaattgtgattatacacttctgatcttgcaattgtgattttacctctccttttttaactttgtgattttatccctatgctttgaaaatgaagctgcgccctctcctttaacaccattagcgaaaatcggaattagtgattaaaaaataaaaaataaggggtaaaattacaattgcaaggggagagaagggtataatcacaatcaaccctgaaaaattcaacattttttcagggttaattatgattatacccttctctcaccttgcaattgtgattttactcctggttttttatttttaatcactaattttgattttcatcagcttcgttttcaaaacagtgtggtaaaatcacaaagttgaaaaacaaggggtaaaatcgaattgcaatgtggtagaagggatcacaattgctccctctcattttcgaaggggatgaagtggagaagggacctgataaatatattcatacgccgaaactcatgaaaaattacgaagtttaaaaatggaagttgcaatggcatgacgaaggtaagattcacgttcccttaaatgattgaacttacaagaagcacattatcaatgaacaagtcataaagtagggaactagatgtgcaactgaagctaagtatgtaaagaccgtgttatcatgtatctttggaagttctcttgacagatgtaaagaccgtgttatcatatatctttggaagttctcttgacaggggatagatacaggagcagggatgtaaaatgccgatctctgtagcagtaacactgttaatctatttataggatcaagctgctatataccttcatatgaaattacaattttgccctccctattacataattgtcatactcagcaacaggagtacaatttcgtcattttatgtctttgacgtgccgtgtacaacttttcttttccctgtactgtaaggctttggcttcattggtcagcgcctttgacttcgaagacattagggcttcggtgctttgaagatgttagggctttggcgcttcaaagatgttagggcttggacgtttcgaagacgttagagctttggcttcttttattcatccgaaggttcagcttcatcattggtccgaagctcacctatgcttcagatttgatttctttcgttaccttgaaactgtaaactggacaggcctctgttaatatggaccttcggtcagggactctccacattaattggtttatataagtttgagactcctgtgacatggcaaaacaaatatgaacattgtgttttgacttttggaccaccctctgctaattagagggctacagcagttaccactggtatgaaaacatcataagttgtcagcagcatggttgaacattgaaccaggcttcaaaagcgtttcaatcccacactctccataaacttgtgtacagattatatcctcacgttgcagtaactattatactattctcatgctgaccagtgaagaagttatcactttgtgtaccctaatctgttgaattgcaatatgttccagatttcagaaagttctactacaaactgaagtttatgatacaccgtcttgtacatatgaaaaatatgtccatgcactggtaaaaggacatggaattgcgaagtataatatatgccacaagtgtggcctgcactgtgcttctccttacactcctggataaccgctgagcattagttgacttggtttgtgtattactttgtgtaaccttgctgctttacatggtttgagacggcatgttatctatgctcgttttgtttcgttggttgtgttctaaaatgaaacattactggattctttagaagtaaggttgagtcctagtcactggatgaaaggaatgacgaataaacacaattgaaccttctcagatatgtagttgtgtgaattgtaagactcgcaccgttgtatatgttctgttttcaaatcatatctgccagtgttcttgtcttgaatccacattgtcgaggtatatcaacatgtggtttttcttgtcaccctgtattactataatccactatagtgttacacaattgttatgcactatgtatggaggagggggtgtatctgttttatttgatggttgcttgtccctgtaggttccaagtgaacagaaactaccatcgctgtatctgcttgacagtattgtaaaaaatattggaaaagattatgtcaaacatttctcagcaagattaccagaggttagtatttcattttgcaccatcttcttgacatgggaacagagatggttcctacccctggaaaccatatttatttattgagaggatgacttggacatcacaataactaagttttctttggttgtatttttcatttttgctaggtgttctgcaaggcatataaacaagttgatccttctatacatcatagcatgaggcatctttttggaacatggaaaggagtgtttcctctacctcctctccaaacgattgagaaggaacttggttttcagtcatctaccaatggattatcaggtgctgcaccatcaaggactgattctcaatcgccgcgtccatccaatagcatacatgtaaatccaaaatatttggaagcgaggcagcagctcaatcaaccaactaaggcaagatattctttctcaagttatgttgatacacctgttcattcctttgacctcttggacataatcaaaatatgtaccatgtgtagcattgtggctgccactttgttcttttgcaccccatgtgattttgttcctggttctgttttccccttactttggacaggggatacttggaagtggtgctaagacagctgtaattgctgatacagacgatgatattgagagggctaataggctaggtactgaaagaagtgcagggagacgattggatgctcctaatgctagacctgtaagttgtctgctctctttggttcttttgacatctcttcatgtaaaatggcaaaaaagtgtcattgctccttgtgttattcacggttttcttcttcttctaatatactacttctaactgtaacatgcagaacatagagcacattcagagggacccttttagcaatcctattcatgaaaaacttgcaggtagagatgtgaggggccttggattctccaacacttcacagcaggctgttgtgggaactggtcaagttcgctcaaaacccagaggccaagatggaattgggggaccctattatgcctctggtgttgtgtcttctgaagaaaaaattgacaagcgcagcaacttttatacaagcaaggatgttcggccatcaggatctgtacatttcgatagtgctcttcttccaactcgatcaattaatgctaatagagttggtaggccttcatcaaacaaaagctggaagcactctgaggaagaagaatatgtttgggatgatgtaccctctaaaacaacagaatatagtggtacccataatataataaaaggagaatggatgtctgatgatggcaatgctaagtttgcaagcctccaaagtgctaaatgggcagatgcaggacctgtggaatgtatagatcccaagacacataaagttgataatgtgtcaagatttggatttgcagcaggtcaagaaagaagaatttcagcatacatggtatgtgtctcaatcattgaaagaaaattatattaagtatcgagtaaatattgatatgctaattgttgctacataataatgccagagatgttttaaacatgcaccttgttggaaagtattataatactacttttcaattgttcaggatcaggaagagtaccttcttggcaagcgtgaggtggaagctagaattgatagggaaattaggccctatggacaacagttcccagcagctcgagattcctctctctgggtgtcccaggaaaaagcactccccgacaatgggttggacccttgggtatcaaagttttcaaatcagccagcagagatgtccatttgtactggcaccatgccagcaagtatcatatcatctgtacctgttaggttatcaggacactatgctggaaggtcaaacctagatactacaaatagtgtgccaataagatcaactgaggcttttgggcagcagaagcatagatattggtcatcttcccctccacaagctcacccaccttcatctactgcaccctttgcacgtcagggttctccaaatcatgacgagtctgatttttatccttctggatcattttctcaattgggccaagatcctcaagaagaatatagccagagagcattgcctgtacttgctaaaggttctcatgttgtggttcacaatgatgaacttactcagggccaacctagtctacaggcaaccctgcagacacaaaagtaccctatcatacagtccaagtcacatattaatccatctgatcaattgcaagcaagctttttgtgtgaaaattctccatcactgttcagaccttccattcagcttggagaagtatctttacccagtgattccacccctataagctcagatctgacaagtgctagtaatttgttggctggtctcataaagagtgggttcaaaccaagtaaccacagtgatgcacagcctcttggtccgagtggctcacttctagtggcttctttatcacttcaaaatgctgctggtgaaaataccattttgcagacacagacaccaaacacttctcgtccaccattgccaccaggtctaccaccacctccatcaacacagagtgcagagaaagctgcgccgctctcgagtcttctcagttccctggttgcaaagggattaatctcttctcctgcctctgattcatctgatgctgtcatttcacagccaaataaagaatcaagcataaatggtaaagatgtcgcagcttcaggtgtgcctttgccagctctgaagtcttcatttggtaaggtgtcatcatctaattctgattcatctgccccaccaaacgcttcgttacccaaagctatcgaaattgagatgggtgacctcattggcctggagttcaagccagagaaacttcgcaagtaccatgagcatgtaattagctacctcgatgaccaaagccatcagtgtaaaatgtgtggtaataggttcgggcttgaagaactgagtttgcacacatcaagctgtgggccaagggagtcaaggactatctatactggtatcgcacctaagagatggtatcctagcaggaacatttacattgatggatcacatgaaattgaggatagcgctgaggcttctgatgcaggtttaggttctgcagaagtgtgtgagtttatggtccatgcagacgagaggcaaattatctgtgcactgtgtggggaaccatttgatgacgtatattcttttgagaaggggaactggatgtacaaggatgcagtattcttggattatgccaagggtaaaaatagctgcggaaacaatgttaagggtgaagagcatgtgcctatagtccatgtaagatgtatgccaagaggctcaaacgatggcatggacgttgactagaatggttgttcacagaagccaagttctcgaggggcctagaggtggatgaggctgaagctggtgtttgcacgaggcaatgtgcatcatcaggtggtctgcggcaactgggcttaagacatacatgtaagatgcttggtggaggtggcaggcgttatcagaaacaaatcttgcgaagatctgcaaccatccatggatttaaccatgccctttacaaagtgcttacacaaagaataaagccccagaggagcacttcggccatgcggttcagaattatcaaaatcttcatatttttggtctccatctgagaaggggctgggtctttatgttgatacaaagagagctttgcaagaggatgtcaaaagaatgtatgatttctttacgtggaaaaggggtatgatatcccgcatgtaagttgagcactgctgttttctgacgatacgtttcttgactgttaggaatttttgcactacgaaactgataatacaacatgatagaggcaggactaaggggaatgtttggtttacatcggatgtttgaataccaaattaaatatagtttaattataaaaatggatgaagacgaaatgaatgagacaaatttattaactataattaaatccacgattagcataatgtgttaccgtttagtcattactccctttgcttccttttagaagtcatttttgacagcatttcacatattaagataga

SEQ ID NO: 71 - DNA, 3315 bp, Zea mays
  source 1..3315: mol_type = other DNA
atgcgcgcctccgcctccgctgtccccatggaggcctccgcctccgcccgcagatccgccgctagccccgaccccggcgcgaagaagccgcgcctcgcgcagccgccggcacgggaccccagatcttacgccgctgccgccgccgcctccaatggcggcgcaagcgccgccgagcaagcgctggttgacgagctgctcggccagtaccggaccgcgcttggggagctcaccttcaattccaagcccatcatcaccaacctcaccatcatcgctggcgagaacctccaggccgctaagcccatcgccgccgtaatctgcgccaacatcctcgaggatcaagctgctatataccttcatatgaaattacaattttgccctccctattacataattgtcatactcagcaacaggagtacaatttcgtcattttatgtctttgacgtgccgtgtacaacttttcttttccctgtactatttcagaaagttctactacaaactgaagtttatgatacaccgtcttgtacatatgaaaaatatgtccatgcactggttccaagtgaacagaaactaccatcgctgtatctgcttgacagtattgtaaaaaatattggaaaagattatgtcaaacatttctcagcaagattaccagaggtgttctgcaaggcatataaacaagttgatccttctatacatcatagcatgaggcatctttttggaacatggaaaggagtgtttcctctacctcctctccaaacgattgagaaggaacttggttttcagtcatctaccaatggattatcaggtgctgcaccatcaaggactgattctcaatcgccgcgtccatccaatagcatacatgggatacttggaagtggtgctaagacagctgtaattgctgatacagacgatgatattgagagggctaataggctaggtactgaaagaagtgcagggagacgattggatgctcctaatgctagacctaacatagagcacattcagagggacccttttagcaatcctattcatgaaaaacttgcaggtagagatgtgaggggccttggattctccaacacttcacagcaggctgttgtgggaactggtcaagttcgctcaaaacccagaggccaagatggaattgggggaccctattatgcctctggtgttgtgtcttctgaagaaaaaattgacaagcgcagcaacttttatacaagcaaggatgttcggccatcaggatctgtacatttcgatagtgctcttcttccaactcgatcaattaatgctaatagagttggtaggccttcatcaaacaaaagctggaagcactctgaggaagaagaatatgtttgggatgatgtaccctctaaaacaacagaatatagtggtacccataatataataaaaggagaatggatgtctgatgatggcaatgctaagtttgcaagcctccaaagtgctaaatgggcagatgcaggacctgtggaatgtatagatcccaagacacataaagttgataatgtgtcaagatttggatttgcagcaggtcaagaaagaagaatttcagcatacatggatcaggaagagtaccttcttggcaagcgtgaggtggaagctagaattgatagggaaattaggccctatggacaacagttcccagcagctcgagattcctctctctgggtgtcccaggaaaaagcactccccgacaatgggttggacccttgggtatcaaagttttcaaatcagccagcagagatgtccatttgtactggcaccatgccagcaagtatcatatcatctgtacctgttaggttatcaggacactatgctggaaggtcaaacctagatactacaaatagtgtgccaataagatcaactgaggcttttgggcagcagaagcatagatattggtcatcttcccctccacaagctcacccaccttcatctactgcaccctttgcacgtcagggttctccaaatcatgacgagtctgatttttatccttctggatcattttctcaattgggccaagatcctcaagaagaatatagccagagagcattgcctgtacttgctaaaggttctcatgttgtggttcacaatgatgaacttactcagggccaacctagtctacaggcaaccctgcagacacaaaagtaccctatcatacagtccaagtcacatattaatccatctgatcaattgcaagcaagctttttgtgtgaaaattctccatcactgttcagaccttccattcagcttggagaagtatctttacccagtgattccacccctataagctcagatctgacaagtgctagtaatttgttggctggtctcataaagagtgggttcaaaccaagtaaccacagtgatgcacagcctcttggtccgagtggctcacttctagtggcttctttatcacttcaaaatgctgctggtgaaaataccattttgcagacacagacaccaaacacttctcgtccaccattgccaccaggtctaccaccacctccatcaacacagagtgcagagaaagctgcgccgctctcgagtcttctcagttccctggttgcaaagggattaatctcttctcctgcctctgattcatctgatgctgtcatttcacagccaaataaagaatcaagcataaatggtaaagatgtcgcagcttcaggtgtgcctttgccagctctgaagtcttcatttggtaaggtgtcatcatctaattctgattcatctgccccaccaaacgcttcgttacccaaagctatcgaaattgagatgggtgacctcattggcctggagttcaagccagagaaacttcgcaagtaccatgagcatgtaattagctacctcgatgaccaaagccatcagtgtaaaatgtgtggtaataggttcgggcttgaagaactgagtttgcacacatcaagctgtgggccaagggagtcaaggactatctatactggtatcgcacctaagagatggtatcctagcaggaacatttacattgatggatcacatgaaattgaggatagcgctgaggcttctgatgcaggtttaggttctgcagaagtgtgtgagtttatggtccatgcagacgagaggcaaattatctgtgcactgtgtggggaaccatttgatgacgtatattcttttgagaaggggaactggatgtacaaggatgcagtattcttggattatgccaagggtaaaaatagctgcggaaacaatgttaagggtgaagagcatgtgcctatagtccatgtaagatgtatgccaagaggctcaaacgatggcatggacgttgactag

SEQ ID NO: 72 - protein, 1104 aa, Zea mays
MRASASAVPMEASASARRSAASPDPGAKKPRLAQPPARDPRSYAAAAAASNGGASAAEQALVDELLGQYRTALGELTFNSKPIITNLTIIAGENLQAAKPIAAVICANILEDQAAIYLHMKLQFCPPYYIIVILSNRSTISSFYVFDVPCTTFLFPVLFQKVLLQTEVYDTPSCTYEKYVHALVPSEQKLPSLYLLDSIVKNIGKDYVKHFSARLPEVFCKAYKQVDPSIHHSMRHLFGTWKGVFPLPPLQTIEKELGFQSSTNGLSGAAPSRTDSQSPRPSNSIHGILGSGAKTAVIADTDDDIERANRLGTERSAGRRLDAPNARPNIEHIQRDPFSNPIHEKLAGRDVRGLGFSNTSQQAVVGTGQVRSKPRGQDGIGGPYYASGVVSSEEKIDKRSNFYTSKDVRPSGSVHFDSALLPTRSINANRVGRPSSNKSWKHSEEEEYVWDDVPSKTTEYSGTHNIIKGEWMSDDGNAKFASLQSAKWADAGPVECIDPKTHKVDNVSRFGFAAGQERRISAYMDQEEYLLGKREVEARIDREIRPYGQQFPAARDSSLWVSQEKALPDNGLDPWVSKFSNQPAEMSICTGTMPASIISSVPVRLSGHYAGRSNLDTTNSVPIRSTEAFGQQKHRYWSSSPPQAHPPSSTAPFARQGSPNHDESDFYPSGSFSQLGQDPQEEYSQRALPVLAKGSHVVVHNDELTQGQPSLQATLQTQKYPIIQSKSHINPSDQLQASFLCENSPSLFRPSIQLGEVSLPSDSTPISSDLTSASNLLAGLIKSGFKPSNHSDAQPLGPSGSLLVASLSLQNAAGENTILQTQTPNTSRPPLPPGLPPPPSTQSAEKAAPLSSLLSSLVAKGLISSPASDSSDAVISQPNKESSINGKDVAASGVPLPALKSSFGKVSSSNSDSSAPPNASLPKAIEIEMGDLIGLEFKPEKLRKYHEHVISYLDDQSHQCKMCGNRFGLEELSLHTSSCGPRESRTIYTGIAPKRWYPSRNIYIDGSHEIEDSAEASDAGLGSAEVCEFMVHADERQIICALCGEPFDDVYSFEKGNWMYKDAVFLDYAKGKNSCGNNVKGEEHVPIVHVRCMPRGSNDGMDVD

SEQ ID NO: 73 - DNA, 9618 bp, Zea mays
  source 1..9618: mol_type = genomic DNA
gaaaaggaaagcatcgctgaaacgagatcctcgccgtttcgcgaacaaatcggtgactgtctcctgctatctccccctttccgccacttcgctccgctcccatgcctcctgccccctctcgccgtggaggcaccccaaaccctagcccctgaaaccctagatcctcctcccctgttccgatggcgggggccgcggcgtccccagcggcgggccacgtggtggagcggttccggtcgcgactgcgcgaggaagcgggcggcggcgagccgggcgccgccgcggtcgtgcacgtgtacgcggaggcgctccgtgagctaaccttcaactgcaagccggtcatcaccgagctcaccatcatcgcgggccagcacgccacgctcgccgccgggggaatcgccgacgccatctgcgcccgcgtcgccgaggtcgttctgctacttctgctctcctatcaggggtgttactcttattttgtcattaaggtggcccgccttacgatttctggggcataaggaagcttgggcttggagcgtcgagtagtctgggcgcttttgccgtgtagtcactagggtttgaggattggtgtgcttgttgtgaaacgtggattgctgtgcttggttttgaaatgtgagaaatcccataaaatggacggaaaaccttttattcccaagttttcctaggttttacaaaagcttcagcctgtacaagatcattcctttttgtaagaagcgttgtgcatacagtttagtatgtttgctctggttgctgtttacagctttggtagcatatttagggggtgtttgaatgcacaagagctaataggtagttggctaaaaaattgctattgaaattagctagctaaatattagctaatttgctaaaagtagctaatagctgaattattagctagagtgtttagacgtctttagctaattttagcagctgactattagctctagtacattcaaacacccctttcctggggtgtagaacctttgtggaactttgtggattaattggtgcttgccattttagggggtgtttggtttgtagggactaatttttagtccctacattttattccactttagttacaaaattatcaaatatagaaactaaaactctattttagtttctatatttgtcaatttatatactaaaatggaacaaaatgaagggactaaacattagtccctagaaaccaaacacccccttagtccctcttaaacttcctgttcattattggtaggatatgcatggaatatgtagcaacatagtaaacagttataccttaagagggtatggacactgacactgcattggaaaaagtggttgctatggttgatgaacttctatttgggatgggaagtttgagagttctgttgaattgagataaacgttaaacgaaaagagctgcactggttcgtgacttacaatgaaaagagttcacaactttgtgtttattattagttattagggcacttgtgaacttaatcagttacctgtttgctagttctgtcttctgtggtcgatttggttttgtttagaaatatgttgtaattttccgtctgcttttcttgaagataagacgctgcaatttcaaatgggaactaatcataagagcaattcacccgtatagtcacccaaaacaatgttttgcactgtagactgtactgctcgcagagtgaagtttgaatataggtatgaggataggtaagctgttggagatagcctaagagcaaggattaagctaccagtgtggtttgcagttttcctttcagaatccgttggttgtttgagattgtttattaattagttttctccctcccacatgcatgatttttaattaattacaactatcaaagttctttcgtgtgatgatcattattctgcgatattcttgcaggtgccaccagatcagatattaccatctttgtatctgctagatagtattgtgaagaacataggtggggaatatgtagagcattttgcaactaggttgcgaagtgtatttgttgatgcatactacagagttcatccaaaccagtatacttcaatgaggcgcctctttcgtacctggtggccagtgttcccttcttctgtgcttcgcagcattgaggatgaccttcaattttcttcctcggagaataaccgccccaccacatcaacaaatctgcatcaaacagagtcgttatctcctaggccatctcatggcatacatgtaaatcctaaatacctggaagcacaacataagttcaagcaggccaatgtggtaagactttttgtgagcaacatgtgtagatactagaatcaccggtgtatttgttgtggttttaggcatgataattgcagtctaataactgtcaatagtgtcgtattgttgcacaacttattgtgttctataacagtctgcgatttaagttaacttcaaacttattgaggatgtttgtccactttatttccttaaaaatcaggtgcaccagccagcagctcgggacactaggcaaatgactgatgtgcaggaggatctgataaatggattagcatcgaatagttcgtgtgaacggccttcaattttccaggtatttaagatcgcctcttcccttgtctctccttgtcatttttgcaaccagtctccaattccaagcctgtcttccagacatcaatgtagtaatgcctgcaggtaaaaaactgtcttttgtttcatttgctctcattattagttgtgaaagaattttttttgtcttaaatatttttgaggaaatactctatttatttaagataccgattctggttactgacagttaattccaagagtttttatgacttgttcctttcaagagttcctacagctctgtacctgatttagtattcttgataacaaacagtcaaacttgtctgacttcttatgtcgcttgaaaaaatgttaggtgctttattttacctcaagtgaaatgaaaccaaagtactcaatctaccccttccattccaaaatgtaggtcattttagttttgtcctaagtcaaatgtttctagcttgaccatgttttagaaaaatgcactggcatgtagagcatcaaattagtttcattaatttcaccatgaaatttgtcttgatagtgctaagctaagcattgtagataatatatttttctataaactccgtcaaagttagagaagtttgattttatttaggttagaaatataataccatattacattacatttttgggatggaagtattacttcaacatccataaatgctcatctgcatttatttttctttcaatctaaattttcaaatgttatttctgcctctggtttactttgtgatggctcatgatatgtgttgtaatcatatgggcagaaatcttctttacagtatgctgatgatcctgatcaacaagagacgtttcggcctcttgctggaacaagggcagcatcaccacatctcttgagtacacatgcatctgatatgaatctagatgggccattagtcaattcaaggagaaacttgtccatatcaccacctcttgatgtgttctctaggaatgtgtcccccaagagagcactagagaggctgccaccatctcattctgtattgggacctgatcccagaaaattcactgatcgaaatggcaggttaagatggggtttggatgatggtgcacaacggcctacaatcagtatgcttgatgaagagtacaggaagcaaagtgcaagggaacttattgatgcatatgggaattgtcaaggacgagatgctgatgaacgacttcctaaagtgcagcgcctggatccaaatggtatggctagcagatctagtgcacgaaattggttaacttccgaggaagaagagtattcatgggaggatatgagcccaactttgactgatcgagtcagtagcagcatgccatcttttcctcctggaaccatgagaactggatttcctggggcaaatgcagggttactggaatctgatgttgggaggcacaatttcccaagccaagctactagattatcggttgatggttcaccgtttaatcttgaagataggttttcaactgcaagtgtatgattgtctgatctctctttaaataatagtgaatcacagcattagcataacactttgtgtgcctatcgttacctcgtgctattcttgaaattattttctccttttcctttaaaagatttatgatgtccagtatgcaaattgattgttgctttctgatctaatatatttgtaaaaatcattgttctaaaaaggctggttgccaactagccatgtgttgtccagttgaattgggctggctaggtggccctaggcgcacatgataagctagttgggccagatgtgccactagagtggcagaagcagtagctagcaaagggccaacaagaccatgaccttttgtgccctctgcttccttcctctcctgctcacatccttagttaaggtcttatgccgatggcccatgtatgcaagttagcctgtgcagatgatgcagtattttgctgcctgcctagagcctagtgcctttagatcaatgataaaaatctgctatacaagtaatataggcttcagctggatgatctagagatattgttgccaaatttcaaatgacatgtatcctgcgacaatgcttgtttgaccgaaagaacaaaagagtagttaggtagaagttctgcaaataaattagttagagaaacaacattgtgttaccacatatgtatgctgttagggaaggaactgtataagcataatttttcagcagtttattcctttgtattttacaattggatataaaataatcaatgccttcagttactcagattttaaagcatgcaccatgagtaggactagcgcaatattttctagtagcctgcagcagtttttaggcactgtatgtattatttaactgttttcttgtcttgtgtttaactgaaaccctttgaacattttttgtccatgcaacaaataggatcatatgcagacttgtcaacatgatagtgctgtctcgatgaatctatgaacacctattgacacaggaacacctttacctcttgcagcatgttaacatgtctacaaggagatatccaagcaattttggtgttcagaatggagctcttttgaaattccagaattctgaacatgccctaaatcatgggagaattgctaccatgccagctccaccttggcaacagcctactggtctgccattgcaaatgcaagcacctcaacattcttcagcacttgatagattaccacagcccgctgacagtgaaatgccagtcaagaggttggacattcgtggcacatacaatggtttgaatgtagatagatccttggcggagaagcataggtcatcacctgctcgtgctccgacagaatggcttccccttcaccatacaccatcacaaactttaccactaattatacctgagacaaaacatgttagaagtgcatcggatagtttagaaattagttcttttgttagccaaggagctagttcatcagtatttgttccgcatcatcagtatgatgccttggaccggaaaacaggtacccgtaacttagctcaaccaccatatcagcaccaagatttgttgctaccaagccaacaaaatcaaggtacaattttgggaaaccaagttcatcctcatctgccccaacagttacatccccattcccatcctcaccatcaggagacttttagaagctttgcccctggcatgactgtatctccattccaaggacaaggaggcaatgcaaccatgacaccagtttccgttctgccaactttttctgtaccacctgctgtgccaccctatggcgtgctgtctgcacctgggtttcctatgccaccattaccgcccggaccaccacctgtcccactgcagatgggttcatcatcatcacaagttggtggccctcaaccatttgtctctggacttttgagtaaccttatgcggcatggtgtaatttcactggaaccacctagccaatcccaggtatttctcatgcttcatgcgttgttatatagtattattctgtttatatttatgctgttaattaaggggaaaattgtgaacattttattgtaggattctgttggagttgacttcaatgtagacctcaaggtacggaatgagcctgtcatcaatgctttgtatcaggatctttcaaggcagtgtaaaacttgtggccttcgatttaaatgccaagaggagcatcgtgctcacatggactggcatgttacaaaaaataggaattcaaagaaccgtaagcaatcttcacgtaaatattttgtcacggtggaggaatggttaagagctgcagaaacagtaggaaacgatggagttcctgcttttgtgccctcagacccagtcccagatagaaaggaagagagagaaattgctgttcctgcggatgaggagcaaacagcctgtgcattatgtcaagagccatttgaagatttctacagtgatgagactgatgagtggatgtatagaggtgcggtctacatgaatgcaccagacggtaatattgatggtctggaaaggtcacggttgggacctattgtccatgctaaatgtcgatctgggcccagcaatacttagggtgggcatggctggtgagtatctatctatttccttgtgctataacaaacatctatggactttaaggtggacataatgttttttagagctagtttgggaacctcattttcccaagggaaattgtatcttgaggttaaccccactgtaaatagaggatagtctccttgctagtatttggctgtatgcaacatgtttttggctacgattatatatgttatctgactaaataaaaatgctggatgtgagttgttgttcattatttttccattgtttgtagttagtgttcaatattatatgaatatatacttctgtcatacatttggacccattcttttttgtttggttttcagttctgctgaaccattttgcagatccatcatatggattttcagaatgtctgattctactggcatactcccacggtccaagaaagaatgcaattctagcagtgtaatgttttagtatcttaagtttgatcaattatagataaaaaatatcaatatttatgataccaaattaatatcactagattaattatgaaatgtattttcgtaataaattgattttgagccataaatgtgaacatcattgactagaaacttgttcaaacatgaaccactttaactggcacacaacccatagctgcattctttttggaacagagtgagtatataataagaggactttcaattgctcacttgaatagttagttacctttgaaggattctgtaggttttaaaatgtaaatgtgaacgctcatgcttgttcaagtaatgcaatttttaattggtgtaactttggtaatgacaaagataatcatgctaactatgtaattttatgctaatgacaactgtaagcatgcaaacccacagttttgtatgtatcatttagtgcgatctgatgatgatttatgaaagctgtaatttcctagcatgactacacttttaatctctcaccttttggtatctaaggactaaggatagaagtaggctttggattaatttgtaaaaaagtggcgataattagttcaggactactatagttcctttttatttgtacttcactatttttgcccatcatggcattgctaatctgcgcaactattttggtatctaggatagatttccttaagcaggttttggtctaatttgtaaaaatgtggcaatagtaaggagttcaggactatcatagttacttttcatttgcaattcaccattttgctcatcatggcactgctaaactgcgcagttgttatgtctgccaatcaaagattcatgcatttagtggaatgacactaatactgtttctgtcatttcctgacagtgatacactggtgcacaggaccttttattttctttattgtttttgtgagttttttaagttttttgtgctagctcttagcatgtttggaagcattgtttgtttttaattatctggtcagtgttcatgcaattagtaagtagcataggcacataagttctggttacctcttgctgttgacaattgacatcacgattcctgttaggatagaattttttctgttcaaatatcaaacagatctgacatgaaatttcttgaagtatttgactgagcaatcttatgtttgttatgttttctttgtcatataattttttccctttggattatgaaatcctcagtgaccggttataatttggaagaccagttataagctggaaggtggtaaaattgagatatactcggcttatttgccgttgcacggtaccatgtaatttagttacacgctgttatatcagccactacctctgttccttagagaggtgcatagccagaatgacagttttttaggaacgaaggtaatagttgcaacaacgccttctttggctccaaaaggatcctttttggtacgcgccaaaatatgaagctaagtgtggttgaaaagcctgctgagttttcctgaatggtgtatgatcctatgagatgggttcattggttgactccttttgccatggtgatgattattttgaatgcataatatttgttttcatatcagttgctgtgatggcttcaattttattcattttccgtccctaattaattgttaataaagccaaaatgaagttaactaagcatattgtatattagcttcgtgtaaacaaatcacttgaactgaaacttgctatgatctaatgttagtaaattccttataagtgcttcattgaaagatgctgctataactatttttatcttcggtgcagggatgacttgatagcttttttttccgttgcggctgtaggcatgacctgtaaactactggagactcagaatttggtatggttagattttttcctatctatggcttcgttaacagccaatgcacagaatgttaatgtgacaatagtgcataactgtaaaatttttctgtaattcagtgattggaagttagtaatcggcacaacattcactattgtcatattgtgcttttgcccctgtcattttgtccttccatagttgcacagtgctgctttttcattacagccgtagtgtgatctatacttaagcttcatgtttagggtttctgcagcacagttgttgtcaggacagcggtttatggagccatggtctcactggacatgggaaacaatatatttcaatagtcctgctttcttcctcgttaatatcttgtgcaggatacataccagttctctatgttggttggtagctttggaagggaccgtgcaagatgaccaagagtgctggccactatttatgcctaagtattgaaagggactagatactgtcattaccttattgccaatttttttagtattttcctttgtaagttgagagcattgtttgtgactattattcttggttgcttgtatagttccatcttgctgatatgtttatactccaaaatgtgggtcagaatgtcttatcagactttattctttattctggttggtatatgctccggcaaactattcaatggaacaatttgggaatattttttcgtcca

SEQ ID NO: 74 - DNA, 2904 bp, Zea mays
  source 1..2904: mol_type = other DNA
atggcgggggccgcggcgtccccagcggcgggccacgtggtggagcggttccggtcgcgactgcgcgaggaagcgggcggcggcgagccgggcgccgccgcggtcgtgcacgtgtacgcggaggcgctccgtgagctaaccttcaactgcaagccggtcatcaccgagctcaccatcatcgcgggccagcacgccacgctcgccgccgggggaatcgccgacgccatctgcgcccgcgtcgccgaggtgccaccagatcagatattaccatctttgtatctgctagatagtattgtgaagaacataggtggggaatatgtagagcattttgcaactaggttgcgaagtgtatttgttgatgcatactacagagttcatccaaaccagtatacttcaatgaggcgcctctttcgtacctggtggccagtgttcccttcttctgtgcttcgcagcattgaggatgaccttcaattttcttcctcggagaataaccgccccaccacatcaacaaatctgcatcaaacagagtcgttatctcctaggccatctcatggcatacatgtaaatcctaaatacctggaagcacaacataagttcaagcaggccaatgtggtgcaccagccagcagctcgggacactaggcaaatgactgatgtgcaggaggatctgataaatggattagcatcgaatagttcgtgtgaacggccttcaattttccagaaatcttctttacagtatgctgatgatcctgatcaacaagagacgtttcggcctcttgctggaacaagggcagcatcaccacatctcttgagtacacatgcatctgatatgaatctagatgggccattagtcaattcaaggagaaacttgtccatatcaccacctcttgatgtgttctctaggaatgtgtcccccaagagagcactagagaggctgccaccatctcattctgtattgggacctgatcccagaaaattcactgatcgaaatggcaggttaagatggggtttggatgatggtgcacaacggcctacaatcagtatgcttgatgaagagtacaggaagcaaagtgcaagggaacttattgatgcatatgggaattgtcaaggacgagatgctgatgaacgacttcctaaagtgcagcgcctggatccaaatggtatggctagcagatctagtgcacgaaattggttaacttccgaggaagaagagtattcatgggaggatatgagcccaactttgactgatcgagtcagtagcagcatgccatcttttcctcctggaaccatgagaactggatttcctggggcaaatgcagggttactggaatctgatgttgggaggcacaatttcccaagccaagctactagattatcggttgatggttcaccgtttaatcttgaagataggttttcaactgcaagtgaacacctttacctcttgcagcatgttaacatgtctacaaggagatatccaagcaattttggtgttcagaatggagctcttttgaaattccagaattctgaacatgccctaaatcatgggagaattgctaccatgccagctccaccttggcaacagcctactggtctgccattgcaaatgcaagcacctcaacattcttcagcacttgatagattaccacagcccgctgacagtgaaatgccagtcaagaggttggacattcgtggcacatacaatggtttgaatgtagatagatccttggcggagaagcataggtcatcacctgctcgtgctccgacagaatggcttccccttcaccatacaccatcacaaactttaccactaattatacctgagacaaaacatgttagaagtgcatcggatagtttagaaattagttcttttgttagccaaggagctagttcatcagtatttgttccgcatcatcagtatgatgccttggaccggaaaacaggtacccgtaacttagctcaaccaccatatcagcaccaagatttgttgctaccaagccaacaaaatcaaggtacaattttgggaaaccaagttcatcctcatctgccccaacagttacatccccattcccatcctcaccatcaggagacttttagaagctttgcccctggcatgactgtatctccattccaaggacaaggaggcaatgcaaccatgacaccagtttccgttctgccaactttttctgtaccacctgctgtgccaccctatggcgtgctgtctgcacctgggtttcctatgccaccattaccgcccggaccaccacctgtcccactgcagatgggttcatcatcatcacaagttggtggccctcaaccatttgtctctggacttttgagtaaccttatgcggcatggtgtaatttcactggaaccacctagccaatcccaggattctgttggagttgacttcaatgtagacctcaaggtacggaatgagcctgtcatcaatgctttgtatcaggatctttcaaggcagtgtaaaacttgtggccttcgatttaaatgccaagaggagcatcgtgctcacatggactggcatgttacaaaaaataggaattcaaagaaccgtaagcaatcttcacgtaaatattttgtcacggtggaggaatggttaagagctgcagaaacagtaggaaacgatggagttcctgcttttgtgccctcagacccagtcccagatagaaaggaagagagagaaattgctgttcctgcggatgaggagcaaacagcctgtgcattatgtcaagagccatttgaagatttctacagtgatgagactgatgagtggatgtatagaggtgcggtctacatgaatgcaccagacggtaatattgatggtctggaaaggtcacggttgggacctattgtccatgctaaatgtcgatctgggcccagcaatacttag

SEQ ID NO: 75 - protein, 967 aa, Zea mays
MAGAAASPAAGHVVERFRSRLREEAGGGEPGAAAVVHVYAEALRELTFNCKPVITELTIIAGQHATLAAGGIADAICARVAEVPPDQILPSLYLLDSIVKNIGGEYVEHFATRLRSVFVDAYYRVHPNQYTSMRRLFRTWWPVFPSSVLRSIEDDLQFSSSENNRPTTSTNLHQTESLSPRPSHGIHVNPKYLEAQHKFKQANVVHQPAARDTRQMTDVQEDLINGLASNSSCERPSIFQKSSLQYADDPDQQETFRPLAGTRAASPHLLSTHASDMNLDGPLVNSRRNLSISPPLDVFSRNVSPKRALERLPPSHSVLGPDPRKFTDRNGRLRWGLDDGAQRPTISMLDEEYRKQSARELIDAYGNCQGRDADERLPKVQRLDPNGMASRSSARNWLTSEEEEYSWEDMSPTLTDRVSSSMPSFPPGTMRTGFPGANAGLLESDVGRHNFPSQATRLSVDGSPFNLEDRFSTASEHLYLLQHVNMSTRRYPSNFGVQNGALLKFQNSEHALNHGRIATMPAPPWQQPTGLPLQMQAPQHSSALDRLPQPADSEMPVKRLDIRGTYNGLNVDRSLAEKHRSSPARAPTEWLPLHHTPSQTLPLIIPETKHVRSASDSLEISSFVSQGASSSVFVPHHQYDALDRKTGTRNLAQPPYQHQDLLLPSQQNQGTILGNQVHPHLPQQLHPHSHPHHQETFRSFAPGMTVSPFQGQGGNATMTPVSVLPTFSVPPAVPPYGVLSAPGFPMPPLPPGPPPVPLQMGSSSSQVGGPQPFVSGLLSNLMRHGVISLEPPSQSQDSVGVDFNVDLKVRNEPVINALYQDLSRQCKTCGLRFKCQEEHRAHMDWHVTKNRNSKNRKQSSRKYFVTVEEWLRAAETVGNDGVPAFVPSDPVPDRKEEREIAVPADEEQTACALCQEPFEDFYSDETDEWMYRGAVYMNAPDGNIDGLERSRLGPIVHAKCRSGPSNT

SEQ ID NO: 76 - DNA, 19 bp, synthetic construct
  source 1..19: mol_type = other DNA
cagaggattatgaccagat

SEQ ID NO: 77 - DNA, 19 bp, synthetic construct
  source 1..19: mol_type = other DNA
cagcaaccgaagaaatcga

SEQ ID NO: 78 - DNA, 19 bp, synthetic construct
  source 1..19: mol_type = other DNA
ccgcgcgttggaattaaca

SEQ ID NO: 79 - DNA, 19 bp, synthetic construct
  source 1..19: mol_type = other DNA
agcctagtcaaccaacgag

SEQ ID NO: 80 - DNA, 19 bp, synthetic construct
  source 1..19: mol_type = other DNA
tcgatactcgaccggttca

SEQ ID NO: 81 - DNA, 19 bp, synthetic construct
  source 1..19: mol_type = other DNA
ttgatagatccagagatcc

SEQ ID NO: 82 - DNA, 19 bp, synthetic construct
  source 1..19: mol_type = other DNA
ttggttcatcgctgagcct

SEQ ID NO: 83 - DNA, 19 bp, synthetic construct
  source 1..19: mol_type = other DNA
gatctctcgatctatcgaa

SEQ ID NO: 84 - DNA, 19 bp, synthetic construct
  source 1..19: mol_type = other DNA
taaatctggacgatttcgt

SEQ ID NO: 85 - DNA, 19 bp, synthetic construct
  source 1..19: mol_type = other DNA
ccaacttgactattattgc

SEQ ID NO: 86 - DNA, 19 bp, synthetic construct
  source 1..19: mol_type = other DNA
gttctcgcgatctatcgaa

SEQ ID NO: 87 - DNA, 19 bp, synthetic construct
  source 1..19: mol_type = other DNA
caagcatcatgaggttgag

SEQ ID NO: 88 - DNA, 20 bp, synthetic construct
  source 1..20: mol_type = other DNA
ccgagctcaccatcatcgcc

SEQ ID NO: 89 - DNA, 20 bp, synthetic construct
  source 1..20: mol_type = other DNA
ccccttggaaccctgagagc

SEQ ID NO: 90 - DNA, 20 bp, synthetic construct
  source 1..20: mol_type = other DNA
ctgggcgaggcgaggcttct

SEQ ID NO: 91 - DNA, 20 bp, synthetic construct
  source 1..20: mol_type = other DNA
cagcaggctgttgtgggaac

SEQ ID NO: 92 - DNA, 19 bp, synthetic construct
  source 1..19: mol_type = other DNA
gtattgtgaagaacatagg

SEQ ID NO: 93 - DNA, 19 bp, synthetic construct
  source 1..19: mol_type = other DNA
gcacaacataagttcaagc

SEQ ID NO: 94 - DNA, 19 bp, synthetic construct
  source 1..19: mol_type = other DNA
gggctacagcagttaccac

SEQ ID NO: 95 - DNA, 19 bp, synthetic construct
  source 1..19: mol_type = other DNA
ttctcagcaagattaccag

SEQ ID NO: 96 - DNA, 793 bp, Arabidopsis thaliana
  source 1..793: mol_type = genomic DNA
agattcgttttatttttcaatcgtttcaatttctaaaattctcctctctcaacgcaatcaccaaatccaaatttagggtttcttttctctttctgcgatcgatctctgaaaactcataactgttgtttaatcgtgaagatggcatcaaacggatctttctctgctcagagaaacgcaaacgctagaacaacgatgaagagaagaagcgacaatcgcggttacggtggcggaatcggaggttatcaagaggagacgaaccgttacgcgccgccgcaaaaacgtttcagatcacaagcgcaacagcaattccgatctcgaggggatggttagcaagtgcatctttatggctttgtgcagcgacgggaggagagacggtagaagtagcatcttttggaggagaaatgcagaagaagaaaggaaaggacgaagaaccaaaacaactcatggtgcctgcggatgaagatcagaagaattgcgcattatgcgtagagccgtttgaagagttctttagccatgaagatgatgattggatgtataaagatgcagtctacttaaccaagaatggtcgtatagttcatgttaagtgtatgcctgaaccccgacctgctaaggatttgagagagccttcgagggtaatgtctgttacagttccatcggttgcgaaagctatagcctgctgattgagatttttaagatctctgttttagtcattcgtttttttaagtaatgtactttacttgaacaagttgtaacacaaatacagttctatcaattgaatctcctattttactct

SEQ ID NO: 97 - DNA, 4759 bp, Arabidopsis thaliana
  source 1..4759: mol_type = genomic DNA
taaaatccccaattcacaaccctaattgatcggagattgtccaaactctctgacatttttgccgaattcgaagcattccttctatcttctccgtcttcttcgcagaatcgcgttatgcatctcaatttttagattgtaacttagggttactgattgttgtaatgctctgaattcccctctaacgaggatttgttgtcactgcaatcgatttttgccgattaggtttaatttagggcattatttgcgtacctcttgtattgtatagttctttggaatggagaatcctcgtagaccgtttgatagatccagagatccaggtccgatgaagaaacccaggctcagcgaggaatcaatccggcctgttaattccaacgcgccgactttgagctcactttcaactcgaagccgataataacgaaccttacaataattgctggagagaatgttcatgctgctaaagccgtcgtaaccgccatttgcaacaacatcctcgaggtgaacacccagttttcatgcccactgttttgttgttataatccgtgctttagttctcaaattcttgtttgagtaataataattgtacgatataaggggaataaggtaaaaggtggtggttgagatttgtgttatctgtatacgaggttcaatgagaaattgagttttttgaatcaaccctagtatccttttctgggatctatgtgtttttatatttcttcatcaagtcgcaaatcatttctttgaaacaaagttcatagtactgacacaatgttgttaacaatctaaaaaatagctgttgcccatgcaaccattttgttcatgttcatatatcaagaatgcatatatcagctcatcatgtgattgtactgagttttggttatatgagtgtgtagtctctttatcagcgttagagttttttgtatctcactttaccatttagcaagggctttcataagctctgtttttattgaggttggattttctgttggcggtttctgcttaccgcatgctctccacaaagccaagtcaacttttattcagtctttgggtagcaaggaagcactcaatagaaggagcctgatccatcaagtctgtgtggagagtgaagacaagaattgctattgcagtgttgtttacatcgtctaatggttatctgtgtctattgttggatttctagttgtcagaatatgtgttagtattagcttgaatgtcatcttggtctgtgcttttgattctactttaagttattgatttctttcattacctttagaggctcatgtggaaatctcgatgttactctctatgagatcaaattggccttttttcttctgggttatctgatttgaagaatatcatgcaggtaccaagtgatcagaagctgccaacactgtacctgttagacagcattgtgaagaacatcggaagggattacattaagtactttggtgccaggctgcctgaggtttggtgctttgttgatagtcatggttatagaatgtttttttcctagcaaattcatctctgatcaccctctttttacttctctgaaggtttttgttaaggcttacaggcaagttgaccctcctatgcattctaacatgcggcatctttttggaacctggaaaggagtctttcatccgcaaacacttcagcttattgagaaagaacttggctttaatgctaaaagtgatggttcagcagctgttgtatccacagccagagctgagccgcaatctcagcgcccaccacatagcatccatgtgaatcccaagtatctagaaaggcagcgccttcagcagtcgggacgggtaagcaatgaccaaagtaaacttgcagccactgaaagttattagtgatgatacaccctaaccttcagctctactagagatttcgctgttctaagtgttcataaatgtaaccttcaggagtactaaacatttctctgttctaagtgttcacaaatgtagggaacatttctttttcggtttagatagcaatcaatttctttcaaagggtcctgggtttctggatataggtagaggctacttatcgactttattaaaatttccctgagtatttgtcggttttctttgcagacaaaaggaatggttactgatgtcccagagaccgcacccaatttgactagggattcagatagactggagagagtttcaagcatagcatctggaggctcgtgggtaggtccagctaaagtaaatgtaagatgatttttccttggcatggttacagtttttttacctaagtgctttccactgattgtgtgtcgaattcagaacattcggcgacctcaaagagatttacttagtgagcccctctacgagaaagatatcgagtcaatagcaggggaatatgattatgcttctgatctaccacataactctagatcggtgattaagaatgttggttcaaggattactgatgacggatgtgagaaacaatggtacggagctacgaacagggatcctgatctgatttctgaccaaagggatgggttacattccaaaagcagaacttccaactatgctacggctagagtagaaaatttggaatcaagtggtcccagtagaaatataggtgtgccttatgatagttggaaaaactctgaggaagaagagtttatgtgggatatgcactcaaggttatccgaaactgatgtagccactatcaacccaaagaacgagcttcatgcaccagatgaatcagagagactggtaagctatgatagtaatttgcattcctttttattgtgatgtatctcattttaatttgttagcttttaaccatctcccatcttgtaggaatctgaaaaccacctcttaaaacgaccaaggttttcagctctcgacccaaggtttgacccagcgaattcaacaaattcatattccagcgagcaaaaagatccaagtagtattgggcactgggcgttttcttctaccaatgcaacctcgactgcaactagaaagggaattcagcctcaaccaagggttgcaagctctggaattttgccaagctcaggatctgggtcagatagacaatcgcctttacatgattcaacttctaaacagaatgtgaccaagcaagatgttagaagagctcactcactcccccaacgtgatcctagggcttcccggtttccagcaaaacaaaatgttcccagggatgactctgtacggctcccatcttcaagtagtcagtttaaaaacacaaatatgcgtgaactaccagtggaaatctttgactctaagtcagcagctgaaaatgccccaggccttactcttgcctcagaagctacaggacaacccaacatgagtgatttgctagaagctgtcatgaagagtggaatcctttctaataattcaacatgtggtgcgatcaaggaggaaagtcatgatgaggtcaacccgggggcactaaccctgccagcagcctctaagcctaagactttgccaatttcattggctactgacaatttattagctcgtttaaaagttgaacaatcctccgcaccacttgtctcatgtgcagcgtctcttactggcatcacctcagtacaaacgtcaaaagagaaaagtaaggcctcagatcctctttcatgcctcttaagctctttagtttcgaagggcttgatatctgcttcgaagacagagctgccatcggctccttccataacgcaggaacacagtcctgatcattccaccaatagctctatgtctgtatctgtggttccagcagatgctcagccatctgttttggtgaaggggccatctactgcccctaaggtaaaagggttagctgcaccatctgaaacatcaaaatctgaacccaaggacctcataggattgaagtttagggcggacaaaatccgtgaacttcacccttcggtcattagtagtctattcgatgaccttccccatctctgcacctcctgcagcgttcgccttaaacaaaaggaagaacttgataggcacatggagctgcatgataagaagaagctcgagttaagtggcacaaacagtaaatgtagggtttggtttccaaaggttgataactggatagcagcaaaagcgggagagttagaacctgaatatgaagaagttctgagtgaacctgagagtgctattgaggattgtcaagccgtagctgctgatgagacccaatgcgcatgtatattatgtggagaagtatttgaggattatttcagtcaagaaatggctcaatggatgttcaagggagcttcttacctgaccaatcctccagccaacagtgaggccagtggtcctatcgtccatacaggttgtctgaccacgagttcattgcagagcttggaagttggaattgcaatcaagcaggtaacttgtagctctagtcaatttgcgacttttggtcattctagggctctctgatctcaaactttgttctttaatcctgcatgaatccacctttacgtgtttgtttctcgttcggttcctattttgctaggaaattgttgagtagcctccatataaagacctatggtggaggaagaagtggcatcacagaggaaacaggttcgtgattcctctgagttcctcatgatgctagttttgtagaaaagtgtcgagcttttgagttaaggaaaaaagatgtgcagcatttttaattttattttttcttatttgtctctcctttttcatcatatacaaatacaaaaagagcaaagaggccttcccaatatcggacatttccatattctatgcctttc

SEQ ID NO: 98 - DNA, 3958 bp, Arabidopsis thaliana
  source 1..3958: mol_type = genomic DNA
atctctctcttcccccacaagggtggcgattcgaggagtttccatggtggaacagaaaccctagagcctttatgctgccactaattatcactcttcactattgaattggggatggactcagagaaaatcctcaatccaagacttgtttcgattaatagcaccagccgcaaaggtatgtccgttgagttgccgcagaaaccgccgccaccgccgtcgttgttagaccggtttaaagctttgcttaatcagcgtgaagatgagttcggtggtggtgaagaggttttgcctccgagtatggatgagattgttcagctttatgaggttgttttgggtgagcttactttcaattctaagcctattatcactgatctcactattatcgctggtgaacaacgggaacatggtgaaggaatcgctaatgctatttgcactcgaatccttgaggtttgctttcttcttatttgtttccactttctagccttattgtatagtatgtaagtgtagaagtgggaaaaaaattaggtttttgattgtcagagattagtgtttggtttgtggtttagtgaaatggctgcttctgagttacctgtgggtgtatatatgcaggctcctgttgagcaaaagttgccatctctgtatcttttagacagcattgttaagaacattgggcgggattatggtcgctatttctcatcccgtctacccgaggtgagcagtctacaagaagatgtttacttaaatgatttgaatagggctgaagcatacccatgagctgttttgatcctattctgctttaggtcttctgccttgcatataggcaagcacatccgagtttacatccgtcaatgcgtcacctgtttgggacttggtcgagtgtttttccaccacctgtactacgcaagattgatatgcaattgcaactttcatctgcagcaaaccaatcttccgtaggggcttctgagcctagtcaaccaacgccgaaaatgtaagcctttctctttgtcattatgaacagagatgacattctttttttaggtgcatctgcaaccttgaagctatggtgtgttaattttatataagtgtactaagtaaaacgggtattagattagtttggcctcatagtggtcttatccattgtatttctttaaagttttgttagttagacgagatagatgtttacattcttcaagtaatcaatcttgactggaagggagttgttgttatggcctctctcagttatacatgatgagtaggtcactgggactctggtacattagaaatgatcttgttagcacatgtagagggtttttttttggttatagtacagtagatcactgtttatgcatcatgtgtcgtttggaactgtttttatatgttttttttcaaagagtgactatacctgagatgcatatttccttaattgctcttgcttgacatgcagaatttgagaggaattaattccagtgcaagagtttatggccaaaattcactcggtgggtataatgatttcgaagatcagttagagagcccatcttcactgtctagtacaccagatggttttaccaggaggtctaatgatggagctaatccttctaatcaagcttttaactatggaatgggtcgagctactagcagagatgatgaacatatggaatggcgaagaaaggaaaacttaggccaaggaaatgatcacgaaagacctagagcgttgatagatgcatatggagttgacaccagcaaacacgtaacgatcaataaaccaattcgcgatatgaatggcatgcacagtaagatggttacaccatggcagaacactgaggaagaagagtttgattgggaagatatgagccctaccctagatcgtagcagagctggtgaatttttgcgatcatctgtaccagcattaggaagtgtaagggcaagacccagagtaggaaacacaagtgattttcatttagattctgacattaagaatggtgtaagtcatcagctacgagaaaattggagtttgtcacaaaattatccacatacatccaaccgtgttgatacaagagcaggaaaagacttgaaagtattggcttcatctgtagggttagtttcatcaaacagtgaattcggggctcctccatttgatagcattcaagatgttaactcacgttttggccgggctcttcctgatgggacatggccccatttgagtgctagaggacccaactctctccctgtaccttctgctcatttgcatcatttggcaaatcccggtaatgcgatgtcaaatcgcttgcaaggtaagcctttatataggcccgaaaaccaggtctctcagagtcatcttaatgatatgactcaacagaatcagatgctagttaattatttgccatcctcatctgctatggctccaaggccaatgcaatcgttgttaacccacgtaagtcatgggtaccctccacatgggtcaaccattagaccatctttatcaatccaaggtggggaagccatgcatcctctttcttcaggagttttatctcagataggagcctctaatcaaccaccaggtggcgcattctctggtctgattggctctctcatggctcaaggtttgatctcattgaacaatcagcctgctggtcaggtaattattcaaatcatgtccttgagagtcggtttgttcttagttattgctttatcttgaattgttggtttatatattcagggaccattggggttggagtttgatgcagacatgctcaagatacgtaatgagtctgcaatcagtgctttatatggggatctcccgaggcaatgcacaacctgtggtctacgcttcaagtgccaagaagagcacagtaagcacatggattggcatgtaaccaagaacaggatgtccaagaaccataagcagaacccttctcggaaatggtttgtaagtgctagcatgtggctcagtggtgcagaagcacttggagctgaagcagtaccagggttcttgcccacggagccaacaacagaaaagaaggatgacgaagatatggcagttcctgcagatgaagaccaaacttcatgtgcattatgtggtgagccatttgaagacttttatagtgatgaaaccgaggaatggatgtacaaaggtgcggtatacatgaatgctcctgaagaatcaacaacagatatggacaagtctcagttaggtcctatagtgcatgcaaagtgcaggccagagtccaatggggtaagccattccatgcattttcattagcttcatttgtgtctagttgaatccattttcattttcgctgctagacatttatccagtgacgacctcttgatttcccgatcatggctttttcagggtgatatggaagagggtagtcagaggaaaaagatgcggagttagatcccttagaccgaattttaggtctttttcttgactacatttaagttacattcttatttcttcatcatctatatgcacttagtcacagaataccaccaatgaagagaaatgatggagagagtcttctttctcctaaaactgtaccattgaaaaaaagaggtttacacccacatatattatcattctttatgcctctaaattcctgttgtgccttaggacatggatatcgtgtaactgttctgcattctctgctacacctaacctggggtgcttgtagtagtaaaatcagatgtcgcaacagtagtttccccatgtaaatggaatacaatgtctctgtttcggattgtctttttctttttcatctttagatattgcagggaaagcccgagaagactgatattcgaatgtgaacagatttcttatgatgaatggatctcatagatgctgttgctaagagaaccagtttcaggtttcttctttagagctttccctgtctgagttttacctttctatgttaggctgttagctaaaaatgttagttaacataactctctttaagcgtat

SEQ ID NO: 99 - DNA, 794 bp, Arabidopsis thaliana
  source 1..794: mol_type = genomic DNA
agattcgttttatttttcaatcgtttcaatttctaaaattctcaacgcgatcaccccaatccaaatttagggtttcttttgtctttctgcgatcgatctctggtaactcataacttgttgttgaatcgcgaagatggcatcaaacggatccttctctgctcagagaaacgcaaacgctggaacaacgatgaagagacgaaacgacaatcgcggttacggtggcggaatcggatgttatcaagaggagaggaaccgttacgcgccgccgcaaaaacgtttcagatcgcaactgcaacagcaattccgatctcgaggggatggttagcgagtgcatctttatggctttgtgcaccaacgggaggagggaccgtagaagtagcatcttttggaggaggagaaatgcagaagaagaatgaaaaggatcaagtacaaaaacaacacatggtgcctgcggatgaagatcagaagaattgcgcgttatgcgtagagccgtttgaagagttctttagccatgaagctgatgactggatgtacaaagatgcagtctacttaaccaagaatggtcgtatagttcatgttaagtgtatgcctgaaccccgacctgctaaggatttgagagagccgtcgagggtaatgtctgttacagttccatcggttgcgaaagctatactctgctgattgagattttttatatctctgttttagtcattcgtttttttaaaagtaaatgtacattacattaatcaagatgtaacacaaatacagttctatcaattgaatctcctattttactc

SEQ ID NO: 100 - DNA, 3169 bp, Brassica napus
  source 1..3169: mol_type = genomic DNA
atgcaatcggagaaaatcctcaacccaagccttcttctcagcaccaccaccggccgcaaatcgatgtccgtcgttaaaccgccgccgccgccgtcgatactcgaccgtcaaggctctgctcaatcagcgcgaggacgagttcggcggcgaggatccgagttcggaagaggttgttcagctctacgaggttgttctgggcgagctcactttcaattccaagcctatcatcactgatctcactatcatcgccgatgagcagcgcgagcacggcgaagggatcgctaatgctatctgcactcgaatcctcgaggtctgggactttgtttccaccgctaaagccttcttcttgtgactgtggtttcgtgtgtatgcaggcaccggttgagcaaaagctgccgtctttgtatcttttagacagtattgtcaagaacattgggagggactatgctcgttacttctcgtctcgtttaccagaggtgagtgagcacagtctatataatgagatccttgtttaattggtttgtgtttgattcttgtctgctgttttaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccggctcaaccaacaaggggcatacatgttaatccgaaatatcttcgtaggctggagccctctgccgctgagagtgtaagcttttctctttgatcactatgaacagagatgacgttcttctatggattcaggaagcctggtataagattactctagcctcatagtggaacccttgttctgtatagataagattacctccagtaatgaagttttgcgtaattgtcgtatttttaagagttagatttactttcttctagtactcaactggagtaaaaaatgagtgttgtggtctctcaattattcatgatgagtaggtcactgggactcaggtgcatttcttttagttagacgcaatagataatgcattgtttttcgttgtgaacttgttttcttataggttttttctaagatgatttacaactaacatgcatttttcttgaactgtgccggcttggtatgcagaacttgagaggaataagttccagtgcaaaagtgtatagtcaaaactcagtcggcgggtatgatgattttgaagatcagttagatagcccatcttctctggccggaccaaggtctagtacagctggaactttgaccctcgcatcttctgcaattggaggtaagaatcttcattctttcagtctcaagacgtgtaaaacttgttattgactttcagtagcccttcatctttcgacttttatgactctgcagcagatgggtttcctaggaggtttagtgatggagttaattcttctaatcaagcctataactatggaatgggtcgggctactggcagagatgatgaacacatggactggcgaagaaaggacaacttcggccaaggaaatgaccatgaaagacccagagctttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgttcttcctgatgggacatggccccatttgggtgttagaggatccaactctctccctgtaccttctgctcagcatcatttggcaaatcctggtaatgcgatgttaaatcacttgaatggtaagcctttatataggccagaaaaccaggtctcccacatgacccaacagaatcaagtgctaggaaattacttaccgtcttcatctgcaatggcaccaagggcaatgcaatcgctgttaccccacggtcatggttaccctccactagggtcaaccattagaccatcgttatcagtccatggtggggaagccatgcatcctcattcttcaggggtttccatctctcagaatccttcgttgggagcctctaaccaaccaccgggcggtgcattctctggtctgattggctctctcatggctcaaggtttgatctcgttgaacaatcagcctactggccaggtaaattatttaaagcatgtcacaccttctaatacatgagagtcggactctttcttctagctttatcttaaataattgattttatatatacagggggcttcggtgatggagtttgatgcggacatgctcaagatacgtaacgagtctgcaataactgctctatatggggatcttcccaggcaatgcacaacatgtggtctacgctttaagtgccaagaagagcacagtaagcacatggattggcatgtaaccaagaacaggatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgagccgtttgaagacttctatagtgatgagacggaggaatggatgtacaaaggtgctgtatacatgaatgctcctgacggatcaacaacagatgtggacaagtctttgttaggtcctattgtacatgctaagtgtaggccagagactaatggggtaagcatttccttgcattctcattagcttcctttggaatacatgtctagttataagtttgattatattatcattttcctgctataagctttatccagtggtagcctcgtgatttccctaagaatggatatttgtttcagggtgatatggaagagggtagtcaaagaaaaaggatgcggagttag

SEQ ID NO: 101 - DNA, 3203 bp, Brassica napus
  source 1..3203: mol_type = genomic DNA
atgcaatcggagaaaatcatcaacccaagccttcttctcggcaccaccaccggccgcaaatcgatgtccgtcgttaaaccgccgccgccgccttcgatactcgaccggtatcaaggctctgcttaatcagcgcgaggatgagttcggtggcgaggatccgagttcggaagaggttgttcagctctacgaggttgttctgggcgagctcactttcaattccaagcctatcatcaccgatctcactatcatcgccgatgagcagcgggagcacggcgaaggaatcgctaatgctatctgcactcgaatcctcgaggtctgggactttgtttccaccactaaagcccttcttctttgtgcaaacatcttgtgactgtggtttcgtgtgtatgcaggcaccagttgagcaaaagctgccatctttgtatcttttagacagtattgtcaagaacattgggagggactatgctcgttacttctcgtctcgtttaccagaggtgagtgagcacagtctatattataatgagatccttgcttaaatggtttgtatttgattcatgtctgctattttaggtcttttgtctagcgtacaggcaagcgcacccgagtttacatccttcaatgcgccacctgtttgggacttggtctggtgttttcccatcacctgtattacgcaagattgagatgcagttgaagcttacttccgcaacgaacagccaatcttccttaggggcttctgagccgactcaaccaacaaggggcatacatgttaacccgaaatatcttcgtaggctggaaccctctgccgctgagagtgtaagctcttttctctttgatcactatgaacagagatgacgttcttctatggatttcttataatattcaggaagcctggtataagattattctagcctcatagtggacccatgttctgtatagataagattgcctccagtaataaaattttgcgtaattgccgtattttaagagttagatttactttcttctagtactcaactggagtaaaaaatgagtgttgtggtctctctcttattcatgatgagtaggtcactgggactccggtgcatttcttttagttagacgcaatagataatgcattgtttttcgttgtgaacttgttttcttataggttttttctaagatgatttacaactaacattcatttttcttgaactgtgccggcttggtatgcagaacttgagaggaataagttccagtgcaaaagtgtatagtcaaaactcagtcggcgggtatgatgattttgaagatcagttagatagcccatcttctctggccggacccaggtctattacagctggaactttgaccctcgcatcttctgcaattggaggtaacaatcttcattctttcagtctcaagacgtgtaaaacttgttattgactttcagtagccctgcatctttcgacttttatgactctgcagcagatgggtttcctaggaggtttagtgatggagttaattcttctaatcaagcctataactatggaatgggtcgggctactggcagagatgatgaacacatggactggcgaagaaaggaaaactttggccaaggaaatgaccatgaaagacccagagcgttgatagatgcatatggagttgacaccagcaaacacacatccatcagtagaccaatccgcaacgtgaatggtatccacagtaagatggttacaccatggcagaacaccgaggaagaagagtttgattgggaagatatgagccctacactagagcgtagtagggctggtgaatttttgcgatcatctgttccagcattaggaagtgtaaggccaagacctagactaggaaacatacatgattttcagttagattctgacattaagaacggtatgaactctcgttttggtcgaggacctgcgatgatatcaagagttgggccttctggtgctgatgctcttcctgatgggacatggccccatttgggtgttagaggatccaactctctccctgtgccttctgctcacgtgcatcatttggcaaatcctggtaatgcgatgttaaatcacttgaatggtaagcctttatataggccagaaaaccaggtctcccacatgacccaacagagtcaagtgctaggaaattacttaccgtcttcatctgcaatggcaccaagggcaatgcaatcgctgttaccccacggtcatggttaccctccactagggtcaaccattagaccatctttatcagtccatggtggggaagccatgcatcctcattcttcaggggtttccatctctcagaatccttcgttgggagcctctaatcaaccaccgggcggtgcattctctggtctgattggctctctcatggctcaaggtttgatctcgttgaacaatcagcctactggccaggtaaattatttaaagcatgtcacaccctctaatacatgagagtcggactctttcttctagctttatcttaaataattgattttatatatacagggggcttcggtgatggagtttgatgcggacatgctcaagatacgtaacgagtctgcaataactgctctatatggggatcttcccaggcaatgcacaacatgtggtctacgcttcaagagccaagaagagcacagtaagcacatggattggcatgtaaccaagaacagaatgtccaagaaccataagcagaagccttctcggaaatggtttgtaagtggtagcatgtggcttagtggcgcagaggcacttggagctgaagcagtgccggggttcttacctgtcgagccaacaacagaaaagaaggatgacgaagagatggcagttcctgcagatgaagaccaaacttcatgcgcattatgtggtgaggcgtttgaagacttctatagtgatgagacggaggaatggatgtacaaaggtgctgtgtacatgaatgctcctgacggatcaacaacagatgtggacaagtctttgttaggtcctattgtacatgctaagtgcaggccagagtctaatggggtaagcatttccttgcattctctcattagcttcctttggaataaatgtctagttataagttaaatggcagttgatcatattatagctttatccagtggtagcctcgtgatttccctaagaatggatattttttttcagggtgatatggaagagggtagtcaaagaaaaaggatgcggagttag

SEQ ID NO: 102 - DNA, 6921 bp, Glycine max
  source 1..6921: mol_type = genomic DNA
gcgtggcacgcttatgacttaactgatgagaggagtgcaaatttggggctccagagttcagtcccatacttccatcccatcccaccccacgctccctcaaaacaaaaagaagggtttcaaatttcaattcataactcaccaatcccatagcgtcgccgtttctacaccgtccgatttccattccccatttccaatttcaattcccctctctatgccgccgcttcctacgccgtaatttcgtaacggcgacgcatcctcccaaaccctaaccctaaccctaacctccccatctctccgaaaccgatatttatttatgttttcacaaaacatgattctccctcccgaaaaccctagacccgcggggttcgcttccaaacccatgggcaatgagatcgcgaagcctccgccgtcgattctcgttggccgcttcaaggctctcctcaagcagcgcgacgatgaactccgagccacatccgtccccgtcccgccgccgtccaccgacgaatcgtccagatttacgaactcctgctgtcggaactcacgtgcaatttgaaacccatcatcaccgatctcacaattatcgccgaacagcagagggagcacgccaaaggtatcgctgacgcaatctgcgctcggattctcgaggtccgtctccgttgccgccgaaatttcttcttctacctcttgagatttgtacaatgtcatggaatcgttttattagttcgggtcaaaattatccctctcttgttgcttcattttttttaatgtcacaattcgtgctccaatgcttcgtttttttttttagttctgcgtttttttttatgcttttggcttttttctgaaaaatgaaggtcgcccataggccgagggaacgattgggttatacgctcaaactgaccgttgcttcgaacgttatattaaagcgtaaagcaaaaataatttgttgctactggtaatttaaagaataggaaatagtacaatctaaatgagtagatgtggggagtttctaacgcattgtgaagattgtgtacaatgtgtttgagggcttttttcaatttcaggaaagtcaagggcatacgaattgaaattgaattttggaatactacttctggaaaggctcaggaatgccattaaaggaaagttgttctttcatttttgctgaaatgaagtaatttctgcaaaataagaaagggttgttgaatgaacgagtatttaattcagtatgaaaacgttttataattctttaataagtatcgggtcatgcataatgagatcatagtatacaaataatacaaaatgtaaagaaattaacgactttttaaatcacccaagtccattgtgagtttttgttgatgcgtagtagaacattagtatttctactttcagaattggaatctctagtttatttatatatctctggctatgccactgtcacatgggaattcaattttttggagatatgcttttgcaggttccagtagatcaaaagcttccttcactctatctgttggacagtattgtgaagaattttgggcaggaatacattagatacttctctttacgtcttcctgaagtaggtcaactagagttgctgtattgtttttaattttattatttattccacaactgaaacgtctgcttcatttttctcctcaggttttctgtgaggcatacaggcaggttcagcctagtttgcattctgcaatgcgccatctttttggtacttggtcaaaagtctttccaccttcagtcctgcacaagattgaagctgagttgcaattttctcaagcagttaatactcaatcatccaccccgaatcctgtcagggcttctgaatcatctcgaccaagtcatggcatacatgttaatccaaaatacttgcggcagttggagcgctctactgtggatagtgtaagcttttttatgacatctctgtcttccacacaaactatacaaggactaggctaagtcttgattagcttgctggtaccatggggttgtatatttcactaatggaattgtttattgttttcatgtaaccataacaaaaatgtaatttttgtggtaaatatctcattttttctcatggtaggatctgtctgtatccatcattcctttcctgcatgtctgtccatctctctgtcttcccaaaacttttccccattttcttttgggataaactatgttctccaaaccttgtatttgctattttgatgttaagtttagcattggggtgggggtggggggggtggaggaatcaatgtagacacaacccatatcttgtgatggattgtaaagctctttggaaaatttattttatttttgcttacctctcgccccctctttccttaagagcttttatgaactatgactaagggcctgtatgtttactatctaaattcaattttacagtgtgacacacgttcaaatggaaaatctggttaaaatgaattggtactagcatcccaataacatttagcagttcaaatacaaaacttccataatcaattttaccaaacattaaaacaaatgatcactaagagcatgtttgcttcctggtttaggttgaatggatccatgtccatcagaagctactacatgtagcttctgcatatttaaggtgttagacatggagagagggtagttgatccatgttaaataacctgttccgaacatgcactaagtcctacacatgcaatcttttgtttatttattgtgatttactgaatttcctaactctaatatgggatattttttccctgattcctctctaaattgattattccggtgtccttagattacagtgacagattaaatacttctgccttgtctccctttgagagcctccaatttgcatattgttaatggcaacaaaatctgtttattgaatagtatgtctggcaacatatcagacagacataatttatctgttcttctctgtattatttttaattattttgacatctgggagatttgtcatcctgtcaagtgatatttttccctcccctccccttctctagtcagccgtatagttttctcttctcatattttatgtctgggggcgaatgaagtaagtactgtatccattgattctaggaagaaactgctgctgaatatttagcattcaatagagaaaattttcaacccttgtttgcaagaaaaaatcttgcaaacagatataatgagtggcattgtgacacagtatttgttttatcctcacaggctagtaagacacatcaatttttatcaagcagtagcaggcttggaatatcctcgtcgtcccctttaagaattggagttgataggcctttgtctgcatctatagatgaatatgcagtggacaatcctggtgttgattatggagtagccaaagcattaggtagagatgtggatttgactgaatggcagcgaaagctgtattctggtgatggtcgtaatcgatttccaacttcttttacgtacagccttagtaatggacatcagcgtcaaagttcaagagctttaattgatgcatatggcagtgacaaaagtcaagaaacttcaagcagtaagtctttgttagttgaacgtctggatagaaatggtatagacaaagtgttatcaacatcatggcagaatactgaagaggaggagtttgactgggaaaatatgagtccgaccttaatagaccatagtaggaataacagtttattgccttcaacttttggtttctctagggaaaggcctggcgttgcagctaatgcaactttgtcagagcaagatactaggaaggggtggtctagtggatctcagcttcctccagtagatgattcttctgccatagcagaagatgcatttgcctcttcaactgtatgtttcctcacttctttactttattctttctgatagatattgttttagtatggtttatgatattgaatttgaatttcccaattaaagtatatacctctaataagatgccattgatgctttgggttctggtgataattttctgatgatgaggatctggatagctttgtgtggataggcttaatcaaatgcatgaaattgtttcatacttgtctggcaattgatctctaatttagttcacggatattcactaatgccacatttttttactgtaagtggatagctttgtgtgggtagggcttaatcaaatgcatgaaattctttcatacttctctggcaattggtctcttctctaattttagttcacacattcactaatactacattttttacagtagaatactggcttttagaattgtttttttggtgcatattctgaatcactatttggcattgactccccttattttattttaataaaaactagaaattagttcaatgtaggttttctgtgtattgtccttgcctcaaaagagcttttgcgtgaggatgtgttggagagagatgtaatataaaaccatttacgtgtcttcacgtaacagtttaaactttagtaacgataatttgttgtgaaacatcattttagtttatttaatttgaaaggaaataggagttgccaataatttgctaatggaattggaaatttgtttattcttgctgcagttttgtcgtgcacccccaggtcaggtacctggatcccaaaatcaaatcaatcatagcctgggctcctctcaacctcatgatgcttggaagatttctcatcatccgtctaacatttttagtaatagggggcgggcaaggaatcttatgatccctccaattgacaacattcgcaatacagataataacccatattgggttcgacctgcagtgtcgaggatggaagctcacccatcagttttgccagcaccttttgaaatgaggccttctgtaaatgtgaatgtcactcggccacccatcataaatccgttgcaaaaacatgttaggagtcagtttgatgccatgaatactagtaatcctattgcgaaccatgttgtaaataaatcttcatttatgccagaacagtcatttgacagtgttgagaacaaggatgcaagtattttaaagatacatcagttgcctaatcagctttctggagtaatttcgtccaaccagcaaaatcatgggcaggcaccacaactacagttttttccttctcaggatccatcaacctcacaatttagtcatgggagttcatcgcagggacatggtgtttctataagtacagccatgtcaaatccattaccagttctaccattccctttaccatttcaaagtatttcaaataaccctttacatttacagggtggagcccatccacctttacctcctggtcgccctcctgctccatctcaaatgataccccatccaaatgctggtgcatttatgccaagccaacagccaactgttggatatactaatttaattagttctctaatgtctcaaggcgtgatctcattggctaaccagctgcctgcacaggtatctaatatttatatctatcagaagctcatttctttcattttttttcctttttgatttgattgcgccacccaaggttaattaaatattttttattaggattctgttggaactgagtttaatccagatattctgaagattcgtcatgagtctgcagtcaatgccttatatggtgatctccctagacaatgcacaacttgtgcgctccgattcaaatgccaagaggaacacagtagtcatatggattggcatgtgactaagaatcggatgtctaaaagccgtaagcagaaaccctctcgtaagtggtttgttagtgaccggatgtggctcagtggtgcagaggcattgggaacagaatcagctccaggttttttgcctaccgaaaccatagaagaaatgaaagatcatgaggagttggcagtgccggcagaagaagaccagaatacatgtgcattatgcggagagccttttgatgagttttacagtgatgaaatggaggaatggatgtatagaggagctgtatacctcaatgcacctttaggaataacagcaggcatggataggtcacagttaggccccataattcatgctaaatgcagatcagaatctaatatggccacctctgaagatcttgggctggatgaaaaggtatctttttctctatctatattattatgcactgcactagcctcagttttttgtcaagcatttgctccattttaatattttttggtcgcaatattttcatctgtatggtgattatgcatgtctgcatatgcttcaatgaatcttcagtaaatattcttctttttttttctttatatgcagggtgccgatgaagagggttcccaaaggaaacgaatgcggagttgaccaatgtaaccagttttgtgcattgtaacttatgattttccattagtgtatcttagagtagagttgaatcttgcaataccatgaccattctgccaaagtcatgtacatgtatagttggactggtaacgcgcatattttcttacagtcaaaggttacattttaacttgaaattattttgtctcttgcaattttgtccccaaatcaaaaactattagtacattgttatttctcggatggggcgcaggattttaaactatgcagcgatatactatcaggttgatcagaaattattatacttgctactgattagctggtaattggcatagaattaattgtttggacctactgtttgtatttttatcgaactcatgcataagtgagagcaaagtcatagatactcttaccaatttcttcaaataagtttgttgtgctagccatgtctctcccttcgcccttattcgcttcattatggttgctagctgtcagggaatacaaatacaggtcgaaggcttcacatggactaattttcttttcatctaatctaggatgaaacatgacattctattcacttcaagatatgcaagcgtattaattttaaaatactaatttatattaatt

SEQ ID NO: 103 - DNA, 4999 bp, Oryza sativa
  source 1..4999: mol_type = genomic DNA
atcgaaacctctcttccccctttcgcctccgcgacgcatatgcctccgcctccgcctcgtcgcggcgcccccaaaccctagcccggcgcggcgagtcccccatcggcggcgggagcggcgggagtaggcgggaaggcgggatctcctccgtcccacatggcggcggcggcggcggttgcgggggcgggcggggtggggcacgtggtggagaggttcagggcgaggctgcgggaggagcaggaggaggaggaggggggaggcggggaggtggcggcggcggcggtggtgcgggtgtacgtggaggtgctctcggagctcacgttcaactgcaagcccatcatcaccgagctcaccatcatagctggatttcttgggccaaacagtggactactggaatctgatattgtgaggcacagttggccaggccaggctcctcgaccagcaattgatgggccaccactaaatctcgaagatagaattcctactaacggggtatgcttgtctgaactctattagttatgattattgttactcatgttgaaatgaactttgtgtatcttagcactaataatatttagtgaaataatttcaatcaatatgccatctcttgctttcaggaggtgtttttttattcttcaagtctattaactgttttttataagaaaaggcttccaatagattttgtgcatatacttagttaattaaagcttaaaagattacaatacggcatttcatgttgggtcttccttggcttcactgtttatggtgattatcttccttagcattacatgcttgcccaaagctactctgtttacttacatatactactaattggaggcatttaacacatttttgtttctgtaatgtatatctttgcatttcagcctttcagaactttggtgcaaagtaatttatgccatttttcaccaagagattttgataatgtttgctactgtatattttgtggtcatatgccatgctggctggcacatgatatctccttcattcttcagaagttttaattttgaatatacactagatattcctttaccattttattaccatgatacttaactgcctctacaagttaacattttgtccttgcaatgaacaaaattcatgcacagttcaacttataagagcggaatgctgtactgatctgaatttactttaattaccttttaattagcgttgccttttttgcagcccgttgacagaactaataacaggagatatccaggcaattttggtgttcagaatggtgcctttttagattaccagagttctgaacatactcttgatcctgggagaactaccatgccagttccaccttggcagcagactattggtcagccgttgcgggtacaagcgccccagcctgcatcaattctgaatagaatgccactgcctactgatagtgaagtgccagtcaagaggttggccactggtggtacatatgatgcgctgaatgtagatattccattgttggagaagcaaaggtcctcacctgctcatgctccaatggaatggcctcttaatacacagtcactaactatacaaccaattccacctgataccaaacatcctagaggcgcatcagatggtctagattctagaccatttattagccaagggtccagttcgtctgtttttgttccacagcatcatgccttggatcggagaacaatgaatgctgatgatttagctcaaccatcatatcagcatccagatttgttgtcattgagtcaacaaaatcagggcacagttttgggaaaccaaggtcaacctcatcacccgccgcaatttcatccccatcctcattctcaccttcaagagacaattagaagctttgcccccagtatgtctgttgcacctccacaaaatatattccatggacaaggaggtagcgcagctgcactcctgccaagttctttccctgtaccacctgcagtaccaccctatggcttgcagtctatgcctggatttcctttgccatcgttaccttcaggaccacctcctccatcgcagataggtccatcatcatcgcaagttgggggccctccacttgtctctggattgttaagtaaccttatgcagcatggtataatctcattgcaaccacccagccaacctcaggtactcatatgtgcacctcattgccagtctctgatattgttctatggatacgcatgcagttgattctatgacttacattatacctgtaatctgttcttttaggattctgtgggtgttgacttcaatgttgacctcaaggtacggaacgagtctgtcattaatgctctgtatcaggatcttccgaggcaatgcaaaacttgtggccttcgttttaaatgccaagaagaacatcgtgctcacatggattggcatgttacgaaaaacagaaattcaaagaatcgtaagcaaacttcacgtaaatattttgtcactgtgggtgaatggttaagagcagcagaaacagtaggaaacgatggtgttccttcttttgagcctgccgaaccagttgcagacgcaaaggaagagaaagaattggctgttcctgctgatgaggaccaaacaacatgtgctttatgtcaagagccatttgaagatttctacagtgatgaaactgaagagtggatgtacaaaggtgcagtttatatgaatgcaccagatggaaatattggtggtctagaaaggtcgcagttgggacctattgtccatgctaaatgtctatctgggccaaacaatacttaggccggcctagctgctggtgagtatccatgcattacttgcaacctcactatgaaagtatagctagtatatgttaatggccatatgctcacatgtgttcaactgctgctgtgatcatattatctgactagattataattttggatgtgatagacttctttaacagtgatctcacagggcatctttcttcattggttggggacagcattttacctcgttaatatgattattcattctacttttgtatattctcttttttttttgtcagttctgtggtacttaaattgtttagcactattctgtagtatctacatgccatctattcacctcagggatcctttggatcatttagtacggaaatgcgtttttgtttgatagtttgatcatttctctgtagatctgctaattaccacaagtaacaagatttctgatccttcagtgtgtagggtggacagaacctttggcatgttttggccaccaagtaaaatccattaactaattcagtctatgtgtgtggttatgattactatgattgtcattatgactgcacttactgtggaactgtcatgtttgctgatgatgtgaagataacatttattgttctgcaatggactgctaaaaataaaacagttgtggtttttaaaaatgaaaattaaatgaaatgagcttttttcatacaggtcgaatgagtggtcaattgttctacaatttagtgttatcaagtgtttcctgcatgcaaaagtaaaattgaaaaatctgagttattctagaacacctgaatactcttttttagtatgattcatgattttcaaaggaagattgggtttgagcatactcttctagtagtattatgaaacaatattcttcttgtatgttactagcacactaaaataacattttgtcaaacaaatgatttgtctttaacgcttggacacccaagacggtcagtctcgtagaattagcgggagttgtccatcaattcccgaaactagttgaaccagctcgtacctttttcctactggaaatgtatttttctgttgtccagaaccggaattatgcgctccattgtgcttttcttgttaactgatttacagccaatgttttatgtgtgccaggtcctttttgtttaagatttgtcatatatgctaatattgtgtttgtaattgttcatgcaatatgtaaaccccagacaattacatactgggaattgggatactgagcagtgtaattttgcatttaatataaatagaatgtgatggcctcaactttgatcaccatttggagctaactgttagcagtgtggatgttatatgggaagttaattccttcgtcctatatagaatgatcggtccttttggttcttgggctactctgggttgaggacttacataaactatgagtacaattatgcttattcacactattaagtatatgttgttacacataatttgaagtttgccagcatctgctgcttaaacccctaggagtgcatcattcaaacagacaaattttcaatatgttgcatgagttaactacttcacaattttattgcaggtactcttgcctcatttgtttcgtggctggggctgtagacaagacatgtaaactactggagagagcagattttggtatgcttagtatttaatcctcaattgacaaccatgggtttgcatagaatgttcatgtgatgatagtgtacaactgtaaaatgccttttgtaatatagtgattgagcatgagcatttttgctcgacagtcactatggtgcatgttcaatcgtttggtcccaccatctcacatgcagtgtgctgcttttccattattatagtatattttgaaatccccatgtctactaggtgataggcagccctagttcagaacggtgcaatcactccactgaacttgcgaaacaatatctttctttcttgtttcccttctttccttttgtgcaggacaatatcagtcggctacctggctggtaacttccaaaatcgaaaggacatgtaaggcgatcaaggatggaagcaaaatcatcagcttaatttccagaaaatgaaggagatgctctcttcttgaatttttcccccttttttcatactcttttgtatggcatggcgttctggtattaacacaacagttcattttggtcgcatgtatgactcatcttgctgatatgtttgcacatgtcaagttcagactagattgtttttaagtatgctgtttaaattgatctctggaactcttgtac

SEQ ID NO: 104 - DNA, 14486 bp, Zea mays
  source 1..14486: mol_type = genomic DNA
gaaacgtgaccctcgccgtttacgaacaaatcgccgacccgtctcctgctctctcccaatttcagtcagttcgctccgctcccacatgcctcctccgcctccctctcgccgtcccggcatacccaatccctaggcaccccgccgaaaccctagattctcctctcctgctccgatggctggggcagcggcgtcccccgtggcggcccacgtggtggaacggttccgctcgcggctgcgcgaggaagcgggcagcggggagccgggcgcctccgcggtcgtgcgcttgtacgcggaggcgctccgcgagcttaccttcaactgcaagccggtcatcaccgagctcaccatcgtcgcaggccagcacgccgcgctcgccgccgggggaatcgccggcgccgtctgcgcccgcgtcgccgaggtcattctgctgctctttgctaccccccatgactgttactcttgttttccagttaaggcggcccgcgttatgatttttggggcataaggaagcttggtcttggagcgtcgagtagtctggcgctcacatggttttggaggttgttgtgcttgttgtgaaatgtggattgcttggctttgttgttgaatgtgagaattcccataaaatggacgggagactttttatttgtaggttttacaatagcgtcagtttgtacaagatcattcctttttgtgagaagcgatgtacgttcagtatgtttgctctgattgctgtttacagttctggtagcatatttagatcctggggtttagaatctttgtggaactttgtagatcaattggtgcttgccattttaatttttcttaaaattcctgctcaaaactagtaggatgctgacatacatgcatgaaatatgtagcaacagagtaaacagttgagcttaagagtgtatgagcactgcattggagaaaatagtcgctatgactgataaacttcctttttgatcggaagttgagagttatcctatcgaattgagataaaccttaaacaatgttatagataaatgcgtcatgtgttacactggttcttattttgacttacatacgaaaagagttcagaattttgggtttattattgcttattaggatagttgtgaactaagtcagttacctgtttacttgttctgtatggtgtggtagatttggttttgtttagaaatatcttgtaattttctggctgcttttcttgaagataagacatacaagtcttcaattttaaatggaaacaatatgggaactaatcataagagcgaggattaagctactagtgggtttgcagtttttctttcagaactgcaggctgtttgagtttgttcatcaaccagttttttttcatatgcatgatttttaattaattacgactttcaaagttcttttgtgtgatgattgttcttctgcgatattcttgcaggtgccacctgatcagatattaccatctttgtatctgctagatagtattgtgaagaacataagcgggtcaatgtggtaagactttgttaacatcatgtatatatatgagaatcactgttgtatttattttggttcaagcatgataattgtagtctaatgactgttaataatttgttgtattgttgcacctgttgcattctgtaacagtctatgcgatttaagttaactttaaatttattgacgatgtttgtccactttatttccttaaaattcaggcgcaccagtcagcagctcaggacactaggcaaatgtctgatgtgcaggaggatctgataaatggactaacatcgaatagttcacaaggacggccttcagtgtttcaggtattcagattgtctcttcccttatcgttcttgtcatttttgcaaccactatccaatttcaagcctggcttgcatgtaaaaaactgtcttctgattcttttgctccccttattggccgagaaaggaaaaacgtttttttgtcttaatctttttggggaaatactcaatttatttaagatactggttctggttactgattgcttcttccaaaagttcctacaacttggttccttccaagagttcctatgactcggttccttccaagagttcctatgactcagttccttccaagagttcctatgactcggttccttccaagagttcctacaactctacctggtttagtattcttgataacaaacaatcccacaccaaagcacatggctaaggcccgaccccggtcacaatcattctcacatgggctatggtgccgctgtgtatgggtggggcaggggttcgggagtttttttgatctgcatgagaagaggtctttttaatgtaatactgtgagggcgatcttacccctttcaggttgagttttttaaacaatcgatcttgtctgacatttttgatgctttattttacctcaagcgaaatgaagcaaaagtactcaaatctactccttccatttcaaaatgttggtcattttagttttgttctaaatcaaatgtctctagctttgaccatgtttagaaaaatgcaccaacatgtagagcatcaaattagtttcgttaaatttctaagagaacgaaaacctaatccattgatagtgggttgcaatatacatattccctaaccctaatgggatacatgggttgggctcgaggcccataacatatagacacacacacacatacaatctaacccctagtggaaactctagccatgacagattgagactggaccaaaactctgagaacaccgaggaggatggaagacccttagtgaagatgtctgcaaactgcgagctcacgaacgaattgaagatcgatgtcaacatgcttcgtgtgctgttgctggaccaagttgatggagaggtagacgacgttgacattgttgtagtagatatgtgtactcttggtgagaggaatatggagctcctaaaggagctagcggagccagttcacttcagccacgccgttggcaacgacactgtacttagccttagcgtgggaataggaggccacatgttggcacttgaaggaccaggagatgaggttgttcccaaggaaaacgatgtagccaaaggtggaccgttgggtctttgggcagcctgcccagtcagcatcggaggagcagacgatcaacacaagggtggggtgtcgaaggagaagttcaaaggtatcgaaagatctgcttgacaaccatgaggtgcagctcttagggtcatgcatgtgaagacaaacttgttagaccacataggcgatgtcaggctggggaaggtgaaatactgtagggcaccaacaaggctccgatagccgtgaggtgcggctcccggggatcattcatgtgaagttggacttgttggaccacataggcgatgtcaggccgggtgaaggtgagatactgtagggcaccaacaaggctccggtagtcagtaggatcaccaactagaggactgtcaccagagaccttggcctatgtgtccaccggagtcgcacacggtttaaactccgcatgccagcatgctttaagatatccaaagtgtactaccactgttgaagaaaaaggccagtcgagtgatgctcaacaacaacacccagtaaatgatggaggcctagatctttcatggtgaactgatgctgaagagttgtgatatttcaatgcagaagagttgaagaagagggagtgaggacaatgtcgtcaacttataggagtagacaatgtcactgtcacactagtagatgaacatgatagtatcagatttagcctcagtgaatcctaggaagagcaagtaggaagcaaatcggctgtacgaagtgttggttgttgctttagttcgtaaaaaggcttgttgagccttaggtccttgcttaagtccatacagagacttgttgagcttgcagacccagtcggggtgagcggagtcaacaaatccagcaggctggctgtaataagttgtctcagtaagggtgcgagaaaaaagcattttgacatctagctgatagactggctaagaatgggacaagcagactgcaccaatcaccattagactgaaggtctcatcataatcgacgctcgggcgctgggtgaatcctcggagaacccaatgagccctgtacctgtccagatagaaatccacttatgggtctaaacccacttgctagccagcacattgccgcatggagggtgcgacaccaagtcccaggtgtggttggcaagaagggtgtcacactcttcctccatggtgtaacactagtgaggatcagtgagggcgcgatggatagaagatgtcacaggggacactaacgaagaggaggatgcgaaaaggacaagacagtcgccaggatgaagaactctagcgtcctgataggtaaccattggttggacgtgacggtgtttgcggtgaagtagagggtgatacaccgccagctcaacctgagagctaggtggggcaccctcttgtgcacgtcgttgataaacatgcataagattgatgaagtggacaaagcacgtgagggtatcgacaacgccacaggtgagctcggggccacgacatgggacgttgggggcgcgtgggtgggggacgacagtgccacatagcgcaatcaggctcagtgcctgggcggcatcgggaccgcagggggtagcggtggagtcggggaacctgctgagaaaaatagaggtggctggctagagggagccatagggtcagttctagaaagacgtctagctcgatagtagagctagagggggatgtggggaagaagtccgtctcgtagaaatataacatggttggagatcaacaagcggttggtgaggttaagacatcgatatcccatatgatcagaagagtacccgagaaacacacaacgagaggatcaacaggaaagcttatgagaagctgtggcatagagactggggtagcacacacacacacccaaagacacgaaggtggtcatagaaaggagtggttccgaagacggcaaaatgaggcgtgtgggagcagagagagctttggtagatagtcaattgagcaggtaggtggcggtgtgaagactctcgacccaatagcgagcaagaacagaggcctgaaacaaggaacacatgatgtcgatagtagcacgaatcatatgctcaaccttgtcggtttgggaagaggatacgagcatgacattctagctgcataccatgtgacagtgacattgttcatccgccgcgctgcctcctcacatcgatgccgcccctgtcttccctcattcacccctaccgattgagagagatccctggtaggatcaggaaccctaccagtccgcctcattgcttaaggcgttgccccaatgcttcgagcggtggcaaggcattaatttgtggatgctttggcctttgtagggtttggggaacgatatagaggtcggaaaagagaggcttacagtgcgagacactccaagcggcgatgagtggaacctccaggcgatggccatcttccttctctccaggcggcgacggtggccctacgcccctgcctcttctctagcggctgatggttcccctccgtgagctcccctacacccctacctcttctccagcggcgggtggcccccctccacgagctccccttgtactccgcccttgcctcctgtgagctcgcgacgagcgagccgggggtgggcgccggtcctgcttctatctcatcctcacaactggaactgggtcacaaggcggtgagatcatgggagggacaatcggtttgtgtggttgcagttgggaactgggtcacggtcttgtatgttcgttgggccatgctgaaattgtttagaaactcagaggcgggtagtaacaagccggcgccgtcctactccggccctcgtgggcaaaatgccctgcatgttgctgtcctaaacaacaaaagaggtgacataaacaaacaatataatatatagtacacatcacggtcaatatataatattgtatggaataattaacactcttctacttgtcctcctagcctaatggcttggttttccaatatatttttatcatcatctttttatattctattggctacacgccttcacatattacttatgttggactttaaatcaaatatgttgatttatgttaaacttgtactcctatttgctacttagtactattatccatatctatttgagactacattcctatattttctatttttatttcttatttctatgtattactaaggcgtcgcctcaccttacatcttaacacttaaaaggtaaaaagggggttggctgccttccctcgtcttaccgccttaaaagggaaaaattgctttttaggtcccttgtcgttgttgcttgccaaaatatcctggcattcgttgcactcgcttaattgccccacttaaacaacgcgtagttgatgaaataccctagtgtagctatttttatattttcatggacccaataattcgaaatgtcacttttaccctctgtatacacaaaacagtgttaagctttgttacttcgatgttacacaatatatggagtcgttaacagcaaacaaaacaaaacattagcaatgcagttaggtagaagcaaagaataaatgagcacattcatttcagggtcaagcagattgggagatgcgttatctgattttttcctggtaactgaaggggaactttgtgaaaaagtgtgcaatctattctgttgatgatctcagtcatgcttcaaggttggagcattctctgtccagcatgaaggaagctagaaattcaattccaaactggaaagtctcacaatgctggctatcaggcctctaaggctgactggctagaccaggtggatggagagtggaacaagtatctatacaagacggcgctatctggactgggagaggaaggatgtgccaaaaacattcacagtagtaagtaatagcgaaccccatattaggtaaggagtaacctgctcacatcatcagttgatagtctgaaacaatggaataaatggaataagacaagagaaataggaccagttgacaccaaagaatcctaacaaaagcactgttaattctgaagcgttttttctaagcattgtgcagacaagttaccatcatgtagtggtactctatgttgaacacaatttcttcaaatcgagccaggtactagaaacaataacagtatagccaggtatagacggtcatgtctaagaatagtgacacataagaactacagatccatgatgatgctcaattcatcctcattatgtgttgctcatattagataagtacctagatagcttacaagagatggtatattcagattttagatatttctcctgaagggctttgcatagtagaggaacatccgagttttcaaaacttggcactccctgtttggctgctgaaagctaatcagagtgtgcaactgatcagaggacgctgtagcacgcctctctgtcatgtttgcagagggtaaaagtggcatttcgaattattgggtcatgaaaaatatatgaatagccacaatggggtattttatcaactacgcgctgcttaagtgggacaattaagtgagtacaacgaatgccggggtattttggcgagcggcaacgacaagcgacctaaaaaaagtaattttccaccttaaaaactatggtggaaacccaagcaaagaagtgcgaaaagagaaatggtggaaaaagtattagacttcaagcgcaaagggaaagtccatgaataatgagataaatcaagtatcaccaagtattatttatacccagaaacattgacaacaaagggatgcccacaaatcagtgtatgataaaaaacatgtatagcctgagaggagctagagaaggggagtcgaacatgctgacctagctaacaggcatgttatagatgctcacgagtaccccaaatacatccattgatagtgggctacatgggctgggctcgaggcccataacacacttaaactctaagtctcacaaattcaccatgaaatgtgtcttgaaagtgcttagcattcaagatgttaatttatttttctataaattcagtcgaagttagagaagtttgattttgtttaggttagaaatattacaacattacatttttgggatgtagtacttcaacatccataactgttcatctgcatttctttttcttccaatccaagttgtgaaatgctatttctgcctctggtttattttgtgatggctcataatctgtgttgtaaccatatgggcagaaatcttctttacagtatgctgatgatcctggtcaacaagagatgtttcgacctcttgctggagcaataagggcaacatcgccacatctcttgagaacacatgcatctgatgtgaatctagatgggccattagccaattcaaggagaaacttgtccagatcaccacctcttgatgtgttccctaggaatgtttccccaaagagaccactagagaggctaccaccatctcattctgtattgggacctgatcccagaaaattgcctgatcgaaatggcaggttaaggtgggctttggacgatggtgcacagtggcctacaataagtatgcttgatgaagagtacaggaagcaaagtgcaagggaacttattgatgcatatggaaattgtcaaggacgagatgctgatgaacgacttcctaaagtacagcgcctggattcaaatggtatgactagcaaatctggtgcacgaaattggttaacttcagaggaagagtattcatgggaggatatgagtccaactttgactgatcgagtcagaagcagcatgccatcttttcctcctggaaccatgagaattggatttcctggggcaaatgcagggctactggaatctgatgttgggaggcacaatttcctaagccaagctactagatcatcagttgatgtttcaccatttaatattgaagataggatttcagctgcaagtgtatggttgtctgatctctctttaaataatagtgaatcataacattagcataacgctttgtgtgtctatcgtcaccacctgcagtcctatgaaataatttttctcccttttatttaaaacaattatggttccacacaattggattgctgctttctattctaatatatctgtaaaaatcgttgttctaaaaggctggatgccaactagccatgtgctgtctagttcaattgggcgggctaggtggccccaggtacgcatgatgagctagttgggccagactgccattggagtggcggaagcagtagcttgcaaagggccaacaagaccatgaccttttgttccatctgcttccttcttctcccgctcatattcttagctaaggtcatataccacatggcccatgtatgcaagttagcctgtgcagacaatgcagtactttgctgccagcctagagcctagtgcccttcagatcaatgatgaagatgtgctatggaaaaactccaagtaacatcagatgtactaatatagacttcagcttgctgatctagagatattattgccaaatttcgactgacatatcctttgacaatccttgtttgactgaaaggacaaaagagtagttttgtaaaaattgtgcaaataaattagtcagagaaacaacattttgtgttaccgcatatgtatgctgttagggaaggaactttataagcgtagtttttcagcagtttattcctttgtattttacaatttgatgtaaaacaatcaatgccttcgcccttcggccactcatagattttaagtaggactagcacattatttcccagtagccttcagcaggttttaggcacgtgtgcattatttaactgttttcttgtcttatacttaactgaaaccctttgaacccttttgtccatgcaccaaatagaattcatatgcaaacttgtcaacatggtagtgttgtctctatgaatctatgaacacctactgacagggacaccttcctcttgcagcatgttgacatttccacaaggagatatccaagcaattttggtgttcagaatggagctcttttggagtaccagaattctgaacatactctaaatcatgggagaattgctaccatgccagctccaccttggcaacagcctattggtcttccattgcgaatacaaggacctgaacatccttcagtacttggtagaataccacagcctgtcgacggtgaaatgccagtcaagaggttggacattagtggcacgtacaatggtttgaatgtagataaacccttggtgaagcataggtcatcacctgctcctgccccgacagaatggctcccccttcaccatgcccaatcacaaactttaccactaattccacctgataccaaacatgttagaagtgcatcagatagtttagaaattagttcttttgttagccatggaggtagttcatcagtatttgttccacggcatcagtatgatgccgtggatcggaaaacaggtaccagtaacttagctcaaccaccatcaccatatcagaaccaagatttgttgccatcaagtcaacaaaatcaaggtacaattttgggaaaccaagctcatcctcatcgtcccctaaagttacatccccatgctcatcctcaccatcaggagacctttagaagctttgcacctggcatgtctgtatctccattccaaggacaaggaggtaatgcaaccatgacaccagtttctgttctgccaacgtctttttctgtacctcctgctgtgccaccctatggcgtgctgtctgcacctgggtttcctatgccaccattaccgcctggaccaccagcacctgtctcactgcagatgggttcatcatcatcacaagttggtggcccccaaccatttgtctctggactgttgagtaaccttatgcggcacggtgtaatttcactggaaccacctagtcaatcccaggtatttctcatgcttcatgcgttgtattatatgacattattctgttcatatttatactgttaattgaggggaaaattgtgaatatttttttgtaggattctgttggagttgacttcaatgtagacctcaaggtacggaatgaatctgtcaccaatgctttgtatcaggatcttccaaggcagtgcaaaacttgtggtcttcgatttaaatgccaagaggagcatcgtgctcacatggactggcatgttacaaagaacaggaattcaaagaatcgtaagaaatcttcacgtaaatattttgtcaccgctgttgaatggttaagagcagcagaaacagtaggaaacgatggagttcctgcttttgtgctctcagacccagtcccagacagacaggaagagaaagaaattgctgttcctgcggatgaggagcaaacatcctgtgcattatgtcaagagccatttgaagatttctacagtgatgagactgacgagtggatgtacagaggtgcagtttatatgaatgcaccagatggtaatatcgatggtttggaatggtcacagttgggacctattgtccatgctaaatgtcgatctgggcccggcaatacttcttagggtgggcatagctggtgagtatctattcattttttgtgctataataaatatatatgaacttttagatgtacatagtttttttcttgaggttaacctcactgtaaacaatggatattatctccttgcgagtatttagctgtattccaacacatttttagctatgattatgtgttatctgactgaacaaaaatgctggatgtgatatgcaatgttcatgaattttccattatttattgtcagtgttctctgttagatgaatatactctcacattcgggacccattattttttgtttggttttgcctggtgcagtggtgagagctgtcttagtgagcactaggttacgggttcgaagcagtctctccacatttgcggaaggaacgcttgcctcggtttatcccttccctagaccccgctaatgtgggagtctctggcactaggtctgccctttttgtcgtgctgaaccattttgcagatccatcatatggattctcaggatgtccaattctactggcatatataatgagaggaccttcagttgctctcttgaatagttagttactgtttgaaggattatgtaggtttaaaaatggaaatgtgaatgctcatgtttgttcaagacttcaagtaatatacttttttaattggtgtaactttggtaatgacgaaggtaatcatgctaattatgtgattttatatgctaatgactatgcttttttataattttctagcatgactatgcttttcatctctcaccttttagtatctaaggaaggataaaagcaggctttggactaatttgtcaaaaatgacgatgtttaggagttcaggactattgtagtcccttttcgttcgtacttgactatttttgagcaggctttgcactaattttgtaaacaatgaccattgtagtcccttttcattcgtacttgactatttttcccatcatggcattactaatctgcacaattattttggcatcttggatatattttctcaagcaggcgtgatctaatttataaaaaagtggcaataatcaggaggtcaggactagcacagttactttctatttgctcttcactaggtttgcccatcatggcatcactaatctgcgcagctattatgtttgccaatcaaagattcatacatttagttgaacgacgctaatactgtgtatctcattttgtcattccctgacaacgatatcctggtgcacagggcctttccttctttttttgtgaggatttttattttgtttttgtgctagctcttagcctgtttggactttggaagcatcgtatgtttttaattatgtagtcagtgttcgtgcaattagtaagctgcataggcacatacaagcacgtaaattctggtgacctctttctgatgacatcacgattcctgtgaggatattgactcatttttctgtcaaatatcgaatggatctgacatgaaattcattgaaagtaattgatgagcaatcatatttgttttacgttttctttgtcctacaattttttccctttggattatgaaatcgtcaatgaccggttataagttggaaggtggtaaaattgagatttactcacctatttgccattgatctgtaccatgtaatttagttccatgctgttgtatcagctactacctcttccttaaagaggtgcatagctagaatgagtcttttagggacttgcaacaaaacctttttcttctttggctccaaaaggatcctttctagtacatgccaaaataggaacctaagggctagtttggaaacctcattttcccacgggtttttcattttcccaagggaagttagaacattttcccttgggaaaataaaaatcctttgggaaaatcgagttcccaaactagccctaagtgtgcttaaagccttaaaaagcctgctgagttttcctgaatggtgtatgatccaatgagatgggttcattggtttactcctttggccatggtgatgatttgaatacataatatttgttttcatattagttgcatgtgatggcttcaattttacactttctgtccctaatttaatgttaataaaggcaccatgaagataacttatcatattgtttattagtcttgcgtgaacaaatcactcgaacttaaacttgtgattatgtacaacaacagcaacaaagccttttatctcaagctaaacttgtgattatctgatggttaataaattcctttagaagtgcctcattgaaagatgttgctatgttgattgataactatttttattttcgttgcagggatgatttgatagtttttttttcccgttgcggctgtaggcatgacctgtaaactaatggagactcagattttggtatggttagcttcttttccatctggctcccttaatagccagtgcatagaatgttttttggaacaaaaaggcaggagccagtgcatagaatgtcaatgtgacaatagtgtataactgtaaaatttttgtgtaattcagttgcacagtgctgctttttcattacaactatagtgtgatctatactgaagcttcatgtttagggtttctgcagcacagttgttgacaacggtatatgtatatgtaaccatggtttcactggacatgggaaacaatatatttcaatagtcctgttttcttccgttaatatcttgtgcagaatacatagcagttctctacgttggttggtagctttggaagggatcgcccaagatgaccatgagtgctggtgactatttatgcctctcctgaagtgaaagggactagatgctgtcattaccttattgccaaaactttttagtattttcctttgtaagttgagagcgttgtttgtgacatattctcggtcgcttatatagttccgtattgctgatatgttcatgctccataatgtggatgagaatgtcttattagactttatactttattctagttggtatatgctctggcgaactattcaatgaaacaatttcgcaatgttttttttgtc

SEQ ID NO: 105 - DNA, 9325 bp, Zea mays
  source 1..9325: mol_type = genomic DNA
gaaaaggaaagcatcgctgaaacgagatcctcgccgtttcgcgaacaaatcggtgactgtctcctgctatctccccctttccgccacttcgctccgctcccatgcctcctgccccctctcgccgtggaggcaccccaaaccctagcccctgaaaccctagatcctcctcccctgttccgatggcgggggccgcggcgtccccagcggcgggccacgtggtggagcggttccggtcgcgactgcgcgaggaagcgggcggcggcgagccgggcgccgccgcggtcgtgcacgtgtacgcggaggcgctccgtgagctaaccttcaactgcaagccggtcatcaccgagctcaccatcatcgcgggccagcacgccacgctcgccgccgggggaatcgccgacgccatctgcgcccgcgtcgccgaggtcgttctgctacttctgctctcctatcaggggtgttactcttattttgtcattaaggtggcccgccttacgatttctggggcataaggaagcttgggcttggagcgtcgagtagtctgggcgcttttgccgtgtagtcactagggtttgaggattggtgtgcttgttgtgaaacgtggattgctgtgcttggttttgaaatgtgagaaatcccataaaatggacggaaaaccttttattcccaagttttcctaggttttacaaaagcttcagcctgtacaagatcattcctttttgtaagaagcgttgtgcatacagtttagtatgtttgctctggttgctgtttacagctttggtagcatatttagggggtgtttgaatgcacaagagctaataggtagttggctaaaaaattgctattgaaattagctagctaaatattagctaatttgctaaaagtagctaatagctgaattattagctagagtgtttagacgtctttagctaattttagcagctgactattagctctagtacattcaaacacccctttcctggggtgtagaacctttgtggaactttgtggattaattggtgcttgccattttagggggtgtttggtttgtagggactaatttttagtccctacattttattccactttagttacaaaattatcaaatatagaaactaaaactctattttagtttctatatttgtcaatttatatactaaaatggaacaaaatgaagggactaaacattagtccctagaaaccaaacacccccttagtccctcttaaacttcctgttcattattggtaggatatgcatggaatatgtagcaacatagtaaacagttataccttaagagggtatggacactgacactgcattggaaaaagtggttgctatggttgatgaacttctatttgggatgggaagtttgagagttctgttgaattgagataaacgttaaacgaaaagagctgcactggttcgtgacttacaatgaaaagagttcacaactttgtgtttattattagttattagggcacttgtgaacttaatcagttacctgtttgctagttctgtcttctgtggtcgatttggttttgtttagaaatatgttgtaattttccgtctgcttttcttgaagataagacgctgcaatttcaaatgggaactaatcataagagcaattcacccgtatagtcacccaaaacaatgttttgcactgtagactgtactgctcgcagagtgaagtttgaatataggtatgaggataggtaagctgttggagatagcctaagagcaaggattaagctaccagtgtggtttgcagttttcctttcagaatccgttggttgtttgagattgtttattaattagttttctccctcccacatgcatgatttttaattaattacaactatcaaagttctttcgtgtgatgatcattattctgcgatattcttgcaggtgccaccagatcagatattaccatctttgtatctgctagatagtattgtgaagaacatagcaggccaatgtggtaagactttttgtgagcaacatgtgtagatactagaatcaccggtgtatttgttgtggttttaggcatgataattgcagtctaataactgtcaatagtgtcgtattgttgcacaacttattgtgttctataacagtctgcgatttaagttaacttcaaacttattgaggatgtttgtccactttatttccttaaaaatcaggtgcaccagccagcagctcgggacactaggcaaatgactgatgtgcaggaggatctgataaatggattagcatcgaatagttcgtgtgaacggccttcaattttccaggtatttaagatcgcctcttcccttgtctctccttgtcatttttgcaaccagtctccaattccaagcctgtcttccagacatcaatgtagtaatgcctgcaggtaaaaaactgtcttttgtttcatttgctctcattattagttgtgaaagaattttttttgtcttaaatatttttgaggaaatactctatttatttaagataccgattctggttactgacagttaattccaagagtttttatgacttgttcctttcaagagttcctacagctctgtacctgatttagtattcttgataacaaacagtcaaacttgtctgacttcttatgtcgcttgaaaaaatgttaggtgctttattttacctcaagtgaaatgaaaccaaagtactcaatctaccccttccattccaaaatgtaggtcattttagttttgtcctaagtcaaatgtttctagcttgaccatgttttagaaaaatgcactggcatgtagagcatcaaattagtttcattaatttcaccatgaaatttgtcttgatagtgctaagctaagcattgtagataatatatttttctataaactccgtcaaagttagagaagtttgattttatttaggttagaaatataataccatattacattacatttttgggatggaagtattacttcaacatccataaatgctcatctgcatttatttttctttcaatctaaattttcaaatgttatttctgcctctggtttactttgtgatggctcatgatatgtgttgtaatcatatgggcagaaatcttctttacagtatgctgatgatcctgatcaacaagagacgtttcggcctcttgctggaacaagggcagcatcaccacatctcttgagtacacatgcatctgatatgaatctagatgggccattagtcaattcaaggagaaacttgtccatatcaccacctcttgatgtgttctctaggaatgtgtcccccaagagagcactagagaggctgccaccatctcattctgtattgggacctgatcccagaaaattcactgatcgaaatggcaggttaagatggggtttggatgatggtgcacaacggcctacaatcagtatgcttgatgaagagtacaggaagcaaagtgcaagggaacttattgatgcatatgggaattgtcaaggacgagatgctgatgaacgacttcctaaagtgcagcgcctggatccaaatggtatggctagcagatctagtgcacgaaattggttaacttccgaggaagaagagtattcatgggaggatatgagcccaactttgactgatcgagtcagtagcagcatgccatcttttcctcctggaaccatgagaactggatttcctggggcaaatgcagggttactggaatctgatgttgggaggcacaatttcccaagccaagctactagattatcggttgatggttcaccgtttaatcttgaagataggttttcaactgcaagtgtatgattgtctgatctctctttaaataatagtgaatcacagcattagcataacactttgtgtgcctatcgttacctcgtgctattcttgaaattattttctccttttcctttaaaagatttatgatgtccagtatgcaaattgattgttgctttctgatctaatatatttgtaaaaatcattgttctaaaaaggctggttgccaactagccatgtgttgtccagttgaattgggctggctaggtggccctaggcgcacatgataagctagttgggccagatgtgccactagagtggcagaagcagtagctagcaaagggccaacaagaccatgaccttttgtgccctctgcttccttcctctcctgctcacatccttagttaaggtcttatgccgatggcccatgtatgcaagttagcctgtgcagatgatgcagtattttgctgcctgcctagagcctagtgcctttagatcaatgataaaaatctgctatacaagtaatataggcttcagctggatgatctagagatattgttgccaaatttcaaatgacatgtatcctgcgacaatgcttgtttgaccgaaagaacaaaagagtagttaggtagaagttctgcaaataaattagttagagaaacaacattgtgttaccacatatgtatgctgttagggaaggaactgtataagcataatttttcagcagtttattcctttgtattttacaattggatataaaataatcaatgccttcagttactcagattttaaagcatgcaccatgagtaggactagcgcaatattttctagtagcctgcagcagtttttaggcactgtatgtattatttaactgttttcttgtcttgtgtttaactgaaaccctttgaacattttttgtccatgcaacaaataggatcatatgcagacttgtcaacatgatagtgctgtctcgatgaatctatgaacacctattgacacaggaacacctttacctcttgcagcatgttaacatgtctacaaggagatatccaagcaattttggtgttcagaatggagctcttttgaaattccagaattctgaacatgccctaaatcatgggagaattgctaccatgccagctccaccttggcaacagcctactggtctgccattgcaaatgcaagcacctcaacattcttcagcacttgatagattaccacagcccgctgacagtgaaatgccagtcaagaggttggacattcgtggcacatacaatggtttgaatgtagatagatccttggcggagaagcataggtcatcacctgctcgtgctccgacagaatggcttccccttcaccatacaccatcacaaactttaccactaattatacctgagacaaaacatgttagaagtgcatcggatagtttagaaattagttcttttgttagccaaggagctagttcatcagtatttgttccgcatcatcagtatgatgccttggaccggaaaacaggtacccgtaacttagctcaaccaccatatcagcaccaagatttgttgctaccaagccaacaaaatcaaggtacaattttgggaaaccaagttcatcctcatctgccccaacagttacatccccattcccatcctcaccatcaggagacttttagaagctttgcccctggcatgactgtatctccattccaaggacaaggaggcaatgcaaccatgacaccagtttccgttctgccaactttttctgtaccacctgctgtgccaccctatggcgtgctgtctgcacctgggtttcctatgccaccattaccgcccggaccaccacctgtcccactgcagatgggttcatcatcatcacaagttggtggccctcaaccatttgtctctggacttttgagtaaccttatgcggcatggtgtaatttcactggaaccacctagccaatcccaggtatttctcatgcttcatgcgttgttatatagtattattctgtttatatttatgctgttaattaaggggaaaattgtgaacattttattgtaggattctgttggagttgacttcaatgtagacctcaaggtacggaatgagcctgtcatcaatgctttgtatcaggatctttcaaggcagtgtaaaacttgtggccttcgatttaaatgccaagaggagcatcgtgctcacatggactggcatgttacaaaaaataggaattcaaagaaccgtaagcaatcttcacgtaaatattttgtcacggtggaggaatggttaagagctgcagaaacagtaggaaacgatggagttcctgcttttgtgccctcagacccagtcccagatagaaaggaagagagagaaattgctgttcctgcggatgaggagcaaacagcctgtgcattatgtcaagagccatttgaagatttctacagtgatgagactgatgagtggatgtatagaggtgcggtctacatgaatgcaccagacggtaatattgatggtctggaaaggtcacggttgggacctattgtccatgctaaatgtcgatctgggcccagcaatacttagggtgggcatggctggtgagtatctatctatttccttgtgctataacaaacatctatggactttaaggtggacataatgttttttagagctagtttgggaacctcattttcccaagggaaattgtatcttgaggttaaccccactgtaaatagaggatagtctccttgctagtatttggctgtatgcaacatgtttttggctacgattatatatgttatctgactaaataaaaatgctggatgtgagttgttgttcattatttttccattgtttgtagttagtgttcaatattatatgaatatatacttctgtcatacatttggacccattcttttttgtttggttttcagttctgctgaaccattttgcagatccatcatatggattttcagaatgtctgattctactggcatactcccacggtccaagaaagaatgcaattctagcagtgtaatgttttagtatcttaagtttgatcaattatagataaaaaatatcaatatttatgataccaaattaatatcactagattaattatgaaatgtattttcgtaataaattgattttgagccataaatgtgaacatcattgactagaaacttgttcaaacatgaaccactttaactggcacacaacccatagctgcattctttttggaacagagtgagtatataataagaggactttcaattgctcacttgaatagttagttacctttgaaggattctgtaggttttaaaatgtaaatgtgaacgctcatgcttgttcaagtaatgcaatttttaattggtgtaactttggtaatgacaaagataatcatgctaactatgtaattttatgctaatgacaactgtaagcatgcaaacccacagttttgtatgtatcatttagtgcgatctgatgatgatttatgaaagctgtaatttcctagcatgactacacttttaatctctcaccttttggtatctaaggactaaggatagaagtaggctttggattaatttgtaaaaaagtggcgataattagttcaggactactatagttcctttttatttgtacttcactatttttgcccatcatggcattgctaatctgcgcaactattttggtatctaggatagatttccttaagcaggttttggtctaatttgtaaaaatgtggcaatagtaaggagttcaggactatcatagttacttttcatttgcaattcaccattttgctcatcatggcactgctaaactgcgcagttgttatgtctgccaatcaaagattcatgcatttagtggaatgacactaatactgtttctgtcatttcctgacagtgatacactggtgcacaggaccttttattttctttattgtttttgtgagttttttaagttttttgtgctagctcttagcatgtttggaagcattgtttgtttttaattatctggtcagtgttcatgcaattagtaagtagcataggcacataagttctggttacctcttgctgttgacaattgacatcacgattcctgttaggatagaattttttctgttcaaatatcaaacagatctgacatgaaatttcttgaagtatttgactgagcaatcttatgtttgttatgttttctttgtcatataattttttccctttggattatgaaatcctcagtgaccggttataatttggaagaccagttataagctggaaggtggtaaaattgagatatactcggcttatttgccgttgcacggtaccatgtaatttagttacacgctgttatatcagccactacctctgttccttagagaggtgcatagccagaatgacagttttttaggaacgaaggtaatagttgcaacaacgccttctttggctccaaaaggatcctttttggtacgcgccaaaatatgaagctaagtgtggttgaaaagcctgctgagttttcctgaatggtgtatgatcctatgagatgggttcattggttgactccttttgccatggtgatgattattttgaatgcataatatttgttttcatatcagttgctgtgatggcttcaattttattcattttccgtccctaattaattgttaataaagccaaaatgaagttaactaagcatattgtatattagcttcgtgtaaacaaatcacttgaactgaaacttgctatgatctaatgttagtaaattccttataagtgcttcattgaaagatgctgctataactatttttatcttcggtgcagggatgacttgatagcttttttttccgttgcggctgtaggcatgacctgtaaactactggagactcagaatttggtatggttagattttttcctatctatggcttcgttaacagccaatgcacagaatgttaatgtgacaatagtgcataactgtaaaatttttctgtaattcagtgattggaagttagtaatcggcacaacattcactattgtcatattgtgcttttgcccctgtcattttgtccttccatagttgcacagtgctgctttttcattacagccgtagtgtgatctatacttaagcttcatgtttagggtttctgcagcacagttgttgtcaggacagcggtttatggagccatggtctcactggacatgggaaacaatatatttcaatagtcctgctttcttcctcgttaatatcttgtgcaggatacataccagttctctatgttggttggtagctttggaagggaccgtgcaagatgaccaagagtgctggccactatttatgcctaagtattgaaagggactagatactgtcattaccttattgccaatttttttagtattttcctttgtaagttgagagcattgtttgtgactattattcttggttgcttgtatagttccatcttgctgatatgtttatactccaaaatgtgggtcagaatgtcttatcagactttattctttattctggttggtatatgctccggcaaactattcaatggaacaatttgggaatattttttcgtcca

SEQ ID NO: 106 - DNA, 544 bp, synthetic construct
  source 1..544: mol_type = other DNA
atatatggtctcgattgacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattgcagaggattatgaccagatgttttagagctagaaatagcaagttaaaataaggctagtccgttatcaacttgaaaaagtggcaccgagtcggtgcttttttttggcaaaaattttcagattttttcttcatctgtagatttctgggtttttttttccgtttcgtgaatcataagtgaagttttggatgcaaatctgcgcgaaaaaagttggacctgcaatgagcttatttagatagctaagacaaagtgattggtccgtagattgagacctatata

SEQ ID NO: 107 - DNA, 545 bp, synthetic construct
  source 1..545: mol_type = other DNA
atatatggtctcgagatgacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattgcagcaaccgaagaaatcgagttttagagctagaaatagcaagttaaaataaggctagtccgttatcaacttgaaaaagtggcaccgagtcggtgcttttttttggcaaaaattttcagattttttcttcatctgtagatttctgggtttttttttccgtttcgtgaatcataagtgaagttttggatgcaaatctgcgcgaaaaaagttggacctgcaatgagcttatttagatagctaagacaaagtgattggtccgttgattgagacctatata

SEQ ID NO: 108 - DNA, 545 bp, synthetic construct
  source 1..545: mol_type = other DNA
atatatggtctcgtgatgacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattgccgcgcgttggaattaacagttttagagctagaaatagcaagttaaaataaggctagtccgttatcaacttgaaaaagtggcaccgagtcggtgcttttttttggcaaaaattttcagattttttcttcatctgtagatttctgggtttttttttccgtttcgtgaatcataagtgaagttttggatgcaaatctgcgcgaaaaaagttggacctgcaatgagcttatttagatagctaagacaaagtgattggtccgttagatgagacctatata

SEQ ID NO: 109 - DNA, 545 bp, synthetic construct
  source 1..545: mol_type = other DNA
atatatggtctcgtagagacggtcattgttttagttccaccacgattatatttgaaatttacgtgagtgtgagtgagacttgcataagaaaataaaatctttagttgggaaaaaattcaataatataaatgggcttgagaaggaagcgagggataggcctttttctaaaataggcccatttaagctattaacaatcttcaaaagtaccacagcgcttaggtaaagaaagcagctgagtttatatatggttagagacgaagtagtgattgagcctagtcaaccaacgaggttttagagctagaaatagcaagttaaaataaggctagtccgttatcaacttgaaaaagtggcaccgagtcggtgcttttttttggcaaaaattttcagattttttcttcatctgtagatttctgggtttttttttccgtttcgtgaatcataagtgaagttttggatgcaaatctgcgcgaaaaaagttggacctgcaatgagcttatttagatagctaagacaaagtgattggtccgtgttttgagacctatata

SEQ ID NO: 110 - DNA, 23 bp, synthetic construct
  source 1..23: mol_type = other DNA
gcaaaaacgtttcagatcacaag

SEQ ID NO: 111 - DNA, 23 bp, synthetic construct
  source 1..23: mol_type = other DNA
cctccaaaagatgctacttctac

SEQ ID NO: 112 - DNA, 22 bp, synthetic construct
  source 1..22: mol_type = other DNA
ctcgtagaccgtttgatagatc

SEQ ID NO: 113 - DNA, 21 bp, synthetic construct
  source 1..21: mol_type = other DNA
tcgaggatgttgttgcaaatg

SEQ ID NO: 114 - DNA, 25 bp, synthetic construct
  source 1..25: mol_type = other DNA
ttctgccttgcatataggcaagcac

SEQ ID NO: 115 - DNA, 20 bp, synthetic construct
  source 1..20: mol_type = other DNA
ccatagcttcaaggttgcag

SEQ ID NO: 116 - DNA, 22 bp, synthetic construct
  source 1..22: mol_type = other DNA
caaccgacataatcacataaac

SEQ ID NO: 117 - DNA, 20 bp, synthetic construct
  source 1..20: mol_type = other DNA
acagactctataactccgga

SEQ ID NO: 118 - DNA, 20 bp, synthetic construct
  source 1..20: mol_type = other DNA
ggacctgatcattaagctcc

SEQ ID NO: 119 - DNA, 20 bp, synthetic construct
  source 1..20: mol_type = other DNA
cacttcttcttcttcgcctg

(19) United States
(12) Patent Application Publication
(10) Pub. No.: US 2025/0146009 A1
(43) Pub. Date: May 8, 2025

(54) PLANT GRAIN SIZE REGULATION GENE AND USE THEREOF

(71) Applicant: CAPITAL NORMAL UNIVERSITY, Beijing (CN)

(72) Inventors: Ligeng MA, Beijing (CN); Ying CAO, Beijing (CN); Jing WU, Beijing (CN)

(73) Assignee: CAPITAL NORMAL UNIVERSITY, Beijing (CN)

(21) Appl. No.: 19/011,969

(22) Filed: Jan. 7, 2025

Related U.S. Application Data
(63) Continuation of application No. PCT/CN2023/110679, filed on Aug. 2, 2023.

(30) Foreign Application Priority Data
Aug. 19, 2022 (CN) 202210999291.4

(51) Int. Cl. C12N 15/82; C07K 14/415; C12N 9/22; C12N 15/11

(52) U.S. Cl. CPC C12N 15/8261; C07K 14/415; C12N 9/22; C12N 15/11; C12N 15/8201; C12N 2310/20

(57) ABSTRACT

Disclosed are a plant grain size regulation gene and use thereof, which belong to the technical field of biology. PCFS gene mutants in different crops are obtained by gene editing, and by means of experiments and statistical analysis, it is found that the mutants have a phenotype of enlarged plant grain and part of the mutants have a phenotype of improved stress resistance. The provided gene, mutants, and method for use same are beneficial to increase the crop yield and improve the quality and enhance the resistance of plants to an adverse environment, provide gene resources and technical support for cultivating new plant varieties with large grain weight and high stress resistance, and thereby have important significance and application value for improvement of agronomic traits of crops and breeding of high-yield and high stress-resistant molecules.

### CROSS-REFERENCE TO RELATED APPLICATIONS

The present application claims the priority of a China patent application with a priority number of 202210999291.4 and an invention name of “PLANT GRAIN SIZE REGULATION GENE AND USE THEREOF”, filed in China National Intellectual Property Administration on Aug. 19, 2022, the entire contents of which are incorporated into the present application by reference.

### TECHNICAL FIELD

The present application belongs to the field of plant biotechnology breeding, in particular to a PCFS family gene, and a method for obtaining plants with improved agronomic traits by mutating the gene.

### BACKGROUND

The current situation of sustained population growth and decreasing cultivated land area poses a severe challenge to the global food security. How to find high-quality gene resources by means of biotechnology and apply them to crop breeding, so as to obtain crop varieties with a high yield, high quality and favorable for mechanized farming is an important topic for all mankind.

The grain size of a plant is a trait of great concern in agricultural production, which directly affects the yield of crops. Increasing the grain size of staple crops such as rice, wheat and maize can enhance the grain yield, and increasing the grain size of oil crops such as Brassica napus and soybean can enhance the oil yield, which are very important for promoting agricultural production. Furthermore, crops are often affected by harsh environments, such as droughts, salt stresses, diseases and pests and the like, during their growths. Therefore, selecting varieties with a strong resistance to droughts, salts, and diseases and pests can effectively enhance the resistance and adaptability of a plant, so as to achieve the goal of a high yield. For problems that Brassica napus have many branches and the pods on the branches mature late, as well as the machine often cannot harvest the grains on the side branches, how to obtain a reasonable plant type is also a topic of great concern in crop breeding. From the above, it can be seen that whether the grains become larger, the branches become less, or the resistance to osmotic stresses increases, they are all favorable traits in agriculture, and are very important for enhancing crop yields and developing modern agriculture.

The homologous gene of PCFS (Pcf11-SIMILAR PROTEIN) was first reported in a yeast. The name of the gene in the yeast is Pcf11 (protein 1 of CF I). By using the protein sequence of Pcf11 in the yeast for gene alignment, related homologous proteins can be found in plants. In higher plants, the PCFS family generally has two or more homologous genes. Previously, the functions of the PCFS family genes in plants were rarely concerned. When exploring the functions of the PCFS family genes, the inventors of the present application edited the PCFS family genes of a plant such as Arabidopsis thaliana, Brassica napus, maize, soybean and rice by CRISPR/Cas9 technology to obtain mutants, and unexpectedly found that the mutation of the PCFS family genes with an intact N-terminus can regulate the grain size of a plant, and make the plant have bigger and heavier grains. Meanwhile, mutations of some of the PCFS genes also have the functions of enhancing the tolerance of a plant to adversity stresses and reducing the number of branches. The gene, mutant and application method provided in the embodiments of the present application are helpful to enhance crop yields and improve qualities, enhance plant resistances to adversity, provide gene resources and technical supports for cultivating new plant varieties with a big grain weight and a strong stress resistance, and have important significance and application value for improving crop agronomic traits and breeding of molecules with a high yield and a strong stress resistance.

### SUMMARY OF THE INVENTION

All references described herein are incorporated herein by reference. Unless indicated to the contrary, all technical and scientific terms used herein have the same meaning as commonly understood by those of ordinary skill in the art to which the present application belongs. Unless otherwise indicated, the techniques used or described herein are standard techniques known to those of ordinary skill in the art. The materials, methods and examples are for illustration only, not for limitation.

The embodiments of the present application provide a gene and an isolated nucleotide sequence of a mutant thereof, wherein a plant containing the gene mutation has bigger grains, and the gene has a nucleotide sequence selected from one of the sequences in the following group:
(a) a nucleotide sequence with a gene ID of AT2G36480 or AT4G04885 in Arabidopsis thaliana;
(b) a nucleotide sequence with a gene ID of BnaA04G0223600WE, BnaA05G0075100WE, BnaA09G0222400WE, BnaC04G0102200WE, BnaC04G0535800WE or BnaC09G0254600WE in Brassica napus;
(c) a nucleotide sequence with a gene ID of Glyma.03G191200, Glyma.10G066300, Glyma. 10G251100, Glyma.19G191800, Glyma.19G191900 or Glyma.20G142500 in soybean;
(d) a nucleotide sequence with a gene ID of LOC_Os08g08830 or LOC_Os09g39270 in rice;
(e) a nucleotide sequence with a gene ID of Zm00001d000023, Zm00001d005350, Zm00001d019856 or Zm00001d049442 in maize;
(f) a nucleotide sequence capable of hybridizing with any one of the nucleotide sequences of (a) to (e) under a stringent condition;
(g) a nucleotide sequence having at least 85%, 90%, 95% or more sequence similarity with a full length of any one of the nucleotide sequences of (a) to (f), and having a function of making a plant have bigger grains after mutation in the plant; or
(h) a nucleotide sequence complementary to any one of the nucleotide sequences of (a) to (g).

The embodiments of the present application provide a gene and an isolated nucleotide sequence of a mutant thereof, the gene has a phenotype of making a plant have bigger and/or heavier grains after mutation, and the gene has a nucleotide sequence selected from one of the sequences in the following group:
(a) a nucleotide sequence as shown in SEQ ID NO: 4, 5, 7, 8, 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74;
(b) a nucleotide sequence encoding an amino acid sequence as shown in SEQ ID NO: 6, 9, 15, 18, 24, 27, 30, 36, 39, 42, 45, 51, 54, 57, 60, 63, 66, 69, 72 or 75;
(c) a nucleotide sequence capable of hybridizing with the sequence in (a) or (b) under a stringent condition;
(d) a nucleotide sequence having at least 85%, 90%, 95% or more sequence similarity with a full length of any one of the nucleotide sequences of (a) to (c), and having a function of making a plant have bigger and/or heavier grains after mutation; or
(e) a nucleotide sequence complementary to the sequence of any one of (a) to (d).

The above genes provided in the embodiments of the present application can be isolated from various plants. Those skilled in the art should know that the gene described in the present application also includes homologous genes which are highly homologous to the nucleotide sequence or protein sequence of the gene and have the same function of having bigger and/or heavier grains after mutation. The homologous gene includes a DNA sequence which is capable of hybridizing with the nucleotide sequence of the gene disclosed in the embodiments of the present application under a stringent condition. As used herein, the “stringent condition” is commonly known, including, such as, hybridizing in a hybridization solution containing 400 mM NaCl, 40 mM PIPES (pH6.4) and 1 mM EDTA, with a hybridization temperature of preferably 53° C.-60° C. and a hybridization time of preferably 12-16 hours, and then washing with a washing solution containing 0.5×SSC and 0.1% SDS, with a washing temperature of preferably 62° C.-68° C. and a washing time of 15-60 minutes.

The homologous gene also includes a DNA sequence having at least 80%, 85%, 90%, 95%, 98%, 99% or higher sequence similarity with the DNA sequence shown in the gene disclosed in the embodiments of the present application, or having at least 80%, 85%, 90%, 95%, 98%, 99% or higher sequence similarity with the amino acid sequence of the functional gene disclosed in the embodiments of the present application, and having a function of making a plant have bigger and/or heavier grains after mutation, which can be isolated from any plant. More specifically, the protein amino acid sequence encoded by the DNA sequence of the PCFS family gene has an intact N-terminus, and the amino acid sequence of the N-terminus is Y . . . L_{X}ELT_{X}N_{X}KP_{X}IT_{X}LTI_{X}A . . . E . . . Q_{X}LP_{X}LYLLDSIVKN_{X}G_{XX}Y . . . F . . . L_{XX}VF_{XX}AY . . . M_{XX}LF_{X}T W_{XX}VF . . . L_{XX}I . . . L . . . IH (_{X} represents any amino acid, . . . represents more than three any random amino acid sequences, and the remaining letters represent specific amino acids). Wherein, the percentage of sequence similarity can be obtained by commonly known bioinformatics algorithms, including the Myers and Miller algorithm, the Needleman-Wunsch global alignment method, the Smith-Waterman local alignment method, the Pearson and Lipman similarity search method, the Karlin and Altschul algorithm, which are commonly known to those skilled in the art.

Those skilled in the art should know that there is single nucleotide polymorphism (SNP) in the same gene among different varieties of the same plant, i.e., the nucleotide sequence of the same gene often has individual base differences, but there are so many varieties in the same crop that it is impossible for the inventors to list them one by one. The present application only provides the sequences of representative varieties in different crops. Therefore, those skilled in the art should know that the nucleotide sequences from different varieties that have SNP with the gene protected by the present application and the nucleotide sequence thereof are also within the protection scope of the present application.

The mutation described in the embodiments of the present application refers to the substitution, deletion and/or addition of one or more nucleotides on the nucleotide sequence of the gene of the present application. The gene has a function of improving agronomic traits after mutation, including making a plant have heavier and/or bigger grains, wherein after single gene mutation of pcfs4 (the gene ID is AT4G04885, and the nucleotide sequence is as shown in SEQ ID NO:7 or 8) or double mutation of pcfs2 and pcfs4, the gene also has a regulation function such as increasing the stress tolerance and/or reducing the number of branches. The mutation described in the embodiments of the present application can be point mutation, and can also be DNA deletion or insertion mutation. The mutation can be obtained by physical mutagenesis, chemical mutagenesis or gene editing. The methods of chemical mutagenesis include mutagenesis by treating with mutagens such as EMS; the methods of gene editing include but are not limited to gene editing methods such as ZFN, TALEN and/or CRISPR/Cas9.

More specifically, when the nucleotide sequence of the gene mutant in the embodiments of the present application is obtained by the CRISPR/Cas9 gene editing method, the target sequence used in the CRISPR/Cas9 gene editing method is selected from one of the sequences in the following group:
(a) a fragment having a sequence conforming to a sequence arrangement rule of 5′-Nx-NGG-3′ in a nucleotide sequence as shown in SEQ ID NO: 4, 5, 7, 8, 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74, wherein N represents any one of A, G, C and T, 14<X<30, and X is an integer, and Nx represents X consecutive nucleotides; or
(b) a polynucleotide complementary to the polynucleotide of (a).

More specifically, when using the CRISPR/Cas9 technology for gene editing, the target sequence for an Arabidopsis thaliana plant is as shown in any one of SEQ ID NOs: 76-79, the target sequence for a Brassica napus plant is as shown in any one of SEQ ID NOs: 80-83, the target sequence for a soybean plant is as shown in any one of SEQ ID NOs: 84-87, the target sequence for a rice plant is as shown in any one of SEQ ID NOs: 88-91, and the target sequence for a maize plant is as shown in any one of SEQ ID NOs: 92-95.

The nucleotide sequence of the gene mutant according to the embodiments of the present application, which is as shown in any one of SEQ ID NOs: 97, 98 and 100-105.

The embodiments of the present application also disclose a method for regulating agronomic traits of a plant, comprising mutating the following genes of the plant to make the plant have bigger and heavier grains, wherein the genes have a nucleotide sequence selected from one of the sequences in the following group:
(a) a nucleotide sequence with a gene ID of AT2G36480 or AT4G04885 in Arabidopsis thaliana;
(b) a nucleotide sequence with a gene ID of BnaA04G0223600WE, BnaA05G0075100WE, BnaA09G0222400WE, BnaC04G0102200WE, BnaC04G0535800WE or BnaC09G0254600WE in Brassica napus;
(c) a nucleotide sequence with a gene ID of Glyma.03G191200, Glyma.10G066300, Glyma.10G251100, Glyma.19G191800, Glyma. 19G191900 or Glyma.20G142500 in soybean;
(d) a nucleotide sequence with a gene ID of LOC_Os08g08830 or LOC_Os09g39270 in rice; or a nucleotide sequence with a gene ID of Zm00001d000023, Zm00001d005350, Zm00001d019856 or Zm00001d049442 in maize;
(e) a DNA sequence capable of hybridizing with any one of the nucleotide sequences of (a) to (e) under a stringent condition; or
(f) a nucleotide sequence having at least 85%, 90%, 95% or more sequence similarity with a full length of any one of the nucleotide sequences of (a) to (f), and having a function of making a plant have bigger grains after mutation in the plant; or
(g) a DNA sequence complementary to any one of the nucleotide sequences of (a) to (f).

Specifically, the method for regulating agronomic traits of a plant disclosed in the embodiments of the present application comprises mutating the following genes of the plant to make the plant have bigger and/or heavier grains, wherein the genes have a nucleotide sequence selected from one of the sequences in the following group:
(a) a nucleotide sequence as shown in SEQ ID NO: 4, 5, 7, 8, 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74;
(b) a nucleotide sequence encoding an amino acid sequence as shown in SEQ ID NO: 6, 9, 15, 18, 24, 27, 30, 36, 39, 42, 45, 51, 54, 57, 60, 63, 66, 69, 72 or 75;
(c) a nucleotide sequence capable of hybridizing with the sequence in (a) or (b) under a stringent condition;
(d) a nucleotide sequence having at least 85%, 90%, 95% or more sequence similarity with a full length of any one of the nucleotide sequences of (a) to (c), and having a function of making a plant have bigger grains after mutation in the plant; or
(e) a DNA sequence complementary to any one of the sequences of (a) to (d).

The method for regulating agronomic traits of a plant provided in the embodiments of the present application, wherein the genes can be isolated from various plants. Those skilled in the art should know that in the method for regulating agronomic traits of a plant described in the present application, the gene used also includes homologous genes which are highly homologous to the nucleotide sequence or protein sequence of the gene and have the same function of having bigger and heavier grains after mutation. The homologous gene includes a DNA sequence which is capable of hybridizing with the nucleotide sequence of the gene disclosed in the embodiments of the present application under a stringent condition. As used herein, the “stringent condition” is commonly known, including, such as, hybridizing in a hybridization solution containing 400 mM NaCl, 40 mM PIPES (pH6.4) and 1 mM EDTA, with a hybridization temperature of preferably 53° C.-60° C. and a hybridization time of preferably 12-16 hours, and then washing with a washing solution containing 0.5×SSC and 0.1% SDS, with a washing temperature of preferably 62° C.-68° C. and a washing time of 15-60 minutes.

The homologous gene also includes a DNA sequence having at least 80%, 85%, 90%, 95%, 98% or 99% sequence similarity with the DNA sequence shown in the PCFS family gene disclosed in the embodiments of the present application, and having the same function of making a plant have bigger and/or heavier grains after mutation, which can be isolated from any plant. More specifically, the protein amino acid sequence encoded by the DNA sequence of the PCFS family gene has an intact N-terminus, and the amino acid sequence of the N-terminus is Y . . . L_{X}ELT_{X}N_{X}KP_{X}IT_{X}LTI_{X}A . . . E . . . Q_{X}LP_{X}LYLLDSIVKN_{X}G_{XX}Y . . . F . . . L_{XX}VF_{XX}AY . . . M_{XX}LF_{X}T W_{XX}VF . . . L_{XX}I . . . L . . . IH (_{X} represents any amino acid, . . . represents more than three any random amino acid sequences, and the remaining letters represent specific amino acids). Wherein, the percentage of sequence similarity can be obtained by commonly known bioinformatics algorithms, including the Myers and Miller algorithm, the Needleman-Wunsch global alignment method, the Smith-Waterman local alignment method, the Pearson and Lipman similarity search method, the Karlin and Altschul algorithm, which are commonly known to those skilled in the art.

In the method for regulating agronomic traits of a plant described in the embodiments of the present application, the mutation refers to the substitution, deletion and/or addition of one or more nucleotides on the nucleotide sequence of the gene disclosed in the present application. The gene has a function of improving agronomic traits after mutation, including making a plant have heavier and/or bigger grains, wherein after single gene mutation of pcfs4 (the gene ID is AT4G04885, and the nucleotide sequence is as shown in SEQ ID NO: 7 or 8) or double gene mutation of pcfs2 and pcfs4, the gene also has a regulation function such as increasing the stress tolerance and/or reducing the number of branches. The mutation described in the embodiments of the present application can be point mutation, and can also be DNA deletion or insertion mutation. The mutation can be obtained by physical mutagenesis, chemical mutagenesis or gene editing. The methods of chemical mutagenesis include mutagenesis by treating with mutagens such as EMS; the methods of gene editing include but are not limited to gene editing methods such as ZEN, TALEN and/or CRISPR/Cas9.

More specifically, when the CRISPR/Cas9 technology is used for plant agronomic traits regulation, the target sequence used is selected from any one of the sequences in the following group:
(a) a fragment having a sequence conforming to a sequence arrangement rule of 5′-Nx-NGG-3′ in a nucleotide sequence as shown in SEQ ID NO: 4, 5, 7, 8, 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74, wherein N represents any one of A, G, C and T, 14<X<30, and X is an integer, and Nx represents X consecutive nucleotides; or
(b) a polynucleotide complementary to the polynucleotide of (a).

More specifically, when using the CRISPR/Cas9 technology for gene editing, the target sequence for an Arabidopsis thaliana plant is as shown in any one of SEQ ID NOs: 76-79, the target sequence for a Brassica napus plant is as shown in any one of SEQ ID NOs: 80-83, the target sequence for a soybean plant is as shown in any one of SEQ ID NOs: 84-87, the target sequence for a rice plant is as shown in any one of SEQ ID NOs: 88-91, and the target sequence for a maize plant is as shown in any one of SEQ ID NOs: 92-95.

The nucleotide sequence of the gene mutant according to the embodiments of the present application, which is as shown in any one of SEQ ID NOs: 97, 98 and 100-105.

The embodiments of the present application also disclose a plant cell, tissue, organ or product which is not used as a propagation material, the plant cell, tissue, organ or product contains the nucleotide sequence of the gene mutant of any one of the foregoing in the present application.

The embodiments of the present application also disclose use of the method of any one of the foregoing and a mutant material obtained thereby in breeding. Specifically, the use in breeding means that the gene mutant is obtained by means of gene mutation or hybridization with the gene mutant materials of the present application to make a plant have a phenotype of bigger and/or heavier grains.

Compared with the related art, the present application has the following beneficial effects:
1) The present application provides a gene for regulating the grain size of a plant, which belongs to the PCFS family gene, and the protein amino acid sequence encoded by the gene has a N-terminus with a conserved domain, and the sequence of the domain is Y . . . L_{X}ELT_{X}N_{X}KP_{X}IT_{X}LTI_{X}A . . . E . . . Q_{X}LP_{X}LYLLDSIVKN_{X}G_{XX}Y . . . F . . . L_{XX}VF_{XX}AY . . . M_{XX}LF_{X}TW_{XX}VF . . . L_{XX}I . . . L . . . IH (_{X} represents any amino acid, . . . represents more than three any random amino acid sequences, and the remaining letters represent specific amino acids).
2) The PCFS gene provided by the present application can make a plant have bigger grains after mutation, thus increasing the yield of the plant and providing new gene resources for high-yield breeding of crops.
3) The PCFS family gene provided by the present application can be used as a gene to control the grain size of crops and improve the yield and quality, and can be applied to the improvement of crop varieties, which is helpful for breeding new crop varieties with high quality traits. Meanwhile, the gene of the present application can also be used in molecular marker technology, serving for practical production and application such as big grain and high yield breeding of crops.
4) The PCFS gene provided by the present application has homologous genes in many plants such as Arabidopsis thaliana, Brassica napus, soybean, maize and rice, and can be used not only for the breeding of the above plants, but also for the cultivation of new varieties of other plants.

### BRIEF DESCRIPTION OF THE DRAWINGS

In order to explain the technical solutions in the embodiments of the present application more clearly, the drawings needed in the description of the embodiments will be briefly introduced below. Obviously, the drawings in the following description are only some embodiments of the present application. For those of ordinary skill in the art, other drawings can be obtained according to these drawings without creative work, in which:

FIG. 1 is a diagram of a gene editing vector. Wherein U6promoter stands for a U6 promoter, U6terminal stands for a terminator, and target stands for a gene editing target.

FIG. 2 is the PCR detection results of gene editing mutants of Arabidopsis thaliana.

FIGS. 3A and 3B are the branch phenotypes and statistical results of different mutants of PCFS family genes in Arabidopsis thaliana. FIG. 3A is a picture of the number of branches of a wild type and mutants after 50 days of growth, and FIG. 3B is a quantitative statistical diagram of the number of branches, wherein the abscissa is the wild type and various mutants, and the ordinate is the ratio of the number of branches of different plants.

FIGS. 4A and 4B are the statistical results of the grain phenotype and thousand kernel weight of different mutants of PCFS family genes in Arabidopsis thaliana. FIG. 4A is a picture of the grains of a wild type and mutants after maturation which is taken by a dissecting microscope, and FIG. 4B is a quantitative statistical diagram of the thousand kernel weight, wherein the abscissa is the wild type and various mutants, and the ordinate is the thousand kernel weight of grains. The significant difference analysis shows that the differences between the mutants and the wild type are extremely significant.

FIGS. 5A and 5B are the phenotypes and statistical results of different mutants of PCFS family genes in Arabidopsis thaliana treated with 300 mM mannitol. FIG. 5A is a phenotype diagram after 6 days of growth in the normal medium and then 8 days of growth in 300 mM mannitol medium, and FIG. 5B is a quantitative statistical diagram of cotyledon yellowing, wherein the abscissa is the wild type and various mutants, and the ordinate is the ratio of the cotyledon color of different plants.

FIGS. 6A, 6B, 6C, and 6D are the grain phenotypes of different crops after PCFS family gene mutation. FIG. 6A is a diagram comparing the grain size of a Brassica napus wild type and a pcfs mutant thereof; FIG. 6B is a diagram comparing the grain size of a wild-type soybean and a pcfs mutant thereof, FIG. 6C is a diagram comparing the grain size of a rice wild type and a pcfs mutant thereof after husking; FIG. 6D is a diagram comparing the grain size of a wild-type maize and a pcfs mutant thereof. WT stands for wild type and mutant stands for mutant.

FIGS. 7A, 7B, 7C, and 7D are the statistics of the thousand kernel weight of the grains of different crops after PCFS family gene mutation. FIG. 7A is the statistical results of the thousand kernel weight of the grains of a Brassica napus wild type and a pcfs mutant thereof;

FIG. 7B is the statistical results of the hundred kernel weight of the grains of a soybean wild type and a pcfs mutant thereof; FIG. 7C is the statistical results of the thousand kernel weight of the grains of a rice wild type and a pcfs mutant thereof; FIG. 7D is the statistical results of the hundred kernel weight of the grains of a maize wild type and a pcfs mutant thereof.

### DETAILED DESCRIPTION OF THE EMBODIMENTS

The following examples are used to illustrate the present application, but are not used to limit the scope of the present application. Without departing from the spirit and essence of the present application, modifications or substitutions to the methods, steps or conditions of the present application are all within the scope of the present application. Unless otherwise specified, the materials and biochemical reagents used in the examples are all conventional commercially available reagents, and the technical means used in the examples are familiar to those skilled in the art.

### Example 1. Construction of PCFS Mutant Plants of Arabidopsis thaliana

In order to study the function of PCFS family genes in plants, the homologous proteins in Arabidopsis thaliana were aligned on the Phytozome website by using the protein sequence of yeast Pcf11 (the website was entered, and then Tools was clicked, BLAST was clicked, the protein sequence of yeast Pcf11 was input, the species Arabidopsis thaliana was selected and go was clicked). There are four homologous proteins in Arabidopsis thaliana, namely PCFS1, PCFS2, PCFS4 and PCFS5, respectively. The corresponding gene sequences, CDS sequences and protein sequences were downloaded on the TAIR website. Wherein the gene IDs corresponding to PCFS1, PCFS2, PCFS4 and PCFS5 are AT1G66500, AT2G36480, AT4G04885 and AT5G43620, respectively, their genomic DNA sequences are as shown in SEQ ID NOs: 1, 4, 7 and 10, respectively, their CDS sequences (coding region sequences) are as shown in SEQ ID NOs: 2, 5, 8 and 11, respectively, and their protein amino acid sequences are as shown in SEQ ID NOs: 3, 6, 9 and 12, respectively.

The inventors intended to obtain Arabidopsis thaliana mutants of the above genes by CRISPR/Cas9 genes editing. Firstly, the efficient targets of gene editing were predicted by using the CRISPR-P V2.0 website (the website was entered, and then PAM was defaulted, U6 was selected as the snoRNA promoter, 19 was selected as the guide sequence length, Arabidopsis thaliana, gene IDs or gene sequences were input, and submit was clicked), and the targets with a high score and located on an exon were selected from the given predicted targets, because such targets could be edited efficiently, so as to achieve the purpose of constructing mutant plants. In this example, four targets were selected to target four PCFS family genes, and the specific information of the four targets is shown in Table 1. These four targets were constructed together into the PHEE401E vector (the vector was purchased from addgene (https://www.addgene.org/), and for information about the vector, please referred to the article: Wang Z P, Xing H L, Dong L, et al. Egg cell-specific promoter-controlled CRISPR/Cas9 efficiently generates homozygous mutants for multiple target genes in Arabidopsis in a single generation. Genome Biol. 2015; 16 (1): 144.), and transformed into E. coli and Agrobacterium tumefaciens. The vector was transformed into wild-type Arabidopsis thaliana with Col-0 background by the floral dip method using Agrobacterium tumefaciens.

Specifically, the method for constructing the target into a gene editing vector was as follows:
1) For the four genes of the PCFS family in Arabidopsis thaliana in Table 1 below, four target were selected and four nucleotide products were synthesized artificially. As shown in Table 2 below, the four nucleotide products comprise the information of the four targets respectively, and their nucleotide sequences are as shown in SEQ ID NO: 106, SEQ ID NO: 107, SEQ ID NO: 108 and SEQ ID NO: 109, respectively, wherein the targets are underlined in Table 2.
2) There were two Bsa I enzyme digestion sites on the gene editing vector, which could be digested by the Bsa I endonuclease. In the case of using the enzymatic digestion and ligation system and the conditions in Table 3, because there were unpaired nucleotide fragments at the 5′ ends of the vector and four gene synthesis fragments, the four gene synthesis fragments could be ligated into the vector according to a specific order based on the principle of base complementary pairing by T4 ligase.

TABLE 1
Information list of PCFS family in Arabidopsis
thaliana

| PCFS family | |
|---|---|
| homologous | |
| genes | Target |
| AT1G66500 | CAGAGGATTATGACCAGAT (SEQ ID NO: 76) |
| AT2G36480 | CAGCAACCGAAGAAATCGA (SEQ ID NO: 77) |
| AT4G04885 | CCGCGCGTTGGAATTAACA (SEQ ID NO: 78) |
| AT5G43620 | AGCCTAGTCAACCAACGAG (SEQ ID NO: 79) |

TABLE 2
Gene synthesis fragments

Gene synthesis fragment 1 (SEQ ID NO: 106):
ATATATGGTCTCGATTGACGGTCATTGTTTTAGTTCCACCACGATTATATTTGAAATTTACGTGAGTGTGA
GTGAGACTTGCATAAGAAAATAAAATCTTTAGTTGGGAAAAAATTCAATAATATAAATGGGCTTGAGAAGGA
AGCGAGGGATAGGCCTTTTTCTAAAATAGGCCCATTTAAGCTATTAACAATCTTCAAAAGTACCACAGCGCTT
AGGTAAAGAAAGCAGCTGAGTTTATATATGGTTAGAGACGAAGTAGTGATTGCAGAGGATTATGACCAGATG
TTTTAGAGCTAGAAATAGCAAGTTAAAATAAGGCTAGTCCGTTATCAACTTGAAAAAGTGGCACCGAGTCGG
TGCTTTTTTTTGGCAAAAATTTTCAGATTTTTTCTTCATCTGTAGATTTCTGGGTTTTTTTTTCCGTTTCGTGAA
TCATAAGTGAAGTTTTGGATGCAAATCTGCGCGAAAAAAGTTGGACCTGCAATGAGCTTATTTAGATAGCTA
AGACAAAGTGATTGGTCCGTAGATTGAGACCTATATA
Gene synthesis fragment 2 (SEQ ID NO: 107):
ATATATGGTCTCGAGATGACGGTCATTGTTTTAGTTCCACCACGATTATATTTGAAATTTACGTGAGTGTG
AGTGAGACTTGCATAAGAAAATAAAATCTTTAGTTGGGAAAAAATTCAATAATATAAATGGGCTTGAGAAGG
AAGCGAGGGATAGGCCTTTTTCTAAAATAGGCCCATTTAAGCTATTAACAATCTTCAAAAGTACCACAGCGCT
TAGGTAAAGAAAGCAGCTGAGTTTATATATGGTTAGAGACGAAGTAGTGATTGCAGCAACCGAAGAAATCG
AGTTTTAGAGCTAGAAATAGCAAGTTAAAATAAGGCTAGTCCGTTATCAACTTGAAAAAGTGGCACCGAGTC
GGTGCTTTTTTTTGGCAAAAATTTTCAGATTTTTTCTTCATCTGTAGATTTCTGGGTTTTTTTTTCCGTTTCGTG
AATCATAAGTGAAGTTTTGGATGCAAATCTGCGCGAAAAAAGTTGGACCTGCAATGAGCTTATTTAGATAGC
TAAGACAAAGTGATTGGTCCGTTGATTGAGACCTATATA
Gene synthesis fragment 3 (SEQ ID NO: 108):
ATATATGGTCTCGTGATGACGGTCATTGTTTTAGTTCCACCACGATTATATTTGAAATTTACGTGAGTGTG
AGTGAGACTTGCATAAGAAAATAAAATCTTTAGTTGGGAAAAAATTCAATAATATAAATGGGCTTGAGAAGG
AAGCGAGGGATAGGCCTTTTTCTAAAATAGGCCCATTTAAGCTATTAACAATCTTCAAAAGTACCACAGCGCT
TAGGTAAAGAAAGCAGCTGAGTTTATATATGGTTAGAGACGAAGTAGTGATTGCCGCGCGTTGGAATTAACA
GTTTTAGAGCTAGAAATAGCAAGTTAAAATAAGGCTAGTCCGTTATCAACTTGAAAAAGTGGCACCGAGTCG
GTGCTTTTTTTTGGCAAAAATTTTCAGATTTTTTCTTCATCTGTAGATTTCTGGGTTTTTTTTTCCGTTTCGTGA
ATCATAAGTGAAGTTTTGGATGCAAATCTGCGCGAAAAAAGTTGGACCTGCAATGAGCTTATTTAGATAGCT
AAGACAAAGTGATTGGTCCGTTAGATGAGACCTATATA
Gene synthesis fragment 4 (SEQ ID NO: 109):
ATATATGGTCTCGTAGAGACGGTCATTGTTTTAGTTCCACCACGATTATATTTGAAATTTACGTGAGTGTG
AGTGAGACTTGCATAAGAAAATAAAATCTTTAGTTGGGAAAAAATTCAATAATATAAATGGGCTTGAGAAGG
AAGCGAGGGATAGGCCTTTTTCTAAAATAGGCCCATTTAAGCTATTAACAATCTTCAAAAGTACCACAGCGCT
TAGGTAAAGAAAGCAGCTGAGTTTATATATGGTTAGAGACGAAGTAGTGATTGAGCCTAGTCAACCAACGAG
GTTTTAGAGCTAGAAATAGCAAGTTAAAATAAGGCTAGTCCGTTATCAACTTGAAAAAGTGGCACCGAGTCG
GTGCTTTTTTTTGGCAAAAATTTTCAGATTTTTTCTTCATCTGTAGATTTCTGGGTTTTTTTTTCCGTTTCGTGA
ATCATAAGTGAAGTTTTGGATGCAAATCTGCGCGAAAAAAGTTGGACCTGCAATGAGCTTATTTAGATAGCT
AAGACAAAGTGATTGGTCCGTGTTTTGAGACCTATATA

TABLE 3
Enzymatic digestion and ligation system and conditions

| | | | |
|---|---|---|---|
| Gene synthesis fragments | 1 | microliter each | 37° C. 5 h |
| Gene editing vector | 2 | microliter | 50° C. 5 min |
| T4 Buffer | 1.5 | microliter | 80° C. 10 min |
| BSA | 1.5 | microliter | |
| BsaI | 1 | microliter | |
| T4 Ligase | 1 | microliter | |

| | |
|---|---|
| ddH2O | Make up to 15 microliters |

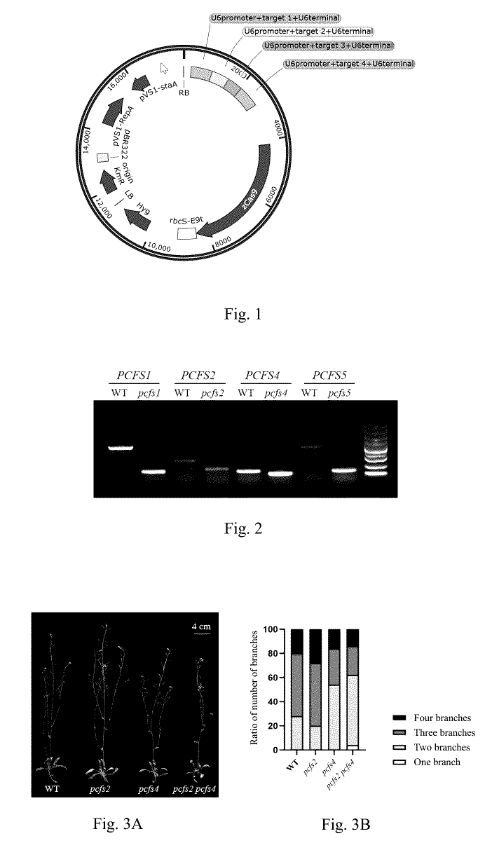

The gene editing vector had a hygromycin resistance gene in plants. The grains of transgenic plants, i.e., the transgenic T₁ generation, were collected and spread on the plant medium with hygromycin, and the plants that could grow normally were successful transgenic plants. DNA was extracted from a small piece of leaf derived from the successful transgenic plants, and the PCFS family genes were specifically amplified by PCR using the primers in Table 4 below. When the PCR amplification products were electrophoresed, the plants with bands different from the wild type on the agarose gel were the mutant plants expected in the present application, and the specific electrophoresis results were as shown in FIG. 2.

TABLE 4
Primers for identification of Arabidopsis thaliana mutants

| | |
|---|---|
| PCFS1 | PCFS1-JD-F: GCAAAAACGTTTCAGATCACAAG (SEQ ID NO: 110) |
| | PCFS1-JD-R: CCTCCAAAAGATGCTACTTCTAC (SEQ ID NO: 111) |
| PCFS2 | PCFS2-JD-F: CTCGTAGACCGTTTGATAGATC (SEQ ID NO: 112) |
| | PCFS2-JD-R: CCTCCAAAAGATGCTACTTCTAC (SEQ ID NO: 113) |
| PCFS4 | PCFS4-JD-F: TTCTGCCTTGCATATAGGCAAGCAC (SEQ ID NO: 114) |
| | PCFS4-JD-R: CCATAGCTTCAAGGTTGCAG (SEQ ID NO: 115) |
| PCFS5 | PCFS5-JD-F3: CAACCGACATAATCACATAAAC (SEQ ID NO: 116) |
| | PCFS5-JD-R3: ACAGACTCTATAACTCCGGA (SEQ ID NO: 117) |

The obtained mutant plants of the transgenic T₁ generation were hybridized with the wild type. Because the transgenic sites were heterozygous, some of the hybrid plants of the F₁ generation contained transgenic insertions and some did not. The Cas genes on the transgenic vector were specifically amplified by PCR using the primers in Table 5, and whether the vector was screened out was judged according to the presence or absence of bands. The plants without amplification bands were plants whose vectors had been screened out. At this time, the PCFS gene of the plants was in a heterozygous state, and the pcfs-related mutant whose vector had been screened out could be obtained by self-crossing for another generation.

TABLE 5
Primers used to identify whether vectors are screened out of plants

| | |
|---|---|
| Free-cas9-F: GGACCTGATCATTAAGCTCC | The primers were designed on the |
| (SEQ ID NO: 118) | sequence of Cas9, which could be |
| Free-cas9-R: CACTTCTTCTTCTTCGCCTG | used as a favorable evidence for |
| (SEQ ID NO: 119) | whether there was a gene editing |
| | vector in plants. |

Through the above experiments, the related mutants of the PCFS family were obtained, which were named pcfs1, pcfs2, pcfs4 and pcfs5, respectively. pcfs1 is the DNA sequence of PCFS1 with the nucleotide sequences from positions 178 to 886 deleted. The nucleotide sequence of the gene after mutation is as shown in SEQ ID NO: 96. The mutation causes the amino acid at position 60 of the encoded protein to begin to change, and meet the stop codon at position 64. pcfs2 is the DNA sequence of PCFS2 with the nucleotide sequences from positions 104 to 293 deleted and 6 bp nucleotides inserted at the same time. The nucleotide sequence of the gene after mutation is as shown in SEQ ID NO: 97. The mutation causes the amino acid at position 35 of the encoded protein to begin to change, and meet the stop codon at position 46. pcfs4 is the DNA sequence of PCFS4 with the nucleotide sequences from positions 867 to 918 deleted. The nucleotide sequence of the gene after mutation is as shown in SEQ ID NO: 98. The mutation causes the amino acid at position 206 of the encoded protein to begin to change, and meet the stop codon at position 210. pcfs5 is the DNA sequence of PCFS5 with the nucleotide sequences from positions 178 to 886 deleted. The nucleotide sequence of the gene after mutation is as shown in SEQ ID NO: 99. The mutation causes the amino acid at position 60 of the encoded protein to begin to change, and meet the stop codon at position 64. The above results show that the mutation affects the normal expression of the gene protein, making the expressed protein polypeptide shorter and inhibiting the normal function of the protein.

### Example 2. Observation of Branch Phenotype and Grain Size Phenotype of Arabidopsis thaliana

The wild-type and mutant grains received in the same batch were disinfected with a 15% sodium hypochlorite solution for 10 minutes respectively, washed twice with sterilized double distilled water, and then placed on a ½ MS plant medium, put in a light incubator, and grown for 2 weeks under the condition of long sunshine (16 hours under the light at 21° C./8 hours in the dark at 19° C.). Then, the germinated seedlings were transferred to the soil and put into a greenhouse with the culture conditions unchanged, and watered once every two days.

Three weeks after bolting of Arabidopsis thaliana, the plant type was completely determined, and the number of branches was counted. As can be seen from FIGS. 3A and 3B that the number of branches of the pcfs2 mutant has no obvious change compared with the wild type, while the number of branches of the pcfs4 mutant is reduced compared with the wild type, and the phenotype of the double mutant is similar to that of pcfs4. This experiment shows that the pcfs4 gene mutation also has a function of reducing the number of branches in a plant.

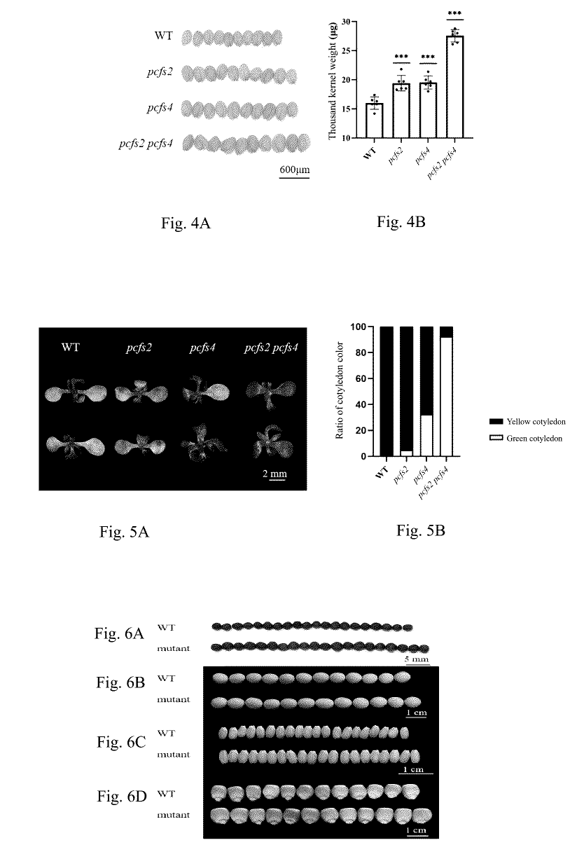

After the grains of Arabidopsis thaliana were completely yellow, the grains were harvested, dried by a drying ball for two days, and observed for the grain size under a microscope, and the thousand kernel weight was weighed by the one-thousandth balance. As can be seen from FIG. 4A that the grains of the pcfs2, pcfs4 and pcfs2pcfs4 double mutation mutants are all bigger than those of the wild type. As can be seen from FIG. 4B that the thousand kernel weights of pcfs2 and pcfs4 are also increased compared with the wild type, with the increase amounts of 21% and 22%, respectively, and the double mutant has an enhanced phenotype of bigger grains, with the thousand kernel weight increased by 70%.

In contrast, neither the pcfs1 mutant nor the pcfs5 mutant has no phenotype of bigger and heavier grains or fewer branch number.

### Example 3. Observation of Stress Resistance Phenotype of Arabidopsis thaliana

The wild-type and mutant grains received in the same batch were disinfected with a 15% sodium hypochlorite solution for 10 minutes, washed twice with sterilized double distilled water, and then placed on a vertical ½ MS plant medium, put in a light incubator, and grown for 6 days under the condition of full sunshine (24 hours under the light at 21° C.), and then transferred to a horizontal ½ MS plant medium containing 300 mM mannitol (which simulated osmotic stress, with the intensity of about-1.0 MPa) for 8 days, and the ratio of cotyledon yellowing was counted. As can be seen from FIGS. 5A and 5B that there is no obvious difference in the resistance of pcfs2 relative to the wild type, but the survival rate of pcfs4 is obviously enhanced, while the survival rate of pcfs2pcfs4 double mutant is greatly enhanced. This experiment shows that the mutation of pcfs4 gene also has a function of stress tolerance, and the plants with simultaneous mutation of pcfs2 and pcfs4 have stronger stress tolerance because of their function redundancy.

### Example 4. Alignment of Homologous Sequences of PCFS Family in Different Crops

From the alignment results of the amino acid sequences of PCFS family members in different plants, it can be seen that the C-terminal zinc finger domains of the PCFS family members in plants such as Arabidopsis thaliana, Brassica napus, soybean, maize and rice is conserved, with an L . . . C_{XX}C . . . E . . . H . . . E_{X}Q_{XX}C_{X}LC_{X}E . . . W . . . I_{X}H_{XX}C (_{X} represents any amino acid, . . . represents more than three any random amino acid sequences, and the remaining letters represent specific amino acids) conserved domain (the above sequences are 100% matched in the given plant species). The zinc finger domains of the above-mentioned species can be predicted by using the SMART website (http://smart.embl-heidelberg.de/) and the InterProScan website (http://www.ebi.ac.uk/interpro/search/sequence/). Some PCFS family members have N-terminus deletion, but the N-terminus of the members with an intact N-terminus is also conserved. The amino acid sequence of the N-terminus is Y . . . L_{X}ELT_{X}N_{X}KP_{X}IT_{X}LTI_{X}A . . . E . . . Q_{X}LP_{X}LYLLDSIVKN_{X}G_{XX}Y . . . F . . . L_{XX}VF_{XX}AY . . . M_{XX}LF_{X}T W_{XX}VF . . . L_{XX}I . . . L . . . IH (the above sequences are 100% matched in the given plant species with an intact N-terminus).

Through the above experimental results and the structural analysis of the gene sequences in Arabidopsis thaliana, it is found that PCFS2 and PCFS4 have a N-terminus conserved sequence, and show a phenotype of bigger grains after mutation; PCFS1 and PCFS5 have N-terminus deletion, and there is no significant difference between them after mutation and the wild type. Therefore, in the gene editing of crops such as Brassica napus, soybean, maize, and rice, the inventors preferred to knock out the PCFS family members with an intact N-terminus.

### Example 5. Construction of PCFS Mutant Plants of Brassica napus

Arabidopsis thaliana and Brassica napus are both Cruciferae plants. The homologous proteins in Brassica napus were aligned on the BNTIR (http://yanglab.hzau.edu.cn/BnTIR/download) website by using the protein sequence of the PCFS family in Arabidopsis thaliana (the website was entered, and then Tools was clicked, next, BLAST was clicked, the gene name of the PCFS family in Arabidopsis thaliana was input according to the prompts to get the homologous sequence in Brassica napus). Brassica napus is a heterodiploid, and has 8 homologous proteins of the PCFS family, 6 of which have an intact N-terminus, namely, BnaA04G0223600WE, BnaA05G0075100WE, BnaA09G0222400WE, BnaC04G0102200WE, BnaC04G0535800WE and BnaC09G0254600WE, respectively. Their genomic DNA sequences are as shown in SEQ ID NOs: 13, 16, 22, 25, 28 and 34, respectively, their CDS sequences are as shown in SEQ ID NOs: 14, 17, 23, 26, 29 and 35, respectively, and their protein amino acid sequences are as shown in SEQ ID NOs: 15, 18, 24, 27, 30 and 36, respectively.

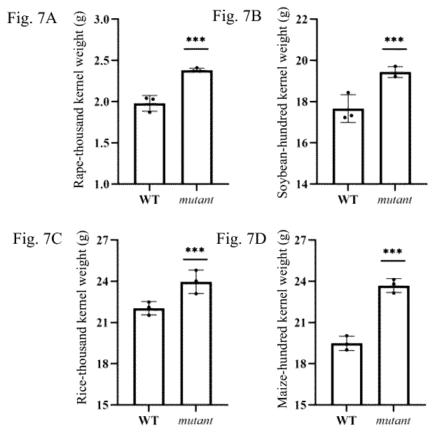

The inventors used highly homologous sequences in genes to design four targets to target these six genes. The target sequences and their corresponding sequence NOs are as shown in Table 6 below. After being transgenic screened, the double mutation mutants of BnaA05G0075100WE and BnaA09G0222400WE were obtained. The nucleotide sites from positions 108 to 109 on the DNA sequence of the BnaA09G0222400WE gene of the mutant are deleted. The nucleotide sequence of the gene after mutation is as shown in SEQ ID NO: 100. The mutation causes the protein sequence to begin to change at the amino acid at position 37, and meet the stop codon at the amino acid at position 78. Meanwhile, a nucleotide is inserted at position 110 of the DNA sequence of the BnaC09G0254600WE gene. The nucleotide sequence of the gene after mutation is as shown in SEQ ID NO: 101. The mutation causes the protein sequence to begin to change at the amino acid at position 37, and meet the stop codon at the amino acid at position 42. It shows that the mutation affects the normal expression of the gene protein, making the expressed protein polypeptide shorter and inhibiting the normal function of the protein. By observing the grain size of the wild-type and the mutants, it is found that the mutants have a phenotype of bigger grains (as shown in FIG. 6A), with a thousand kernel weight increased by 20% compared with the wild-type (as shown in FIG. 7A).

TABLE 6
Information list of PCFS family in Brassica napus

| PCFS family | Gene with intact | Target sequence | |
|---|---|---|---|
| homologous genes | N-terminus | (sequence NO) | Resulting mutant |
| BnaA04G0223600WE | BnaA04G0223600WE | TCGATACTCGACCGGTTCA | BnaA09G0222400WE |
| BnaA05G0075100WE | BnaA05G0075100WE | (SEQ ID NO: 80) | BnaC09G0254600WE |
| BnaA07G0282000WE | BnaA09G0222400WE | TTGATAGATCCAGAGATCC | Double mutant |
| BnaA09G0222400WE | BnaC04G0102200WE | (SEQ ID NO: 81) | with mutations in |
| BnaC04G0102200WE | BnaC04G0535800WE | TTGGTTCATCGCTGAGCCT | both genes |
| BnaC04G0535800WE | BnaC09G0254600WE | (SEQ ID NO: 82) | |
| BnaC06G0351800WE | | GATCTCTCGATCTATCGAA | |
| BnaC09G0254600WE | | (SEQ ID NO: 83) | |

### Example 6. Construction of PCFS Mutant Plants of Soybean, Maize and Rice Respectively

The gene IDs of PCFS family homologous genes in soybean, maize and rice are shown in Table 7 below, respectively. Wherein, the IDs of the genes with an intact N-terminus in soybean are Glyma.03G191200, Glyma.10G066300, Glyma. 10G251100, Glyma. 19G191800, Glyma. 19G191900 and Glyma.20G142500, respectively, and their gene sequences, CDS sequences and protein sequences are obtained from the website SoyBase (https://www.soybase.org/). Their genomic DNA sequences are as shown in SEQ ID NO: 37, 40, 43, 49, 52 and 55, respectively, their CDS sequences are as shown in SEQ ID NOs: 38, 41, 44, 50, 53 and 56, respectively, and their protein amino acid sequences are as shown in SEQ ID NOs: 39, 42, 45, 51, 54 and 57, respectively. The gene editing target sites designed in the present application for the PCFS family homologous genes in soybean are as shown in SEQ ID NOs: 84-87, respectively (Table 7).

The PCFS family homologous genes in rice are LOC_Os08g08830 and LOC_Os09g39270, respectively, and their gene sequences, CDS sequences and protein sequences are obtained from the website Phytozome (https://phytozome-next.jgi.doe.gov/). Their genomic DNA sequences are as shown in SEQ ID NOs: 58 and 61, respectively, their CDS sequences are as shown in SEQ ID NOs: 59 and 62, respectively, and their protein amino acid sequences are as shown in SEQ ID NOs: 60 and 63, respectively. The gene editing target sites designed in the present application for the PCFS family homologous genes in rice are as shown in SEQ ID NOs: 88-91, respectively (Table 7).

The PCFS family homologous genes in maize are Zm00001d000023, Zm00001d005350, Zm00001d019856, Zm00001d049442, respectively, and their gene sequences, CDS sequences and protein sequences are obtained from the website Phytozome (https://phytozome-next.jgi.doe.gov/). Their genomic DNA sequences are as shown in SEQ ID NOs: 64, 67, 70 and 73, respectively, their CDS sequences are as shown in SEQ ID NOs: 65, 68, 71 and 74, respectively, and their protein amino acid sequences are as shown in SEQ ID NOs: 66, 69, 72 and 75, respectively. The gene editing target sites designed in the present application for the PCFS family homologous genes in maize are as shown in SEQ ID NOs: 92-95, respectively (Table 7).

TABLE 7
Information list of PCFS family in crops

| PCFS family | Gene with intact | Target sequence | |
|---|---|---|---|
| homologous genes | N-terminus | (sequence NO) | Resulting mutant |
| Glyma.03G191200 | Glyma.03G191200 | TAAATCTGGACGATTTCGT (SEQ ID NO: 84) | Glyma.10G251100 |
| Glyma.10G066300 | Glyma.10G066300 | CCAACTTGACTATTATTGC (SEQ ID NO: 85) | is mutated |
| Glyma.10G251100 | Glyma.10G251100 | GTTCTCGCGATCTATCGAA (SEQ ID NO: 86) | |
| Glyma.10G251200 | Glyma.19G191800 | CAAGCATCATGAGGTTGAG (SEQ ID NO: 87) | |
| Glyma.19G191800 | Glyma.19G191900 | | |
| Glyma.19G191900 | Glyma.20G142500 | | |
| Glyma.20G142500 | | | |
| LOC_Os08g08830 | LOC_Os08g08830 | CCGAGCTCACCATCATCGCC (SEQ ID NO: 88) | LOC_Os08g08830 |
| LOC_Os09g39270 | LOC_Os09g39270 | CCCCTTGGAACCCTGAGAGC (SEQ ID NO: 89) | is mutated |
| | | CTGGGCGAGGCGAGGCTTCT (SEQ ID NO: 90) | |
| | | CAGCAGGCTGTTGTGGGAAC (SEQ ID NO: 91) | |
| Zm00001d000023 | Zm00001d000023 | GTATTGTGAAGAACATAGG (SEQ ID NO: 92) | Zm00001d000023 |
| Zm00001d005350 | Zm00001d005350 | GCACAACATAAGTTCAAGC (SEQ ID NO: 93) | Zm00001d049442 |
| Zm00001d019856 | Zm00001d019856 | GGGCTACAGCAGTTACCAC (SEQ ID NO: 94) | Double mutant |
| Zm00001d049442 | Zm00001d049442 | TTCTCAGCAAGATTACCAG (SEQ ID NO: 95) | with mutations |
| | | | in both genes |

After being transgenic screened, the mutant plants of the above crops were obtained. A mutant with mutation in the Glyma. 10G251100 gene is obtained in soybean, wherein the nucleotide at position 193 on the DNA sequence of the mutant is deleted, the nucleotide sequence of the gene after mutation is as shown in SEQ ID NO: 102, and the mutation causes the protein sequence to begin to change at the amino acid at position 65, and meet the stop codon at the amino acid at position 80; a mutant with mutation in the LOC_Os08g08830 gene is obtained in rice, wherein the nucleotides from positions 203 to 4604 on the DNA sequence of the mutant are deleted, the nucleotide sequence of the gene after mutation is as shown in SEQ ID NO: 103, and the mutation causes the amino acids from positions 71 to 449 on the protein sequence to be deleted; A mutant with mutations in both Zm00001d000023 and Zm00001d049442 is obtained in maize, wherein the nucleotide sites from positions 1318-1609 on the DNA sequence of the Zm00001d000023 gene of the mutant are deleted, the nucleotide sequence of the gene after mutation is as shown in SEQ ID NO: 104, and the mutation causes the protein sequence to begin to change at the amino acid at position 103, and meet the stop codon at the amino acid at position 126, and meanwhile, the nucleotide sites from positions 1789-2081 on the DNA sequence of the Zm00001d049442 gene of the mutant are deleted, the nucleotide sequence of the gene after mutation is as shown in SEQ ID NO: 105, and the mutation causes the protein sequence to begin to change at the amino acid at position 103, and meet the stop codon at the amino acid at position 116. The above experimental results show that the expression of the gene protein of the present application is affected by mutation, including shortening of the expressed protein polypeptide, thus inhibiting the normal function of the gene protein of the present application.

The grain phenotypes of different crops and the statistical results of the thousand kernel weight or hundred kernel weight thereof are shown in FIGS. 6A-6D and FIGS. 7A-7D. As can be seen from the figures, the PCFS family genes with an intact N-terminus have a function of regulating the grain size in crops such as Brassica napus, rice, soybean, maize, etc. In Brassica napus and maize, the PCFS family genes with an intact N-terminus have bigger grains after mutation, with the thousand/hundred kernel weight thereof increased by about 20%; in rice and soybean, the PCFS family genes with an intact N-terminus also have bigger grains after mutation, with the thousand/hundred kernel weight thereof increased by about 10%. The present example further proves that the PCFS family genes with an intact N-terminus have a function of regulating grain size of a plant. After the normal function of the gene protein is inhibited by technical means such as mutation, the plant can have bigger grains, thus obtaining crop varieties with a high yield. Those skilled in the art should know that inhibiting the normal function of the gene protein described in the present application includes affecting the normal function of the gene protein through mutation, and also includes affecting the regulatory factors of the gene protein, thereby inhibiting the normal function of the PCFS family genes with an intact N-terminus in the present application and obtaining the phenotype of making a plant have bigger grains. Although the embodiments of the present application have been described in detail by general description, specific examples and experiments, it is obvious to those skilled in the art that some modifications or improvements can be made on the basis of the present application. Therefore, these modifications or improvements made without deviating from the spirit of the present application all belong to the scope claimed by the present application.

1. An isolated polynucleotide of a gene mutant, wherein a plant containing the gene mutation has bigger grains, and wherein said gene has an isolated polynucleotide selected from one of the sequences in the following group:
(a) a nucleotide sequence with a gene ID of BnaA04G0223600WE, BnaA05G0075100WE, BnaA09G0222400WE, BnaC04G0102200WE, BnaC04G0535800WE or BnaC09G0254600WE in Brassica napus;
(b) a nucleotide sequence with a gene ID of Glyma.03G191200, Glyma.10G066300, Glyma.10G251100, Glyma.19G191800, Glyma.19G191900 or Glyma.20G142500 in soybean;
(c) a nucleotide sequence with a gene ID of LOC_Os08g08830 or LOC_Os09g39270 in rice; or
(d) a nucleotide sequence with a gene ID of Zm00001d000023, Zm00001d005350, Zm00001d019856 or Zm00001d049442 in maize; or
(e) an isolated polynucleotide capable of hybridizing with any one of the isolated polynucleotide of (a) to (d) under a stringent condition;
(f) a nucleotide sequence having at least 85%, 90%, 95% or more sequence similarity with a full length of any one of the isolated polynucleotide of (a) to (e), and having a function of making a plant have bigger grains after mutation in the plant; or
(g) a nucleotide sequence complementary to any one of the nucleotide sequences of (a) to (f).

2. The isolated polynucleotide of the gene mutant of claim 1, wherein said gene has an isolated polynucleotide selected from one of the sequences in the following group:
(a) a nucleotide sequence as shown in SEQ ID NO: 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73, 74;
(b) a nucleotide sequence encoding an amino acid sequence as shown in SEQ ID NO: 15, 18, 24, 27, 30, 36, 39, 42, 45, 51, 54, 57, 60, 63, 66, 69, 72, 75;
(c) a nucleotide sequence capable of hybridizing with the nucleotide sequence in (a) or (b) under a stringent condition;
(d) a nucleotide sequence having at least 85%, 90%, 95% or more sequence similarity with a full length of any one of the nucleotide sequences of (a) to (c), and having a function of making a plant have bigger grains after mutation; or
(e) a DNA sequence complementary to any one of the sequences of (a) to (d).

3. The isolated polynucleotide of the gene mutant of claim 1, wherein said gene mutation comprises substitution, deletion and/or addition of one or more nucleotides on the nucleotide sequence of said gene.

4. The isolated polynucleotide of the gene mutant of claim 1, wherein said mutation is obtained by technologies such as physical mutagenesis, chemical mutagenesis, ZFN, TALEN and/or CRISPR/Cas9.

5. The isolated polynucleotide of the gene mutant of claim 4, wherein a target sequence used in the CRISPR/Cas9 technology is selected from one of the sequences in the following group:
(a) a fragment having a sequence conforming to a sequence arrangement rule of 5′-Nx-NGG-3′ in a nucleotide sequence as shown in SEQ ID NO:13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74, wherein N represents any one of A, G, C and T, 14<X<30, and X is an integer, and Nx represents X consecutive nucleotides; or
(b) a polynucleotide complementary to the polynucleotide of (a).

6. The isolated polynucleotide of the gene mutant of claim 5, wherein the target sequence used in the CRISPR/Cas9 technology is a sequence as shown in any one of SEQ ID NOs: 80-95.

7. The isolated polynucleotide of the gene mutant of claim 1, which is as shown in any one of SEQ ID NOs: 100-105.

8. A method for regulating agronomic traits of a plant, comprising mutating a PCFS family gene of the plant to make the plant have bigger grains, wherein said gene has a polynucleotide selected from one of the sequences in the following group:
(a) a nucleotide sequence with a gene ID of BnaA04G0223600WE, BnaA05G0075100WE, BnaA09G0222400WE, BnaC04G0102200WE, BnaC04G0535800WE or BnaC09G0254600WE in Brassica napus;
(b) a nucleotide sequence with a gene ID of Glyma.03G191200, Glyma.10G066300, Glyma.10G251100, Glyma.19G191800, Glyma.19G191900 or Glyma.20G142500 in soybean;
(c) a nucleotide sequence with a gene ID of LOC_Os08g08830 or LOC_Os09g39270 in rice; or
(d) a nucleotide sequence with a gene position ID of Zm00001d000023, Zm00001d005350, Zm00001d019856 or Zm00001d049442 in maize;
(e) a nucleotide sequence capable of hybridizing with any one of the nucleotide sequences of (a) to (d) under a stringent condition;
(f) a nucleotide sequence having at least 85%, 90%, 95% or more sequence similarity with a full length of any one of the nucleotide sequences of (a) to (e), and having a function of making a plant have bigger grains after mutation in the plant; or
(g) a DNA sequence complementary to any one of the nucleotide sequences of (a) to (f).

9. The method of claim 8, wherein said gene has a nucleotide sequence selected from one of the sequences in the following group:
(a) a nucleotide sequence as shown in SEQ ID NO: 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73, 74;
(b) a nucleotide sequence encoding an amino acid sequence as shown in SEQ ID NO: 15, 18, 24, 27, 30, 36, 39, 42, 45, 51, 54, 57, 60, 63, 66, 69, 72, 75;
(c) a nucleotide sequence capable of hybridizing with the sequence in (a) or (b) under a stringent condition; or
(d) a nucleotide sequence having at least 85%, 90%, 95% or more sequence similarity with a full length of any one of the nucleotide sequences of (a) to (c), and having a function of making a plant have bigger grains after mutation;
(e) a DNA sequence complementary to any one of the sequences of (a) to (d).

10. The method of claim 8, wherein said gene mutation comprises substitution, deletion and/or addition of one or more nucleotides on the nucleotide sequence of the gene.

11. The method of claim 8, wherein said mutation is obtained by technologies such as physical mutagenesis, chemical mutagenesis, ZFN, TALEN and/or CRISPR/Cas9.

12. The method of claim 11, wherein a target sequence used in the CRISPR/Cas9 technology is selected from one of the sequences in the following group:
(a) a fragment having a sequence conforming to a sequence arrangement rule of 5′-Nx-NGG-3′ in a nucleotide sequence as shown in SEQ ID NO: 13, 14, 16, 17, 22, 23, 25, 26, 28, 29, 34, 35, 37, 38, 40, 42, 43, 44, 49, 50, 52, 53, 55, 56, 58, 59, 61, 62, 64, 65, 67, 68, 70, 71, 73 or 74, wherein N represents any one of A, G, C and T, 14<X<30, and X is an integer, and Nx represents X consecutive nucleotides;
(b) a polynucleotide complementary to the polynucleotide of (a).

13. The method of claim 12, wherein said target sequence used in the CRISPR/Cas9 technology is a sequence as shown in any one of SEQ ID NOs: 80-95.

14. The method of claim 8, wherein the nucleotide sequence of said gene after mutation is as shown in any one of SEQ ID NOs: 100-105.

15. (canceled)

16. A method of plant breeding comprising allowing a plant to obtain a gene mutant obtained by the method of claim 8 causing the plant to have a phenotype of bigger grains by means of gene mutation or hybridization with the mutant material.
